SEQUENCE LISTING (1043 sequences)

SEQ ID NO: 1 - protein, 242 aa, synthetic construct
  REGION 1..242: note = Y13-259
QVQLQQSGGGLVQPGRSLKLSCVVSGFTFSNYGMNWIRQTPGKGLEWVAYISSGSSYLYYAETVKGRFTISRDNAKNTLYLQMTSLRSEDTALYYCARHEGTGTDFFDYWGQGTTVTVSSGGGGSGGGGSGGGGSDIQLTQSPHSLSASLGETVSIECLASEGISNYLAWYQQKPGKSPQLLIYYASSLQDGVPSRFSGSGSGTQFSLKISNMQPEDEGVYYCQQAYKYPSTFGAGTKLEIK

SEQ ID NO: 2 - protein, 32 aa, synthetic construct
  REGION 1..32: note = Tag-1-1
AHSSSAESESTSDSDSSSDSESESSSSDSEGS

SEQ ID NO: 3 - protein, 32 aa, synthetic construct
  REGION 1..32: note = Tag-1-2
AHSLSAELESTIDSDCSSDWESELSSSDSEGS

SEQ ID NO: 4 - protein, 32 aa, synthetic construct
  REGION 1..32: note = Tag-1-3
AQSSSAESESGSDSDSSSDSESESSSSDSEGS

SEQ ID NO: 5 - protein, 50 aa, synthetic construct
  REGION 1..50: note = Tag-4-1
NEGYREAFDEDYEQQDEDFAEQDPDGNEAFEGEYDGPNQDEYPDEAQNFE

SEQ ID NO: 6 - protein, 52 aa, synthetic construct
  REGION 1..52: note = Tag-2-1
DEAGSSGAPADEAGSSGAPADEAGSSGAPADEAGSSGAPADEAGSSGAPAGS

SEQ ID NO: 7 - protein, 52 aa, synthetic construct
  REGION 1..52: note = Tag-2-2
DEVGISLAPTDEVGISLAPTDEVGISLAPTDEVGISLAPTDEVGISLAPTGS

SEQ ID NO: 8 - protein, 52 aa, synthetic construct
  REGION 1..52: note = Tag-2-3
DEVMISLWPTDEVMISLWPTDEVMISLWPTDEVMISLWPTDEVMISLWPTGS

SEQ ID NO: 9 - protein, 52 aa, synthetic construct
  REGION 1..52: note = Tag-3-1
DFAGSSGAPADFAGSSGAPADFAGSSGAPADFAGSSGAPADFAGSSGAPAGS

SEQ ID NO: 10 - protein, 52 aa, synthetic construct
  REGION 1..52: note = Tag-3-2
DTAVSSIAPLDTAVSSIAPLDTAVSSIAPLDTAVSSIAPLDTAVSSIAPLGS

SEQ ID NO: 11 - protein, 52 aa, synthetic construct
  REGION 1..52: note = Tag-3-3
DTWVSLIAILDTWVSLIAILDTWVSLIAILDTWVSLIAILDTWVSLIAILGS

SEQ ID NO: 12 - protein, 10 aa, synthetic construct
  REGION 1..10: note = Myc tag
EQKLISEEDL

SEQ ID NO: 13 - protein, 9 aa, synthetic construct
  REGION 1..9: note = HA tag
YPYDVPDYA

SEQ ID NO: 14 - protein, 15 aa, synthetic construct
  REGION 1..15: note = S-tag
KETAAAKFERQHMDS

SEQ ID NO: 15 - protein, 20 aa, synthetic construct
  REGION 1..20: note = Membrane-localization signal
KLNPPDESGPGCMSCKCVLS

SEQ ID NO: 16 - protein, 20 aa, synthetic construct
  REGION 1..20: note = extracellular scFv Tag
EFGGAPEFPKPSTPPGSSGL

SEQ ID NO: 17 - protein, 49 aa, synthetic construct
  REGION 1..49: note = hMst1
DYEFLKSWTVEDLQKRLLALDPMMEQEIEEIRQKYQSKRQPILDAIEAK

SEQ ID NO: 18 - protein, 49 aa, synthetic construct
  REGION 1..49: note = hMST2
DFDFLKNLSLEELQMRLKALDPMMEREIEELRQRYTAKRQPILDAMDAK

SEQ ID NO: 19 - protein, 49 aa, synthetic construct
  REGION 1..49: note = hRaf1
GEVNWDAFSMPELHNFLRILQREEEEHLRQILQKYSYSRQKIQEALHAS

SEQ ID NO: 20 - protein, 49 aa, synthetic construct
  REGION 1..49: note = hRaf5
GEVEWDAFSIPELQNFLTILEKEEQDKIQQVQKKYDKFRQKLEEALRES

SEQ ID NO: 21 - protein, 49 aa, synthetic construct
  REGION 1..49: note = hSAV1
HILKWELFQLADLDTYQGMLKLLFMKELEQIVKMYEAYRQALLTELENR

SEQ ID NO: 22 - protein, 61 aa, synthetic construct
  REGION 1..61: note = T7C
LEDPFQSGVMLGVASTVAASPEEASVTSTEETLTPAQEAARTRAANKARKEAELAAATAEQ

SEQ ID NO: 23 - protein, 44 aa, synthetic construct
  REGION 1..44: note = T7B
LEDPEEASVTSTEETLTPAQEAARTRAANKARKEAELAAATAEQ

SEQ ID NO: 24 - protein, 44 aa, synthetic construct
  REGION 1..44: note = T7B1
LEDPEEASVTSTEETLTPAQEAARTRPPNKARKEAELAAATAEQ

SEQ ID NO: 25 - protein, 44 aa, synthetic construct
  REGION 1..44: note = T7B2
LEDPEEASVTSTEETLTPAQEAARTRGGNKARKEAELAAATAEQ

SEQ ID NO: 26 - protein, 41 aa, synthetic construct
  REGION 1..41: note = T7B3
LEDPEEASVTSTEETLTPAQEAARTRAANKARKEAELTAEQ

SEQ ID NO: 27 - protein, 44 aa, synthetic construct
  REGION 1..44: note = T7B4
LEDPEEASVTSTEETLTPAQEAARTRAANKARKEAELEAETAEQ

SEQ ID NO: 28 - protein, 44 aa, synthetic construct
  REGION 1..44: note = T7B5
LEDPEEASVTSTEETLTPAQEAARTRAAAKARKEAELAAATAEQ

SEQ ID NO: 29 - protein, 41 aa, synthetic construct
  REGION 1..41: note = T7B6
LEDPEEASVTSTEETLTPAQEAARTRKARKEAELAAATAEQ

SEQ ID NO: 30 - protein, 39 aa, synthetic construct
  REGION 1..39: note = T7B7
LEDPEEASVTSTEETLTPAQEAARTRAANKARKEAELAA

SEQ ID NO: 31 - protein, 40 aa, synthetic construct
  REGION 1..40: note = T7B8
LEDPEEASVTSTEETLTPAQEAARTRAANKARKEAELAAA

SEQ ID NO: 32 - protein, 44 aa, synthetic construct
  REGION 1..44: note = T7B9
LEDPEEASVTSTEETLTPAQEAAETEAANKARKEAELEAETAEQ

SEQ ID NO: 33 - protein, 32 aa, synthetic construct
  REGION 1..32: note = T7B10
LEDPTPAQEAARTRAANKARKEAELAAATAEQ

SEQ ID NO: 34 - protein, 22 aa, synthetic construct
  REGION 1..22: note = T7A
LEDPAANKARKEAELAAATAEQ

SEQ ID NO: 35 - protein, 22 aa, synthetic construct
  REGION 1..22: note = T7A1
LEDPERNKERKEAELAAATAEQ

SEQ ID NO: 36 - protein, 22 aa, synthetic construct
  REGION 1..22: note = T7A2
LEDPERNKERKEAELEAATAEQ

SEQ ID NO: 37 - protein, 22 aa, synthetic construct
  REGION 1..22: note = T7A3
LEDPERNKERKEAELEAETAEQ

SEQ ID NO: 38 - protein, 43 aa, synthetic construct
  REGION 1..43: note = T3
LEDPAVWEAGKVVAKGVGTADITATTSNGLIASCKVIVNAATS

SEQ ID NO: 39 - protein, 43 aa, synthetic construct
  REGION 1..43: note = T3A
LEDPAVWEAGKVVAKGVGTADITATTSNGLIASSEEADNAATS

SEQ ID NO: 40 - protein, 89 aa, synthetic construct
  REGION 1..89: note = Zif628
ERPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 41 - protein, 52 aa, synthetic construct
  REGION 1..52: note = HinR
GRPRAITKHEQEQISRLLEKGHPRQQLAIIFGIGVSTLYRYFPASSIKKRMN

SEQ ID NO: 42 - protein, 114 aa, synthetic construct
  REGION 1..114: note = TrpR
  REGION 17..18: note = misc_feature - Xaa can be any naturally occurring amino acid
  REGION 35..36: note = misc_feature - Xaa can be any naturally occurring amino acid
  REGION 53..54: note = misc_feature - Xaa can be any naturally occurring amino acid
MAQQSPYSAAMAEQRHXXQEWLRFVDLLKNAYQNXXDLHLPLLNLMLTPDERXXEALGTRVRIVEELLRGEMSQRELKNELGAGIATITRGSNSLKAAPVELRQWLEEVLLKSD

SEQ ID NO: 43 - protein, 18 aa, synthetic construct
  REGION 1..18: note = NE-1
TKENPRSNQEESYDDNES

SEQ ID NO: 44 - protein, 18 aa, synthetic construct
  REGION 1..18: note = NE-8
TKENPRTNQEESYDDNES

SEQ ID NO: 45 - protein, 18 aa, synthetic construct
  REGION 1..18: note = NE-9
TKENPRSNQDESYDDNES

SEQ ID NO: 46 - protein, 18 aa, synthetic construct
  REGION 1..18: note = NE-10
TKENPRSNQPPSYDDNES

SEQ ID NO: 47 - protein, 12 aa, synthetic construct
  REGION 1..12: note = PA12-tag
GVAMPGAEDDVV

SEQ ID NO: 48 - protein, 14 aa, synthetic construct
  REGION 1..14: note = PA14-tag
EGGVAMPGAEDDVV

SEQ ID NO: 49 - protein, 123 aa, synthetic construct
  REGION 1..123: note = FATT-tag
DYKDDDDVEAEESDNVDSADAEEDDSDVWWGGADTDYADGSEDKVVEVAEEEEVAEVEEEEADDDEDDEDGDEVEEEAEEPYEEATERTTSIATTTTTTTESVEEVYPGQVGYPGQVGYPGQV

SEQ ID NO: 50 - protein, 36 aa, synthetic construct
GGSAQLEKELQALEKENAQLEWELQALEKELAQGAT

SEQ ID NO: 51 - protein, 288 aa, synthetic construct
GEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEG

SEQ ID NO: 52 - protein, 21 aa, synthetic construct
ASPAAPAPASPAAPAPSAPAA

SEQ ID NO: 53 - protein, 21 aa, synthetic construct
ASAAAPAAASAAASAPSAAAA

SEQ ID NO: 54 - protein, 25 aa, synthetic construct
AASPAAPSAPPAAASPAAPSAPPAA

SEQ ID NO: 55 - protein, 58 aa, synthetic construct [sequence omitted]

SEQ ID NO: 56 - protein, 58 aa, synthetic construct
VDNKFNKEQQNAEYEIEHLPNLNEEQENAFIQSLEDDPSQSANLLAEAKKLNDAQAPK

SEQ ID NO: 57 - protein, 58 aa, synthetic construct
VDNKFNKEEEEAEEEIEHLPNLNEEQEEAFIESLEDDPSQSANLLAEAKKLNDAQAPK

SEQ ID NO: 58 - protein, 20 aa, synthetic construct
KLNPPDESGPGCMSCKCVLS

SEQ ID NO: 59 - protein, 50 aa, synthetic construct; identical to SEQ ID NO: 5

SEQ ID NO: 60 - protein, 60 aa, synthetic construct
NEGSYREAFDSEDYEQSQDSEDFSAEQDPDGNSEAFEGSEYSDGPNSQDEYPDEAQNFSE

SEQ ID NO: 61 - protein, 70 aa, synthetic construct
NEGSYRESAFDSEDYEQSQDSSEDFSAEQDSSPDGNSSEAFEGSSEYSDGPNSQDEYPDEASSQNFSSSE

SEQ ID NO: 62 - protein, 20 aa, synthetic construct
YDNPYFEPQYGFPPEEDEDE

SEQ ID NO: 63 - protein, 30 aa, synthetic construct
SYDSNPSYFSEPSQYGSFPPSESEDSEDSE

SEQ ID NO: 64 - protein, 40 aa, synthetic construct
SSYSDSNPSYFSEPSSQSSYGSSFPPSESEDSSESDSSSE

SEQ ID NO: 65 - protein, 60 aa, synthetic construct
NEGQYREAFDQEDYEQQQDQEDFQAEQDPDGNQEAFEGQEYDGPNQQDEYQPDEAQNFQE

SEQ ID NO: 66 - protein, 70 aa, synthetic construct
NEGQYREQAFDQEDYEQQQDQQEDFQAEQDQQPDGNQQEAFEGQQEYQDGPNQQDEYPDEAQQQNFQQQE

SEQ ID NO: 67 - protein, 30 aa, synthetic construct
QYDQNPQYFQEPQQYGQFPPQEQEDQEDQE

SEQ ID NO: 68 - protein, 40 aa, synthetic construct
QQYQDQNPQYFQEPQQQQYGQQFPQPQEQEDQQEQDQQQE

SEQ ID NO: 69 - protein, 60 aa, synthetic construct
NEGNYREAFDNEDYEQNQDNEDFNAEQDPDGNNEAFEGNEYNDGPNNQDEYPDEAQNFNE

SEQ ID NO: 70 - protein, 70 aa, synthetic construct
NEGNYRENAFDNEDYEQNQDNNEDFNAEQDNNPDGNNNEAFEGNNEYNDGPNNQDEYPDEANNQNFNNNE

SEQ ID NO: 71 - protein, 30 aa, synthetic construct
NYDNNPNYFNEPNQYGNFPPNENEDNEDNE

SEQ ID NO: 72 - protein, 40 aa, synthetic construct
NNYNDNNPNYFNEPNNQNNYGNNFPPNENEDNNENDNNNE

SEQ ID NO: 73 - protein, 60 aa, synthetic construct
NEGPYREAFDPEDYEQPQDPEDFPAEQDPDGNPEAFEGPEYPDGPNPQDEYPDEAQNFPE

SEQ ID NO: 74 - protein, 70 aa, synthetic construct
NEGPYREPAFDPEDYEQPQDPPEDFPAEQDPPPDGNPPEAFEGPPEYPDGPNPQDEYPDEAPPQNFPPPE

SEQ ID NO: 75 - protein, 30 aa, synthetic construct
PYDPNPPYFPEPPQYGPFPPPEPEDPEDPE

SEQ ID NO: 76 - protein, 40 aa, synthetic construct
PPYPDPNPPYFPEPPPQPPYGPPFPPPEPEDPPEPDPPPE

SEQ ID NO: 77 - protein, 60 aa, synthetic construct
NEGFYREAFDFEDYEQFQDFEDFFAEQDPDGNFEAFEGFEYFDGPNFQDEYPDEAQNFFE

SEQ ID NO: 78 - protein, 70 aa, synthetic construct
NEGFYREFAFDFEDYEQFQDFFEDFFAEQDFFPDGNFFEAFEGFFEYFDGPNFQDEYPDEAFFQNFFFFE

SEQ ID NO: 79 - protein, 30 aa, synthetic construct
FYDFNPFYFFEPFQYGFFPPFEFEDFEDFE

SEQ ID NO: 80 - protein, 40 aa, synthetic construct
FFYFDFNPFYFFEPFFQFFYGFFFPPFEFEDFFEFDFFFE

SEQ ID NO: 81 - protein, 60 aa, synthetic construct
NEGYYREAFDYEDYEQYQDYEDFYAEQDPDGNYEAFEGYEYYDGPNYQDEYPDEAQNFYE

SEQ ID NO: 82 - protein, 70 aa, synthetic construct
NEGYYREYAFDYEDYEQYQDYYEDFYAEQDYYPDGNYYEAFEGYYEYYDGPNYQDEYPDEAYYQNFYYYE

SEQ ID NO: 83 - protein, 30 aa, synthetic construct
YYDYNPYYFYEPYQYGYFPPYEYEDYEDYE

SEQ ID NO: 84 - protein, 40 aa, synthetic construct
YYYYDYNPYYFYEPYYQYYYGYYFPPYEYEDYYEYDYYYE

SEQ ID NO: 85 - protein, 25 aa, synthetic construct
GNNQDSSDSDNEADEASDDEDNDGN

SEQ ID NO: 86 - protein, 50 aa, synthetic construct
NEGNREASDEDSEQQDEDNAEQDPDGNEANEGESDGPNQDENPDEAQNSE

SEQ ID NO: 87 - protein, 50 aa, synthetic construct
NEGNREANDEDNEQQDEDNAEQDPDGNEANEGENDGPNQDENPDEAQNNE

SEQ ID NO: 88 - protein, 50 aa, synthetic construct; identical to SEQ ID NO: 87

SEQ ID NO: 89 - protein, 50 aa, synthetic construct
NEGNREANDEDNEQQDEDNAEQDQDGNEANEGENDGQNQDENPDEAQNNE

SEQ ID NO: 90 - protein, 32 aa, synthetic construct
DNNESADDNNENPEDNNKNTDDNEENPNNNEN

SEQ ID NO: 91 - protein, 32 aa, synthetic construct
DNSESADDNSENPEDNSKNTDDSEENPSNSES

SEQ ID NO: 92 - protein, 32 aa, synthetic construct
DSSESADDSSESPEDSSKSTDDSEESPSSSES

SEQ ID NO: 93 - protein, 32 aa, synthetic construct
DNQESADDNQENPEDNQKNTDDQEENPQNQEQ

SEQ ID NO: 94 - protein, 32 aa, synthetic construct
DQQESADDQQEQPEDQQKQTDDQEEQPQQQEQ

SEQ ID NO: 95 - protein, 32 aa, synthetic construct
DNFESADDNFENPEDNFKNTDDFEENPFNFEF

SEQ ID NO: 96 - protein, 32 aa, synthetic construct
DFFESADDFFEFPEDFFKFTDDFEEFPFFFEF

SEQ ID NO: 97 - protein, 32 aa, synthetic construct
DNPESADDNPENPEDNPKNTDDPEENPPNPEP

SEQ ID NO: 98 - protein, 32 aa, synthetic construct
DPPESADDPPEPPEDPPKPTDDPEEPPPPPEP

SEQ ID NO: 99 - protein, 32 aa, synthetic construct
DNYESADDNYENPEDNYKNTDDYEENPYNYEY

SEQ ID NO: 100 - protein, 32 aa, synthetic construct
DYYESADDYYEYPEDYYKYTDDYEEYPYYYEY

SEQ ID NO: 101 - protein, 20 aa, synthetic construct
NDNNNNENQNGNNNEEDEDE

SEQ ID NO: 102 - protein, 20 aa, synthetic construct
NDNPNNEPQNGNPPEEDEDE

SEQ ID NO: 103 - protein, 50 aa, synthetic construct
ENESEDNDEEEPNQNADDGDPNANPAAEQGGDSSDDEEGQENDQSRENEQ

SEQ ID NO: 104 - protein, 50 aa, synthetic construct
QNENNGDDQDQEEGSEEQQGESDRSNEESDNEPAADDNAPEGAEEPDDNN

SEQ ID NO: 105 - protein, 20 aa, synthetic construct
TDNNESADDNNENPEDNNKN

SEQ ID NO: 106 - protein, 20 aa, synthetic construct
DNNESADDNNENPEDNNKNT

SEQ ID NO: 107 - protein, 20 aa, synthetic construct
NNESADDNNENPEDNNKNTD

SEQ ID NO: 108 - protein, 20 aa, synthetic construct
NESADDNNENPEDNNKNTDD

SEQ ID NO: 109 - protein, 20 aa, synthetic construct
ESADDNNENPEDNNKNTDDN

SEQ ID NO: 110 - protein, 20 aa, synthetic construct
SADDNNENPEDNNKNTDDNE

SEQ ID NO: 111 - protein, 20 aa, synthetic construct
DNNENPEDNNKNTDDNEENP

SEQ ID NO: 112 - protein, 20 aa, synthetic construct
NNENPEDNNKNTDDNEENPN

SEQ ID NO: 113 - protein, 20 aa, synthetic construct
NENPEDNNKNTDDNEENPNN

SEQ ID NO: 114 - protein, 20 aa, synthetic construct
ENPEDNNKNTDDNEENPNNN

SEQ ID NO: 115 - protein, 20 aa, synthetic construct
NPEDNNKNTDDNEENPNNNE

SEQ ID NO: 116 - protein, 20 aa, synthetic construct
PEDNNKNTDDNEENPNNNEN

SEQ ID NO: 117 - protein, 21 aa, synthetic construct
DNNESADDNNENPEDNNKNTD

SEQ ID NO: 118 - protein, 21 aa, synthetic construct
NNESADDNNENPEDNNKNTDD

SEQ ID NO: 119 - protein, 21 aa, synthetic construct
NESADDNNENPEDNNKNTDDN

SEQ ID NO: 120 - protein, 21 aa, synthetic construct
ESADDNNENPEDNNKNTDDNE

SEQ ID NO: 121 - protein, 21 aa, synthetic construct
SADDNNENPEDNNKNTDDNEE

SEQ ID NO: 122 - protein, 21 aa, synthetic construct
ADDNNENPEDNNKNTDDNEEN

SEQ ID NO: 123 - protein, 21 aa, synthetic construct
DDNNENPEDNNKNTDDNEENP

SEQ ID NO: 124 - protein, 21 aa, synthetic construct
DNNENPEDNNKNTDDNEENPN

SEQ ID NO: 125 - protein, 21 aa, synthetic construct
NNENPEDNNKNTDDNEENPNN

SEQ ID NO: 126 - protein, 21 aa, synthetic construct
NENPEDNNKNTDDNEENPNNN

SEQ ID NO: 127 - protein, 21 aa, synthetic construct
ENPEDNNKNTDDNEENPNNNE

SEQ ID NO: 128 - protein, 21 aa, synthetic construct
NPEDNNKNTDDNEENPNNNEN

SEQ ID NO: 129 - protein, 22 aa, synthetic construct
DNNESADDNNENPEDNNKNTDD

SEQ ID NO: 130 - protein, 22 aa, synthetic construct
NNESADDNNENPEDNNKNTDDN

SEQ ID NO: 131 - protein, 22 aa, synthetic construct
NESADDNNENPEDNNKNTDDNE

SEQ ID NO: 132 - protein, 22 aa, synthetic construct
SADDNNENPEDNNKNTDDNEEN

SEQ ID NO: 133 - protein, 22 aa, synthetic construct
ADDNNENPEDNNKNTDDNEENP

SEQ ID NO: 134 - protein, 22 aa, synthetic construct
DDNNENPEDNNKNTDDNEENPN

SEQ ID NO: 135 - protein, 22 aa, synthetic construct
DNNENPEDNNKNTDDNEENPNN

SEQ ID NO: 136 - protein, 22 aa, synthetic construct
NNENPEDNNKNTDDNEENPNNN

SEQ ID NO: 137 - protein, 22 aa, synthetic construct
NENPEDNNKNTDDNEENPNNNE

SEQ ID NO: 138 - protein, 22 aa, synthetic construct
ENPEDNNKNTDDNEENPNNNEN

SEQ ID NO: 139 - protein, 23 aa, synthetic construct
DNNESADDNNENPEDNNKNTDDN

SEQ ID NO: 140 - protein, 23 aa, synthetic construct
NNESADDNNENPEDNNKNTDDNE

SEQ ID NO: 141 - protein, 23 aa, synthetic construct
NESADDNNENPEDNNKNTDDNEE

SEQ ID NO: 142 - protein, 23 aa, synthetic construct
ESADDNNENPEDNNKNTDDNEEN

SEQ ID NO: 143 - protein, 23 aa, synthetic construct
SADDNNENPEDNNKNTDDNEENP

SEQ ID NO: 144 - protein, 23 aa, synthetic construct
ADDNNENPEDNNKNTDDNEENPN

SEQ ID NO: 145 - protein, 23 aa, synthetic construct
DDNNENPEDNNKNTDDNEENPNN

SEQ ID NO: 146 - protein, 23 aa, synthetic construct
DNNENPEDNNKNTDDNEENPNNN

SEQ ID NO: 147 - protein, 23 aa, synthetic construct
NNENPEDNNKNTDDNEENPNNNE

SEQ ID NO: 148 - protein, 23 aa, synthetic construct
NENPEDNNKNTDDNEENPNNNEN

SEQ ID NO: 149 - protein, 23 aa, synthetic construct
HGNNQDSSDSDNEADEASDDEDN

SEQ ID NO: 150 - protein, 24 aa, synthetic construct
DNNESADDNNENPEDNNKNTDDNE

SEQ ID NO: 151 - protein, 24 aa, synthetic construct
NNESADDNNENPEDNNKNTDDNEE

SEQ ID NO: 152 - protein, 24 aa, synthetic construct
NESADDNNENPEDNNKNTDDNEEN

SEQ ID NO: 153 - protein, 24 aa, synthetic construct
ESADDNNENPEDNNKNTDDNEENP

SEQ ID NO: 154 - protein, 24 aa, synthetic construct
SADDNNENPEDNNKNTDDNEENPN

SEQ ID NO: 155 - protein, 24 aa, synthetic construct
ADDNNENPEDNNKNTDDNEENPNN

SEQ ID NO: 156 - protein, 24 aa, synthetic construct
DDNNENPEDNNKNTDDNEENPNNN

SEQ ID NO: 157 - protein, 24 aa, synthetic construct
DNNENPEDNNKNTDDNEENPNNNE

SEQ ID NO: 158 - protein, 24 aa, synthetic construct
NNENPEDNNKNTDDNEENPNNNEN

SEQ ID NO: 159 - protein, 24 aa, synthetic construct
SHGNNQDSSDSDNEADEASDDEDN

SEQ ID NO: 160 - protein, 25 aa, synthetic construct
DNNESADDNNENPEDNNKNTDDNEE

SEQ ID NO: 161 - protein, 25 aa, synthetic construct
NNESADDNNENPEDNNKNTDDNEEN

SEQ ID NO: 162 - protein, 25 aa, synthetic construct
NESADDNNENPEDNNKNTDDNEENP

SEQ ID NO: 163 - protein, 25 aa, synthetic construct
ESADDNNENPEDNNKNTDDNEENPN

SEQ ID NO: 164 - protein, 25 aa, synthetic construct
SADDNNENPEDNNKNTDDNEENPNN

SEQ ID NO: 165 - protein, 25 aa, synthetic construct
ADDNNENPEDNNKNTDDNEENPNNN

SEQ ID NO: 166 - protein, 25 aa, synthetic construct
DDNNENPEDNNKNTDDNEENPNNNE

SEQ ID NO: 167 - protein, 25 aa, synthetic construct
DNNENPEDNNKNTDDNEENPNNNEN

SEQ ID NO: 168 - protein, 25 aa, synthetic construct
GSHGNNQDSSDSDNEADEASDDEDN

SEQ ID NO: 169 - protein, 25 aa, synthetic construct
SHGNNQDSSDSDNEADEASDDEDND

SEQ ID NO: 170 - protein, 25 aa, synthetic construct
HGNNQDSSDSDNEADEASDDEDNDG

SEQ ID NO: 171 - protein, 25 aa, synthetic construct
GNNQDSSDSDNEADEASDDEDNDGN

SEQ ID NO: 172 - protein, 26 aa, synthetic construct
DNNESADDNNENPEDNNKNTDDNEEN

SEQ ID NO: 173 - protein, 26 aa, synthetic construct
NNESADDNNENPEDNNKNTDDNEENP

SEQ ID NO: 174 - protein, 26 aa, synthetic construct
NESADDNNENPEDNNKNTDDNEENPN

SEQ ID NO: 175 - protein, 26 aa, synthetic construct
ESADDNNENPEDNNKNTDDNEENPNN

SEQ ID NO: 176 - protein, 26 aa, synthetic construct
SADDNNENPEDNNKNTDDNEENPNNN

SEQ ID NO: 177 - protein, 26 aa, synthetic construct
ADDNNENPEDNNKNTDDNEENPNNNE

SEQ ID NO: 178 - protein, 26 aa, synthetic construct
DDNNENPEDNNKNTDDNEENPNNNEN

SEQ ID NO: 179 - protein, 26 aa, synthetic construct
WGSHGNNQDSSDSDNEADEASDDEDN

SEQ ID NO: 180 - protein, 26 aa, synthetic construct
GSHGNNQDSSDSDNEADEASDDEDND

SEQ ID NO: 181 - protein, 26 aa, synthetic construct
SHGNNQDSSDSDNEADEASDDEDNDG

SEQ ID NO: 182 - protein, 26 aa, synthetic construct
HGNNQDSSDSDNEADEASDDEDNDGN

SEQ ID NO: 183 - protein, 27 aa, synthetic construct
DNNESADDNNENPEDNNKNTDDNEENP

SEQ ID NO: 184 - protein, 27 aa, synthetic construct
NNESADDNNENPEDNNKNTDDNEENPN

SEQ ID NO: 185 - protein, 27 aa, synthetic construct
NESADDNNENPEDNNKNTDDNEENPNN

SEQ ID NO: 186 - protein, 27 aa, synthetic construct
ESADDNNENPEDNNKNTDDNEENPNNN

SEQ ID NO: 187 - protein, 27 aa, synthetic construct
SADDNNENPEDNNKNTDDNEENPNNNE

SEQ ID NO: 188 - protein, 27 aa, synthetic construct
ADDNNENPEDNNKNTDDNEENPNNNEN

SEQ ID NO: 189 - protein, 27 aa, synthetic construct
SHGNNQDSSDSDNEADEASDDEDNDGN

SEQ ID NO: 190 - protein, 27 aa, synthetic construct
HGNNQDSSDSDNEADEASDDEDNDGNE

SEQ ID NO: 191 - protein, 28 aa, synthetic construct
DNNESADDNNENPEDNNKNTDDNEENPN

SEQ ID NO: 192 - protein, 28 aa, synthetic construct
NNESADDNNENPEDNNKNTDDNEENPNN

SEQ ID NO: 193 - protein, 28 aa, synthetic construct
NESADDNNENPEDNNKNTDDNEENPNNN

SEQ ID NO: 194 - protein, 28 aa, synthetic construct
ESADDNNENPEDNNKNTDDNEENPNNNE

SEQ ID NO: 195 - protein, 28 aa, synthetic construct
SADDNNENPEDNNKNTDDNEENPNNNEN

SEQ ID NO: 196 - protein, 28 aa, synthetic construct
SHGNNQDSSDSDNEADEASDDEDNDGNE

SEQ ID NO: 197 - protein, 29 aa, synthetic construct
DNNESADDNNENPEDNNKNTDDNEENPNN

SEQ ID NO: 198 - protein, 29 aa, synthetic construct
NNESADDNNENPEDNNKNTDDNEENPNNN

SEQ ID NO: 199 - protein, 29 aa, synthetic construct
NESADDNNENPEDNNKNTDDNEENPNNNE

SEQ ID NO: 200 - protein, 29 aa, synthetic construct
ESADDNNENPEDNNKNTDDNEENPNNNEN

SEQ ID NO: 201 - protein, 30 aa, synthetic construct
DNNESADDNNENPEDNNKNTDDNEENPNNN

SEQ ID NO: 202 - protein, 30 aa, synthetic construct
NNESADDNNENPEDNNKNTDDNEENPNNNE

SEQ ID NO: 203 - protein, 30 aa, synthetic construct
NESADDNNENPEDNNKNTDDNEENPNNNEN

SEQ ID NO: 204 - protein, 31 aa, synthetic construct
DNNESADDNNENPEDNNKNTDDNEENPNNNE

SEQ ID NO: 205 - protein, 31 aa, synthetic construct
NNESADDNNENPEDNNKNTDDNEENPNNNEN

SEQ ID NO: 206 - protein, 31 aa, synthetic construct
FWGSHGNNQDSSDSDNEADEASDDEDNDGNE

SEQ ID NO: 207 - protein, 31 aa, synthetic construct
SHGNNQDSSDSDNEADEASDDEDNDGNEGDN

SEQ ID NO: 208 - protein, 32 aa, synthetic construct; identical to SEQ ID NO: 90

SEQ ID NO: 209 - protein, 32 aa, synthetic construct
SHGNNQDSSDSDNEADEASDDEDNDGNEGDNE

SEQ ID NO: 210 - protein, 20 aa, synthetic construct
LEDNNEEPRDPQSPPDPPNE

SEQ ID NO: 211 - protein, 20 aa, synthetic construct
EDNNEEPRDPQSPPDPPNEF

SEQ ID NO: 212 - protein, 20 aa, synthetic construct
DNNEEPRDPQSPPDPPNEFG

SEQ ID NO: 213 - protein, 20 aa, synthetic construct
EGEQQLKPNNSNAPNEDQEE

SEQ ID NO: 214 - protein, 20 aa, synthetic construct
GEQQLKPNNSNAPNEDQEEE

SEQ ID NO: 215 - protein, 20 aa, synthetic construct
KPNNSNAPNEDQEEEIQQSE

SEQ ID NO: 216 - protein, 20 aa, synthetic construct
PNNSNAPNEDQEEEIQQSEQ

SEQ ID NO: 217 - protein, 20 aa, synthetic construct
NNSNAPNEDQEEEIQQSEQH

SEQ ID NO: 218 - protein, 21 aa, synthetic construct
EGEQQLKPNNSNAPNEDQEEE

SEQ ID NO: 219 - protein, 22 aa, synthetic construct
SEGEQQLKPNNSNAPNEDQEEE

SEQ ID NO: 220 - protein, 20 aa, synthetic construct
EQLNFSDDDEQGSNSPKENN

SEQ ID NO: 221 - protein, 20 aa, synthetic construct
LNFSDDDEQGSNSPKENNSE

SEQ ID NO: 222 - protein, 20 aa, synthetic construct
NFSDDDEQGSNSPKENNSED

SEQ ID NO: 223 - protein, 20 aa, synthetic construct
FSDDDEQGSNSPKENNSEDQ

SEQ ID NO: 224 - protein, 21 aa, synthetic construct
LNFSDDDEQGSNSPKENNSED

SEQ ID NO: 225 - protein, 21 aa, synthetic construct
NFSDDDEQGSNSPKENNSEDQ

SEQ ID NO: 226 - protein, 22 aa, synthetic construct
EQLNFSDDDEQGSNSPKENNSE

SEQ ID NO: 227 - protein, 22 aa, synthetic construct
QLNFSDDDEQGSNSPKENNSED

SEQ ID NO: 228 - protein, 22 aa, synthetic construct
LNFSDDDEQGSNSPKENNSEDQ

SEQ ID NO: 229 - protein, 22 aa, synthetic construct
NFSDDDEQGSNSPKENNSEDQG

SEQ ID NO: 230 - protein, 23 aa, synthetic construct
EQLNFSDDDEQGSNSPKENNSED

SEQ ID NO: 231 - protein, 23 aa, synthetic construct
QLNFSDDDEQGSNSPKENNSEDQ

SEQ ID NO: 232 - protein, 23 aa, synthetic construct
LNFSDDDEQGSNSPKENNSEDQG

SEQ ID NO: 233 - protein, 23 aa, synthetic construct
NFSDDDEQGSNSPKENNSEDQGS

SEQ ID NO: 234 - protein, 24 aa, synthetic construct
EQLNFSDDDEQGSNSPKENNSEDQ

SEQ ID NO: 235 - protein, 25 aa, synthetic construct
EQLNFSDDDEQGSNSPKENNSEDQG

SEQ ID NO: 236 - protein, 26 aa, synthetic construct
EQLNFSDDDEQGSNSPKENNSEDQGS

SEQ ID NO: 237 - protein, 20 aa, synthetic construct
EEKNENDENSLSSSSDSSED

SEQ ID NO: 238 - protein, 20 aa, synthetic construct
NENDENSLSSSSDSSEDKDE

SEQ ID NO: 239 - protein, 21 aa, synthetic construct
KETNNSNAQNPSEEEGEGQDE

SEQ ID NO: 240 - protein, 21 aa, synthetic construct
ETNNSNAQNPSEEEGEGQDED

SEQ ID NO: 241 - protein, 22 aa, synthetic construct
KETNNSNAQNPSEEEGEGQDED

SEQ ID NO: 242 - protein, 20 aa, synthetic construct
ENANDSSDDSGEETDESFNP

SEQ ID NO: 243 - protein, 22 aa, synthetic construct
DDNESNSESAENGWDSGSNFSE

SEQ ID NO: 244 - protein, 22 aa, synthetic construct
DNESNSESAENGWDSGSNFSEE

SEQ ID NO: 245 - protein, 23 aa, synthetic construct
SDDNESNSESAENGWDSGSNFSE

SEQ ID NO: 246 - protein, 23 aa, synthetic construct
DDNESNSESAENGWDSGSNFSEE

SEQ ID NO: 247 - protein, 24 aa, synthetic construct
SDDNESNSESAENGWDSGSNFSEE

SEQ ID NO: 248 - protein, 25 aa, synthetic construct
SSDDNESNSESAENGWDSGSNFSEE

SEQ ID NO: 249 - protein, 20 aa, synthetic construct
SEENASSGDSEENTNSDHES

SEQ ID NO: 250 - protein, 20 aa, synthetic construct
EENASSGDSEENTNSDHESE

SEQ ID NO: 251 - protein, 20 aa, synthetic construct
ENASSGDSEENTNSDHESEQ

SEQ ID NO: 252 - protein, 20 aa, synthetic construct
DENSENNWRNEYPEEESSDG

SEQ ID NO: 253 - protein, 20 aa, synthetic construct
ENSENNWRNEYPEEESSDGD

SEQ ID NO: 254 - protein, 20 aa, synthetic construct
NSENNWRNEYPEEESSDGDE

SEQ ID NO: 255 - protein, 21 aa, synthetic construct
DDENSENNWRNEYPEEESSDG

SEQ ID NO: 256 - protein, 21 aa, synthetic construct
DENSENNWRNEYPEEESSDGD

SEQ ID NO: 257 - protein, 21 aa, synthetic construct
ENSENNWRNEYPEEESSDGDE

SEQ ID NO: 258 - protein, 21 aa, synthetic construct
NSENNWRNEYPEEESSDGDED

SEQ ID NO: 259 - protein, 22 aa, synthetic construct
NSENNWRNEYPEEESSDGDEDS

SEQ ID NO: 260 - protein, 23 aa, synthetic construct
ENSENNWRNEYPEEESSDGDEDS

SEQ ID NO: 261 - protein, 20 aa, synthetic construct
EQQNEASEENNDQQSQEVPE

SEQ ID NO: 262 - protein, 20 aa, synthetic construct
QQNEASEENNDQQSQEVPEK

SEQ ID NO: 263 - protein, 20 aa, synthetic construct
SKMQEDEFDQGNQEQEDNSN

SEQ ID NO: 264 - protein, 20 aa, synthetic construct
KMQEDEFDQGNQEQEDNSNA

SEQ ID NO: 265 - protein, 20 aa, synthetic construct
MQEDEFDQGNQEQEDNSNAE

SEQ ID NO: 266 - protein, 20 aa, synthetic construct
QEDEFDQGNQEQEDNSNAEM

SEQ ID NO: 267 - protein, 20 aa, synthetic construct
FDQGNQEQEDNSNAEMEEEN

SEQ ID NO: 268 - protein, 21 aa, synthetic construct
EFDQGNQEQEDNSNAEMEEEN

SEQ ID NO: 269 - protein, 21 aa, synthetic construct
FDQGNQEQEDNSNAEMEEENA

SEQ ID NO: 270 - protein, 21 aa, synthetic construct
DQGNQEQEDNSNAEMEEENAS

SEQ ID NO: 271 - protein, 21 aa, synthetic construct
QGNQEQEDNSNAEMEEENASN

SEQ ID NO: 272 - protein, 22 aa, synthetic construct
EFDQGNQEQEDNSNAEMEEENA

SEQ ID NO: 273 - protein, 22 aa, synthetic construct
FDQGNQEQEDNSNAEMEEENAS

SEQ ID NO: 274 - protein, 22 aa, synthetic construct
DQGNQEQEDNSNAEMEEENASN

SEQ ID NO: 275 - protein, 23 aa, synthetic construct
DEFDQGNQEQEDNSNAEMEEENA

SEQ ID NO: 276 - protein, 23 aa, synthetic construct
EFDQGNQEQEDNSNAEMEEENAS

SEQ ID NO: 277 - protein, 23 aa, synthetic construct
FDQGNQEQEDNSNAEMEEENASN

SEQ ID NO: 278 - protein, 24 aa, synthetic construct
DEFDQGNQEQEDNSNAEMEEENAS

SEQ ID NO: 279 - protein, 24 aa, synthetic construct
EFDQGNQEQEDNSNAEMEEENASN

SEQ ID NO: 280 - protein, 25 aa, synthetic construct
QEDEFDQGNQEQEDNSNAEMEEENA

SEQ ID NO: 281 - protein, 25 aa, synthetic construct
EDEFDQGNQEQEDNSNAEMEEENAS

SEQ ID NO: 282 - protein, 25 aa, synthetic construct
DEFDQGNQEQEDNSNAEMEEENASN

SEQ ID NO: 283 - protein, 26 aa, synthetic construct
QEDEFDQGNQEQEDNSNAEMEEENAS

SEQ ID NO: 284 - protein, 26 aa, synthetic construct
EDEFDQGNQEQEDNSNAEMEEENASN

SEQ ID NO: 285 - protein, 27 aa, synthetic construct
QEDEFDQGNQEQEDNSNAEMEEENASN

SEQ ID NO: 286 - protein, 21 aa, synthetic construct
PSENENSQSEDSVGGDNDSEN

SEQ ID NO: 287 - protein, 20 aa, synthetic construct
EVEESNPSAKEDSNPNSSGE

SEQ ID NO: 288 - protein, 20 aa, synthetic construct
VEESNPSAKEDSNPNSSGED

SEQ ID NO: 289 - protein, 20 aa, synthetic construct
KEENSESPLNENSDESYSEE

SEQ ID NO: 290 - protein, 20 aa, synthetic construct
QPGPNHEEDADSYENMDNPD

SEQ ID NO: 291 - protein, 20 aa, synthetic construct
PNHEEDADSYENMDNPDGPD

SEQ ID NO: 292 - protein, 20 aa, synthetic construct
NHEEDADSYENMDNPDGPDP

SEQ ID NO: 293 - protein, 20 aa, synthetic construct
DDPNSSDESNGNDDANSESD

SEQ ID NO: 294 - protein, 20 aa, synthetic construct
DPNSSDESNGNDDANSESDN

SEQ ID NO: 295 - protein, 20 aa, synthetic construct
PNSSDESNGNDDANSESDNN

SEQ ID NO: 296 - protein, 20 aa, synthetic construct
NSSDESNGNDDANSESDNNS

SEQ ID NO: 297 - protein, 20 aa, synthetic construct
SSDESNGNDDANSESDNNSS

SEQ ID NO: 298 - protein, 20 aa, synthetic construct
SDESNGNDDANSESDNNSSS

SEQ ID NO: 299 - protein, 20 aa, synthetic construct
DESNGNDDANSESDNNSSSR

SEQ ID NO: 300 - protein, 21 aa, synthetic construct
MQGDDPNSSDESNGNDDANSE

SEQ ID NO: 301 - protein, 21 aa, synthetic construct
QGDDPNSSDESNGNDDANSES

SEQ ID NO: 302 - protein, 21 aa, synthetic construct
GDDPNSSDESNGNDDANSESD

SEQ ID NO: 303 - protein, 21 aa, synthetic construct
DDPNSSDESNGNDDANSESDN

SEQ ID NO: 304 - protein, 21 aa, synthetic construct
DPNSSDESNGNDDANSESDNN

SEQ ID NO: 305 - protein, 22 aa, synthetic construct
DKSMQGDDPNSSDESNGNDDAN

SEQ ID NO: 306 - protein, 22 aa, synthetic construct
SMQGDDPNSSDESNGNDDANSE

SEQ ID NO: 307 - protein, 22 aa, synthetic construct
MQGDDPNSSDESNGNDDANSES

SEQ ID NO: 308 - protein, 22 aa, synthetic construct
QGDDPNSSDESNGNDDANSESD

SEQ ID NO: 309 - protein, 22 aa, synthetic construct
GDDPNSSDESNGNDDANSESDN

SEQ ID NO: 310 - protein, 22 aa, synthetic construct
DDPNSSDESNGNDDANSESDNN

SEQ ID NO: 311 - protein, 22 aa, synthetic construct
DPNSSDESNGNDDANSESDNNS

SEQ ID NO: 312 - protein, 22 aa, synthetic construct
DESNGNDDANSESDNNSSSRGD

SEQ ID NO: 313 - protein, 23 aa, synthetic construct
DDKSMQGDDPNSSDESNGNDDAN

SEQ ID NO: 314 - protein, 23 aa, synthetic construct
DKSMQGDDPNSSDESNGNDDANS

SEQ ID NO: 315 - protein, 23 aa, synthetic construct
KSMQGDDPNSSDESNGNDDANSE

SEQ ID NO: 316 - protein, 23 aa, synthetic construct
SMQGDDPNSSDESNGNDDANSES

SEQ ID NO: 317 - protein, 23 aa, synthetic construct
MQGDDPNSSDESNGNDDANSESD

SEQ ID NO: 318 - protein, 23 aa, synthetic construct
QGDDPNSSDESNGNDDANSESDN

SEQ ID NO: 319 - protein, 23 aa, synthetic construct
GDDPNSSDESNGNDDANSESDNN

SEQ ID NO: 320 - protein, 23 aa, synthetic construct
DDPNSSDESNGNDDANSESDNNS

SEQ ID NO: 321 - protein, 23 aa, synthetic construct
DPNSSDESNGNDDANSESDNNSS

SEQ ID NO: 322 - protein, 23 aa, synthetic construct
SDESNGNDDANSESDNNSSSRGD

SEQ ID NO: 323 - protein, 23 aa, synthetic construct
DESNGNDDANSESDNNSSSRGDA

SEQ ID NO: 324 - protein, 23 aa, synthetic construct
DDANSESDNNSSSRGDASYNSDE

SEQ ID NO: 325 - protein, 24 aa, synthetic construct
FDDKSMQGDDPNSSDESNGNDDAN

SEQ ID NO: 326 - protein, 24 aa, synthetic construct
DDKSMQGDDPNSSDESNGNDDANS

SEQ ID NO: 327 - protein, 24 aa, synthetic construct
DKSMQGDDPNSSDESNGNDDANSE

SEQ ID NO: 328 - protein, 24 aa, synthetic construct
SMQGDDPNSSDESNGNDDANSESD

SEQ ID NO: 329 - protein, 24 aa, synthetic construct
MQGDDPNSSDESNGNDDANSESDN

SEQ ID NO: 330 - protein, 24 aa, synthetic construct
QGDDPNSSDESNGNDDANSESDNN

SEQ ID NO: 331 - protein, 24 aa, synthetic construct
GDDPNSSDESNGNDDANSESDNNS

SEQ ID NO: 332 - protein, 24 aa, synthetic construct
DDPNSSDESNGNDDANSESDNNSS

SEQ ID NO: 333 - protein, 25 aa, synthetic construct
DFDDKSMQGDDPNSSDESNGNDDAN

SEQ ID NO: 334 - protein, 25 aa, synthetic construct
FDDKSMQGDDPNSSDESNGNDDANS

SEQ ID NO: 335 - protein, 25 aa, synthetic construct
DDKSMQGDDPNSSDESNGNDDANSE

SEQ ID NO: 336 - protein, 25 aa, synthetic construct
DKSMQGDDPNSSDESNGNDDANSES

SEQ ID NO: 337 - protein, 25 aa, synthetic construct
KSMQGDDPNSSDESNGNDDANSESD

SEQ ID NO: 338 - protein, 25 aa, synthetic construct
SMQGDDPNSSDESNGNDDANSESDN

SEQ ID NO: 339 - protein, 25 aa, synthetic construct
MQGDDPNSSDESNGNDDANSESDNN

SEQ ID NO: 340 - protein, 25 aa, synthetic construct
QGDDPNSSDESNGNDDANSESDNNS

SEQ ID NO: 341 - protein, 25 aa, synthetic construct
GDDPNSSDESNGNDDANSESDNNSS

SEQ ID NO: 342 - protein, 25 aa, synthetic construct
DDPNSSDESNGNDDANSESDNNSSS

SEQ ID NO: 343 - protein, 26 aa, synthetic construct
DFDDKSMQGDDPNSSDESNGNDDANS

SEQ ID NO: 344 - protein, 26 aa, synthetic construct
FDDKSMQGDDPNSSDESNGNDDANSE

SEQ ID NO: 345 - protein, 26 aa, synthetic construct
DDKSMQGDDPNSSDESNGNDDANSES

SEQ ID NO: 346 - protein, 26 aa, synthetic construct
DKSMQGDDPNSSDESNGNDDANSESD

SEQ ID NO: 347 - protein, 26 aa, synthetic construct
KSMQGDDPNSSDESNGNDDANSESDN

SEQ ID NO: 348 - protein, 26 aa, synthetic construct
SMQGDDPNSSDESNGNDDANSESDNN

SEQ ID NO: 349 - protein, 26 aa, synthetic construct
MQGDDPNSSDESNGNDDANSESDNNS

SEQ ID NO: 350 - protein, 26 aa, synthetic construct
QGDDPNSSDESNGNDDANSESDNNSS

SEQ ID NO: 351 - protein, 26 aa, synthetic construct
GDDPNSSDESNGNDDANSESDNNSSS

SEQ ID NO: 352 - protein, 26 aa, synthetic construct
DDPNSSDESNGNDDANSESDNNSSSR

SEQ ID NO: 353 - protein, 27 aa, synthetic construct
DKSMQGDDPNSSDESNGNDDANSESDN

SEQ ID NO: 354 - protein, 28 aa, synthetic construct
DDKSMQGDDPNSSDESNGNDDANSESDN

SEQ ID NO: 355 - protein, 28 aa, synthetic construct
DKSMQGDDPNSSDESNGNDDANSESDNN

SEQ ID NO: 356 - protein, 28 aa, synthetic construct
DDPNSSDESNGNDDANSESDNNSSSRGD

SEQ ID NO: 357 - protein, 29 aa, synthetic construct
FDDKSMQGDDPNSSDESNGNDDANSESDN

SEQ ID NO: 358 - protein, 29 aa, synthetic construct
DDKSMQGDDPNSSDESNGNDDANSESDNN

SEQ ID NO: 359 - protein, 29 aa, synthetic construct
DKSMQGDDPNSSDESNGNDDANSESDNNS

SEQ ID NO: 360 - protein, 29 aa, synthetic construct
DDPNSSDESNGNDDANSESDNNSSSRGDA

SEQ ID NO: 361 - protein, 29 aa, synthetic construct
DESNGNDDANSESDNNSSSRGDASYNSDE

SEQ ID NO: 362 - protein, 30 aa, synthetic construct
DFDDKSMQGDDPNSSDESNGNDDANSESDN

SEQ ID NO: 363 - protein, 30 aa, synthetic construct
FDDKSMQGDDPNSSDESNGNDDANSESDNN

SEQ ID NO: 364 - protein, 30 aa, synthetic construct
DDKSMQGDDPNSSDESNGNDDANSESDNNS

SEQ ID NO: 365 - protein, 30 aa, synthetic construct
DKSMQGDDPNSSDESNGNDDANSESDNNSS

SEQ ID NO: 366 - protein, 30 aa, synthetic construct
DDPNSSDESNGNDDANSESDNNSSSRGDAS

SEQ ID NO: 367 - protein, 30 aa, synthetic construct
SDESNGNDDANSESDNNSSSRGDASYNSDE

SEQ ID NO: 368 - protein, 30 aa, synthetic construct
DESNGNDDANSESDNNSSSRGDASYNSDES

SEQ ID NO: 369 - protein, 31 aa, synthetic construct
DFDDKSMQGDDPNSSDESNGNDDANSESDNN

SEQ ID NO: 370 - protein, 31 aa, synthetic construct
FDDKSMQGDDPNSSDESNGNDDANSESDNNS

SEQ ID NO: 371 - protein, 31 aa, synthetic construct
DDKSMQGDDPNSSDESNGNDDANSESDNNSS

SEQ ID NO: 372 - protein, 32 aa, synthetic construct
DFDDKSMQGDDPNSSDESNGNDDANSESDNNS

SEQ ID NO: 373 - protein, 32 aa, synthetic construct
FDDKSMQGDDPNSSDESNGNDDANSESDNNSS

SEQ ID NO: 374 - protein, 32 aa, synthetic construct
DDKSMQGDDPNSSDESNGNDDANSESDNNSSS

SEQ ID NO: 375 - protein, 33 aa, synthetic construct
DFDDKSMQGDDPNSSDESNGNDDANSESDNNSS

SEQ ID NO: 376 - protein, 33 aa, synthetic construct
FDDKSMQGDDPNSSDESNGNDDANSESDNNSSS

SEQ ID NO: 377 - protein, 34 aa, synthetic construct
DFDDKSMQGDDPNSSDESNGNDDANSESDNNSSS

SEQ ID NO: 378 - protein, 35 aa, synthetic construct
DDPNSSDESNGNDDANSESDNNSSSRGDASYNSDE

SEQ ID NO: 379 - protein, 36 aa, synthetic construct
GDDPNSSDESNGNDDANSESDNNSSSRGDASYNSDE

SEQ ID NO: 380 - protein, 36 aa, synthetic construct
DDPNSSDESNGNDDANSESDNNSSSRGDASYNSDES

SEQ ID NO: 381 - protein, 40 aa, synthetic construct
DSYDFDDKSMQGDDPNSSDESNGNDDANSESDNNSSSRGD

SEQ ID NO: 382 - protein, 40 aa, synthetic construct
SYDFDDKSMQGDDPNSSDESNGNDDANSESDNNSSSRGDA

SEQ ID NO: 383 - protein, 40 aa, synthetic construct
YDFDDKSMQGDDPNSSDESNGNDDANSESDNNSSSRGDAS

SEQ ID NO: 384 - protein, 40 aa, synthetic construct
DFDDKSMQGDDPNSSDESNGNDDANSESDNNSSSRGDASY

SEQ ID NO: 385 - protein, 40 aa, synthetic construct
QGDDPNSSDESNGNDDANSESDNNSSSRGDASYNSDESKD

SEQ ID NO: 386 - protein, 40 aa, synthetic construct
GDDPNSSDESNGNDDANSESDNNSSSRGDASYNSDESKDN

SEQ ID NO: 387 - protein, 40 aa, synthetic construct
DDPNSSDESNGNDDANSESDNNSSSRGDASYNSDESKDNG

SEQ ID NO: 388 - protein, 42 aa, synthetic construct
DDKSMQGDDPNSSDESNGNDDANSESDNNSSSRGDASYNSDE

SEQ ID NO: 389 - protein, 43 aa, synthetic construct
DFDDKSMQGDDPNSSDESNGNDDANSESDNNSSSRGDASYNSD

SEQ ID NO: 390 - protein, 43 aa, synthetic construct
FDDKSMQGDDPNSSDESNGNDDANSESDNNSSSRGDASYNSDE

SEQ ID NO: 391 - protein, 43 aa, synthetic construct
DDKSMQGDDPNSSDESNGNDDANSESDNNSSSRGDASYNSDES

SEQ ID NO: 392 - protein, 44 aa, synthetic construct
DFDDKSMQGDDPNSSDESNGNDDANSESDNNSSSRGDASYNSDE

SEQ ID NO: 393 - protein, 45 aa, synthetic construct
DFDDKSMQGDDPNSSDESNGNDDANSESDNNSSSRGDASYNSDES

SEQ ID NO: 394 - protein, 60 aa, synthetic construct
DDPNSSDESNGNDDANSESDNNSSSRGDASYNSDESKDNGNGSDSKGAEDDDSDSTSDTN

SEQ ID NO: 395 - protein, 60 aa, synthetic construct
DPNSSDESNGNDDANSESDNNSSSRGDASYNSDESKDNGNGSDSKGAEDDDSDSTSDTNN

SEQ ID NO: 396 - protein, 60 aa, synthetic construct
NSSDESNGNDDANSESDNNSSSRGDASYNSDESKDNGNGSDSKGAEDDDSDSTSDTNNSD

SEQ ID NO: 397 - protein, 60 aa, synthetic construct
SSDESNGNDDANSESDNNSSSRGDASYNSDESKDNGNGSDSKGAEDDDSDSTSDTNNSDS

SEQ ID NO: 398 - protein, 60 aa, synthetic construct
SDESNGNDDANSESDNNSSSRGDASYNSDESKDNGNGSDSKGAEDDDSDSTSDTNNSDSN

SEQ ID NO: 399 - protein, 60 aa, synthetic construct
AEDDDSDSTSDTNNSDSNGNGNNGNDDNDKSDSGKGKSDSSDSDSSDSSNSSDSSDSSDS

SEQ ID NO: 400 - protein, 60 aa, synthetic construct
EDDDSDSTSDTNNSDSNGNGNNGNDDNDKSDSGKGKSDSSDSDSSDSSNSSDSSDSSDSD

SEQ ID NO: 401 - protein, 60 aa, synthetic construct
DDDSDSTSDTNNSDSNGNGNNGNDDNDKSDSGKGKSDSSDSDSSDSSNSSDSSDSSDSDS

SEQ ID NO: 402 - protein, 61 aa, synthetic construct
GDDPNSSDESNGNDDANSESDNNSSSRGDASYNSDESKDNGNGSDSKGAEDDDSDSTSDTN

SEQ ID NO: 403 - protein, 61 aa, synthetic construct
DDPNSSDESNGNDDANSESDNNSSSRGDASYNSDESKDNGNGSDSKGAEDDDSDSTSDTNN

SEQ ID NO: 404 - protein, 62 aa, synthetic construct
QGDDPNSSDESNGNDDANSESDNNSSSRGDASYNSDESKDNGNGSDSKGAEDDDSDSTSDTN

SEQ ID NO: 405 - protein, 62 aa, synthetic construct
GDDPNSSDESNGNDDANSESDNNSSSRGDASYNSDESKDNGNGSDSKGAEDDDSDSTSDTNN

SEQ ID NO: 406 - protein, 62 aa, synthetic construct
DDPNSSDESNGNDDANSESDNNSSSRGDASYNSDESKDNGNGSDSKGAEDDDSDSTSDTNNS

SEQ ID NO: 407 - protein, 62 aa, synthetic construct
DPNSSDESNGNDDANSESDNNSSSRGDASYNSDESKDNGNGSDSKGAEDDDSDSTSDTNNSD

SEQ ID NO: 408 - protein, 63 aa, synthetic construct
MQGDDPNSSDESNGNDDANSESDNNSSSRGDASYNSDESKDNGNGSDSKGAEDDDSDSTSDTN

SEQ ID NO: 409 - protein, 63 aa, synthetic construct
QGDDPNSSDESNGNDDANSESDNNSSSRGDASYNSDESKDNGNGSDSKGAEDDDSDSTSDTNN

SEQ ID NO: 410 - protein, 63 aa, synthetic construct
GDDPNSSDESNGNDDANSESDNNSSSRGDASYNSDESKDNGNGSDSKGAEDDDSDSTSDTNNS

SEQ ID NO: 411 - protein, 63 aa, synthetic construct
DDPNSSDESNGNDDANSESDNNSSSRGDASYNSDESKDNGNGSDSKGAEDDDSDSTSDTNNSD

SEQ ID NO: 412 - protein, 63 aa, synthetic construct
DPNSSDESNGNDDANSESDNNSSSRGDASYNSDESKDNGNGSDSKGAEDDDSDSTSDTNNSDS

SEQ ID NO: 413 - protein, 64 aa, synthetic construct
GDDPNSSDESNGNDDANSESDNNSSSRGDASYNSDESKDNGNGSDSKGAEDDDSDSTSDTNNSD

SEQ ID NO: 414 - protein, 64 aa, synthetic construct
DDPNSSDESNGNDDANSESDNNSSSRGDASYNSDESKDNGNGSDSKGAEDDDSDSTSDTNNSDS

SEQ ID NO: 415 - protein, 65 aa, synthetic construct
QGDDPNSSDESNGNDDANSESDNNSSSRGDASYNSDESKDNGNGSDSKGAEDDDSDSTSDTNNSD

SEQ ID NO: 416 - protein, 65 aa, synthetic construct
GDDPNSSDESNGNDDANSESDNNSSSRGDASYNSDESKDNGNGSDSKGAEDDDSDSTSDTNNSDS

SEQ ID NO: 417 - protein, 65 aa, synthetic construct
DDPNSSDESNGNDDANSESDNNSSSRGDASYNSDESKDNGNGSDSKGAEDDDSDSTSDTNNSDSN

SEQ ID NO: 418 - protein, 65 aa, synthetic construct
EDDDSDSTSDTNNSDSNGNGNNGNDDNDKSDSGKGKSDSSDSDSSDSSNSSDSSDSSDSDSSDSN

SEQ ID NO: 419 - protein, 66 aa, synthetic construct
MQGDDPNSSDESNGNDDANSESDNNSSSRGDASYNSDESKDNGNGSDSKGAEDDDSDSTSDTNNSD

SEQ ID NO: 420 - protein, 66 aa, synthetic construct
QGDDPNSSDESNGNDDANSESDNNSSSRGDASYNSDESKDNGNGSDSKGAEDDDSDSTSDTNNSDS

SEQ ID NO: 421 - protein, 66 aa, synthetic construct
GDDPNSSDESNGNDDANSESDNNSSSRGDASYNSDESKDNGNGSDSKGAEDDDSDSTSDTNNSDSN

SEQ ID NO: 422 - protein, 66 aa, synthetic construct
DDPNSSDESNGNDDANSESDNNSSSRGDASYNSDESKDNGNGSDSKGAEDDDSDSTSDTNNSDSNG

SEQ ID NO: 423 - protein, 66 aa, synthetic construct
AEDDDSDSTSDTNNSDSNGNGNNGNDDNDKSDSGKGKSDSSDSDSSDSSNSSDSSDSSDSDSSDSN

SEQ ID NO: 424 - protein, 66 aa, synthetic construct
EDDDSDSTSDTNNSDSNGNGNNGNDDNDKSDSGKGKSDSSDSDSSDSSNSSDSSDSSDSDSSDSNS

SEQ ID NO: 425 - protein, 21 aa, synthetic construct
LEEDDNNENAGEDGDNDFSPS

SEQ ID NO: 426 - protein, 21 aa, synthetic construct
DNNENAGEDGDNDFSPSDEEL

SEQ ID NO: 427 - protein, 21 aa, synthetic construct
NNENAGEDGDNDFSPSDEELA

SEQ ID NO: 428 - protein, 21 aa, synthetic construct
NENAGEDGDNDFSPSDEELAN

SEQ ID NO: 429 - protein, 22 aa, synthetic construct
DNNENAGEDGDNDFSPSDEELA

SEQ ID NO: 430 - protein, 22 aa, synthetic construct
NNENAGEDGDNDFSPSDEELAN

SEQ ID NO: 431 - protein, 23 aa, synthetic construct
AELEEDDNNENAGEDGDNDFSPS

SEQ ID NO: 432 - protein, 23 aa, synthetic construct
DDNNENAGEDGDNDFSPSDEELA

SEQ ID NO: 433 - protein, 23 aa, synthetic construct
DNNENAGEDGDNDFSPSDEELAN

SEQ ID NO: 434 - protein, 24 aa, synthetic construct
DDNNENAGEDGDNDFSPSDEELAN

SEQ ID NO: 435 - protein, 25 aa, synthetic construct
EDDNNENAGEDGDNDFSPSDEELAN

SEQ ID NO: 436 - protein, 24 aa, synthetic construct
NPADDPNNQGEDEFEEAEQVREEN

SEQ ID NO: 437 - protein, 21 aa, synthetic construct
NEENTEPGAESSENADDPNKD

SEQ ID NO: 438 - protein, 21 aa, synthetic construct
ENADDPNKDTSENADGQSDEN

SEQ ID NO: 439 - protein, 22 aa, synthetic construct
SENADDPNKDTSENADGQSDEN

SEQ ID NO: 440 - protein, 23 aa, synthetic construct
SSENADDPNKDTSENADGQSDEN

SEQ ID NO: 441 - protein, 24 aa, synthetic construct
ESSENADDPNKDTSENADGQSDEN

SEQ ID NO: 442 - protein, 20 aa, synthetic construct
DRDPEMENEEQPSSENDSQN

SEQ ID NO: 443 - protein, 20 aa, synthetic construct
RDPEMENEEQPSSENDSQNQ

SEQ ID NO: 444 - protein, 20 aa, synthetic construct
DPEMENEEQPSSENDSQNQS

SEQ ID NO: 445 - protein, 20 aa, synthetic construct
PEMENEEQPSSENDSQNQSG

SEQ ID NO: 446 - protein, 20 aa, synthetic construct
EMENEEQPSSENDSQNQSGE

SEQ ID NO: 447 - protein, 20 aa, synthetic construct
MENEEQPSSENDSQNQSGEQ

SEQ ID NO: 448 - protein, 20 aa, synthetic construct
ENEEQPSSENDSQNQSGEQI

SEQ ID NO: 449 - protein, 21 aa, synthetic construct
DSESANVSDKEAGSNENDDQN

SEQ ID NO: 450 - protein, 20 aa, synthetic construct
NYNDGSQEDRDWQDDQSDNQ

SEQ ID NO: 451 - protein, 20 aa, synthetic construct
RENTNEASSEGNSSDDSEDE

SEQ ID NO: 452 - protein, 20 aa, synthetic construct
ENTNEASSEGNSSDDSEDER

SEQ ID NO: 453 - protein, 20 aa, synthetic construct
QENDNGNETESEQPKESNEN

SEQ ID NO: 454 - protein, 20 aa, synthetic construct
ENDNGNETESEQPKESNENQ

SEQ ID NO: 455 - protein, 20 aa, synthetic construct
NDNGNETESEQPKESNENQE

SEQ ID NO: 456 - protein, 21 aa, synthetic construct
QENDNGNETESEQPKESNENQ

SEQ ID NO: 457 - protein, 21 aa, synthetic construct
ENDNGNETESEQPKESNENQE

SEQ ID NO: 458 - protein, 22 aa, synthetic construct
QENDNGNETESEQPKESNENQE

SEQ ID NO: 459 - protein, 20 aa, synthetic construct
QNEENPGDEEAKNQVNSESD

SEQ ID NO: 460 - protein, 20 aa, synthetic construct
NEENPGDEEAKNQVNSESDS

SEQ ID NO: 461 - protein, 20 aa, synthetic construct
EENPGDEEAKNQVNSESDSD

SEQ ID NO: 462 - protein, 20 aa, synthetic construct
ENPGDEEAKNQVNSESDSDS

SEQ ID NO: 463 - protein, 20 aa, synthetic construct
NPGDEEAKNQVNSESDSDSE

SEQ ID NO: 464 - protein, 21 aa, synthetic construct
QNEENPGDEEAKNQVNSESDS

SEQ ID NO: 465 - protein, 21 aa, synthetic construct
NEENPGDEEAKNQVNSESDSD

SEQ ID NO: 466 - protein, 22 aa, synthetic construct
QNEENPGDEEAKNQVNSESDSD

SEQ ID NO: 467 - protein, 22 aa, synthetic construct
NEENPGDEEAKNQVNSESDSDS

SEQ ID NO: 468 - protein, 23 aa, synthetic construct
QNEENPGDEEAKNQVNSESDSDS

SEQ ID NO: 469 - protein, 23 aa, synthetic construct
NEENPGDEEAKNQVNSESDSDSE

SEQ ID NO: 470 - protein, 24 aa, synthetic construct
QNEENPGDEEAKNQVNSESDSDSE

SEQ ID NO: 471 - protein, 24 aa, synthetic construct
NEENPGDEEAKNQVNSESDSDSEE

SEQ ID NO: 472 - protein, 25 aa, synthetic construct
QNEENPGDEEAKNQVNSESDSDSEE

SEQ ID NO: 473 - protein, 25 aa, synthetic construct
NEENPGDEEAKNQVNSESDSDSEES

SEQ ID NO: 474 - protein, 26 aa, synthetic construct
QNEENPGDEEAKNQVNSESDSDSEES

SEQ ID NO: 475 - protein, 21 aa, synthetic construct
YNGGNANPRPANNEEEEDEED

SEQ ID NO: 476 - protein, 21 aa, synthetic construct
NGGNANPRPANNEEEEDEEDE

SEQ ID NO: 477 - protein, 21 aa, synthetic construct
GGNANPRPANNEEEEDEEDEY

SEQ ID NO: 478 - protein, 22 aa, synthetic construct
YNGGNANPRPANNEEEEDEEDE

SEQ ID NO: 479 - protein, 22 aa, synthetic construct
NGGNANPRPANNEEEEDEEDEY

SEQ ID NO: 480 - protein, 23 aa, synthetic construct
NGGNANPRPANNEEEEDEEDEYD

SEQ ID NO: 481 - protein, 20 aa, synthetic construct
GASENEEEDDDYNKPLDPNS

SEQ ID NO: 482 - protein, 20 aa, synthetic construct
LQNQKEAEEPGPDSENSQEN

SEQ ID NO: 483 - protein, 20 aa, synthetic construct
QNQKEAEEPGPDSENSQENP

SEQ ID NO: 484 - protein, 20 aa, synthetic construct
NQKEAEEPGPDSENSQENPP

SEQ ID NO: 485 - protein, 21 aa, synthetic construct
KESVSPENNEEGGNDNQDNEN

SEQ ID NO: 486 - protein, 21 aa, synthetic construct
ESVSPENNEEGGNDNQDNENP

SEQ ID NO: 487 - protein, 22 aa, synthetic construct
KESVSPENNEEGGNDNQDNENP

SEQ ID NO: 488 - protein, 20 aa, synthetic construct
ASPQPREPSDDENSDNSNEC

SEQ ID NO: 489 - protein, 21 aa, synthetic construct
NNSQDEDGFQELNENGNAKDE

SEQ ID NO: 490 - protein, 21 aa, synthetic construct
NSQDEDGFQELNENGNAKDEN

SEQ ID NO: 491 - protein, 22 aa, synthetic construct
NNSQDEDGFQELNENGNAKDEN

SEQ ID NO: 492 - protein, 20 aa, synthetic construct
SSDSPKDQSPPEDSGESEAD

SEQ ID NO: 493 - protein, 20 aa, synthetic construct
PGPEPEPEPEPEPEPAPEPE

SEQ ID NO: 494 - protein, 20 aa, synthetic construct
GPEPEPEPEPEPEPAPEPEP

SEQ ID NO: 495 - protein, 20 aa, synthetic construct
PEPEPEPEPEPAPEPEPEPK

SEQ ID NO: 496 - protein, 20 aa, synthetic construct
EPEPEPEPEPAPEPEPEPKP

SEQ ID NO: 497 - protein, 20 aa, synthetic construct
PEPEPEPEPAPEPEPEPKPG

SEQ ID NO: 498 - protein, 21 aa, synthetic construct
AGPGPEPEPEPEPEPEPAPEP

SEQ ID NO: 499 - protein, 21 aa, synthetic construct
GPGPEPEPEPEPEPEPAPEPE

SEQ ID NO: 500 - protein, 21 aa, synthetic construct
PGPEPEPEPEPEPEPAPEPEP

SEQ ID NO: 501 - protein, 21 aa, synthetic construct
GPEPEPEPEPEPEPAPEPEPE

SEQ ID NO: 502 - protein, 21 aa, synthetic construct
PEPEPEPEPEPEPAPEPEPEP

SEQ ID NO: 503 - protein, 21 aa, synthetic construct
EPEPEPEPEPEPAPEPEPEPK

SEQ ID NO: 504 - protein, 21 aa, synthetic construct
PEPEPEPEPEPAPEPEPEPKP

SEQ ID NO: 505 - protein, 21 aa, synthetic construct
EPEPEPEPEPAPEPEPEPKPG

SEQ ID NO: 506 - protein, 21 aa, synthetic construct
PEPEPEPEPAPEPEPEPKPGA

SEQ ID NO: 507 - protein, 21 aa, synthetic construct
EPEPEPEPAPEPEPEPKPGAG

SEQ ID NO: 508 - protein, 22 aa, synthetic construct
AGPGPEPEPEPEPEPEPAPEPE

SEQ ID NO: 509 - protein, 22 aa, synthetic construct
GPGPEPEPEPEPEPEPAPEPEP

SEQ ID NO: 510 - protein, 22 aa, synthetic construct
PGPEPEPEPEPEPEPAPEPEPE

SEQ ID NO: 511 - protein, 22 aa, synthetic construct
GPEPEPEPEPEPEPAPEPEPEP

SEQ ID NO: 512 - protein, 22 aa, synthetic construct
PEPEPEPEPEPEPAPEPEPEPK

SEQ ID NO: 513 - protein, 22 aa, synthetic construct
EPEPEPEPEPEPAPEPEPEPKP

SEQ ID NO: 514 - protein, 22 aa, synthetic construct
PEPEPEPEPEPAPEPEPEPKPG

SEQ ID NO: 515 - protein, 22 aa, synthetic construct
EPEPEPEPEPAPEPEPEPKPGA

SEQ ID NO: 516 - protein, 22 aa, synthetic construct
PEPEPEPEPAPEPEPEPKPGAG

SEQ ID NO: 517 - protein, 23 aa, synthetic construct
AGPGPEPEPEPEPEPEPAPEPEP

SEQ ID NO: 518 - protein, 23 aa, synthetic construct
GPGPEPEPEPEPEPEPAPEPEPE

SEQ ID NO: 519 - protein, 23 aa, synthetic construct
PGPEPEPEPEPEPEPAPEPEPEP

SEQ ID NO: 520 - protein, 23 aa, synthetic construct
GPEPEPEPEPEPEPAPEPEPEPK

SEQ ID NO: 521 - protein, 23 aa, synthetic construct
PEPEPEPEPEPEPAPEPEPEPKP

SEQ ID NO: 522 - protein, 23 aa, synthetic construct
EPEPEPEPEPEPAPEPEPEPKPG

SEQ ID NO: 523 - protein, 23 aa, synthetic construct
PEPEPEPEPEPAPEPEPEPKPGA

SEQ ID NO: 524 - protein, 23 aa, synthetic construct
EPEPEPEPEPAPEPEPEPKPGAG

SEQ ID NO: 525 - protein, 24 aa, synthetic construct
AGPGPEPEPEPEPEPEPAPEPEPE

SEQ ID NO: 526 - protein, 24 aa, synthetic construct
GPGPEPEPEPEPEPEPAPEPEPEP

SEQ ID NO: 527 - protein, 24 aa, synthetic construct
PGPEPEPEPEPEPEPAPEPEPEPK

SEQ ID NO: 528 - protein, 24 aa, synthetic construct
GPEPEPEPEPEPEPAPEPEPEPKP

SEQ ID NO: 529 - protein, 24 aa, synthetic construct
PEPEPEPEPEPEPAPEPEPEPKPG

SEQ ID NO: 530 - protein, 24 aa, synthetic construct
EPEPEPEPEPEPAPEPEPEPKPGA

SEQ ID NO: 531 - protein, 24 aa, synthetic construct
PEPEPEPEPEPAPEPEPEPKPGAG

SEQ ID NO: 532 - protein, 25 aa, synthetic construct
AGPGPEPEPEPEPEPEPAPEPEPEP

SEQ ID NO: 533 - protein, 25 aa, synthetic construct
GPGPEPEPEPEPEPEPAPEPEPEPK

SEQ ID NO: 534 - protein, 25 aa, synthetic construct
PGPEPEPEPEPEPEPAPEPEPEPKP

SEQ ID NO: 535 - protein, 25 aa, synthetic construct
GPEPEPEPEPEPEPAPEPEPEPKPG

SEQ ID NO: 536 - protein, 25 aa, synthetic construct
PEPEPEPEPEPEPAPEPEPEPKPGA

SEQ ID NO: 537 - protein, 25 aa, synthetic construct
EPEPEPEPEPEPAPEPEPEPKPGAG

SEQ ID NO: 538 - protein, 26 aa, synthetic construct
AGPGPEPEPEPEPEPEPAPEPEPEPK

SEQ ID NO: 539 - protein, 26 aa, synthetic construct
GPGPEPEPEPEPEPEPAPEPEPEPKP

SEQ ID NO: 540 - protein, 26 aa, synthetic construct
PGPEPEPEPEPEPEPAPEPEPEPKPG

SEQ ID NO: 541 - protein, 26 aa, synthetic construct
GPEPEPEPEPEPEPAPEPEPEPKPGA

SEQ ID NO: 542 - protein, 26 aa, synthetic construct
PEPEPEPEPEPEPAPEPEPEPKPGAG

SEQ ID NO: 543 - protein, 27 aa, synthetic construct
AGPGPEPEPEPEPEPEPAPEPEPEPKP

SEQ ID NO: 544 - protein, 27 aa, synthetic construct
PGPEPEPEPEPEPEPAPEPEPEPKPGA

SEQ ID NO: 545 - protein, 20 aa, synthetic construct
DPEEDGSPDPEPSPEPEPKP

SEQ ID NO: 546 - protein, 20 aa, synthetic construct
PEEDGSPDPEPSPEPEPKPS

SEQ ID NO: 547 - protein, 21 aa, synthetic construct
EDPEEDGSPDPEPSPEPEPKP

SEQ ID NO: 548 - protein, 21 aa, synthetic construct
DPEEDGSPDPEPSPEPEPKPS

SEQ ID NO: 549 - protein, 22 aa, synthetic construct
EDPEEDGSPDPEPSPEPEPKPS

SEQ ID NO: 550 - protein, 23 aa, synthetic construct
DEDPEEDGSPDPEPSPEPEPKPS

SEQ ID NO: 551 - protein, 21 aa, synthetic construct
PDDDGSDDSSPPSASPAESEP

SEQ ID NO: 552 - protein, 21 aa, synthetic construct
DDDGSDDSSPPSASPAESEPQ

SEQ ID NO: 553 - protein, 22 aa, synthetic construct
PDDDGSDDSSPPSASPAESEPQ

SEQ ID NO: 554 - protein, 21 aa, synthetic construct
PPPEEQGQGDAPPQHEDEEPA

SEQ ID NO: 555 - protein, 20 aa, synthetic construct
PSPAPSPDSDSDSDSDGEEE

SEQ ID NO: 556 - protein, 21 aa, synthetic construct
GAPSPAPSPDSDSDSDSDGEE

SEQ ID NO: 557 - protein, 21 aa, synthetic construct
APSPAPSPDSDSDSDSDGEEE

SEQ ID NO: 558 - protein, 21 aa, synthetic construct
PSPAPSPDSDSDSDSDGEEEE

SEQ ID NO: 559 - protein, 22 aa, synthetic construct
NGAPSPAPSPDSDSDSDSDGEE

SEQ ID NO: 560 - protein, 22 aa, synthetic construct
GAPSPAPSPDSDSDSDSDGEEE

SEQ ID NO: 561 - protein, 22 aa, synthetic construct
APSPAPSPDSDSDSDSDGEEEE

SEQ ID NO: 562 - protein, 23 aa, synthetic construct
QNGAPSPAPSPDSDSDSDSDGEE

SEQ ID NO: 563 - protein, 23 aa, synthetic construct
NGAPSPAPSPDSDSDSDSDGEEE

SEQ ID NO: 564 - protein, 23 aa, synthetic construct
GAPSPAPSPDSDSDSDSDGEEEE

SEQ ID NO: 565 - protein, 23 aa, synthetic construct
APSPAPSPDSDSDSDSDGEEEEE

SEQ ID NO: 566 - protein, 24 aa, synthetic construct
QNGAPSPAPSPDSDSDSDSDGEEE

SEQ ID NO: 567 - protein, 24 aa, synthetic construct
NGAPSPAPSPDSDSDSDSDGEEEE

SEQ ID NO: 568 - protein, 24 aa, synthetic construct
GAPSPAPSPDSDSDSDSDGEEEEE

SEQ ID NO: 569 - protein, 25 aa, synthetic construct
QNGAPSPAPSPDSDSDSDSDGEEEE

SEQ ID NO: 570 - protein, 25 aa, synthetic construct
NGAPSPAPSPDSDSDSDSDGEEEEE

SEQ ID NO: 571 - protein, 25 aa, synthetic construct
GAPSPAPSPDSDSDSDSDGEEEEEE

SEQ ID NO: 572 - protein, 26 aa, synthetic construct
QNGAPSPAPSPDSDSDSDSDGEEEEE

SEQ ID NO: 573 - protein, 26 aa, synthetic construct
NGAPSPAPSPDSDSDSDSDGEEEEEE

SEQ ID NO: 574 - protein, 27 aa, synthetic construct
QNGAPSPAPSPDSDSDSDSDGEEEEEE

SEQ ID NO: 575 - protein, 27 aa, synthetic construct
NGAPSPAPSPDSDSDSDSDGEEEEEEE

SEQ ID NO: 576 - protein, 28 aa, synthetic construct
QNGAPSPAPSPDSDSDSDSDGEEEEEEE

SEQ ID NO: 577 - protein, 31 aa, synthetic construct
QNGAPSPAPSPDSDSDSDSDGEEEEEEEGER

SEQ ID NO: 578 - protein, 32 aa, synthetic construct
QNGAPSPAPSPDSDSDSDSDGEEEEEEEGERD

SEQ ID NO: 579 - protein, 20 aa, synthetic construct
EPPAPPPPPPPEEDPEQDSG

SEQ ID NO: 580 - protein, 20 aa, synthetic construct
PAPPPPPPPEEDPEQDSGPE

SEQ ID NO: 581 - protein, 20 aa, synthetic construct
APPPPPPPEEDPEQDSGPED

SEQ ID NO: 582 - protein, 21 aa, synthetic construct
PAPPPPPPPEEDPEQDSGPED

SEQ ID NO: 583 - protein, 22 aa, synthetic construct
EPPAPPPPPPPEEDPEQDSGPE

SEQ ID NO: 584 - protein, 22 aa, synthetic construct
PPAPPPPPPPEEDPEQDSGPED

SEQ ID NO: 585 - protein, 23 aa, synthetic construct
AEPPAPPPPPPPEEDPEQDSGPE

SEQ ID NO: 586 - protein, 23 aa, synthetic construct
EPPAPPPPPPPEEDPEQDSGPED

SEQ ID NO: 587 - protein, 24 aa, synthetic construct
AEPPAPPPPPPPEEDPEQDSGPED

SEQ ID NO: 588 - protein, 20 aa, synthetic construct
PPPPPQAPPEEENESEPEEP

SEQ ID NO: 589 - protein, 20 aa, synthetic construct
PPPPQAPPEEENESEPEEPS

SEQ ID NO: 590 - protein, 20 aa, synthetic construct
PPPQAPPEEENESEPEEPSG

SEQ ID NO: 591 - protein, 21 aa, synthetic construct
PPPPPQAPPEEENESEPEEPS

SEQ ID NO: 592 - protein, 21 aa, synthetic construct
PPPPQAPPEEENESEPEEPSG

SEQ ID NO: 593 - protein, 22 aa, synthetic construct
PPPPPQAPPEEENESEPEEPSG

SEQ ID NO: 594 - protein, 20 aa, synthetic construct
PQDSSSKSPEPSADESPDND

SEQ ID NO: 595 - protein, 20 aa, synthetic construct
QSSDNSEDEEEPPDNADSKS

SEQ ID NO: 596 - protein, 21 aa, synthetic construct
EEEEEQPGKAPDPQDPQDAES

SEQ ID NO: 597 - protein, 21 aa, synthetic construct
EEEEQPGKAPDPQDPQDAESD

SEQ ID NO: 598 - protein, 21 aa, synthetic construct
EEEQPGKAPDPQDPQDAESDS

SEQ ID NO: 599 - protein, 22 aa, synthetic construct
EEEEQPGKAPDPQDPQDAESDS

SEQ ID NO: 600 - protein, 23 aa, synthetic construct
EEEEEQPGKAPDPQDPQDAESDS

SEQ ID NO: 601 - protein, 20 aa, synthetic construct
PPPPPSEEEGPEEPPKASPE

SEQ ID NO: 602 - protein, 20 aa, synthetic construct
PPPPSEEEGPEEPPKASPES

SEQ ID NO: 603 - protein, 20 aa, synthetic construct
PPPSEEEGPEEPPKASPESE

SEQ ID NO: 604 - protein, 21 aa, synthetic construct
PPPPSEEEGPEEPPKASPESE

SEQ ID NO: 605 - protein, 21 aa, synthetic construct
PPPSEEEGPEEPPKASPESEA

SEQ ID NO: 606 - protein, 22 aa, synthetic construct
PPPPPSEEEGPEEPPKASPESE

SEQ ID NO: 607 - protein, 22 aa, synthetic construct
PPPPSEEEGPEEPPKASPESEA

SEQ ID NO: 608 - protein, 23 aa, synthetic construct
PPPPPSEEEGPEEPPKASPESEA

SEQ ID NO: 609 - protein, 20 aa, synthetic construct
DDAEEPESPPPPPRSPSPEP

SEQ ID NO: 610 - protein, 20 aa, synthetic construct
SGEASSSEEEPPSPDDKENQ

SEQ ID NO: 611 - protein, 21 aa, synthetic construct
SGEASSSEEEPPSPDDKENQA

SEQ ID NO: 612 - protein, 21 aa, synthetic construct
GEASSSEEEPPSPDDKENQAP

SEQ ID NO: 613 - protein, 22 aa, synthetic construct
SGEASSSEEEPPSPDDKENQAP

SEQ ID NO: 614 - protein, 20 aa, synthetic construct
PQPPPPPPPEESSDSEPEAE

SEQ ID NO: 615 - protein, 20 aa, synthetic construct
QPPPPPPPEESSDSEPEAEP

SEQ ID NO: 616 - protein, 20 aa, synthetic construct
PPPPPPPEESSDSEPEAEPG

SEQ ID NO: 617 - protein, 20 aa, synthetic construct
PPPPPPEESSDSEPEAEPGS

SEQ ID NO: 618 - protein, 20 aa, synthetic construct
PPPPPEESSDSEPEAEPGSP

SEQ ID NO: 619 - protein, 20 aa, synthetic construct
PPPPEESSDSEPEAEPGSPQ

SEQ ID NO: 620 - protein, 20 aa, synthetic construct
PPPEESSDSEPEAEPGSPQK

SEQ ID NO: 621 - protein, 21 aa, synthetic construct
SDPEPPDAGEDSKSENGENAP

SEQ ID NO: 622 - protein, 20 aa, synthetic construct
SDSESEDPPRNQASDSENEE

SEQ ID NO: 623 - protein, 21 aa, synthetic construct
GPGEDAEPDEDPQSEDSEAPS

SEQ ID NO: 624 - protein, 21 aa, synthetic construct
PGEDAEPDEDPQSEDSEAPSS

SEQ ID NO: 625 - protein, 22 aa, synthetic construct
GPGEDAEPDEDPQSEDSEAPSS

SEQ ID NO: 626 - protein, 23 aa, synthetic construct
ESESSSSDSEANEPSQSASPEPE

SEQ ID NO: 627 - protein, 25 aa, synthetic construct
DSESESSSSDSEANEPSQSASPEPE

SEQ ID NO: 628 - protein, 26 aa, synthetic construct
SDSESESSSSDSEANEPSQSASPEPE

SEQ ID NO: 629 - protein, 26 aa, synthetic construct
DSESESSSSDSEANEPSQSASPEPEP

SEQ ID NO: 630 - protein, 23 aa, synthetic construct
HQEDSEEESQEEEAEGASEPPPP

SEQ ID NO: 631 - protein, 25 aa, synthetic construct
GDHQEDSEEESQEEEAEGASEPPPP

SEQ ID NO: 632 - protein, 20 aa, synthetic construct
PSQPPEEPEPDEAESSPDPQ

SEQ ID NO: 633 - protein, 21 aa, synthetic construct
DPAPSQPPEEPEPDEAESSPD

SEQ ID NO: 634 - protein, 21 aa, synthetic construct
PAPSQPPEEPEPDEAESSPDP

SEQ ID NO: 635 - protein, 21 aa, synthetic construct
APSQPPEEPEPDEAESSPDPQ

SEQ ID NO: 636 - protein, 21 aa, synthetic construct
PSQPPEEPEPDEAESSPDPQA

SEQ ID NO: 637 - protein, 22 aa, synthetic construct
DPAPSQPPEEPEPDEAESSPDP

SEQ ID NO: 638 - protein, 22 aa, synthetic construct
PAPSQPPEEPEPDEAESSPDPQ

SEQ ID NO: 639 - protein, 23 aa, synthetic construct
DPAPSQPPEEPEPDEAESSPDPQ

SEQ ID NO: 640 - protein, 21 aa, synthetic construct
PGSQPQASSGPEAEEEEEDDE

SEQ ID NO: 641 - protein, 20 aa, synthetic construct
DSPDSQEEQKGESSASSPEE

SEQ ID NO: 642 - protein, 20 aa, synthetic construct
SPDSQEEQKGESSASSPEEP

SEQ ID NO: 643 - protein, 20 aa, synthetic construct
PDSQEEQKGESSASSPEEPE

SEQ ID NO: 644 - protein, 20 aa, synthetic construct
DSQEEQKGESSASSPEEPEE

SEQ ID NO: 645 - protein, 21 aa, synthetic construct
DSPDSQEEQKGESSASSPEEP

SEQ ID NO: 646 - protein, 21 aa, synthetic construct
SPDSQEEQKGESSASSPEEPE

SEQ ID NO: 647 - protein, 21 aa, synthetic construct
PDSQEEQKGESSASSPEEPEE

SEQ ID NO: 648 - protein, 22 aa, synthetic construct
PADSPDSQEEQKGESSASSPEE

SEQ ID NO: 649 - protein, 22 aa, synthetic construct
ADSPDSQEEQKGESSASSPEEP

SEQ ID NO: 650 - protein, 22 aa, synthetic construct
DSPDSQEEQKGESSASSPEEPE

SEQ ID NO: 651 - protein, 22 aa, synthetic construct
SPDSQEEQKGESSASSPEEPEE

SEQ ID NO: 652 - protein, 23 aa, synthetic construct
PADSPDSQEEQKGESSASSPEEP

SEQ ID NO: 653 - protein, 23 aa, synthetic construct
ADSPDSQEEQKGESSASSPEEPE

SEQ ID NO: 654 - protein, 23 aa, synthetic construct
DSPDSQEEQKGESSASSPEEPEE

SEQ ID NO: 655 - protein, 24 aa, synthetic construct
PADSPDSQEEQKGESSASSPEEPE

SEQ ID NO: 656 - protein, 24 aa, synthetic construct
ADSPDSQEEQKGESSASSPEEPEE

SEQ ID NO: 657 - protein, 25 aa, synthetic construct
PADSPDSQEEQKGESSASSPEEPEE

SEQ ID NO: 658 - protein, 20 aa, synthetic construct
SPSPEDESSSSSSSSSSEDE

SEQ ID NO: 659 - protein, 20 aa, synthetic construct
PSPEDESSSSSSSSSSEDEE

SEQ ID NO: 660 - protein, 23 aa, synthetic construct
PRSPSPEDESSSSSSSSSSEDEE

SEQ ID NO: 661 - protein, 24 aa, synthetic construct
PRSPSPEDESSSSSSSSSSEDEEE

SEQ ID NO: 662 - protein, 20 aa, synthetic construct
SSSDSSDSDSSEDDEAPSKP

SEQ ID NO: 663 - protein, 21 aa, synthetic construct
PSPGSPRGQPQDQDDDEDDEE

SEQ ID NO: 664 - protein, 20 aa, synthetic construct
QQAEDHPQNPPEDPNQDPPE

SEQ ID NO: 665 - protein, 20 aa, synthetic construct
QAEDHPQNPPEDPNQDPPED

SEQ ID NO: 666 - protein, 20 aa, synthetic construct
AEDHPQNPPEDPNQDPPEDD

SEQ ID NO: 667 - protein, 20 aa, synthetic construct
EDHPQNPPEDPNQDPPEDDS

SEQ ID NO: 668 - protein, 21 aa, synthetic construct
QQAEDHPQNPPEDPNQDPPED

SEQ ID NO: 669 - protein, 21 aa, synthetic construct
QAEDHPQNPPEDPNQDPPEDD

SEQ ID NO: 670 - protein, 21 aa, synthetic construct
AEDHPQNPPEDPNQDPPEDDS

SEQ ID NO: 671 - protein, 22 aa, synthetic construct
PQQAEDHPQNPPEDPNQDPPED

SEQ ID NO: 672 - protein, 22 aa, synthetic construct
QQAEDHPQNPPEDPNQDPPEDD

SEQ ID NO: 673 - protein, 22 aa, synthetic construct
QAEDHPQNPPEDPNQDPPEDDS

SEQ ID NO: 674 - protein, 23 aa, synthetic construct
DGEAGPQQAEDHPQNPPEDPNQD

SEQ ID NO: 675 - protein, 23 aa, synthetic construct
GPQQAEDHPQNPPEDPNQDPPED

SEQ ID NO: 676 - protein, 23 aa, synthetic construct
PQQAEDHPQNPPEDPNQDPPEDD

SEQ ID NO: 677 - protein, 23 aa, synthetic construct
QQAEDHPQNPPEDPNQDPPEDDS

SEQ ID NO: 678 - protein, 24 aa, synthetic construct
GPQQAEDHPQNPPEDPNQDPPEDD

SEQ ID NO: 679 - protein, 24 aa, synthetic construct
PQQAEDHPQNPPEDPNQDPPEDDS

SEQ ID NO: 680 - protein, 25 aa, synthetic construct
EAGPQQAEDHPQNPPEDPNQDPPED

SEQ ID NO: 681 - protein, 25 aa, synthetic construct
AGPQQAEDHPQNPPEDPNQDPPEDD

SEQ ID NO: 682 - protein, 25 aa, synthetic construct
GPQQAEDHPQNPPEDPNQDPPEDDS

SEQ ID NO: 683 - protein, 26 aa, synthetic construct
DGEAGPQQAEDHPQNPPEDPNQDPPE

SEQ ID NO: 684 - protein, 26 aa, synthetic construct
GEAGPQQAEDHPQNPPEDPNQDPPED

SEQ ID NO: 685 - protein, 26 aa, synthetic construct
EAGPQQAEDHPQNPPEDPNQDPPEDD

SEQ ID NO: 686 - protein, 26 aa, synthetic construct
AGPQQAEDHPQNPPEDPNQDPPEDDS

SEQ ID NO: 687 - protein, 27 aa, synthetic construct
DGEAGPQQAEDHPQNPPEDPNQDPPED

SEQ ID NO: 688 - protein, 27 aa, synthetic construct
GEAGPQQAEDHPQNPPEDPNQDPPEDD

SEQ ID NO: 689 - protein, 27 aa, synthetic construct
EAGPQQAEDHPQNPPEDPNQDPPEDDS

SEQ ID NO: 690 - protein, 28 aa, synthetic construct
DGEAGPQQAEDHPQNPPEDPNQDPPEDD

SEQ ID NO: 691 - protein, 28 aa, synthetic construct
GEAGPQQAEDHPQNPPEDPNQDPPEDDS

SEQ ID NO: 692 - protein, 29 aa, synthetic construct
DGEAGPQQAEDHPQNPPEDPNQDPPEDDS

SEQ ID NO: 693 - protein, 31 aa, synthetic construct
PAGDGEAGPQQAEDHPQNPPEDPNQDPPEDD

SEQ ID NO: 694 - protein, 31 aa, synthetic construct
AGDGEAGPQQAEDHPQNPPEDPNQDPPEDDS

SEQ ID NO: 695 - protein, 32 aa, synthetic construct
PAGDGEAGPQQAEDHPQNPPEDPNQDPPEDDS

SEQ ID NO: 696 - protein, 20 aa, synthetic construct
QNQKEAEEPGPDSENSQENP

SEQ ID NO: 697 - protein, 20 aa, synthetic construct
NQKEAEEPGPDSENSQENPP

SEQ ID NO: 698 - protein, 20 aa, synthetic construct
DQNESQSPQEPEEGPSEDDK

SEQ ID NO: 699 - protein, 20 aa, synthetic construct
QNESQSPQEPEEGPSEDDKA

SEQ ID NO: 700 - protein, 20 aa, synthetic construct
NESQSPQEPEEGPSEDDKAE

SEQ ID NO: 701 - protein, 21 aa, synthetic construct
DQNESQSPQEPEEGPSEDDKA

SEQ ID NO: 702 - protein, 21 aa, synthetic construct
QNESQSPQEPEEGPSEDDKAE

SEQ ID NO: 703 - protein, 21 aa, synthetic construct
NESQSPQEPEEGPSEDDKAEG

SEQ ID NO: 704 - protein, 21 aa, synthetic construct
ESQSPQEPEEGPSEDDKAEGE

SEQ ID NO: 705 - protein, 21 aa, synthetic construct
SQSPQEPEEGPSEDDKAEGEE

SEQ ID NO: 706 - protein, 22 aa, synthetic construct
DQNESQSPQEPEEGPSEDDKAE

SEQ ID NO: 707 - protein, 22 aa, synthetic construct
QNESQSPQEPEEGPSEDDKAEG

SEQ ID NO: 708 - protein, 22 aa, synthetic construct
NESQSPQEPEEGPSEDDKAEGE

SEQ ID NO: 709 - protein, 23 aa, synthetic construct
DQNESQSPQEPEEGPSEDDKAEG

SEQ ID NO: 710 - protein, 23 aa, synthetic construct
QNESQSPQEPEEGPSEDDKAEGE

SEQ ID NO: 711 - protein, 23 aa, synthetic construct
NESQSPQEPEEGPSEDDKAEGEE

SEQ ID NO: 712 - protein, 24 aa, synthetic construct
DQNESQSPQEPEEGPSEDDKAEGE

SEQ ID NO: 713 - protein, 24 aa, synthetic construct
QNESQSPQEPEEGPSEDDKAEGEE

SEQ ID NO: 714 - protein, 25 aa, synthetic construct
DQNESQSPQEPEEGPSEDDKAEGEE

SEQ ID NO: 715 - protein, 25 aa, synthetic construct
QNESQSPQEPEEGPSEDDKAEGEEE

SEQ ID NO: 716 - protein, 21 aa, synthetic construct
XEASSSEEEPPSPDDKENQAP

SEQ ID NO: 717 - protein, 21 aa, synthetic construct
PASSSSNPEEGPEEDREAESE

SEQ ID NO: 718 - protein, 20 aa, synthetic construct
DKPEEEDDEAQQPQPQSGPE

SEQ ID NO: 719 - protein, 20 aa, synthetic construct
KPEEEDDEAQQPQPQSGPEE

SEQ ID NO: 720 - protein, 21 aa, synthetic construct
AGEGDKPEEEDDEAQQPQPQS

SEQ ID NO: 721 - protein, 21 aa, synthetic construct
EGDKPEEEDDEAQQPQPQSGP

SEQ ID NO: 722 - protein, 21 aa, synthetic construct
GDKPEEEDDEAQQPQPQSGPE

SEQ ID NO: 723 - protein, 21 aa, synthetic construct
DKPEEEDDEAQQPQPQSGPEE

SEQ ID NO: 724 - protein, 21 aa, synthetic construct
KPEEEDDEAQQPQPQSGPEEA

SEQ ID NO: 725 - protein, 21 aa, synthetic construct
PEEEDDEAQQPQPQSGPEEAE

SEQ ID NO: 726 - protein, 21 aa, synthetic construct
EEDDEAQQPQPQSGPEEAEEG

SEQ ID NO: 727 - protein, 21 aa, synthetic construct
EDDEAQQPQPQSGPEEAEEGE

SEQ ID NO: 728 - protein, 21 aa, synthetic construct
DDEAQQPQPQSGPEEAEEGEE

SEQ ID NO: 729 - protein, 21 aa, synthetic construct
DEAQQPQPQSGPEEAEEGEEE

SEQ ID NO: 730 - protein, 21 aa, synthetic construct
EAQQPQPQSGPEEAEEGEEEE

SEQ ID NO: 731 - protein, 21 aa, synthetic construct
QQPQPQSGPEEAEEGEEEEAE

SEQ ID NO: 732 - protein, 21 aa, synthetic construct
QPQPQSGPEEAEEGEEEEAER

SEQ ID NO: 733 - protein, 22 aa, synthetic construct
EGDKPEEEDDEAQQPQPQSGPE

SEQ ID NO: 734 - protein, 22 aa, synthetic construct
GDKPEEEDDEAQQPQPQSGPEE

SEQ ID NO: 735 - protein, 22 aa, synthetic construct
DKPEEEDDEAQQPQPQSGPEEA

SEQ ID NO: 736 - protein, 22 aa, synthetic construct
KPEEEDDEAQQPQPQSGPEEAE

SEQ ID NO: 737 - protein, 22 aa, synthetic construct
QQPQPQSGPEEAEEGEEEEAER

SEQ ID NO: 738 - protein, 23 aa, synthetic construct
EGDKPEEEDDEAQQPQPQSGPEE

SEQ ID NO: 739 - protein, 23 aa, synthetic construct
GDKPEEEDDEAQQPQPQSGPEEA

SEQ ID NO: 740 - protein, 23 aa, synthetic construct
DKPEEEDDEAQQPQPQSGPEEAE

SEQ ID NO: 741 - protein, 23 aa, synthetic construct
KPEEEDDEAQQPQPQSGPEEAEE

SEQ ID NO: 742 - protein, 23 aa, synthetic construct
PEEEDDEAQQPQPQSGPEEAEEG

SEQ ID NO: 743 - protein, 24 aa, synthetic construct
EGDKPEEEDDEAQQPQPQSGPEEA

SEQ ID NO: 744 - protein, 24 aa, synthetic construct
GDKPEEEDDEAQQPQPQSGPEEAE

SEQ ID NO: 745 - protein, 24 aa, synthetic construct
KPEEEDDEAQQPQPQSGPEEAEEG

SEQ ID NO: 746 - protein, 25 aa, synthetic construct
EGDKPEEEDDEAQQPQPQSGPEEAE

SEQ ID NO: 747 - protein, 25 aa, synthetic construct
GDKPEEEDDEAQQPQPQSGPEEAEE

SEQ ID NO: 748 - protein, 25 aa, synthetic construct
DKPEEEDDEAQQPQPQSGPEEAEEG

SEQ ID NO: 749 - protein, 25 aa, synthetic construct
KPEEEDDEAQQPQPQSGPEEAEEGE

SEQ ID NO: 750 - protein, 20 aa, synthetic construct
PEEEHAPGEDESSPQPSQPS

SEQ ID NO: 751 - protein, 20 aa, synthetic construct
XPPPEESSDSEPEAEPGSPQ

SEQ ID NO: 752 - protein, 20 aa, synthetic construct
PPPEESSDSEPEAEPGSPQK

SEQ ID NO: 753 - protein, 20 aa, synthetic construct
KEPKEEKKDDDEEAPKPSSD

SEQ ID NO: 754 - protein, 20 aa, synthetic construct
SEEEKPPEEDKEEEEEKKAP

SEQ ID NO: 755 - protein, 21 aa, synthetic construct
PAEEDEDDPEQEKEAGEPGRP

SEQ ID NO: 756 - protein, 20 aa, synthetic construct
PEPEPEPEPEPEPAPEPEPE

SEQ ID NO: 757 - protein, 20 aa, synthetic construct
EPEPEPEPEPEPAPEPEPEP

SEQ ID NO: 758 - protein, 20 aa, synthetic construct
DEDPEEDGSPDPEPSPEPEP

SEQ ID NO: 759 - protein, 20 aa, synthetic construct
EDPEEDGSPDPEPSPEPEPK

SEQ ID NO: 760 - protein, 21 aa, synthetic construct
DEDPEEDGSPDPEPSPEPEPK

SEQ ID NO: 761 - protein, 22 aa, synthetic construct
DEDPEEDGSPDPEPSPEPEPKP

SEQ ID NO: 762 - protein, 20 aa, synthetic construct
KPEDKDPRDPEESKEPKEEK

SEQ ID NO: 763 - protein, 20 aa, synthetic construct
PEDKDPRDPEESKEPKEEKQ

SEQ ID NO: 764 - protein, 20 aa, synthetic construct
EDKDPRDPEESKEPKEEKQR

SEQ ID NO: 765 - protein, 20 aa, synthetic construct
KRNDSEEEERERDEEQEPPP

SEQ ID NO: 766 - protein, 20 aa, synthetic construct
PEEEPDDQDAPDEHEPSPSE

SEQ ID NO: 767 - protein, 20 aa, synthetic construct
EEEPDDQDAPDEHEPSPSED

SEQ ID NO: 768 - protein, 21 aa, synthetic construct
PEEEPDDQDAPDEHEPSPSED

SEQ ID NO: 769 - protein, 21 aa, synthetic construct
EEEPDDQDAPDEHEPSPSEDA

SEQ ID NO: 770 - protein, 21 aa, synthetic construct
EEPDDQDAPDEHEPSPSEDAP

SEQ ID NO: 771 - protein, 22 aa, synthetic construct
PEEEPDDQDAPDEHEPSPSEDA

SEQ ID NO: 772 - protein, 22 aa, synthetic construct
EEEPDDQDAPDEHEPSPSEDAP

SEQ ID NO: 773 - protein, 23 aa, synthetic construct
PEEEPDDQDAPDEHEPSPSEDAP

SEQ ID NO: 774 - protein, 20 aa, synthetic construct
SPAPSPDSDSDSDSDGEEEE

SEQ ID NO: 775 - protein, 20 aa, synthetic construct
PAPSPDSDSDSDSDGEEEEE

SEQ ID NO: 776 - protein, 22 aa, synthetic construct
PSPAPSPDSDSDSDSDGEEEEE

SEQ ID NO: 777 - protein, 23 aa, synthetic construct
PSPAPSPDSDSDSDSDGEEEEEE

SEQ ID NO: 778 - protein, 24 aa, synthetic construct
APSPAPSPDSDSDSDSDGEEEEEE

SEQ ID NO: 779 - protein, 24 aa, synthetic construct
PSPAPSPDSDSDSDSDGEEEEEEE

SEQ ID NO: 780 - protein, 25 aa, synthetic construct
APSPAPSPDSDSDSDSDGEEEEEEE

SEQ ID NO: 781 - protein, 25 aa, synthetic construct
PSPAPSPDSDSDSDSDGEEEEEEEG

SEQ ID NO: 782 - protein, 26 aa, synthetic construct
GAPSPAPSPDSDSDSDSDGEEEEEEE

SEQ ID NO: 783 - protein, 26 aa, synthetic construct
APSPAPSPDSDSDSDSDGEEEEEEEG

SEQ ID NO: 784 - protein, 26 aa, synthetic construct
PSPAPSPDSDSDSDSDGEEEEEEEGE

SEQ ID NO: 785 - protein, 21 aa, synthetic construct
EKNDEDEPQKPEDKGDPEGPE

SEQ ID NO: 786 - protein, 22 aa, synthetic construct
EKNDEDEPQKPEDKGDPEGPEA

SEQ ID NO: 787 - protein, 20 aa, synthetic construct
EDEEEEEEEEEEEDEGPAPP

SEQ ID NO: 788 - protein, 20 aa, synthetic construct
DEEEEEEEEEEEDEGPAPPS

SEQ ID NO: 789 - protein, 20 aa, synthetic construct
EREPDPPDDRDASDGEDEKP

SEQ ID NO: 790 - protein, 20 aa, synthetic construct
REPDPPDDRDASDGEDEKPP

SEQ ID NO: 791 - protein, 21 aa, synthetic construct
GEREPDPPDDRDASDGEDEKP

SEQ ID NO: 792 - protein, 21 aa, synthetic construct
EREPDPPDDRDASDGEDEKPP

SEQ ID NO: 793 - protein, 22 aa, synthetic construct
EGEREPDPPDDRDASDGEDEKP

SEQ ID NO: 794 - protein, 22 aa, synthetic construct
GEREPDPPDDRDASDGEDEKPP

SEQ ID NO: 795 - protein, 23 aa, synthetic construct
EGEREPDPPDDRDASDGEDEKPP

SEQ ID NO: 796 - protein, 22 aa, synthetic construct
EEEEEQPGKAPDPQDPQDAESD

SEQ ID NO: 797 - protein, 20 aa, synthetic construct
QEPEEKQEPEEKQEPEEKQK

SEQ ID NO: 798 - protein, 20 aa, synthetic construct
EPEEKQEPEEKQEPEEKQKP

SEQ ID NO: 799 - protein, 20 aa, synthetic construct
PEEKQEPEEKQEPEEKQKPE

SEQ ID NO: 800 - protein, 20 aa, synthetic construct
EEKQEPEEKQEPEEKQKPEA

SEQ ID NO: 801 - protein, 21 aa, synthetic construct
EPEEKQEPEEKQEPEEKQKPE

SEQ ID NO: 802 - protein, 22 aa, synthetic construct
QEPEEKQEPEEKQEPEEKQKPE

SEQ ID NO: 803 - protein, 22 aa, synthetic construct
EPEEKQEPEEKQEPEEKQKPEA

SEQ ID NO: 804 - protein, 20 aa, synthetic construct
AGDDDDDDDDSPDPESPDDS

SEQ ID NO: 805 - protein, 20 aa, synthetic construct
GDDDDDDDDSPDPESPDDSE

SEQ ID NO: 806 - protein, 20 aa, synthetic construct
DDDDDDDDSPDPESPDDSES

SEQ ID NO: 807 - protein, 20 aa, synthetic construct
DDDDDDDSPDPESPDDSESD

SEQ ID NO: 808 - protein, 20 aa, synthetic construct
DDDDDDSPDPESPDDSESDS

SEQ ID NO: 809 - protein, 20 aa, synthetic construct
DDDDDSPDPESPDDSESDSE

SEQ ID NO: 810 - protein, 20 aa, synthetic construct
DDDDSPDPESPDDSESDSES

SEQ ID NO: 811 - protein, 20 aa, synthetic construct
DDDSPDPESPDDSESDSESE

SEQ ID NO: 812 - protein, 20 aa, synthetic construct
DDSPDPESPDDSESDSESEK

SEQ ID NO: 813 - protein, 20 aa, synthetic construct
DSPDPESPDDSESDSESEKE

SEQ ID NO: 814 - protein, 20 aa, synthetic construct
SPDPESPDDSESDSESEKEE

SEQ ID NO: 815 - protein, 20 aa, synthetic construct
PDPESPDDSESDSESEKEES

SEQ ID NO: 816 - protein, 21 aa, synthetic construct
DPDQPREDPAEEEKEEKDAPE

SEQ ID NO: 817 - protein, 20 aa, synthetic construct
EGKPENESEPKHEEEPKPEE

SEQ ID NO: 818 - protein, 20 aa, synthetic construct
PENESEPKHEEEPKPEEKPE

SEQ ID NO: 819 - protein, 20 aa, synthetic construct
ENESEPKHEEEPKPEEKPEE

SEQ ID NO: 820 - protein, 20 aa, synthetic construct
NESEPKHEEEPKPEEKPEEE

SEQ ID NO: 821 - protein, 20 aa, synthetic construct
ESEPKHEEEPKPEEKPEEEE

SEQ ID NO: 822 - protein, 20 aa, synthetic construct
SEPKHEEEPKPEEKPEEEEK

SEQ ID NO: 823 - protein, 21 aa, synthetic construct
PENESEPKHEEEPKPEEKPEE

SEQ ID NO: 824 - protein, 21 aa, synthetic construct
ENESEPKHEEEPKPEEKPEEE

SEQ ID NO: 825 - protein, 21 aa, synthetic construct
NESEPKHEEEPKPEEKPEEEE

SEQ ID NO: 826 - protein, 21 aa, synthetic construct
ESEPKHEEEPKPEEKPEEEEK

SEQ ID NO: 827 - protein, 22 aa, synthetic construct
KPENESEPKHEEEPKPEEKPEE

SEQ ID NO: 828 - protein, 22 aa, synthetic construct
PENESEPKHEEEPKPEEKPEEE

SEQ ID NO: 829 - protein, 22 aa, synthetic construct
ENESEPKHEEEPKPEEKPEEEE

SEQ ID NO: 830 - protein, 22 aa, synthetic construct
NESEPKHEEEPKPEEKPEEEEK

SEQ ID NO: 831 - protein, 23 aa, synthetic construct
KPENESEPKHEEEPKPEEKPEEE

SEQ ID NO: 832 - protein, 23 aa, synthetic construct
PENESEPKHEEEPKPEEKPEEEE

SEQ ID NO: 833 - protein, 23 aa, synthetic construct
ENESEPKHEEEPKPEEKPEEEEK

SEQ ID NO: 834 - protein, 24 aa, synthetic construct
EGKPENESEPKHEEEPKPEEKPEE

SEQ ID NO: 835 - protein, 24 aa, synthetic construct
GKPENESEPKHEEEPKPEEKPEEE

SEQ ID NO: 836 - protein, 24 aa, synthetic construct
KPENESEPKHEEEPKPEEKPEEEE

SEQ ID NO: 837 - protein, 24 aa, synthetic construct
PENESEPKHEEEPKPEEKPEEEEK

SEQ ID NO: 838 - protein, 25 aa, synthetic construct
EGKPENESEPKHEEEPKPEEKPEEE

SEQ ID NO: 839 - protein, 25 aa, synthetic construct
GKPENESEPKHEEEPKPEEKPEEEE

SEQ ID NO: 840 - protein, 25 aa, synthetic construct
KPENESEPKHEEEPKPEEKPEEEEK

SEQ ID NO: 841 - protein, 26 aa, synthetic construct
EGKPENESEPKHEEEPKPEEKPEEEE

SEQ ID NO: 842 - protein, 26 aa, synthetic construct
GKPENESEPKHEEEPKPEEKPEEEEK

SEQ ID NO: 843 - protein, 27 aa, synthetic construct
EGKPENESEPKHEEEPKPEEKPEEEEK

SEQ ID NO: 844 - protein, 20 aa, synthetic construct
KPPEEDPEEQAEENPEGEQP

SEQ ID NO: 845 - protein, 20 aa, synthetic construct
PPEEDPEEQAEENPEGEQPE

SEQ ID NO: 846 - protein, 20 aa, synthetic construct
PEEDPEEQAEENPEGEQPEE

SEQ ID NO: 847 - protein, 21 aa, synthetic construct
KPPEEDPEEQAEENPEGEQPE

SEQ ID NO: 848 - protein, 21 aa, synthetic construct
PPEEDPEEQAEENPEGEQPEE

SEQ ID NO: 849 - protein, 22 aa, synthetic construct
KPPEEDPEEQAEENPEGEQPEE

SEQ ID NO: 850 - protein, 20 aa, synthetic construct
PDDDDESEDHDDPDNAHESP

SEQ ID NO: 851 - protein, 20 aa, synthetic construct
GEPEPEPEPEPEPEPESEPE

SEQ ID NO: 852 - protein, 20 aa, synthetic construct
EPEPEPEPEPEPEPESEPEP

SEQ ID NO: 853 - protein, 20 aa, synthetic construct
PEPEPEPEPEPEPESEPEPE

SEQ ID NO: 854 - protein, 20 aa, synthetic construct
EPEPEPEPEPEPESEPEPEP

SEQ ID NO: 855 - protein, 20 aa, synthetic construct
PEPEPEPEPEPESEPEPEPE

SEQ ID NO: 856 - protein, 21 aa, synthetic construct
GGEPEPEPEPEPEPEPESEPE

SEQ ID NO: 857 - protein, 21 aa, synthetic construct
GEPEPEPEPEPEPEPESEPEP

SEQ ID NO: 858 - protein, 21 aa, synthetic construct
EPEPEPEPEPEPEPESEPEPE

SEQ ID NO: 859 - protein, 21 aa, synthetic construct
PEPEPEPEPEPEPESEPEPEP

SEQ ID NO: 860 - protein, 21 aa, synthetic construct
EPEPEPEPEPEPESEPEPEPE

SEQ ID NO: 861 - protein, 22 aa, synthetic construct
GGEPEPEPEPEPEPEPESEPEP

SEQ ID NO: 862 - protein, 22 aa, synthetic construct
GEPEPEPEPEPEPEPESEPEPE

SEQ ID NO: 863 - protein, 22 aa, synthetic construct
EPEPEPEPEPEPEPESEPEPEP

SEQ ID NO: 864 - protein, 22 aa, synthetic construct
PEPEPEPEPEPEPESEPEPEPE

SEQ ID NO: 865 - protein, 23 aa, synthetic construct
GGEPEPEPEPEPEPEPESEPEPE

SEQ ID NO: 866 - protein, 23 aa, synthetic construct
GEPEPEPEPEPEPEPESEPEPEP

SEQ ID NO: 867 - protein, 23 aa, synthetic construct
EPEPEPEPEPEPEPESEPEPEPE

SEQ ID NO: 868 - protein, 24 aa, synthetic construct
GGEPEPEPEPEPEPEPESEPEPEP

SEQ ID NO: 869 - protein, 24 aa, synthetic construct
GEPEPEPEPEPEPEPESEPEPEPE

SEQ ID NO: 870 - protein, 25 aa, synthetic construct
GGEPEPEPEPEPEPEPESEPEPEPE

SEQ ID NO: 871 - protein, 20 aa, synthetic construct
PEEEPDDQDAPDEHESPPPE

SEQ ID NO: 872 - protein, 20 aa, synthetic construct
EEEPDDQDAPDEHESPPPED

SEQ ID NO: 873 - protein, 21 aa, synthetic construct
PEEEPDDQDAPDEHESPPPED

SEQ ID NO: 874 - protein, 21 aa, synthetic construct
EEEPDDQDAPDEHESPPPEDA

SEQ ID NO: 875 - protein, 21 aa, synthetic construct
EEPDDQDAPDEHESPPPEDAP

SEQ ID NO: 876 - protein, 22 aa, synthetic construct
PEEEPDDQDAPDEHESPPPEDA

SEQ ID NO: 877 - protein, 22 aa, synthetic construct
EEEPDDQDAPDEHESPPPEDAP

SEQ ID NO: 878 - protein, 23 aa, synthetic construct
PEEEPDDQDAPDEHESPPPEDAP

SEQ ID NO: 879 - protein, 20 aa, synthetic construct
GPSSDDENEEESKPEKEDEP

SEQ ID NO: 880 - protein, 20 aa, synthetic construct
PSSDDENEEESKPEKEDEPQ

SEQ ID NO: 881 - protein, 20 aa, synthetic construct
SDDSDSEKRRPEEQEEEPQP

SEQ ID NO: 882 - protein, 20 aa, synthetic construct
DDSDSEKRRPEEQEEEPQPR

SEQ ID NO: 883 - protein, 20 aa, synthetic construct
DPREGEEEEEEDEPDPEAPE

SEQ ID NO: 884 - protein, 20 aa, synthetic construct
PREGEEEEEEDEPDPEAPEN

SEQ ID NO: 885 - protein, 21 aa, synthetic construct
DEEPGDPREGEEEEEEDEPDP

SEQ ID NO: 886 - protein, 21 aa, synthetic construct
EEPGDPREGEEEEEEDEPDPE

SEQ ID NO: 887 - protein, 21 aa, synthetic construct
EPGDPREGEEEEEEDEPDPEA

SEQ ID NO: 888 - protein, 21 aa, synthetic construct
PGDPREGEEEEEEDEPDPEAP

SEQ ID NO: 889 - protein, 21 aa, synthetic construct
GDPREGEEEEEEDEPDPEAPE

SEQ ID NO: 890 - protein, 21 aa, synthetic construct
DPREGEEEEEEDEPDPEAPEN

SEQ ID NO: 891 - protein, 21 aa, synthetic construct
PREGEEEEEEDEPDPEAPENG

SEQ ID NO: 892 - protein, 22 aa, synthetic construct
EDEEPGDPREGEEEEEEDEPDP

SEQ ID NO: 893 - protein, 22 aa, synthetic construct
DEEPGDPREGEEEEEEDEPDPE

SEQ ID NO: 894 - protein, 22 aa, synthetic construct
EEPGDPREGEEEEEEDEPDPEA

SEQ ID NO: 895 - protein, 22 aa, synthetic construct
EPGDPREGEEEEEEDEPDPEAP

SEQ ID NO: 896 - protein, 22 aa, synthetic construct
PGDPREGEEEEEEDEPDPEAPE

SEQ ID NO: 897 - protein, 22 aa, synthetic construct
GDPREGEEEEEEDEPDPEAPEN

SEQ ID NO: 898 - protein, 22 aa, synthetic construct
DPREGEEEEEEDEPDPEAPENG

SEQ ID NO: 899 - protein, 22 aa, synthetic construct
PREGEEEEEEDEPDPEAPENGS

SEQ ID NO: 900 - protein, 23 aa, synthetic construct
PEEEDEEPGDPREGEEEEEEDEP

SEQ ID NO: 901 - protein, 23 aa, synthetic construct
EEDEEPGDPREGEEEEEEDEPDP

SEQ ID NO: 902 - protein, 23 aa, synthetic construct
EDEEPGDPREGEEEEEEDEPDPE

SEQ ID NO: 903 - protein, 23 aa, synthetic construct
DEEPGDPREGEEEEEEDEPDPEA

SEQ ID NO: 904 - protein, 23 aa, synthetic construct
EEPGDPREGEEEEEEDEPDPEAP

SEQ ID NO: 905 - protein, 23 aa, synthetic construct
EPGDPREGEEEEEEDEPDPEAPE

SEQ ID NO: 906 - protein, 23 aa, synthetic construct
PGDPREGEEEEEEDEPDPEAPEN

SEQ ID NO: 907 - protein, 23 aa, synthetic construct
DPREGEEEEEEDEPDPEAPENGS

SEQ ID NO: 908 - protein, 24 aa, synthetic construct
APEEEDEEPGDPREGEEEEEEDEP

SEQ ID NO: 909 - protein, 24 aa, synthetic construct
PEEEDEEPGDPREGEEEEEEDEPD

SEQ ID NO: 910 - protein, 24 aa, synthetic construct
EEEDEEPGDPREGEEEEEEDEPDP

SEQ ID NO: 911 - protein, 24 aa, synthetic construct
EEDEEPGDPREGEEEEEEDEPDPE

SEQ ID NO: 912 - protein, 24 aa, synthetic construct
EDEEPGDPREGEEEEEEDEPDPEA

SEQ ID NO: 913 - protein, 24 aa, synthetic construct
DEEPGDPREGEEEEEEDEPDPEAP

SEQ ID NO: 914 - protein, 24 aa, synthetic construct
EEPGDPREGEEEEEEDEPDPEAPE

SEQ ID NO: 915 - protein, 24 aa, synthetic construct
EPGDPREGEEEEEEDEPDPEAPEN

SEQ ID NO: 916 - protein, 25 aa, synthetic construct
AAPEEEDEEPGDPREGEEEEEEDEP

SEQ ID NO: 917 - protein, 25 aa, synthetic construct
APEEEDEEPGDPREGEEEEEEDEPD

SEQ ID NO: 918 - protein, 25 aa, synthetic construct
PEEEDEEPGDPREGEEEEEEDEPDP

SEQ ID NO: 919 - protein, 25 aa, synthetic construct
EEEDEEPGDPREGEEEEEEDEPDPE

SEQ ID NO: 920 - protein, 25 aa, synthetic construct
EEDEEPGDPREGEEEEEEDEPDPEA

SEQ ID NO: 921 - protein, 25 aa, synthetic construct
EDEEPGDPREGEEEEEEDEPDPEAP

SEQ ID NO: 922 - protein, 25 aa, synthetic construct
DEEPGDPREGEEEEEEDEPDPEAPE

SEQ ID NO: 923 - protein, 25 aa, synthetic construct
EEPGDPREGEEEEEEDEPDPEAPEN

SEQ ID NO: 924 - protein, 26 aa, synthetic construct
AAPEEEDEEPGDPREGEEEEEEDEPD

SEQ ID NO: 925 - protein, 26 aa, synthetic construct
APEEEDEEPGDPREGEEEEEEDEPDP

SEQ ID NO: 926 - protein, 26 aa, synthetic construct
PEEEDEEPGDPREGEEEEEEDEPDPE

SEQ ID NO: 927 - protein, 26 aa, synthetic construct
EEEDEEPGDPREGEEEEEEDEPDPEA

SEQ ID NO: 928 - protein, 26 aa, synthetic construct
EEDEEPGDPREGEEEEEEDEPDPEAP

SEQ ID NO: 929 - protein, 26 aa, synthetic construct
EDEEPGDPREGEEEEEEDEPDPEAPE

SEQ ID NO: 930 - protein, 26 aa, synthetic construct
DEEPGDPREGEEEEEEDEPDPEAPEN

SEQ ID NO: 931 - protein, 27 aa, synthetic construct
AAPEEEDEEPGDPREGEEEEEEDEPDP

SEQ ID NO: 932 - protein, 27 aa, synthetic construct
APEEEDEEPGDPREGEEEEEEDEPDPE

SEQ ID NO: 933 - protein, 27 aa, synthetic construct
PEEEDEEPGDPREGEEEEEEDEPDPEA

SEQ ID NO: 934 - protein, 27 aa, synthetic construct
EEEDEEPGDPREGEEEEEEDEPDPEAP

SEQ ID NO: 935 - protein, 27 aa, synthetic construct
EEDEEPGDPREGEEEEEEDEPDPEAPE

SEQ ID NO: 936 - protein, 27 aa, synthetic construct
EDEEPGDPREGEEEEEEDEPDPEAPEN

SEQ ID NO: 937 - protein, 28 aa, synthetic construct
AAPEEEDEEPGDPREGEEEEEEDEPDPE

SEQ ID NO: 938 - protein, 28 aa, synthetic construct
APEEEDEEPGDPREGEEEEEEDEPDPEA

SEQ ID NO: 939 - protein, 28 aa, synthetic construct
PEEEDEEPGDPREGEEEEEEDEPDPEAP

SEQ ID NO: 940 - protein, 28 aa, synthetic construct
EEEDEEPGDPREGEEEEEEDEPDPEAPE

SEQ ID NO: 941 - protein, 28 aa, synthetic construct
EEDEEPGDPREGEEEEEEDEPDPEAPEN

SEQ ID NO: 942 - protein, 29 aa, synthetic construct
APEEEDEEPGDPREGEEEEEEDEPDPEAP

SEQ ID NO: 943 - protein, 29 aa, synthetic construct
PEEEDEEPGDPREGEEEEEEDEPDPEAPE

SEQ ID NO: 944 - protein, 29 aa, synthetic construct
EEEDEEPGDPREGEEEEEEDEPDPEAPEN

SEQ ID NO: 945 - protein, 30 aa, synthetic construct
APEEEDEEPGDPREGEEEEEEDEPDPEAPE

SEQ ID NO: 946 - protein, 30 aa, synthetic construct
PEEEDEEPGDPREGEEEEEEDEPDPEAPEN

SEQ ID NO: 947 - protein, 31 aa, synthetic construct
PRGAAAPEEEDEEPGDPREGEEEEEEDEPDP

SEQ ID NO: 948 - protein, 31 aa, synthetic construct
RGAAAPEEEDEEPGDPREGEEEEEEDEPDPE

SEQ ID NO: 949 - protein, 31 aa, synthetic construct
AAPEEEDEEPGDPREGEEEEEEDEPDPEAPE

SEQ ID NO: 950 - protein, 31 aa, synthetic construct
APEEEDEEPGDPREGEEEEEEDEPDPEAPEN

SEQ ID NO: 951 - protein, 31 aa, synthetic construct
PEEEDEEPGDPREGEEEEEEDEPDPEAPENG

SEQ ID NO: 952 - protein, 31 aa, synthetic construct
EEEDEEPGDPREGEEEEEEDEPDPEAPENGS

SEQ ID NO: 953 - protein, 32 aa, synthetic construct
PRGAAAPEEEDEEPGDPREGEEEEEEDEPDPE

SEQ ID NO: 954 - protein, 32 aa, synthetic construct
AAPEEEDEEPGDPREGEEEEEEDEPDPEAPEN

SEQ ID NO: 955 - protein, 32 aa, synthetic construct
APEEEDEEPGDPREGEEEEEEDEPDPEAPENG

SEQ ID NO: 956 - protein, 32 aa, synthetic construct
PEEEDEEPGDPREGEEEEEEDEPDPEAPENGS

SEQ ID NO: 957 - protein, 33 aa, synthetic construct
AAPEEEDEEPGDPREGEEEEEEDEPDPEAPENG

SEQ ID NO: 958 - protein, 33 aa, synthetic construct
APEEEDEEPGDPREGEEEEEEDEPDPEAPENGS

SEQ ID NO: 959 - protein, 34 aa, synthetic construct
AAPEEEDEEPGDPREGEEEEEEDEPDPEAPENGS

SEQ ID NO: 960 - protein, 20 aa, synthetic construct
KPPPSEGSDEEEEEEDEEDE

SEQ ID NO: 961 - protein, 20 aa, synthetic construct
PPPSEGSDEEEEEEDEEDEE

SEQ ID NO: 962 - protein, 24 aa, synthetic construct
PPPSEGSDEEEEEEDEEDEEERKP

SEQ ID NO: 963 - protein, 25 aa, synthetic construct
KPPPSEGSDEEEEEEDEEDEEERKP

SEQ ID NO: 964 - protein, 25 aa, synthetic construct
PPPSEGSDEEEEEEDEEDEEERKPQ

SEQ ID NO: 965 - protein, 26 aa, synthetic construct
KPPPSEGSDEEEEEEDEEDEEERKPQ

SEQ ID NO: 966 - protein, 20 aa, synthetic construct
EEEEEEEEEEEEEEEEAPPP

SEQ ID NO: 967 - protein, 20 aa, synthetic construct
EEEEEEEEEEEEEEEAPPPP

SEQ ID NO: 968 - protein, 20 aa, synthetic construct
EEEEEEEEEEEEEEAPPPPR

SEQ ID NO: 969 - protein, 21 aa, synthetic construct
EEEEEEEEEEEEEEEEAPPPP

SEQ ID NO: 970 - protein, 21 aa, synthetic construct
EEEEEEEEEEEEEEEAPPPPR

SEQ ID NO: 971 - protein, 22 aa, synthetic construct
EEEEEEEEEEEEEEEEEAPPPP

SEQ ID NO: 972 - protein, 22 aa, synthetic construct
EEEEEEEEEEEEEEEEAPPPPR

SEQ ID NO: 973 - protein, 23 aa, synthetic construct
EEEEEEEEEEEEEEEEEEAPPPP

SEQ ID NO: 974 - protein, 23 aa, synthetic construct
EEEEEEEEEEEEEEEEEAPPPPR

SEQ ID NO: 975 - protein, 24 aa, synthetic construct
EEEEEEEEEEEEEEEEEEEAPPPP

SEQ ID NO: 976 - protein, 24 aa, synthetic construct
EEEEEEEEEEEEEEEEEEAPPPPR

SEQ ID NO: 977 - protein, 25 aa, synthetic construct
EEEEEEEEEEEEEEEEEEEEAPPPP

SEQ ID NO: 978 - protein, 25 aa, synthetic construct
EEEEEEEEEEEEEEEEEEEAPPPPR

SEQ ID NO: 979 - protein, 26 aa, synthetic construct
EEEEEEEEEEEEEEEEEEEEEAPPPP

SEQ ID NO: 980 - protein, 26 aa, synthetic construct
EEEEEEEEEEEEEEEEEEEEAPPPPR

SEQ ID NO: 981 - protein, 33 aa, synthetic construct
PDDDEEDEEEEEEEEEEEEEEEEEEEEEAPPPP

SEQ ID NO: 982 - protein, 21 aa, synthetic construct
EDSEEESQEEEAEGASEPPPP

SEQ ID NO: 983 - protein, 22 aa, synthetic construct
QEDSEEESQEEEAEGASEPPPP

SEQ ID NO: 984 - protein, 24 aa, synthetic construct
DHQEDSEEESQEEEAEGASEPPPP

SEQ ID NO: 985 - protein, 20 aa, synthetic construct
DQSEEEEEEEKHPPKPAKPE

SEQ ID NO: 986 - protein, 21 aa, synthetic construct
PAPAHRPPEDEGEENEGEEDE

SEQ ID NO: 987 - protein, 22 aa, synthetic construct
PAPAHRPPEDEGEENEGEEDEE

SEQ ID NO: 988 - protein, 20 aa, synthetic construct
QENGQREEEEEEKEPEAEPP

SEQ ID NO: 989 - protein, 24 aa, synthetic construct
PAEGQENGQREEEEEEKEPEAEPP

SEQ ID NO: 990 - protein, 20 aa, synthetic construct
EQEPEPEPEPEPEPEPEPEP

SEQ ID NO: 991 - protein, 20 aa, synthetic construct
QEPEPEPEPEPEPEPEPEPE

SEQ ID NO: 992 - protein, 20 aa, synthetic construct
EPEPEPEPEPEPEPEPEPEQ

SEQ ID NO: 993 - protein, 21 aa, synthetic construct
EQEPEPEPEPEPEPEPEPEPE

SEQ ID NO: 994 - protein, 21 aa, synthetic construct
QEPEPEPEPEPEPEPEPEPEQ

SEQ ID NO: 995 - protein, 22 aa, synthetic construct
EQEPEPEPEPEPEPEPEPEPEQ

SEQ ID NO: 996 - protein, 20 aa, synthetic construct
PEEEEEEEEEEEPASPPERK

SEQ ID NO: 997 - protein, 22 aa, synthetic construct
PSPGSPRGQPQDQDDDEDDEED

SEQ ID NO: 998 - protein, 23 aa, synthetic construct
PSPGSPRGQPQDQDDDEDDEEDE

SEQ ID NO: 999 - protein, 24 aa, synthetic construct
RPSPGSPRGQPQDQDDDEDDEEDE

SEQ ID NO: 1000 - protein, 24 aa, synthetic construct
PSPGSPRGQPQDQDDDEDDEEDEA

SEQ ID NO: 1001 - protein, 20 aa, synthetic construct
AEDDDEEDEEEEEEEPDPDP

SEQ ID NO: 1002 - protein, 20 aa, synthetic construct
EDDDEEDEEEEEEEPDPDPE

SEQ ID NO: 1003 - protein, 20 aa, synthetic construct
KQEPPDPEEDKEENKDDSAS

SEQ ID NO: 1004 - protein, 20 aa, synthetic construct
QEPPDPEEDKEENKDDSASK

SEQ ID NO: 1005 - protein, 20 aa, synthetic construct
EDEDEDESSEEDSEDEEPPP

SEQ ID NO: 1006 - protein, 20 aa, synthetic construct
DEDEDESSEEDSEDEEPPPK

SEQ ID NO: 1007 - protein, 20 aa, synthetic construct
EDEDESSEEDSEDEEPPPKR

SEQ ID NO: 1008 - protein, 20 aa, synthetic construct
DEDESSEEDSEDEEPPPKRR

SEQ ID NO: 1009 - protein, 26 aa, synthetic construct
PDDSRDEDEDEDESSEEDSEDEEPPP

SEQ ID NO: 1010 - protein, 30 aa, synthetic construct
PKKEPDDSRDEDEDEDESSEEDSEDEEPPP

SEQ ID NO: 1011 - protein, 31 aa, synthetic construct
PKKEPDDSRDEDEDEDESSEEDSEDEEPPPK

SEQ ID NO: 1012 - protein, 32 aa, synthetic construct
PKKEPDDSRDEDEDEDESSEEDSEDEEPPPKR

SEQ ID NO: 1013 - protein, 33 aa, synthetic construct
PKKEPDDSRDEDEDEDESSEEDSEDEEPPPKRR

SEQ ID NO: 1014 - protein, 21 aa, synthetic construct
PEEEAAEEEEEEEERPKPSRP

SEQ ID NO: 1015 - protein, 22 aa, synthetic construct
QPEEEAAEEEEEEEERPKPSRP

SEQ ID NO: 1016 - protein, 23 aa, synthetic construct
EQPEEEAAEEEEEEEERPKPSRP

SEQ ID NO: 1017 - protein, 24 aa, synthetic construct
EEQPEEEAAEEEEEEEERPKPSRP

SEQ ID NO: 1018 - protein, 25 aa, synthetic construct
EEEQPEEEAAEEEEEEEERPKPSRP

SEQ ID NO: 1019 - protein, 22 aa, synthetic construct
PEEEDDEAQQPQPQSGPEEAEE

SEQ ID NO: 1020 - protein, 24 aa, synthetic construct
DKPEEEDDEAQQPQPQSGPEEAEE

SEQ ID NO: 1021 - protein, 24 aa, synthetic construct
PEEEDDEAQQPQPQSGPEEAEEGE

SEQ ID NO: 1022 - protein, 25 aa, synthetic construct
PEEEDDEAQQPQPQSGPEEAEEGEE

SEQ ID NO: 1023 - protein, 26 aa, synthetic construct
EGDKPEEEDDEAQQPQPQSGPEEAEE

SEQ ID NO: 1024 - protein, 26 aa, synthetic construct
DKPEEEDDEAQQPQPQSGPEEAEEGE

SEQ ID NO: 1025 - protein, 26 aa, synthetic construct
KPEEEDDEAQQPQPQSGPEEAEEGEE

SEQ ID NO: 1026 - protein, 26 aa, synthetic construct
PEEEDDEAQQPQPQSGPEEAEEGEEE

SEQ ID NO: 1027 - protein, 32 aa, synthetic construct
PEEEDDEAQQPQPQSGPEEAEEGEEEEAERGP

SEQ ID NO: 1028 - protein, 33 aa, synthetic construct
KPEEEDDEAQQPQPQSGPEEAEEGEEEEAERGP

SEQ ID NO: 1029 - protein, 20 aa, synthetic construct
NNSEEEEDDDDEEEEPDKPP

SEQ ID NO: 1030 - protein, 20 aa, synthetic construct
NSEEEEDDDDEEEEPDKPPA

SEQ ID NO: 1031 - protein, 20 aa, synthetic construct
SEEEEDDDDEEEEPDKPPAN

SEQ ID NO: 1032 - protein, 125 aa, synthetic construct
QVQLQQSGGGLVQPGGSLRLSCAASGFTLDYYAIGWFRQAPGKEREGVLCISSSGGSTNYADSVKGRFTISRDNAKNTVYLQMNSLKPEDTAVYYCAADDLRCGSNWSSYFRGSWGQGTQVTVSS

SEQ ID NO: 1033 - protein, 241 aa, synthetic construct
AEVQLLESGGGLVQPGGSLRLSCAASGFTFSSYAMSWVRQAPGKGLEWVSYIASGGDTTNYADSVKGRFTISRDNSKNTLYLQMNSLRAEDTAVYYCAKGASAFDYWGQGTLVTVSSGGGGSGGGGSGGGGSTDIQMTQSPSSLSASVGDRVTITCRASQSISSYLNWYQQKPGKAPKLLIYAASYLQSGVPSRFSGSGSGTDFTLTISSLQPEDFATYYCQQSSNDPYTFGQGTKVEIKR

SEQ ID NO: 1034 - protein, 22 aa, synthetic construct
MARVKKDQAEPLHRKFERQPPG

SEQ ID NO: 1035 - protein, 242 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMRLGLFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQRGDVPPTFGQGTKVEIKAAA

SEQ ID NO: 1036 - protein, 49 aa, synthetic construct
YPYDVPDYAGSPQPVEDGEDEFCTPMACEANIQSGDSAAPMSAVHRHRL

SEQ ID NO: 1037 - protein, 49 aa, synthetic construct
NNYDVPDNAGSPQPQEDGEDEFNNPQANEANQQSGDSNNPNSAVNRHNN

SEQ ID NO: 1038 - protein, 50 aa, synthetic construct
MTRGNQRELARQKNMKKQSDSVKGKRRDDGLSAAARKQRDSEIMQQKQKK

SEQ ID NO: 1039 - protein, 83 aa, synthetic construct
MSGPNGDLGMPVEAGAEGEEDGFGEAEYAAINSMLDQINSCLDHLEEKNDHLHARLQELLESNRQTRLEFQQQLGEAPSDASP

SEQ ID NO: 1040 - protein, 99 aa, synthetic construct
MAQGQRKFQAHKPAKSKTAAAASEKNRGPRKGGRVIAPKKARVVQQQKLKKNLEVGIRKKIEHDVVMKASSSLPKKLALLKAPAKKKGAAAATSSKTPS

SEQ ID NO: 1041 - protein, 35 aa, synthetic construct
NDEYSDFEDSDFDGDYKDSDEDYKDDSENFDDGFE

SEQ ID NO: 1042 - protein, 35 aa, synthetic construct
PDEPPDPEDPDPDPDPKDPDEDPKDDPEPPDDPPE

SEQ ID NO: 1043 - protein, 35 aa, synthetic construct
NDEPPDPEDSDPDGDPKDSDEDPKDDSEPPDDGPE

(19) United States
(12) Patent Application Publication
(10) Pub. No.: US 2025/0258180 A1
(43) Pub. Date: Aug. 14, 2025

(54) PEPTIDE TAG AND NUCLEIC ACID ENCODING SAME

(71) Applicant: STAND Therapeutics Co., Ltd., Minato-ku, Tokyo (JP)

(72) Inventor: Hiroyuki KABAYAMA, Tokyo (JP)

(73) Assignee: STAND Therapeutics Co., Ltd., Minato-ku, Tokyo (JP)

(21) Appl. No.: 18/292,562

(22) PCT Filed: Jul. 26, 2022
(86) PCT No.: PCT/JP2022/028746

§ 371 Date: Jan. 26, 2024

(30) Foreign Application Priority Data
Jul. 27, 2021 (JP) 2021-122510

(51) Int. Cl. G01N 33/68; C07K 16/18

(52) U.S. Cl. CPC G01N 33/6845; C07K 16/18; C07K 2317/622; C07K 2317/80; C07K 2319/00

(57) ABSTRACT

The present disclosure provides a peptide tag, and a nucleic acid encoding the peptide tag. The peptide tag of the present disclosure can reduce an aggregation property of a protein in a cell. Specifically, the peptide tag of the present disclosure can be a peptide tag in which 5% or more and less than 45% of amino acids contained in an amino acid sequence thereof are acidic amino acids, and (b) 20% or more of the amino acids contained in the amino acid sequence are amino acids selected from the group consisting of F, P, Y, G, S, Q, N, and A.

### TECHNICAL FIELD

The present disclosure relates to a peptide tag, and a nucleic acid encoding the peptide tag.

### BACKGROUND ART

An antibody functioning in a cell, namely, an intrabody (intracellular antibody) can affect the function of the cell by recognizing and binding to an antigen (target molecule) in the cell of a higher organism. Such an antigen can be a significant intracellular therapeutic target that can be inactivated by binding to an intracellular antibody. As a research method, use of an intracellular antibody attracts attention as means for specifically inhibiting the function of a protein directly by binding to the antibody in the cell.

In case of an intracellular antibody, a hybridoma producing a monoclonal antibody recognizing an antigen is first produced by a standard method, and from the cDNA thereof, an intracellular expression vector containing a DNA encoding a single chain antibody (single chain Fv: scFv) is constructed to obtain a complex of a heavy chain (VH) and a light chain (VL) as an intracellular antibody. Recently, a phage library for presenting a human scFv from an antibody isolated from a human B cell is produced, and is used for isolation of a scFv binding to an intracellular antigen in some cases.

An antibody usually moves around in an extracellular space such as blood in a body, and recognizes an extracellular antigen to function, and hence works in the extracellular space as a premise. Accordingly, if an antibody is expressed in the cytoplasm, there arise problems of reduction of the expression level, folding causing limitation of a half-life of an antibody domain, and stability. The problem of stability of an intracellular antibody in the cytoplasm can lead to formation of an aggregation of the intracellular antibody in the cytoplasm. The formation of the aggregation can lead to reduction of a production amount of the intracellular antibody, and inhibition of expression of normal function. The same applies to a protein except for the intracellular antibody. The intracellular antibody has a characteristic of easily aggregating in particular, but a protein except for the intracellular antibody also can form an aggregation in the cytoplasm when produced in the cytoplasm.

By contrast, it has been shown that a peptide tag having an amino acid sequence containing 45% or more of acidic amino acids improves stability of an intracellular antibody (Patent Literature 1, and Non Patent Literature 1). In proposing the effectiveness of a peptide tag having an amino acid sequence containing 45% or more of acidic amino acids, Patent Literature 1 and Non Patent Literature 1 point out, as a design guideline for the peptide tag, significance of designing the peptide tag in such a manner that a charge value and a pI value are sufficiently low based not on the pH environment of the cytoplasm but on the pH environment on the surface of an endosome on the side of the cytoplasm. In Non Patent Literature 3, a membrane localization signal of HRAS is added to a heavy chain variable region of an antibody.

### CITATION LIST

### Patent Literature

Patent Literature 1: WO2019/004213

### Non Patent Literature

Non Patent Literature 1: Kabayama et al., 2020, Nature Communication, 11, 336
Non Patent Literature 2: Shubhada et al., 2012, Biochemical genetics, Vol. 50, No. 7-8, pp. 625-41
Non Patent Literature 3: Tanaka et al., 2007, EMBO Journal, 26:3250-3259

### SUMMARY OF INVENTION

The present disclosure provides a peptide tag, and a nucleic acid encoding the peptide tag. The peptide tag of the present disclosure can reduce the aggregation property of a protein in a cell.

The present inventors made earnest studies on peptide tags having various amino acid sequences, resulting in finding a peptide tag having an effect of reducing an aggregation property of a protein in a cell.

The present disclosure provides the following inventions:

[1] A peptide having an amino acid sequence with a length of, for example, 600 amino acids or less, for example, 10 to 200 amino acids (for example, 10 to 90 amino acids),
wherein (a) 5% or more and less than 45% of amino acids contained in the amino acid sequence are acidic amino acids, and
(b) 20% or more, and preferably 30% or more of the amino acids contained in the amino acid sequence are amino acids selected from the group consisting of F, P, Y, G, S, Q, N, and A, and
the peptide is preferably capable of reducing an aggregation property in a cell of a protein linked to the peptide, wherein 10% or 15% or more of the amino acids contained in the amino acid sequence are N or P.

[2] The peptide according to [1] above, wherein 30% or less, preferably 20% or less, more preferably 15% or less, and further preferably 10% or less of the amino acids contained in the amino acid sequence are amino acids selected from the group consisting of M, T, W, C, I, V, and L.

[3] The peptide according to [1] or [2] above, wherein each of A and G constitutes less than 10% of the amino acids contained in the amino acid sequence thereof.

[4] The peptide according to any one of [1] to [3] above, wherein
(a) 20% or more and less than 45% of the amino acids contained in the amino acid sequence are acidic amino acids,
(b) 30% or more and less than 70% of the amino acids contained in the amino acid sequence are amino acids selected from the group consisting of F, P, Y, G, S, Q, N, and A,
(c) 20% or less of the amino acids contained in the amino acid sequence are amino acids selected from the group consisting of M, T, W, C, I, V, and L, and
(d) each of A and G constitutes less than 10% of the amino acids contained in the amino acid sequence.

[5] A peptide having an amino acid sequence set forth in any one of SEQ ID NOs: 2 to 11.

[6] A nucleic acid encoding the peptide according to any one of [1] to [5] above.

[7] A protein expression vector comprising: the nucleic acid according to [6] above operably linked to a regulatory sequence; and a nucleic acid encoding a protein of interest in-frame to the nucleic acid according to [6] above.

[8] The protein expression vector according to [7] above, wherein the protein of interest is an antibody, or an antigen-binding fragment of an antibody.

[9] The protein expression vector according to [8] above, wherein the antigen-binding fragment of the antibody is a single chain Fv (scFv).

[10] A fusion protein of the peptide according to any one of [1] to [5] above and a protein of interest.

[11] The fusion protein according to [10] above, wherein the protein of interest is an antibody, or an antigen-binding fragment of an antibody.

[12] The fusion protein according to [11] above, wherein the antigen-binding fragment of the antibody is a single chain Fv (scFv).

[13] A protein-producing cell comprising: the nucleic acid according to [6] above operably linked to a regulatory sequence; and a nucleic acid encoding a protein of interest in-frame to the nucleic acid according to [6] above.

[14] A method for selecting or identifying an amino acid sequence having a length of 600 amino acids or less, for example, 10 to 200 amino acids (for example, 10 to 90 amino acids), the method comprising:
acquiring, from an amino acid sequence (group) (that can include an amino acid sequence (group) having a length of 10 to 200 amino acids (for example, 10 to 90 amino acids)), an amino acid sequence (group) in which (a) 5% or more and less than 45% of amino acids contained in the amino acid sequence are acidic amino acids, and (b) 20% or more of the amino acids contained in the amino acid sequence are amino acids selected from the group consisting of F, P, Y, G, S, Q, N, and A;
selecting or identifying an amino acid sequence of a peptide tag that, when the fusion protein of a peptide tag having the selected or identified amino acid sequence and a reference protein is expressed in a mammal cell (preferably in a human cell), provides reduction of a proportion of cells in which the fusion protein forms an aggregation (for example, the proportion which is not more than a predetermined value); and
obtaining a peptide tag having the amino acid sequence, or a nucleic acid encoding the peptide tag.

[15] The method according to claim 14, wherein the amino acid sequence group to be acquired is the peptide according to any one of [1] to [5] above.

[16] The method according to [14] or [15] above, wherein the amino acid sequence group to be acquired is a group of amino acid sequences encoded by coding regions of human genome.

[17] The method according to any one of [14] to [16] above, wherein the amino acid sequence to be acquired contains a neo-antigen.

[18] A peptide satisfying one or more selected from the group consisting of (a) to (h) described below, and capable of reducing an aggregation property in a cell of a protein linked to the peptide.

[19] A peptide selected from the group consisting of (A) to (AE) and (AF) to (AU) described below, and capable of reducing an aggregation property in a cell of a protein linked to the peptide.

[20] A nucleic acid encoding the peptide according to [18] above.

[21] A protein expression vector comprising: the nucleic acid according to [20] above operably linked to a regulatory sequence; and a nucleic acid encoding a protein of interest in-frame to the nucleic acid according to [20] above.

[22] A fusion protein of the peptide according to [18] above and a protein of interest.

[23] The fusion protein according to [22] above, wherein the protein of interest is an antibody, or an antigen-binding fragment of an antibody.

[24] The fusion protein according to [23] above, wherein the antigen-binding fragment of the antibody is a single chain Fv (scFv).

[25] A nucleic acid encoding the peptide according to [19] above.

[26] A protein expression vector comprising: the nucleic acid according to [25] above operably linked to a regulatory sequence; and a nucleic acid encoding a protein of interest in-frame to the nucleic acid according to [25] above.

[27] A fusion protein of the peptide according to [25] above, and a protein of interest.

[28] The fusion protein according to [26] above, wherein the protein of interest is an antibody, or an antigen-binding fragment of an antibody.

[29] The fusion protein according to [27] above, wherein the antigen-binding fragment of the antibody is a single chain Fv (scFv).

[30] The method according to [14] above, wherein the amino acid sequence (group) includes the peptide according to [18] above.

[31] The method according to [14] above, wherein the amino acid sequence (group) includes the peptide according to [19] above.

[32] The method according to [14] above, wherein the reference protein is a scFv, and the predetermined value is a value of 30% or less.

[33] The method according to [14] above, wherein the reference protein is a scFv, and the predetermined value is a value of 20% or less.

[34] The method according to [14] above, wherein the reference protein is a scFv, and the predetermined value is a value of 15% or less.

[35] The method according to [14] above, wherein the reference protein is a scFv, and the predetermined value is a value of 10% or less.

[36] The method according to [14] above, wherein the reference protein is a scFv, and the predetermined value is a value of 5% or less.

[37] The method according to [14] above, wherein the reference protein is a scFv, the amino acid sequence group includes the peptide according to (A) described below, and the amino acid sequence to be acquired preferably further satisfies a condition of (A) described below.

[38] The method according to [14] above, wherein the reference protein is a scFv, the amino acid sequence group includes the peptide according to (B) described below, and the amino acid sequence to be acquired preferably further satisfies a condition of (B) described below.

[39] The method according to [14] above, wherein the reference protein is a scFv, the amino acid sequence group includes the peptide according to (C) described below, and the amino acid sequence to be acquired preferably further satisfies a condition of (C) described below.

[40] The method according to [14] above, wherein the reference protein is a scFv, the amino acid sequence group includes the peptide according to (D) described below, and the amino acid sequence to be acquired preferably further satisfies a condition of (D) described below.

[41] The method according to [14] above, wherein the reference protein is a scFv, the amino acid sequence group includes the peptide according to (E) described below, and the amino acid sequence to be acquired preferably further satisfies a condition of (E) described below.

[42] The method according to [14] above, wherein the reference protein is a scFv, the amino acid sequence group includes the peptide according to (F) described below, and the amino acid sequence to be acquired preferably further satisfies a condition of (F) described below.

[43] The method according to [14] above, wherein the reference protein is a scFv, the amino acid sequence group includes the peptide according to (G) described below, and the amino acid sequence to be acquired preferably further satisfies a condition of (G) described below.

[44] The method according to [14] above, wherein the reference protein is a scFv, the amino acid sequence group includes the peptide according to (H) described below, and the amino acid sequence to be acquired preferably further satisfies a condition of (H) described below.

[45] The method according to [14] above, wherein the reference protein is a scFv, the amino acid sequence group includes the peptide according to (I) described below, and the amino acid sequence to be acquired preferably further satisfies a condition of (I) described below.

[46] The method according to [14] above, wherein the reference protein is a scFv, the amino acid sequence group includes the peptide according to (J) described below, and the amino acid sequence to be acquired preferably further satisfies a condition of (J) described below.

[47] The method according to [14] above, wherein the reference protein is a scFv, the amino acid sequence group includes the peptide according to (K) described below, and the amino acid sequence to be acquired preferably further satisfies a condition of (K) described below.

[48] The method according to [14] above, wherein the reference protein is a scFv, the amino acid sequence group includes the peptide according to (L) described below, and the amino acid sequence to be acquired preferably further satisfies a condition of (K) described below.

[49] The method according to [14] above, wherein the reference protein is a scFv, the amino acid sequence group includes the peptide according to (M) described below, and the amino acid sequence to be acquired preferably further satisfies a condition of (M) described below.

[50] The method according to [14] above, wherein the reference protein is a scFv, the amino acid sequence group includes the peptide according to (N) described below, and the amino acid sequence to be acquired preferably further satisfies a condition of (N) described below.

[51] The method according to [14] above, wherein the reference protein is a scFv, the amino acid sequence group includes the peptide according to (O) described below, and the amino acid sequence to be acquired preferably further satisfies a condition of (O) described below.

[52] The method according to [14] above, wherein the reference protein is a scFv, the amino acid sequence group includes the peptide according to (P) described below, and the amino acid sequence to be acquired preferably further satisfies a condition of (P) described below.

[53] The method according to [14] above, wherein the reference protein is a scFv, the amino acid sequence group includes the peptide according to (Q) described below, and the amino acid sequence to be acquired preferably further satisfies a condition of (Q) described below.

[54] The method according to [14] above, wherein the reference protein is a scFv, the amino acid sequence group includes the peptide according to (R) described below, and the amino acid sequence to be acquired preferably further satisfies a condition of (R) described below.

[55] The method according to [14] above, wherein the reference protein is a scFv, the amino acid sequence group includes the peptide according to (S) described below, and the amino acid sequence to be acquired preferably further satisfies a condition of (S) described below.

[56] The method according to [14] above, wherein the reference protein is a scFv, the amino acid sequence group includes the peptide according to (T) described below, and the amino acid sequence to be acquired preferably further satisfies a condition of (T) described below.

[57] The method according to [14] above, wherein the reference protein is a scFv, the amino acid sequence group includes the peptide according to (U) described below, and the amino acid sequence to be acquired preferably further satisfies a condition of (U) described below.

[58] The method according to [14] above, wherein the reference protein is a scFv, the amino acid sequence group includes the peptide according to (V) described below, and the amino acid sequence to be acquired preferably further satisfies a condition of (V) described below.

[59] The method according to [14] above, wherein the reference protein is a scFv, the amino acid sequence group includes the peptide according to (W) described below, and the amino acid sequence to be acquired preferably further satisfies a condition of (W) described below.

[60] The method according to [14] above, wherein the reference protein is a scFv, the amino acid sequence group includes the peptide according to (X) described below, and the amino acid sequence to be acquired preferably further satisfies a condition of (X) described below.

[61] The method according to [14] above, wherein the reference protein is a scFv, the amino acid sequence group includes the peptide according to (Y) described below, and the amino acid sequence to be acquired preferably further satisfies a condition of (Y) described below.

[62] The method according to [14] above, wherein the reference protein is a scFv, the amino acid sequence group includes the peptide according to (Z) described below, and the amino acid sequence to be acquired preferably further satisfies a condition of (Z) described below.

[63] The method according to [14] above, wherein the reference protein is a scFv, the amino acid sequence group includes the peptide according to (AA) described below, and the amino acid sequence to be acquired preferably further satisfies a condition of (AA) described below.

[64] The method according to [14] above, wherein the reference protein is a scFv, the amino acid sequence group includes the peptide according to (AB) described below, and the amino acid sequence to be acquired preferably further satisfies a condition of (AB) described below.

[65] The method according to [14] above, wherein the reference protein is a scFv, the amino acid sequence group includes the peptide according to (AC) described below, and the amino acid sequence to be acquired preferably further satisfies a condition of (AC) described below.

[66] The method according to [14] above, wherein the reference protein is a scFv, the amino acid sequence group includes the peptide according to (AD) described below, and the amino acid sequence to be acquired preferably further satisfies a condition of (AD) described below.

[67] The method according to [14] above, wherein the reference protein is a scFv, the amino acid sequence group includes the peptide according to (AE) described below, and the amino acid sequence to be acquired preferably further satisfies a condition of (AE) described below.

[67A] The method according to [14] above, wherein the reference protein is a scFv, the amino acid sequence group includes the peptide according to (AF) described below, and the amino acid sequence to be acquired preferably further satisfies a condition of (AF) described below.

[67B] The method according to [14] above, wherein the reference protein is a scFv, the amino acid sequence group includes the peptide according to (AG) described below, and the amino acid sequence to be acquired preferably further satisfies a condition of (AG) described below.

[67C] The method according to [14] above, wherein the reference protein is a scFv, the amino acid sequence group includes the peptide according to (AH) described below, and the amino acid sequence to be acquired preferably further satisfies a condition of (AH) described below.

[67D] The method according to [14] above, wherein the reference protein is a scFv, the amino acid sequence group includes the peptide according to (AI) described below, and the amino acid sequence to be acquired preferably further satisfies a condition of (AI) described below.

[67E] The method according to [14] above, wherein the reference protein is a scFv, the amino acid sequence group includes the peptide according to (AJ) described below, and the amino acid sequence to be acquired preferably further satisfies a condition of (AJ) described below.

[67F] The method according to [14] above, wherein the reference protein is a scFv, the amino acid sequence group includes the peptide according to (AK) described below, and the amino acid sequence to be acquired preferably further satisfies a condition of (AK) described below.

[67G] The method according to [14] above, wherein the reference protein is a scFv, the amino acid sequence group includes the peptide according to (AL) described below, and the amino acid sequence to be acquired preferably further satisfies a condition of (AL) described below.

[67H] The method according to [14] above, wherein the reference protein is a scFv, the amino acid sequence group includes the peptide according to (AM) described below, and the amino acid sequence to be acquired preferably further satisfies a condition of (AM) described below.

[67I] The method according to [14] above, wherein the reference protein is a scFv, the amino acid sequence group includes the peptide according to (AN) described below, and the amino acid sequence to be acquired preferably further satisfies a condition of (AN) described below.

[67J] The method according to [14] above, wherein the reference protein is a scFv, the amino acid sequence group includes the peptide according to (AO) described below, and the amino acid sequence to be acquired preferably further satisfies a condition of (AO) described below.

[67K] The method according to [14] above, wherein the reference protein is a scFv, the amino acid sequence group includes the peptide according to (AP) described below, and the amino acid sequence to be acquired preferably further satisfies a condition of (AP) described below.

[67L] The method according to [14] above, wherein the reference protein is a scFv, the amino acid sequence group includes the peptide according to (AQ) described below, and the amino acid sequence to be acquired preferably further satisfies a condition of (AQ) described below.

[67M] The method according to [14] above, wherein the reference protein is a scFv, the amino acid sequence group includes the peptide according to (AR) described below, and the amino acid sequence to be acquired preferably further satisfies a condition of (AR) described below.

[67N] The method according to [14] above, wherein the reference protein is a scFv, the amino acid sequence group includes the peptide according to (AS) described below, and the amino acid sequence to be acquired preferably further satisfies a condition of (AS) described below.

[670] The method according to [14] above, wherein the reference protein is a scFv, the amino acid sequence group includes the peptide according to (AT) described below, and the amino acid sequence to be acquired preferably further satisfies a condition of (AT) described below.

[67P] The method according to [14] above, wherein the reference protein is a scFv, the amino acid sequence group includes the peptide according to (AU) described below, and the amino acid sequence to be acquired preferably further satisfies a condition of (AU) described below.

[68] The method according to any one of [37] to [67] and [67A] to [67N] above, wherein the reference protein is a scFv, and the predetermined value is a value of 15% or less.

[69] The method according to any one of [37] to [67] and [67A] to [67N] above, wherein the reference protein is a scFv, and the predetermined value is a value of 10% or less.

[70] The method according to any one of [37] to [67] and [67A] to [67N] above, wherein the reference protein is a scFv, and the predetermined value is a value of 5% or less.

[71] The method according to any one of [37] to [70] above, wherein a proportion of cells in which the reference protein forms an aggregation is a value more than 30%.

[72] The method according to any one of [37] to [70] above, wherein a proportion of cells in which the reference protein forms an aggregation is a value in a range of 30 to 40%.

[73] The method according to any one of [37] to [70] above, wherein a rate of cells in which the reference protein forms an aggregation is a value in a range of 40 to 50%.

[74] The method according to any one of [37] to [70] above, wherein a rate of cells in which the reference protein forms an aggregation is a value in a range of 50 to 60%.

[75] The method according to any one of [37] to [70] above, wherein a rate of cells in which the reference protein forms an aggregation is a value in a range of 60 to 70%.

[76] The method according to any one of [37] to [70] above, wherein a rate of cells in which the reference protein forms an aggregation is a value in a range of 70 to 80%.

[77] The method according to any one of [37] to [70] above, wherein a rate of cells in which the reference protein forms an aggregation is a value in a range of 80 to 90%.

[78] The method according to any one of [37] to [70] above, wherein a rate of cells in which the reference protein forms an aggregation is a value in a range of 90 to 95%.

[79] The method according to any one of [37] to [70] above, wherein a rate of cells in which the reference protein forms an aggregation is a value in a range of 95 to 99%.

[80] The method according to any one of [37] to [70] above, wherein a rate of cells in which the reference protein forms an aggregation is a value in a range of 99 to 99.9%.

[81] The method according to any one of [37] to [70] above, wherein a rate of cells in which the reference protein forms an aggregation is a value in a range of 99.9 to 100%.

[82] The method according to [69] above, wherein a proportion of cells in which the reference protein forms an aggregation is a value more than 30%.

[83] The method according to [69] above, wherein a proportion of cells in which the reference protein forms an aggregation is a value in a range of 30 to 40%.

[84] The method according to [69] above, wherein a rate of cells in which the reference protein forms an aggregation is a value in a range of 40 to 50%.

[85] The method according to [69] above, wherein a rate of cells in which the reference protein forms an aggregation is a value in a range of 50 to 60%.

[86] The method according to [69] above, wherein a rate of cells in which the reference protein forms an aggregation is a value in a range of 60 to 70%.

[87] The method according to [69] above, wherein a rate of cells in which the reference protein forms an aggregation is a value in a range of 70 to 80%.

[88] The method according to [69] above, wherein a rate of cells in which the reference protein forms an aggregation is a value in a range of 80 to 90%.

[89] The method according to [69] above, wherein a rate of cells in which the reference protein forms an aggregation is a value in a range of 90 to 95%.

[91] The method according to [69] above, wherein a rate of cells in which the reference protein forms an aggregation is a value in a range of 99 to 99.9%.

[92] The method according to [69] above, wherein a rate of cells in which the reference protein forms an aggregation is a value in a range of 99.9 to 100%.

[93] The method according to [70] above, wherein a proportion of cells in which the reference protein forms an aggregation is a value more than 30%.

[94] The method according to [70] above, wherein a proportion of cells in which the reference protein forms an aggregation is a value in a range of 30 to 40%.

[95] The method according to [70] above, wherein a rate of cells in which the reference protein forms an aggregation is a value in a range of 40 to 50%.

[96] The method according to [70] above, wherein a rate of cells in which the reference protein forms an aggregation is a value in a range of 50 to 60%.

[97] The method according to [70] above, wherein a rate of cells in which the reference protein forms an aggregation is a value in a range of 60 to 70%.

[98] The method according to [70] above, wherein a rate of cells in which the reference protein forms an aggregation is a value in a range of 70 to 80%.

[99] The method according to [70] above, wherein a rate of cells in which the reference protein forms an aggregation is a value in a range of 80 to 90%.

[100] The method according to [70] above, wherein a rate of cells in which the reference protein forms an aggregation is a value in a range of 90 to 95%.

[101] The method according to [70] above, wherein a rate of cells in which the reference protein forms an aggregation is a value in a range of 95 to 99%.

[102] The method according to [70] above, wherein a rate of cells in which the reference protein forms an aggregation is a value in a range of 99 to 99.9%.

[103] The method according to [70] above, wherein a rate of cells in which the reference protein forms an aggregation is a value in a range of 99.9 to 100%.

[104] The peptide according to any one of [1] to [5] above, wherein a peptide tag is capable of reducing an aggregation property of a scFv having at least an amino acid sequence set forth in SEQ ID NO: 1.

[105] The nucleic acid according to [6] above, wherein a peptide tag is capable of reducing an aggregation property of a scFv having at least an amino acid sequence set forth in SEQ ID NO: 1.

[106] The protein expression vector according to any one of [7] to [9] above, wherein a peptide tag is capable of reducing an aggregation property of a scFv having at least an amino acid sequence set forth in SEQ ID NO: 1.

[107] The protein expression vector according to any one of [7] to [9] above, wherein the protein expression vector is a virus vector.

[108] The protein expression vector according to [107] above, wherein the virus vector is selected from the group consisting of a retrovirus vector, a lentivirus vector, an adenovirus vector, an adeno-associated virus vector, a herpes simplex virus vector, a vaccinia virus vector, a Sendai virus vector, and a vesicular stomatitis virus vector.

[109] The nucleic acid according to [6] above, wherein the nucleic acid is an mRNA.

[110] The nucleic acid according to [109] above, wherein the nucleic acid has a cap structure at the 5′ end, and a poly A chain at the 3′ UTR.

[111] The nucleic acid according to [109] or [110] above, wherein the nucleic acid contains pseudouridine as U.

[112] A nanoparticle, comprising the nucleic acid according to any one of [109] to [111] above.

[113] The nanoparticle according to [112], wherein the nanoparticle is a lipid nanoparticle.

[114] The method according to any one of [14] to [17] and [30] to [103] above, wherein the reference protein has an amino acid sequence set forth in SEQ ID NO: 1.

[115] The method, the peptide, the fusion protein, the nucleic acid, or the vector according to any one of those described above, wherein the cell is a eukaryotic cell.

[116] The method, the peptide, the fusion protein, the nucleic acid, or the vector according to any one of those described above, wherein the cell is a human cell.

[117] The method, the peptide, the fusion protein, the nucleic acid, or the vector according to any one of those described above, wherein a peptide tag does not prevent free localization of a protein of interest.

A peptide tag of the present disclosure can cause an intracellular stability of a tagged protein. Accordingly, the peptide tag of the present disclosure can be a more highly biocompatible peptide tag.

### BRIEF DESCRIPTION OF DRAWINGS

FIG. 1 illustrates an effect of a peptide tag Tag4-1 on an aggregation property of a single chain Fv (scFv) in a cell.

FIG. 2A illustrates a scheme for constructing a model of an intracellular accumulation of α-synuclein, that is, an amyloid.

FIG. 2B illustrates fluorescence microscope images showing influence on intracellular synuclein fibril caused by intracellular expression of scFv-E6-CMA peptide fusion protein having Tag18-1, that is, one of peptide tags of the present disclosure.

FIG. 2C illustrates an effect of removing synuclein fibril by intracellular expression of scFv-E6-CMA peptide fusion protein having Tag4-8 or Tag18-1, that is, one of peptide tags of the present disclosure.

FIG. 3A illustrates fluorescence microscope images showing intracellular localization of a scFv-C2 itself having Tag18-1, that is, one of peptide tags of the present disclosure expressed in the cell.

FIG. 3B illustrates a stabilizing action of the scFv-C2 having Tag18-1, that is, one of peptide tags of the present disclosure.

### DESCRIPTION OF EMBODIMENTS

In the present invention, the term “subject” is a vertebrate, examples include birds and mammals, and specific examples include mammals such as a mouse, a rat, a hamster, a guinea pig, a horse, a cow, a pig, a goat, sheep, a donkey, a dog, and a cat, and primates such as a monkey, a chimpanzee, a gorilla, an orangutan, a bonobo, and a human, and particularly a human. Herein, the term “subject” is used in the meaning including a human as described above, and when a human is excluded, the term “non-human” is used.

Herein, the term “antibody” means an immunoglobulin, and refers to a protein having a structure in which two heavy chains (H chains) and two light chains (L chains) stabilized through a disulfide bond are associated with each other. The heavy chain contains a heavy chain variable region VH, heavy chain constant regions CH1, CH2, and CH3, and a hinge region positioned between the CH1 and the CH2, and the light chain contains a light chain variable region VL (wherein VL can be Vκ or Vλ), and a light chain constant region CL. Among these regions, a variable region fragment (Fv) consisting of the VH and the VL is a region directly involved in an antigen bond, and imparting variety to the antibody. An antigen binding region consisting of the VL, the CL, the VH, and the CH1 is designated as a Fab region, and a region consisting of the hinge region, the CH2 and the CH3 is designated as a Fc region.

Among the variable regions, a region directly contacting an antigen is particularly largely changed, and is designated as a complementarity-determining region (CDR). A portion except for the CDRs that is comparatively less mutated is designated as a framework region (FR). There are three CDRs in each variable region of the heavy chain and the light chain, and these are designated, successively from the N terminal side, heavy chain CDR1 to CDR3, and light chain CDR1 to CDR3, respectively. Each CDR is incorporated into the framework regions. The heavy chain variable region of the antibody includes, from the N terminal side to the C terminal side, a heavy chain framework region 1, the heavy chain CDR1, a heavy chain framework region 2, the heavy chain CDR2, a heavy chain framework region 3, the heavy chain CDR3, and a heavy chain framework region 4 in the stated order. The light chain variable region of the antibody includes, from the N terminal side to the C terminal side, a light chain framework region 1, the light chain CDR1, a light chain framework region 2, the light chain CDR2, a light chain framework region 3, the light chain CDR3, and a light chain framework region 4 in the stated order. The antibody may be a recombinant protein (recombinant antibody), and can be produced in an animal cell such as a Chinese hamster ovarian cell (CHO cell). The derivation of the antibody is not especially limited, and examples include an antibody of a non-human animal, an antibody of a non-human mammal (such as a mouse antibody, a rat antibody, or a camel antibody), and a human antibody. The antibody may be a chimeric antibody, a humanized antibody, or a fully humanized antibody. The antibody may be a polyclonal antibody or a monoclonal antibody, and is preferably a monoclonal antibody. A “chimeric antibody” refers to an antibody in which a heavy chain variable region and a light chain variable region are respectively linked to a heavy chain constant region and a light chain constant region of different species. A humanized antibody means an antibody in which an amino acid sequence characteristic to a non-human-derived antibody is substituted in the corresponding position of a human antibody, and an example includes an antibody having heavy chain CDR1 to CDR3 and light chain CDR1 to CDR3 of an antibody produced by immunizing a mouse or a rat, and having the other regions including four framework regions (FR) each of the heavy chain and the light chain all derived from a human antibody. Such an antibody is designated as a CDR-grafted antibody in some cases. A “humanized antibody” encompasses a human chimeric antibody in some cases. A “human chimeric antibody” refers to a non-human-derived antibody in which a constant region of the non-human-derived antibody is substituted with a constant region of a human antibody. The antibody can be an isolated antibody, or a purified antibody. The antibody can be, for example, an IgG.

A variable region of an immunoglobulin chain generally has the same entire structure including relatively preserved framework regions (FR) linked through three hypervariable regions (more frequently designated as “complementarity-determining regions” or CDRs). The CDRs obtained from the two chains of each heavy chain/light chain pair are typically arranged parallel by the framework region for forming a structure specifically binding to a specific epitope on a protein of interest (such as PCSK9). Light chain and heavy chain variable regions present in nature all usually have these elements in the following order from the N terminal to the C terminal: FR1, CDR1, FR2, CDR2, FR3, CDR3 and FR4. In order to assign numbers to amino acids positioned in these respective domains, a numbering system has been devised. This numbering system is defined in “Kabat Sequences of Proteins of Immunological Interest (1987 and 1991, NIH, Bethesda, MD)”, or “Chothia & Lesk, 1987, J. Mol. Biol. 196: 901-917; Chothia et al., 1989, Nature, 342: 878-883”.

Herein, the antibody encompasses an antigen-binding fragment of an antibody. Herein, an antibody not fragmented may be referred to as a full length antibody. A full length antibody can contain the full length of the antibody excluding a signal sequence.

Herein, the term “antigen-binding fragment” means a part of an antibody maintaining a binding property to an antigen. The antigen-binding fragment can contain either or both of a heavy chain variable region and a light chain variable region of the antibody of the present disclosure. The antigen-binding fragment may be chimerized or humanized. Examples of the antigen-binding fragment include Fab, Fab′, F(ab′)₂, and Fv. The antigen-binding fragment may contain a bonded product or functional equivalent produced by recombination (for example, a part of another antibody in the form of a scFv (single chain Fv), a diabody, a scDb, a tandem scFv, a leucine zipper type, or a sc(Fv)₂ (single chain (Fv)₂)). Such an antigen-binding fragment of an antibody can be obtained, for example, by treating the antibody with an enzyme, although not especially limited. For example, when an antibody is digested with papain, a Fab can be obtained. Alternatively, when an antibody is digested with pepsin, a F(ab′)₂ can be obtained, and when this is further reduced, a Fab′ can be obtained. Herein, such an antigen-binding fragment of the antibody can be used. In an scFv, the VL and the VH are linked via an artificial polypeptide linker, and thus, the same antigen specificity as that of the original antibody can be maintained. The VL and the VH can be linked in the order of the VH and the VL, or the VL and the VH from the N terminal side. The linker can have a length of about 10 to 25 amino acids. The linker may contain glycine in a large amount, and may contain an amino acid such as serine or threonine for purpose of increasing water solubility.

Herein, the term “intracellular antibody” (intrabody) refers to an antibody expressed in a cell (for example, in the cytoplasm or in the nucleus). Although an antibody is extracellularly secreted to function, an intracellular antibody is different in that it is designed to be expressed in a cell to function. The intracellular antibody can affect the function of an intracellular protein, and can inhibit the function thereof in the cytoplasm, the nucleus, or the secretory pathway. A cancer gene product can be a target of the intracellular antibody (Biocca, S., Pierandrei-Amaldi, P., and Cattaneo, A. (1993), Biochem Biophys Res Commun, Vol. 197, p. 422 to 427; Biocca, S., Pierandrei-Amaldi, P., Campioni, N., and Cattaneo, A. (1994), Biotechnology (NY), Vol. 12, p. 396 to 399; Cochet, O. et al., (1998), Cancer Res, Vol. 58, p. 1170 to 1176). The intracellular antibody directly binds to a protein for purpose of inhibiting the protein function. The bond may directly inhibit the function of the protein in some cases, and may inhibit the protein from binding to another protein in other cases.

Examples of the intracellular antibody include various antibodies and antigen-binding fragments thereof, and although not especially limited, a scFv, a tandem scFv, a VHH antibody (nanobody), a minibody, and a diabody can be preferably used. A scFv is typically an antibody fragment having a heavy chain variable region and a light chain variable region of an antibody, and the heavy chain variable region and the light chain variable region are linked via a linker. A tandem scFv is typically an antibody fragment having two scFvs having different antigen specificities, and these are linked via a linker. A diabody is typically a dimer of a scFv. Diabodies are roughly divided into bivalent monospecific diabodies and bispecific diabodies. A minibody is typically dimerized two fusion proteins each of a dimerized domain and a scFv via the dimerized domain. A VHH antibody is an antibody fragment containing a heavy chain variable domain of a heavy chain antibody. The VHH antibody is typically a heavy chain variable domain of a heavy chain antibody derived from a camelid (such as a camel, a llama, or an alpaca). Although a general antibody is extracellularly expressed, and hence can be caused to function only extracellularly, the intracellular antibody is superior because it can be caused to exhibit the antibody function in a cell. The intracellular antibody can be used in various applications in a cell such as activation and inactivation of a target protein, and neutralization and block of protein-protein interaction. A scFv tends to exhibit an aggregation property when expressed in a cell. Accordingly, in such a case, it is useful to reduce the aggregation property by obtaining a fusion protein by linking a peptide tag of the present disclosure to the intracellular antibody. When the aggregation property of a protein is reduced, the protein can be caused to exhibit functions inherent to the protein in the cell.

Herein, the term “peptide tag” refers to one that labels a protein of interest, or changes a biochemical property of the protein of interest when fused with the protein of interest. Examples of the peptide tag include various tags such as a FLAG tag, a 3×FLAG tag, a Myc tag, an HA tag, T7, a 6×His tag, a PA tag, an S tag, an E tag, VSV-G, Glu-Glu, Strep-tag II, a HSV tag, a Chitin Binding Domain (CBD) tag, a Calmodulin Binding Peptide (CBP) tag, a V5 tag, a GST tag, a maltose binding protein (MBP) tag, a thioredoxin (Trx) tag, and a mini-AID tag. These can be used for affinity purification of a protein of interest by utilizing affinity for the tag, or for detection of the protein of interest with an antibody to the tag produced. An antibody recognizing a tag is generally designated as a tag antibody, and a tag sequence corresponding to an epitope of the tag antibody is designated as an epitope tag. A tag can have a polypeptide chain generally with a length of several amino acids to several tens amino acids.

Herein, the term “protein of interest” refers to a protein to be expressed in a cell. The protein of interest may be an aggregating protein or a non-aggregating protein. In either case, when the peptide tag of the present disclosure is added thereto, the stability is further increased, and robustness against formation of aggregation can be obtained. Even when added to an aggregating protein, however, the peptide tag of the present disclosure can reduce the aggregation property thereof in a cell, and therefore, the protein of interest can be preferably an aggregating protein. Even when the protein of interest is a secretory protein, aggregation may be formed in a cell before the secretory protein is secreted extracellularly in some cases. The peptide tag of the present disclosure can be advantageously used also for a secretory protein, preferably a secretory protein having an aggregation property.

Herein, the term “aggregating protein” refers to a protein that forms aggregation (particularly, an insoluble aggregation) in a cell. Herein, the term “aggregation property” means a property of forming aggregation, and the term “non-aggregation property” means a property of not forming aggregation. Attenuation of the aggregation property can be promotion of the non-aggregation property, and promotion of the aggregation property can be attenuation of the non-aggregation property. Herein, the term “non-aggregation property” is used interchangeably with the term “stability”. Aggregation can be observed, for example, as a bright point under a microscope by immunocytochemistry (IC). An aggregation rate can be calculated, for example, as a proportion of cells exhibiting aggregation in cells forcedly expressing a protein. Reduction of the aggregation rate thus calculated means increase of cells that forcedly express a protein and are not affected by the aggregation, and therefore can be an index of physiological favorability. Reduction of the aggregation property (for example, reduction of the aggregation rate) and increase of solubility are different indexes. The increase of solubility means increase of a concentration in an aqueous solution of available protein, and does not directly lead to the number of aggregations, or a proportion of cells having the aggregations. Accordingly, the increase of solubility does not always mean the reduction of the aggregation property (for example, the reduction of the aggregation rate).

Herein, an amino acid sequence is described by one letter amino acid code. Specifically, A denotes alanine, R denotes arginine, N denotes asparagine, D denotes aspartic acid, C denotes cysteine, Q denotes glutamine, E denotes glutamic acid, G denotes glycine, H denotes histidine, I denotes isoleucine, L denotes leucine, K denotes lysine, M denotes methionine, F denotes phenylalanine, P denotes proline, S denotes serine, T denotes threonine, W denotes tryptophan, Y denotes tyrosine, and V denotes valine. Amino acids are usually 20 types of L-amino acids mentioned above.

Herein, the term “regulatory sequence” refers to a sequence having activity of driving a gene operably linked thereto to transcribe RNA from the gene. The regulatory sequence is, for example, a promoter. Examples of the promoter include a class I promoter (usable for transcription of an rRNA precursor), a class II promoter (containing a core promoter and an upstream promoter element, and usable for transcription of an mRNA), and a class III promoter (further roughly divided into type I, type II, and type III).

The present invention provides a peptide tag that reduces aggregation tendency of an aggregating protein. The present invention provides a protein expression vector operably linked to a regulatory sequence, and containing a gene encoding the peptide tag. The present invention provides a protein of interest fused with the peptide tag. The present invention provides a protein expression vector operably linked to a regulatory sequence, and containing a gene encoding a protein of interest fused with the peptide tag. The protein of interest can be an intracellular protein in one embodiment. The protein of interest can be an intracellular antibody in one embodiment. The protein of interest can be an scFv in one embodiment.

Hereinafter, the peptide tag of the present disclosure that reduces aggregation tendency of an aggregating protein will be described in detail. The peptide tag of the present disclosure can reduce aggregation tendency of a protein of interest in a eukaryotic cell, particularly, in a human cell. In examination of pharmaceutical application and the like, it can be useful to reduce the aggregation tendency of a protein of interest in a human cell.

The length of the peptide tag of the present disclosure is not especially limited, and can be, for example, 600 amino acids or less, 500 amino acids or less, 400 amino acids or less, 300 amino acids or less, or 200 amino acids or less, and for example, 5 amino acids to 100 amino acids, such as 10 amino acids to 90 amino acids, 20 amino acids to 80 amino acids, 30 amino acids to 70 amino acids, 40 amino acids to 60 amino acids, 10 amino acids to 50 amino acids, 10 amino acids to 40 amino acids, or 10 amino acids to 30 amino acids. In this embodiment, the lower limit of the length of the peptide tag of the present disclosure can be 5 amino acids or more, 10 amino acids or more, 15 amino acids or more, 20 amino acids or more, 30 amino acids or more, 40 amino acids or more, 50 amino acids or more, 60 amino acids or more, 70 amino acids or more, or 80 amino acids or more, and/or the upper limit can be 100 amino acids or less, 90 amino acids or less, 80 amino acids or less, 70 amino acids or less, 60 amino acids or less, 50 amino acids or less, 40 amino acids or less, 30 amino acids or less, or 20 amino acids or less.

(a) The peptide tag of the present disclosure can contain acidic amino acids (amino acids belonging to Element 1) in the following ratio.

In the peptide tag of the present disclosure, 45% or more of amino acids contained in the amino acid sequence thereof can be acidic amino acids.

In the peptide tag of the present disclosure, less than 45% of amino acids contained in the amino acid sequence thereof can be acidic amino acids. In a preferable embodiment, in the peptide tag of the present disclosure, 5% or more and less than 45% of amino acids contained in the amino acid sequence thereof can be acidic amino acids, more preferably, 10% or more and less than 45% of amino acids contained in the amino acid sequence thereof can be acidic amino acids, further preferably, 20% or more and less than 45% of amino acids contained in the amino acid sequence thereof can be acidic amino acids, further preferably, 30% or more and less than 45% of amino acids contained in the amino acid sequence thereof can be acidic amino acids, still further preferably, 35% or more and less than 45% of amino acids contained in the amino acid sequence thereof can be acidic amino acids, and particularly preferably, 40% or more and less than 45% of amino acids contained in the amino acid sequence thereof can be acidic amino acids. The acidic amino acids are D or E. For example, an acidic amino acid content in the peptide tag of the present disclosure can be 44% or less, 43.5% or less, 43% or less, 42.5% or less, 42% or less, 41.5% or less, 41% or less, 40% or less, 35% or less, 30% or less, 25% or less, or 20% or less. Thus, in one embodiment, a risk of occurrence of unexpected interaction with an intracellular molecule or the like having a positive charge based on a high acidic amino acid ratio in the peptide tag can be reduced.

(b) The peptide tag of the present disclosure can contain basic amino acids (amino acids belonging to Element 2) in the following ratio.

The peptide tag of the present disclosure can contain basic amino acids in a rate of preferably 25% or less or 20% or less, and more preferably can contain basic amino acids in a rate of 15% or less of amino acids, can contain basic amino acids further preferably in a rate of 10% or less, can contain basic amino acids further preferably in a rate of 5% or less, and can contain basic amino acids particularly preferably in a rate less than 3%, less than 2%, or less than 1%. In a most preferable embodiment, the peptide tag of the present disclosure does not contain a basic amino acid in the amino acid sequence thereof. The basic amino acids are K, R, or H.

(c) The peptide tag of the present disclosure can contain amino acids belonging to Element 3 in the following ratio.

The amino acids belonging to the Element 3 can be F, P, Y, G, S, Q, N, and A.

In the peptide tag of the present disclosure, 10% or more, preferably 20% or more, more preferably 30% or more, or 40% or more of amino acids contained in the amino acid sequence thereof can be preferably the amino acids of the Element 3. In the peptide tag of the present disclosure, 50% or more, 60% or more, or 70% or more of amino acids contained in the amino acid sequence thereof can be amino acids of the Element 3. In the peptide tag of the present disclosure, preferably 80% or less, more preferably 70% or less, and further preferably 60% or less of amino acids contained in the amino acid sequence thereof can be the amino acids of the Element 3. 50% or less, 40% or less, 30% or less, or 20% or less of amino acids contained in the amino acid sequence thereof can be the amino acids of the Element 3. In a preferable embodiment, in the peptide tag of the present disclosure, 20% or more and 80% or less, 30% or more and 70% or less, 30% or more and 60% or less, 30% or more and 50% or less, 30% or more and 40% or less, 40% or more and 70% or less, 40% or more and 60% or less, 40% or more and 50% or less, 50% or more and 70% or less, 50% or more and 60% or less, 60% or more and 80% or less, or 60% or more and 70% or less of amino acids contained in the amino acid sequence thereof can be the amino acids of the Element 3.

In a particularly preferable embodiment, in the peptide tag of the present disclosure, 5% or more, 10% or more, 15% or more, or 20% or more (preferably 21% or more, 25% or more, or 30% or more) of amino acids contained in the amino acid sequence thereof are either N or P. In the peptide tag of the present disclosure, for example, 90% or less, 80% or less, 70% or less, 60% or less, 50% or less, 45% or less, 40% or less, 35% or less, 30% or less, or 25% or less of amino acids contained in the amino acid sequence thereof can be either N or P. In one embodiment, in the peptide tag of the present disclosure, 10% or more and 20% or less of amino acids contained in the amino acid sequence thereof can be either N or P. In one embodiment, in the peptide tag of the present disclosure, 55% or more and 90% or less of amino acids contained in the amino acid sequence thereof can be either N or P. In one embodiment, in the peptide tag of the present disclosure, more than 10% and 20% or less of amino acids contained in the amino acid sequence thereof can be either N or P. In one embodiment, in the peptide tag of the present disclosure, more than 20% and 30% or less of amino acids contained in the amino acid sequence thereof can be either N or P. In one embodiment, in the peptide tag of the present disclosure, more than 30% and 40% or less of amino acids contained in the amino acid sequence thereof can be either N or P. In one embodiment, in the peptide tag of the present disclosure, more than 40% and 50% or less of amino acids contained in the amino acid sequence thereof can be either N or P. In one embodiment, in the peptide tag of the present disclosure, more than 50% and 60% or less of amino acids contained in the amino acid sequence thereof can be either N or P.

For example, in the peptide tag of the present disclosure, 5% or more, 10% or more, 15% or more, or 20% or more (preferably 21% or more, 25% or more, or 30% or more) of amino acids contained in the amino acid sequence thereof are N. In the peptide tag of the present disclosure, for example, 90% or less, 80% or less, 70% or less, 60% or less, 50% or less, 45% or less, 40% or less, 35% or less, 30% or less, or 25% or less of amino acids contained in the amino acid sequence thereof can be N. In one embodiment, in the peptide tag of the present disclosure, 10% or more and 20% or less of amino acids contained in the amino acid sequence thereof can be N. In one embodiment, in the peptide tag of the present disclosure, 55% or more and 90% or less of amino acids contained in the amino acid sequence thereof can be N. In one embodiment, in the peptide tag of the present disclosure, more than 10% and 20% or less of amino acids contained in the amino acid sequence thereof can be N. In one embodiment, in the peptide tag of the present disclosure, more than 20% and 30% or less of amino acids contained in the amino acid sequence thereof can be N. In one embodiment, in the peptide tag of the present disclosure, more than 30% and 40% or less of amino acids contained in the amino acid sequence thereof can be N. In one embodiment, in the peptide tag of the present disclosure, more than 40% and 50% or less of amino acids contained in the amino acid sequence thereof can be N. In one embodiment, in the peptide tag of the present disclosure, more than 50% and 60% or less of amino acids contained in the amino acid sequence thereof can be N.

For example, in the peptide tag of the present disclosure, 5% or more, 6% or more, 7% or more, 8% or more, 9% or more, 10% or more, 15% or more, or 20% or more (preferably 21% or more, 25% or more, or 30% or more) of amino acids contained in the amino acid sequence thereof are P. In the peptide tag of the present disclosure, for example, 90% or less, 80% or less, 70% or less, 60% or less, 50% or less, 45% or less, 40% or less, 35% or less, 30% or less, or 25% or less of amino acids contained in the amino acid sequence thereof can be P. In one embodiment, in the peptide tag of the present disclosure, 55% or more and 90% or less of amino acids contained in the amino acid sequence thereof can be P. In one embodiment, in the peptide tag of the present disclosure, 10% or more and 20% or less of amino acids contained in the amino acid sequence thereof can be P. In one embodiment, in the peptide tag of the present disclosure, more than 10% and 20% or less of amino acids contained in the amino acid sequence thereof can be P. In one embodiment, in the peptide tag of the present disclosure, more than 20% and 30% or less of amino acids contained in the amino acid sequence thereof can be P. In one embodiment, in the peptide tag of the present disclosure, more than 30% and 40% or less of amino acids contained in the amino acid sequence thereof can be P. In one embodiment, in the peptide tag of the present disclosure, more than 40% and 50% or less of amino acids contained in the amino acid sequence thereof can be P. In one embodiment, in the peptide tag of the present disclosure, more than 50% and 60% or less of amino acids contained in the amino acid sequence thereof can be P.

In a particularly preferable embodiment, in the peptide tag of the present disclosure, 5% or less, 10% or less, 15% or less, or 20% or less of amino acids contained in the amino acid sequence thereof are F or Y. In a particularly preferable embodiment, in the peptide tag of the present disclosure, 5% or less, 10% or less, 15% or less, or 20% or less of amino acids contained in the amino acid sequence thereof are F and/or Y. In a particularly preferable embodiment, in the peptide tag of the present disclosure, 5% or less, 10% or less, 15% or less, or 20% or less of amino acids contained in the amino acid sequence thereof are F and Y. In a particularly preferable embodiment, 5% or more, 10% or more, 15% or more, or 20% or more of amino acids contained in the amino acid sequence thereof are either N or P, and 5% or less, 10% or less, 15% or less, or 20% or less thereof are F and/or Y.

(d) The peptide tag of the present disclosure can contain amino acids belonging to Element 4 in the following ratio.

The amino acids belonging to the Element 4 can be amino acids that are none of an acidic amino acid, a basic amino acid, and the amino acids of the Element 3. The amino acids of the Element 4 can be, for example, M, T, W, C, I, V, and L.

In the peptide tag of the present disclosure, 80% or less, 70% or less, 60% or less, 50% or less, 40% or less, 30% or less, 20% or less, 15% or less, 10% or less, or 5% or less of amino acids contained in the amino acid sequence thereof can be preferably the amino acids of the Element 4. In a preferable embodiment, the peptide tag of the present disclosure does not contain the amino acids of the Element 4.

(e) In the peptide tag of the present disclosure, 40% or less, 30% or less, 20% or less, 15% or less, 10% or less, or 5% or less of amino acids contained in the amino acid sequence thereof can be preferably G. In one embodiment, the peptide tag of the present disclosure does not contain G.

(f) In the peptide tag of the present disclosure, 40% or less, 30% or less, 20% or less, 15% or less, 10% or less, or 5% or less of amino acids contained in the amino acid sequence thereof can be preferably A. In one embodiment, the peptide tag of the present disclosure does not contain A.

(g) In the peptide tag of the present disclosure, 40% or less, 30% or less, 20% or less, 15% or less, 10% or less, or 5% or less of amino acids contained in the amino acid sequence thereof can be preferably G, and 40% or less, 30% or less, 20% or less, 15% or less, 10% or less, or 5% or less of amino acids contained in the amino acid sequence thereof can be A.

(h) The peptide tag of the present disclosure can preferably contain S. The peptide tag of the present disclosure preferably does not contain S. The peptide tag of the present disclosure can contain S in a rate of 10% or more, 20% or more, 30% or more, 40% or more, or 50% or more of amino acids contained in the amino acid sequence thereof. The peptide tag of the present disclosure can contain S in a rate of 60% or less, 50% or less, 40% or less, 30% or less, 20% or less, 15% or less, 10% or less, or 5% or less of amino acids contained in the amino acid sequence thereof.

(A) In the peptide tag of the present disclosure, less than 45% of amino acids contained in the amino acid sequence thereof can be acidic amino acids, and 20% or more and 80% or less (preferably 30% or more and 70% or less) of the amino acids contained in the amino acid sequence thereof can be the amino acids of the Element 3.

(B) In the peptide tag of the present disclosure, less than 45% of amino acids contained in the amino acid sequence thereof can be acidic amino acids, 20% or more and 80% or less (preferably 30% or more and 70% or less) of the amino acids contained in the amino acid sequence thereof can be the amino acids of the Element 3, and 10% or less of the amino acids contained in the amino acid sequence thereof can be A.

(C) In the peptide tag of the present disclosure, less than 45% of amino acids contained in the amino acid sequence thereof can be acidic amino acids, 20% or more and 80% or less (preferably 30% or more and 70% or less) of the amino acids contained in the amino acid sequence thereof can be the amino acids of the Element 3, and 10% or less of the amino acids contained in the amino acid sequence thereof can be G.

(D) In the peptide tag of the present disclosure, less than 45% of amino acids contained in the amino acid sequence thereof can be acidic amino acids, 20% or more and 80% or less (preferably 30% or more and 70% or less) of the amino acids contained in the amino acid sequence thereof can be the amino acids of the Element 3, 10% or less of the amino acids contained in the amino acid sequence thereof can be A, and 10% or less of the amino acids contained in the amino acid sequence thereof can be G.

(E) In the peptide tag of the present disclosure, less than 45% of amino acids contained in the amino acid sequence thereof can be acidic amino acids, 20% or more and 80% or less (preferably 30% or more and 70% or less) of the amino acids contained in the amino acid sequence thereof can be the amino acids of the Element 3, and 30% or less of the amino acids contained in the amino acid sequence thereof can be the amino acids of the Element 4.

(F) In the peptide tag of the present disclosure, less than 45% of amino acids contained in the amino acid sequence thereof can be acidic amino acids, 20% or more and 80% or less (preferably 30% or more and 70% or less) of the amino acids contained in the amino acid sequence thereof can be the amino acids of the Element 3, 30% or less of the amino acids contained in the amino acid sequence thereof can be the amino acids of the Element 4, and 10% or less of the amino acids contained in the amino acid sequence thereof can be A.

(G) In the peptide tag of the present disclosure, less than 45% of amino acids contained in the amino acid sequence thereof can be acidic amino acids, 20% or more and 80% or less (preferably 30% or more and 70% or less) of the amino acids contained in the amino acid sequence thereof can be the amino acids of the Element 3, 30% or less of the amino acids contained in the amino acid sequence thereof can be the amino acids of the Element 4, and 10% or less of the amino acids contained in the amino acid sequence thereof can be G.

(H) In the peptide tag of the present disclosure, less than 45% of amino acids contained in the amino acid sequence thereof can be acidic amino acids, 20% or more and 80% or less (preferably 30% or more and 70% or less) of the amino acids contained in the amino acid sequence thereof can be the amino acids of the Element 3, 30% or less of the amino acids contained in the amino acid sequence thereof can be the amino acids of the Element 4, 10% or less of the amino acids contained in the amino acid sequence thereof can be A, and 10% or less of the amino acids contained in the amino acid sequence thereof can be G.

(I) In the peptide tag of the present disclosure, less than 45% of amino acids contained in the amino acid sequence thereof can be acidic amino acids, and 30% or more and 70% or less of the amino acids contained in the amino acid sequence thereof can be the amino acids of the Element 3.

(J) In the peptide tag of the present disclosure, less than 45% of amino acids contained in the amino acid sequence thereof can be acidic amino acids, 30% or more and 70% or less of the amino acids contained in the amino acid sequence thereof can be the amino acids of the Element 3, and 10% or less of the amino acids contained in the amino acid sequence thereof can be A.

(K) In the peptide tag of the present disclosure, less than 45% of amino acids contained in the amino acid sequence thereof can be acidic amino acids, 30% or more and 70% or less of the amino acids contained in the amino acid sequence thereof can be the amino acids of the Element 3, and 10% or less of the amino acids contained in the amino acid sequence thereof can be G.

(L) In the peptide tag of the present disclosure, less than 45% of amino acids contained in the amino acid sequence thereof can be acidic amino acids, 30% or more and 70% or less of the amino acids contained in the amino acid sequence thereof can be the amino acids of the Element 3, 10% or less of the amino acids contained in the amino acid sequence thereof can be A, and 10% or less of the amino acids contained in the amino acid sequence thereof can be G.

(M) In the peptide tag of the present disclosure, less than 45% of amino acids contained in the amino acid sequence thereof can be acidic amino acids, 30% or more and 70% or less of the amino acids contained in the amino acid sequence thereof can be the amino acids of the Element 3, and 30% or less of the amino acids contained in the amino acid sequence thereof can be the amino acids of the Element 4.

(N) In the peptide tag of the present disclosure, less than 45% of amino acids contained in the amino acid sequence thereof can be acidic amino acids, 30% or more and 70% or less of the amino acids contained in the amino acid sequence thereof can be the amino acids of the Element 3, 30% or less of the amino acids contained in the amino acid sequence thereof can be the amino acids of the Element 4, and 10% or less of the amino acids contained in the amino acid sequence thereof can be A.

(O) In the peptide tag of the present disclosure, less than 45% of amino acids contained in the amino acid sequence thereof can be acidic amino acids, 30% or more and 70% or less of the amino acids contained in the amino acid sequence thereof can be the amino acids of the Element 3, 30% or less of the amino acids contained in the amino acid sequence thereof can be the amino acids of the Element 4, and 10% or less of the amino acids contained in the amino acid sequence thereof can be G.

(P) In the peptide tag of the present disclosure, less than 45% of amino acids contained in the amino acid sequence thereof can be acidic amino acids, 30% or more and 70% or less of the amino acids contained in the amino acid sequence thereof can be the amino acids of the Element 3, 30% or less of the amino acids contained in the amino acid sequence thereof can be the amino acids of the Element 4, 10% or less of the amino acids contained in the amino acid sequence thereof can be A, and 10% or less of the amino acids contained in the amino acid sequence thereof can be G.

(Q) In the above-described peptide tag, preferably 20% or more and less than 45% of amino acids contained in the amino acid sequence thereof can be acidic amino acids, more preferably 30% or more and less than 45% of the amino acids contained in the amino acid sequence thereof can be acidic amino acids, further preferably 35% or more and less than 45% of the amino acids contained in the amino acid sequence thereof can be acidic amino acids, and particularly preferably 40% or more and less than 45% of the amino acids contained in the amino acid sequence thereof can be acidic amino acids.

(R) In the above-described peptide tag, 10% or less of the amino acids contained in the amino acid sequence thereof are preferably basic amino acids. The peptide tag can contain preferably 5% or less of basic amino acids, and particularly preferably less than 3%, less than 2%, or less than 1% of basic amino acids.

Alternatively, the peptide tag does not contain a basic amino acid in a preferable embodiment.

(S) In the above-described peptide tag, 40% or more and 60% or less of the amino acids contained in the amino acid sequence thereof are preferably the amino acids of the Element 3.

(T) In the above-described peptide tag, 20% or less, 15% or less, 10% or less, or 5% or less of the amino acids contained in the amino acid sequence thereof are preferably the amino acids of the Element 4. In a preferable embodiment, the above-described peptide tag does not contain the amino acids of the Element 4.

(U) In the peptide tag of the present disclosure, less than 45% of amino acids contained in the amino acid sequence thereof can be acidic amino acids, 10% or less of the amino acids contained in the amino acid sequence thereof can be basic amino acids, 20% or more and 80% or less (preferably 30% or more and 70% or less) of the amino acids contained in the amino acid sequence thereof can be the amino acids of the Element 3, 30% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A. In this embodiment, preferably 5% or more and less than 45% of the amino acids contained in the amino acid sequence thereof can be acidic amino acids, more preferably 10% or more and less than 45% of the amino acids contained in the amino acid sequence thereof can be acidic amino acids, further preferably 20% or more and less than 45% of the amino acids contained in the amino acid sequence thereof can be acidic amino acids, further preferably 30% or more and less than 45% of the amino acids contained in the amino acid sequence thereof can be acidic amino acids, still further preferably 35% or more and less than 45% of the amino acids contained in the amino acid sequence thereof can be acidic amino acids, and particularly preferably 40% or more and less than 45% of the amino acids contained in the amino acid sequence thereof can be acidic amino acids.

(V) In the peptide tag of the present disclosure, less than 45% of amino acids contained in the amino acid sequence thereof can be acidic amino acids, 5% or less of the amino acids contained in the amino acid sequence thereof can be basic amino acids, 20% or more and 80% or less (preferably 30% or more and 70% or less) of the amino acids contained in the amino acid sequence thereof can be the amino acids of the Element 3, 30% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A. In this embodiment, in the peptide tag of the present disclosure, it is preferable that less than 3%, less than 2%, or less than 1% of the amino acids contained in the amino acid sequence thereof can be basic amino acids, or that it does not contain a basic amino acid.

(W) In the peptide tag of the present disclosure, less than 45% of amino acids contained in the amino acid sequence thereof can be acidic amino acids, 10% or less of the amino acids contained in the amino acid sequence thereof can be basic amino acids, 30% or more and 70% or less of the amino acids contained in the amino acid sequence thereof can be the amino acids of the Element 3, 30% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A. In this embodiment, in the peptide tag of the present disclosure, 40% or more and 60% or less of the amino acids contained in the amino acid sequence thereof can be preferably the amino acids of the Element 3.

(X) In the peptide tag of the present disclosure, less than 45% of amino acids contained in the amino acid sequence thereof can be acidic amino acids, 10% or less of the amino acids contained in the amino acid sequence thereof can be basic amino acids, 20% or more and 80% or less (preferably 30% or more and 70% or less) of the amino acids contained in the amino acid sequence thereof can be the amino acids of the Element 3, 30% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A. In this embodiment, in the peptide tag of the present disclosure, 20% or less, 15% or less, 10% or less, or 5% or less of the amino acids contained in the amino acid sequence thereof can be preferably the amino acids of the Element 4. In a preferable embodiment, the peptide tag of the present disclosure does not contain the amino acids of the Element 4.

(Y) In the peptide tag of the present disclosure, 20% or more and less than 45% of amino acids contained in the amino acid sequence thereof can be acidic amino acids, 10% or less of the amino acids contained in the amino acid sequence thereof can be basic amino acids, 20% or more and 80% or less (preferably 30% or more and 70% or less) of the amino acids contained in the amino acid sequence thereof can be the amino acids of the Element 3, 30% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A. In this embodiment, preferably 30% or more and less than 45% of the amino acids contained in the amino acid sequence thereof are acidic amino acids, more preferably 35% or more and less than 45% of the amino acids of the amino acid sequence are acidic amino acids, and further preferably 40% or more and less than 45% of the amino acids contained in the amino acid sequence thereof are acidic amino acids.

(Z) In the peptide tag of the present disclosure, 20% or more and less than 45% of amino acids contained in the amino acid sequence thereof can be acidic amino acids, 10% or less of the amino acids contained in the amino acid sequence thereof can be basic amino acids, 30% or more and 70% or less of the amino acids contained in the amino acid sequence thereof can be the amino acids of the Element 3, 30% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A. In this embodiment, preferably 40% or more and 60% or less of the amino acids contained in the amino acid sequence thereof are the amino acids of the Element 3.

(AA) In the peptide tag of the present disclosure, 20% or more and less than 45% of amino acids contained in the amino acid sequence thereof can be acidic amino acids, 10% or less of the amino acids contained in the amino acid sequence thereof can be basic amino acids, 20% or more and 80% or less (preferably 30% or more and 70% or less) of the amino acids contained in the amino acid sequence thereof can be the amino acids of the Element 3, 20% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A. In this embodiment, it is preferable that 15% or less of the amino acids contained in the amino acid sequence thereof are the amino acids of the Element 4, that 10% or less of the amino acids contained in the amino acid sequence thereof are the amino acids of the Element 4, and that 5% or less of the amino acids contained in the amino acid sequence thereof are the amino acids of the Element 4.

(AB) In the peptide tag of the present disclosure, 30% or more and less than 45% of amino acids contained in the amino acid sequence thereof can be acidic amino acids, 10% or less of the amino acids contained in the amino acid sequence thereof can be basic amino acids, 30% or more and 70% or less of the amino acids contained in the amino acid sequence thereof can be the amino acids of the Element 3, 30% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A. In this embodiment, preferably 40% or more and 60% or less of the amino acids contained in the amino acid sequence thereof are the amino acids of the Element 3. In this embodiment, more preferably 35% or more and less than 45% of the amino acids contained in the amino acid sequence thereof can be acidic amino acids, and further preferably 40% or more and less than 45% of the amino acids contained in the amino acid sequence thereof can be acidic amino acids. In this embodiment, preferably 40% or more and 60% or less of the amino acids contained in the amino acid sequence thereof are the amino acids of the Element 3, and 35% or more and less than 45% of the amino acids contained in the amino acid sequence thereof are acidic amino acids, and further preferably 40% or more and 60% or less of the amino acids contained in the amino acid sequence thereof can be the amino acids of the Element 3, and 40% or more and less than 45% of the amino acids contained in the amino acid sequence thereof can be acidic amino acids.

(AC) In the peptide tag of the present disclosure, 30% or more and less than 45% of amino acids contained in the amino acid sequence thereof can be acidic amino acids, 10% or less of the amino acids contained in the amino acid sequence thereof can be basic amino acids, 30% or more and 70% or less of the amino acids contained in the amino acid sequence thereof can be the amino acids of the Element 3, 20% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A. In this embodiment, preferably 15% or less, 10% or less, or 5% or less of the amino acids contained in the amino acid sequence thereof can be G. In one embodiment, the peptide tag of the present disclosure does not contain G.

(AD) In the peptide tag of the present disclosure, 35% or more and less than 45% of amino acids contained in the amino acid sequence thereof can be acidic amino acids, 10% or less of the amino acids contained in the amino acid sequence thereof can be basic amino acids, 30% or more and 70% or less of the amino acids contained in the amino acid sequence thereof can be the amino acids of the Element 3, 10% or less of the amino acids contained in the amino acid sequence thereof can be the amino acids of the Element 4, 10% or less of the amino acids contained in the amino acid sequence thereof can be G, and 10% or less of the amino acids contained in the amino acid sequence thereof can be A.

(AE) The peptide tag of the present disclosure can have, for example, an amino acid sequence set forth in any of SEQ ID NOs: 2 to 11. The peptide tag of the present disclosure can have preferably an amino acid sequence of SEQ ID NO: 5.

(AF) The peptide tag of the present disclosure can have, for example, any one of amino acid sequences shown in Tables 1 to 11, Table 12-1, Table 12-2, Table 13-1, Table 13-2, Table 14-1, and Table 14-2.

(AG) The peptide tag of the present disclosure may have one or more selected from the group consisting of addition and insertion of one or more amino acids selected from the group consisting of N and P, and substitution with the amino acids (for example, substitution of one to about 30% of amino acids, such as substitution with 1 to 20, 10, or several amino acids) in any amino acids of any amino acid sequences of (AE) and (AF) described above (for example, the amino acids of the Element 1, the Element 2, the Element 3, or the Element 4, or for example, the amino acids of the Element 2, the Element 3, or the Element 4) The peptide tag of the present disclosure may have addition and insertion of S in any amino acids of any amino acid sequences of (AF) and (AG) described above (for example, the amino acids of the Element 1, the Element 2, the Element 3, or the Element 4, or for example, amino acids of the Element 2, the Element 3, or the Element 4), and substitution of S with the amino acids (for example, substitution of one to about 30% of amino acids, such as substitution with 1 to 20, 10, or several amino acids). The peptide tag of the present disclosure may have deletion of an arbitrary amino acid of any one of the amino acid sequences of (AF) and (AG) described above (for example, the amino acids of the Element 1, the Element 2, the Element 3, or the Element 4, for example, the amino acids of the Element 2, the Element 3 (particularly, F and/or Y), and the Element 4 (A or G).

(AH) In the peptide tag of the present disclosure, preferably 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, or 20% or more of amino acids contained in the amino acid sequence thereof can be either N or P, or N and P. In the peptide tag of the present disclosure, preferably 5% or more, 6% or more, 7% or more, 8% or more, 9% or more, 10% or more, 11% or more, 12% or more, 13% or more, 14% or more, 15% or more, 16% or more, 17% or more, 18% or more, 190% or more, 20% or more, 21% or more, 25% or more, or 30% or more of the amino acids contained in the amino acid sequence thereof can be P. In the peptide tag of the present disclosure, preferably 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 25% or more, or 30% or more of the amino acids contained in the amino acid sequence thereof can be N.

(AI) In the peptide tag of the present disclosure, 45% or more of amino acids contained in the amino acid sequence thereof can be acidic amino acids, and 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 25% or more, or 30% or more thereof can be N or P. In this embodiment, less than 10% (preferably less than 5%, and more preferably 0%) of the amino acids contained in the amino acid sequence thereof can be G, less than 10% (preferably less than 5%, and more preferably 0%) thereof can be A, and/or less than 10% (preferably less than 5%, and more preferably 0%) thereof can be F and Y.

(AJ) In the peptide tag of the present disclosure,
(a) 5% or more and less than 45% of amino acids contained in the amino acid sequence can be acidic amino acids,
(b) 30% or more and less than 70% of the amino acids contained in the amino acid sequence can be amino acids selected from the group consisting of F, P, Y, G, S, Q, N, and A, and 60% or less of the amino acids contained in the amino acid sequence can be S,
(c) 10% or less of the amino acids contained in the amino acid sequence can be amino acids selected from the group consisting of M, T, W, C, I, V, and L, and (d) each of A and G can constitute less than 10% of the amino acids contained in the amino acid sequence. In this embodiment, the amino acid sequence can have a length of 10 to 200 amino acids (such as 10 to 90 amino acids).

(AK) In the peptide tag of the present disclosure,
(a) 5% or more and less than 45% of amino acids contained in the amino acid sequence can be acidic amino acids,
(b) 30% or more and less than 70% of the amino acids contained in the amino acid sequence can be amino acids selected from the group consisting of F, P, Y, G, S, Q, N, and A, 60% or less of the amino acids contained in the amino acid sequence can be S, and 10% or more of the amino acids contained in the amino acid sequence can be N or P,
(c) 10% or less of the amino acids contained in the amino acid sequence can be amino acids selected from the group consisting of M, T, W, C, I, V, and L, and
(d) each of A and G can constitute less than 10% of the amino acids contained in the amino acid sequence. In this embodiment, the amino acid sequence can have a length of 10 to 200 amino acids (such as 10 to 90 amino acids).

(AJ) In the peptide tag of the present disclosure,
(a) 5% or more and less than 45% of amino acids contained in the amino acid sequence can be acidic amino acids,
(b) 30% or more and less than 70% of the amino acids contained in the amino acid sequence can be amino acids selected from the group consisting of F, P, Y, G, S, Q, N, and A, 60% or less of the amino acids contained in the amino acid sequence can be S, and 10% or less of the amino acids contained in the amino acid sequence can be F and/or Y,
(c) 10% or less of the amino acids contained in the amino acid sequence can be amino acids selected from the group consisting of M, T, W, C, I, V, and L, and
(d) each of A and G can constitute less than 10% of the amino acids contained in the amino acid sequence. In this embodiment, the amino acid sequence can have a length of 10 to 200 amino acids (such as 10 to 90 amino acids).

(AM) In the peptide tag of the present disclosure,
(a) 5% or more and less than 45% of amino acids contained in the amino acid sequence can be acidic amino acids,
(b) 30% or more and less than 70% of the amino acids contained in the amino acid sequence can be amino acids selected from the group consisting of F, P, Y, G, S, Q, N, and A, 60% or less of the amino acids contained in the amino acid sequence can be S, 10% or more of the amino acids contained in the amino acid sequence can be N or P, and 10% or less of the amino acids contained in the amino acid sequence can be F and/or Y,
(c) 10% or less of the amino acids contained in the amino acid sequence can be amino acids selected from the group consisting of M, T, W, C, I, V, and L, and
(d) each of A and G can constitute less than 10% of the amino acids contained in the amino acid sequence. In this embodiment, the amino acid sequence can have a length of 10 to 200 amino acids (such as 10 to 90 amino acids).

(AN) In the peptide tag of the present disclosure,
(a) 5% or more and less than 45% of amino acids contained in the amino acid sequence can be acidic amino acids,
(b) 30% or more and less than 65% of the amino acids contained in the amino acid sequence can be amino acids selected from the group consisting of F, P, Y, G, S, Q, N, and A, 60% or less of the amino acids contained in the amino acid sequence can be S, and 10% or less of the amino acids contained in the amino acid sequence can be F and/or Y,
(c) 10% or less of the amino acids contained in the amino acid sequence can be amino acids selected from the group consisting of M, T, W, C, I, V, and L, and
(d) each of A and G can constitute less than 10% of the amino acids contained in the amino acid sequence. In this embodiment, the amino acid sequence can have a length of 10 to 200 amino acids (such as 10 to 90 amino acids).

(AN) In the peptide tag of the present disclosure,
(a) 5% or more and less than 45% of amino acids contained in the amino acid sequence can be acidic amino acids,
(b) 30% or more and less than 65% of the amino acids contained in the amino acid sequence can be amino acids selected from the group consisting of F, P, Y, G, S, Q, N, and A, 60% or less of the amino acids contained in the amino acid sequence can be S, 10% or more of the amino acids contained in the amino acid sequence can be N or P,
(c) 10% or less of the amino acids contained in the amino acid sequence can be amino acids selected from the group consisting of M, T, W, C, I, V, and L, and
(d) each of A and G can constitute less than 10% of the amino acids contained in the amino acid sequence. In this embodiment, the amino acid sequence can have a length of 10 to 200 amino acids (such as 10 to 90 amino acids).

(AO) In the peptide tag of the present disclosure,
(a) 5% or more and less than 45% of amino acids contained in the amino acid sequence can be acidic amino acids,
(b) 30% or more and less than 65% of the amino acids contained in the amino acid sequence can be amino acids selected from the group consisting of F, P, Y, G, S, Q, N, and A, 60% or less of the amino acids contained in the amino acid sequence can be S, 10% or more of the amino acids contained in the amino acid sequence can be N or P, and 10% or less of the amino acids contained in the amino acid sequence can be F and/or Y,
(c) 10% or less of the amino acids contained in the amino acid sequence can be amino acids selected from the group consisting of M, T, W, C, I, V, and L, and
(d) each of A and G can constitute less than 10% of the amino acids contained in the amino acid sequence. In this embodiment, the amino acid sequence can have a length of 10 to 200 amino acids (such as 10 to 90 amino acids).

(AP) In the peptide tag of the present disclosure,
(a) 5% or more and less than 45% of amino acids contained in the amino acid sequence can be acidic amino acids, (b1) 55% or more and less than 90% of the amino acids contained in the amino acid sequence can be either of N and P, and
the rest of the amino acids contained in the amino acid sequence can be other amino acids. In this embodiment, 20% or less (preferably 15% or less, 10% or less, 5% or less, 4% or less, 3% or less, 2% or less, 1% or less, or 0%) of the amino acids contained in the amino acid sequence are neither an acidic amino acid nor N and P.

(AQ) In the peptide tag of the present disclosure,
(a) 45% or more of amino acids contained in the amino acid sequence can be acidic amino acids, (b1) 21% or more of the amino acids contained in the amino acid sequence can be N, and/or 7% or more thereof can be P, and
20% or less (preferably 15% or less, 10% or less, 5% or less, 4% or less, 3% or less, 2% or less, 1% or less, or 0%) of the amino acids contained in the amino acid sequence can be other amino acids.

(AR) In the (AK) to (AQ) above, a rate of the Element 4 can be preferably 0%.

(AS) In the (AK) to (AQ) above, a rate of A or G can be 0%. In the (AK) to (AO) above, a rate of A and G is preferably 0%.

(AT) In the (AK) to (AQ) above, preferably, a rate of the Element 4 is 0%, and a rate of A and G is 0%.

(AU) In the (AR) to (AT) above, a rate of the Element 2 is preferably 0%.

In the (a) above, preferably 10% or more and less than 45% of the amino acids contained in the amino acid sequence are acidic amino acids, more preferably 15% or more and less than 45% thereof are acidic amino acids, further preferably 20% or more and less than 45% thereof are acidic amino acids, still further preferably 25% or more and less than 45% thereof are acidic amino acids, and particularly preferably 30% or more and less than 45% thereof are acidic amino acids. In these examples, the upper limit of the acidic amino acid content can be, for example, less than 45%, 40% or less, or 35% or less.

The peptide tag of the present disclosure can be added to a protein (such as an intracellular aggregating protein). Accordingly, the present disclosure provides a fusion protein of the peptide tag of the present disclosure and an intracellular aggregating protein. The peptide tag of the present disclosure may be added to an intracellular non-aggregating protein. When the tag is added thereto, toughness of the non-aggregating protein against a non-aggregation property can be increased. The protein can be, for example, an intracellular antibody. The intracellular antibody can be an antigen-binding fragment of an antibody. The peptide tag of the present disclosure may be added to a fusion protein of an intracellular antibody and a degradation-inducing sequence. Thus, selective degradation of a target to which the intracellular antibody binds can be induced. Examples of the intracellular antibody include the above-described antibody fragments. Other examples of the intracellular antibody include antibodies that bind to a-synuclein, LRRK2, Tau, β-amyloid, amyloid precursor protein (APP), C9orf72, superoxide dismutase 1 (SOD1), TAR DNA-binding protein 43 (TDP43), Fused in Sarcoma (FUS), and a prion protein, and pathological forms thereof. Another example of the intracellular antibody includes an antibody inhibiting protein-protein interaction (PPI). Still another example of the intracellular antibody includes one in the form of a fusion protein with a degradation-inducing sequence that binds to a target. Other examples of the intracellular antibody include intracellular antibodies described in Molecular Therapy, 29(2): 859-872, 2021 (such as CP13 iB, PHF1 iB, and Tau5 iB), and intracellular antibodies each having all CDR sequences of these intracellular antibodies. “iB” is an abbreviation of “intrabody”, and specifically means an intracellular antibody. The present disclosure provides a fusion protein of, for example, such an intracellular antibody and the peptide tag of the present disclosure. The intracellular antibody may preferably further include a degradation-inducing sequence. Other examples of the intracellular antibody include intracellular antibodies described in Molecular Therapy, 30(4): 1484-1499, 2022 (such as VHH E4-1, and VHHZ70), and intracellular antibodies each having all CDR sequences of these intracellular antibodies. The intracellular antibody may preferably further include a degradation-inducing sequence. Still other examples of the intracellular antibody include an intracellular antibody described in J. Biol. Chem., 295(31): 10662-10676, 2020 (such as M204-scFv), and an intracellular antibody having all CDR sequences of this intracellular antibody. The intracellular antibody may preferably further include a degradation-inducing sequence. Still other examples of the intracellular antibody include an intracellular antibody described in WO2018/231254 (such as BIIB092 antibody), an intracellular antibody described in WO2016/207245, an antibody described in WO2018/011073 (such as C10-2), intracellular antibodies described in WO2015/114538 (such as VHH tau A2, VHH tau A2-SH, and VHH tau A2var-SH), intracellular antibodies described in WO2014/059442 (such as F9T, D11C, D4G, G12C, H2A and H7T), and JP2020/515233 (such as IE4, 9B11, 3A9, 10F10, 11F11, AC8, AE8, AA9, DG5, AD2, AD7, DG11, DG8, and DA9) and intracellular antibodies each having all CDR sequences of these intracellular antibodies. The intracellular antibody may preferably further include a degradation-inducing sequence. Examples of the intracellular antibody further include an intracellular antibodies capable of degrading and removing abnormal TDP-43 (such as SEQ ID NOs: 21 to 24) described in WO2019177138, and an intracellular antibody having all CDR sequences of this intracellular antibody. The intracellular antibody may preferably further include a degradation-inducing sequence. Thus, an intracellular antibody (such as a scFv or VHH) binding to tau, an intracellular antibody (such as a scFv or VHH) binding to α-synuclein, and other intracellular antibodies (such as a scFv or VHH) against amyloid causing cytotoxicity in a cell are preferred, and can be linked to the tag to form a fusion protein with the tag.

In one embodiment, the peptide tag of the present disclosure does not have a CAAX motif (such as SEQ ID NO: 58: KLNPPDESGPGCMSCKCVLS). In one embodiment, the peptide of the present disclosure does not have a membrane localization signal. In one embodiment, the peptide tag of the present disclosure does not have a signal peptide sequence for extracellular secretion from the viewpoint of expressing a protein of interest in a cell. In one embodiment, the peptide tag of the present disclosure may have a signal sequence for extracellular secretion from the viewpoint of promoting extracellular secretion of a protein of interest. When a secretory protein has an aggregation property, the peptide tag of the present disclosure containing a signal sequence in the sequence thereof, or the peptide tag of the present disclosure linked tandem to the signal sequence can be advantageous. In one embodiment, the peptide tag of the present disclosure can contain a nuclear localization signal. In one embodiment, the peptide tag of the present disclosure does not have a sequence preventing protein localization in the cytoplasm. In one embodiment, the peptide tag of the present disclosure promotes free distribution in a cell of the protein of interest. In one embodiment, the peptide tag of the present disclosure can promote intracellular bond of the protein of interest to an original binding partner, and co-localization with the binding partner. In one embodiment, the peptide tag of the present disclosure can have a sequence that imposes unique constraints on the distribution in a cell (or a sequence that prevents free distribution) of the protein of interest, but is possible not to have such a sequence.

In any embodiment, the peptide tag of the present disclosure does not have the following sequence (Enzymol. 326, 362-267 (2000)): S-tag: KETAAAKFERQHMDS (SEQ ID NO: 14). In one embodiment, the peptide tag of the present disclosure can have a sequence in which a rate of the Element 2 is 10% or less, and/or a rate of A is 10% or less.

In any embodiment, the peptide tag of the present disclosure does not have KLNPPDESGPGCMSCKCVLS (SEQ ID NO: 15) (Tanaka et al., 2007, EMBO Journal, 26: 3250-3259). In one embodiment, the peptide tag of the present disclosure does not have a sequence having 90% or more sequence identity to this sequence.

In any embodiment, the peptide tag of the present disclosure does not have EFGGAPEFPKPSTPPGSSGL (SEQ ID NO: 16), and a sequence having 90% or more sequence identity to this sequence (Paolo et al., 2003, Clinical Cancer Research, 9: 2837-2848). In one embodiment, the peptide tag of the present disclosure does not have a sequence having 90% or more sequence identity to this sequence.

In any embodiment, the peptide tag of the present disclosure does not have any one of the following sequences (Arimori et al., 2017, Structures, 25: 1611-1622):
hMst1: DYEFLKSWTVEDLQKRLLALDPMMEQEIEEIRQKYQSKRQPILDAIEAK (SEQ ID NO: 17);
hMST2: DFDFLKNLSLEELQMRLKALDPMMEREIEELRQRYTAKRQPILDAMDAK (SEQ ID NO: 18);
hRaf1: GEVNWDAFSMPELHNFLRILQREEEEHLRQILQKYSYSRQKIQEALHAS (SEQ ID NO: 19);
hRaf5: GEVEWDAFSIPELQNFLTILEKEEQDKIQQVQKKYDKFRQKLEEALRES (SEQ ID NO: 20);
hSAV1: HILKWELFQLADLDTYQGMLKLLFMKELEQIVKMYEAYRQALLTELENR (SEQ ID NO: 21). In one embodiment, the peptide tag of the present disclosure does not have a sequence having 90% or more sequence identity to any one of these sequences.

In any embodiment, the peptide tag of the present disclosure have none of the following (Zhang et al., 2004, Protein Expression and Purification, 36(2): 207-216):

### T7C:

LEDPFQSGVMLGVASTVAASPEEASVTSTEETLTPAQEAARTRAANKARKEAELAAA TAEQ (SEQ ID NO: 22);
T7B: LEDPEEASVTSTEETLTPAQEAARTRAANKARKEAELAAATAEQ (SEQ ID NO: 23);
T7B1: LEDPEEASVTSTEETLTPAQEAARTRPPNKARKEAELAAATAEQ (SEQ ID NO: 24);
T7B2: LEDPEEASVTSTEETLTPAQEAARTRGGNKARKEAELAAATAEQ (SEQ ID NO: 25);
T7B3: LEDPEEASVTSTEETLTPAQEAARTRAANKARKEAELTAEQ (SEQ ID NO: 26);
T7B4: LEDPEEASVTSTEETLTPAQEAARTRAANKARKEAELEAETAEQ (SEQ ID NO: 27);
T7B5: LEDPEEASVTSTEETLTPAQEAARTRAAAKARKEAELAAATAEQ (SEQ ID NO: 28);
T7B6: LEDPEEASVTSTEETLTPAQEAARTRKARKEAELAAATAEQ (SEQ ID NO: 29);
T7B7: LEDPEEASVTSTEETLTPAQEAARTRAANKARKEAELAA (SEQ ID NO: 30):
T7B8: LEDPEEASVTSTEETLTPAQEAARTRAANKARKEAELAAA (SEQ ID NO: 31);
T7B9: LEDPEEASVTSTEETLTPAQEAAETEAANKARKEAELEAETAEQ (SEQ ID NO: 32);
T7B10: LEDPTPAQEAARTRAANKARKEAELAAATAEQ (SEQ ID NO: 33);
T7A: LEDPAANKARKEAELAAATAEQ (SEQ ID NO: 34);
T7A1: LEDPERNKERKEAELAAATAEQ (SEQ ID NO: 35);
T7A2: LEDPERNKERKEAELEAATAEQ (SEQ ID NO: 36);
T7A3: LEDPERNKERKEAELEAETAEQ (SEQ ID NO: 37);
T3: LEDPAVWEAGKVVAKGVGTADITATTSNGLIASCKVIVNAATS (SEQ ID NO: 38);
T3A: LEDPAVWEAGKVVAKGVGTADITATTSNGLIASSEEADNAATS (SEQ ID NO: 39). In one embodiment, the peptide tag of the present disclosure does not have a sequence having 90% or more sequence identity to any one of these sequences.

In any embodiment, the peptide tag of the present disclosure have none of the following (Japanese Patent Laid-Open No. 2015-97519):

Zif628:
(SEQ ID NO: 40)
ERPYACPVESCDRRFSRSDELTRHIRIHTGQKPFQCRICMR
NFSRSDHLTTHIRTHTGEKPFACDICGRKFARSDERKRHTK
IHLRQKD;
HinR:
(SEQ ID NO: 41)
GRPRAITKHEQEQISRLLEKGHPROQLAIIFGIGVSTLYRY
FPASSIKKRMN;
and
TrpR:
(SEQ ID NO: 42)
MAQQSPYSAAMAEQRHXXQEWLRFVDLLKNAYQNXXDLHLP
LLNLMLTPDERXXEALGTRVRIVEELLRGEMSQRELKNELG
AGIATITRGSNSLKAAPVELROWLEEVLLKSD.

In a preferable embodiment, the peptide tag of the present disclosure can be a natural sequence found in a non-human living thing. In a preferable embodiment, the peptide tag of the present disclosure can be a non-natural sequence or a part thereof. In either embodiment, the peptide tag of the present disclosure is none of the following (WO2010/034183):

NE-1:
(SEQ ID NO: 43)
TKENPRSNQEESYDDNES;
NE-8:
(SEQ ID NO: 44)
TKENPRTNQEESYDDNES;
NE-9:
(SEQ ID NO: 45)
TKENPRSNQDESYDDNES;
NE-10:
(SEQ ID NO: 46)
TKENPRSNOPPSYDDNES.

In one embodiment, the peptide tag of the present disclosure is none of the following (WO2011/034605): ACID.P1: GGSAQLEKELQALEKENAQLEWELQALEKELAQGAT (SEQ ID NO: 50).

In one embodiment, the peptide tag of the present disclosure is none of the following (WO2009/023270):

rPEG_K288-GFP:
(SEQ ID NO: 51)
(GEGEGEGEG)₃₂

In one embodiment, the peptide tag of the present disclosure is none of the following (WO2020/059228):

Hero7:
(SEQ ID NO: 1038)
MTRGNQRELARQKNMKKQSDSVKGKRRDDGLSAAARKQRDS
EIMQQKOKKANEKKEEPK;
Hero9:
(SEQ ID NO: 1039)
MSGPNGDLGMPVEAGAEGEEDGFGEAEYAAINSMLDQINSC
LDHLEEKNDHLHARLQELLESNRQTRLEFQQQLGEAPSDAS
P;
Hero11:
(SEQ ID NO: 1040)
MAQGQRKFQAHKPAKSKTAAAASEKNRGPRKGGRVIAPKKA
RVVQQQKLKKNLEVGIRKKIEHDVVMKASSSLPKKLALLKA
PAKKKGAAAATSSKTPS.

In one embodiment, the peptide tag of the present disclosure is none of the following (Protein Engineering, Design & Selection, 26(8): 490-501, 2013):

PAS#1:
(SEQ ID NO: 52)
ASPAAPAPASPAAPAPSAPAA;
1P2:
(SEQ ID NO: 53)
ASAAAPAAASAAASAPSAAAA;
PAS#5:
(SEQ ID NO: 54)
AASPAAPSAPPAAASPAAPSAPPAA;

and repeated sequences of these (the number of repetition being, for example, 200±20 times, 400±40 times, or 600±60 times).

In one embodiment, the peptide tag of the present disclosure is none of the following (Protein Engineering, Design & Selection, 17(11): 779-786, 2004):

Z (W):
(SEQ ID NO: 55)
VDNKFNKEQQNAFYEILHLPNLNEEQRNAFIQ
SLKDDPSQSANLLAEAKKLNDAQAPK;
Z (a1):
(SEQ ID NO: 56)
VDNKFNKEQQNAEYEIEHLPNLNEEQENAFIQ
SLEDDPSQSANLLAEAKKLNDAQAPK;
Z (a2):
(SEQ ID NO: 57)
VDNKFNKEEEEAEEEIEHLPNLNEEQEEAFIE
SLEDDPSQSANLLAEAKKLNDAQAPK

In one embodiment, the peptide tag of the present disclosure may have a mutation selected from the group consisting of substitution, insertion, deletion, addition, and elimination of one or more, preferably two or more amino acids in any one of the amino acid sequences of SEQ ID NOs: 43 to 46 and 47 to 58. In one embodiment, the peptide tag of the present disclosure can have less than 90%, 85% or less, 80% or less, 75% or less, 70% or less, 60% or less, 50% or less, 40% or less, 30% or less, 20% or less, or 10% or less sequence identity to any one of the amino acid sequences of SEQ ID NOs: 44 to 47 and 47 to 58.

In any embodiment, the peptide tag of the present disclosure does not contain the following sequences (Protein Science (2019) 28, 823-836):

PA12-tag:
(SEQ ID NO: 47)
GVAMPGAEDDVV;
PA14-tag:
(SEQ ID NO: 48)
EGGVAMPGAEDDVV.

In any embodiment, the peptide tag of the present disclosure does not contain the following sequences:

(SEQ ID NO: 49)
DYKDDDDVEAEESDNVDSADAEEDDSDVWWGGADTDY
ADGSEDKVVEVAEEEEVAEVEEEEADDDEDDEDGDEV
EEEAEEPYEEATERTTSIATTTTTTTESVEEVYPGQV
GYPGQVGYPGQV.

In one embodiment, the peptide tag of the present disclosure has less than 90%, 80% or less, 70% or less, 60% or less, 50% or less, 40% or less, 30% or less, 20% or less, or 10% or less sequence identity to any one of SEQ ID NOs: 14 to 48 and 49 to 58.

In one embodiment, the peptide tag of the present disclosure can have a sequence satisfying one, two or all of the following (i) to (iii): (i) a rate of the Element 2 is 10% or less, (ii) a rate of A is 10% or less, and (iii) a rate of G is 10% or less.

In a preferable embodiment, the peptide tag of the present disclosure does not have a sequence consecutively containing 5 or more As. In a preferable embodiment, the peptide tag of the present disclosure does not have a sequence consecutively containing 5 or more Qs. In a preferable embodiment, the peptide tag of the present disclosure does not have a sequence consecutively containing 5 or more Ss. In a preferable embodiment, the peptide tag of the present disclosure does not have a sequence consecutively containing 5 or more Ns. In a preferable embodiment, the content of a specific single amino acid in the amino acid sequence of the peptide tag does not exceed 50%, 40%, 35%, 30%, 25%, or 20%. In a preferable embodiment, the peptide tag of the present disclosure does not contain an amino acid sequence having a length of 3 to 8 amino acids described in Table 1 in WO2002/092132, or does not contain a consecutive repeat (for example, consecutive repeat of 2 or more, 3 or more, 4 or more, 5 or more, 6 or more, 7 or more, 8 or more, 9 or more, or 10 or more times) of the amino acid sequence.

For example, the peptide tag of the present disclosure can inhibit an intracellular antibody from forming aggregation to promote uniform distribution in a cell of the antibody, and/or inhibit aggregation formation to promote bond of the intracellular antibody to an antigen in a cell, and co-localization with the antigen. In one embodiment, the peptide tag of the present disclosure is possible not to have a sequence that imposes unique constraints on the distribution in a cell (or a sequence that prevents free distribution) of the intracellular antibody, and/or is possible not to have bond in a cell of the intracellular antibody to an antigen, and co-localization with the antigen.

All the peptide tags of the present disclosure can mitigate, inhibit, or improve aggregation tendency; increase, promote, or improve a non-aggregation property; or increase, promote, or improve stability of a tagged protein. The peptide tag of the present disclosure is possible not to have a sequence that imposes unique constraints on the distribution in a cell (or a sequence that prevents free distribution) of the intracellular antibody, and/or is possible not to have bond in a cell of the intracellular antibody to an antigen and co-localization with the antigen. When the protein of interest is an antigen-binding fragment of an antibody, the peptide tag of the present disclosure can promote co-localization with an antigen through bond of the protein to the antigen.

The peptide tag of the present disclosure can be, for example, a gene product encoded by a gene of a living thing, or a fragment thereof, and here, can be a gene product encoded by a gene of a non-human living thing (for example, a microorganism such as a bacteria, an alga, or a fungus, an animal such as a mammal, a bird, or fish, or a plant), or a fragment thereof. Alternatively, in a preferable embodiment, the peptide tag of the present disclosure can be a gene product encoded by a gene of a human, or a fragment thereof.

When fused with an aggregating protein, the peptide tag of the present disclosure can mitigate, inhibit or improve the aggregation tendency of the aggregating protein, or can increase, promote, or improve the non-aggregation property of the aggregating protein. Aggregation of a protein can adversely affect a cell in which the protein is expressed, and in addition, can adversely affect the protein production amount and functionality by the aggregation. Accordingly, the mitigation, inhibition, or improvement of the aggregation tendency reduces the influence of the aggregation on the cell, and can lead to reduction of the influence on the protein production amount and the functionality. In this manner, the peptide tag of the present disclosure can be beneficial in improvement of the expression level of an aggregating protein expressed in a cell and/or improvement of the functionality, and accordingly, can be useful for forced expression of the aggregating protein in vivo.

Accordingly, the peptide tag of the present disclosure may be fused with an aggregating protein. The aggregating protein can be a protein that forms aggregation in 30% or more, 40% or more, 50% or more, 60% or more, 70% or more, 80% or more, or 90% or more of cells when intracellularly expressed. The aggregating protein may be a protein that forms aggregation in 90% or less, 80% or less, 70% or less, 60% or more, or 50% or less of cells when intracellularly expressed. The fusion can be performed, for example, on the N terminal and/or the C terminal (preferably, both the N terminal and the C terminal) of the aggregating protein. The fusion can be conducted by, for example, linking a nucleic acid encoding the peptide tag of the present disclosure and a nucleic acid encoding the aggregating protein, in-frame (in such a manner as to match the reading frames of codons). The peptide tag of the present disclosure may be fused with a non-aggregating protein. Thus, the non-aggregation property of the non-aggregating protein can be further increased. The non-aggregating protein may be a protein that forms aggregation in 20% or less, 15% or less, 10% or less, 9% or less, 8% or less, 7% or less, 6% or less, 5% or less, 4% or less, 3% or less, 2% or less, or 1% or less of cells or a protein that does not form aggregation, when intracellularly expressed.

When fused with a protein, the peptide tag of the present disclosure can increase, promote, or improve the stability under an intracellular environment of the protein. The stability under an intracellular environment of a protein is beneficial in both an aggregating protein and a non-aggregating protein.

In one embodiment, the protein of interest may be linked to a second peptide tag. The second peptide tag can be added to the protein of interest for purpose of, for example, detection or purification. In this case, the peptide tag of the present disclosure can be used for reducing, inhibiting, or improving aggregation tendency; increasing, promoting, or improving a non-aggregation property; or increasing, promoting, or improving stability of a fusion protein of the protein of interest and the second peptide tag. As the second peptide tag, a usual peptide tag, such as an HA tag, can be used.

The aggregating protein is not especially limited, and can be, for example, an antigen-binding fragment of an antibody. The aggregating protein can be preferably a single chain Fv (scFv) or a VHH antibody. A scFv is a fusion protein containing a heavy chain variable region and a light chain variable region of an antibody in which the heavy chain variable region and the light chain variable region are linked via a linker (preferably, a flexible linker). For example, there is an undruggable therapeutic target in a cell. This is conspicuous, for example, when a site for binding to a low molecular weight compound cannot be found in a therapeutic target. An antibody can bind to the target with strong binding affinity with different principles from the low molecular weight compound, and hence can effectively work on the therapeutic target regarded as undruggable with the low molecular weight compound. An antibody is, however, usually extracellularly secreted, and extracellularly functions. Therefore, in order to express, in a cell, a secretory protein (protein extracellularly secreted), a gene can be designed to express a secretory protein (intracellular antibody) in a cell. For example, in order to express a secretory protein (intracellular antibody), a signal sequence of the protein can be disrupted, preferably removed or the like. In particular, a scFv can exhibit an aggregation property in a cell. Accordingly, the tag of the present disclosure can be fused with an aggregating protein, particularly a secretory protein, particularly an antigen-binding fragment of an antibody, and with preferably a scFv. A secretory protein exhibits an aggregation property in a cell in some cases. For stabilizing such a secretory protein before secretion in a cell, the tag of the present disclosure can be effective.

The antibody, or the antigen-binding fragment of the antibody can have binding affinity (KD) to an antigen thereof of, for example, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. A test of the binding property and a test of the binding affinity can be performed, for example, in a buffered saline.

An example of the antigen includes an intracellular antigen such as an intracellular protein. Examples of the intracellular protein include an intracytoplasmic protein (such as an intracellular extravesicular cytoplasmic protein), a nuclear protein (in this case, the peptide tag or the fusion protein may contain a nuclear localization signal), a nuclear transcription factor, a protein binding to a transcription factor, a protein binding to a genomic DNA, a protein binding to a protein binding to a genomic DNA, a constituent protein of chromatin, a protein binding to chromatin, an intracellular cell skeleton, and a protein binding to an intracellular cell skeleton. The intracellular protein is not especially limited, and other examples include a gene product of a cancer driver gene, a protein in an activated signal cascade (particularly in an activated immune cell of a patient having a cancer cell or an immune-related disease), and a gene product of a tumor suppressor gene (particularly under regulation of a binding partner for negative regulation thereof). In one embodiment, the antigen can be Kras.

Introduction of the protein into a cell can be conducted by introducing, into the cell, a protein expression vector containing a nucleic acid encoding a fusion protein (a fusion protein of the peptide tag and the protein of interest) operably linked to a regulatory sequence. The protein expression vector is introduced into a protein-producing cell or a mammal cell, and can express the fusion protein in the cell.

The regulatory sequence can be a promoter capable of transcribing an mRNA, and for example, various types of pol II promoters can be used. The pol II promoters are not especially limited, and examples include a CMV promoter, an EF1 promoter (EF1α promoter), an SV40 promoter, an MSCV promoter, an hTERT promoter, a β actin promoter, a CAG promoter, and a CBh promoter. Further, a promoter driving bacteriophage-derived RNA polymerase, such as a T7 promoter, a T3 promoter, or an SP6 promoter, and a pol III promoter such as a U6 promoter can be used as the promoter. For a cyclic DNA, the T7 promoter can be preferably used, and for a linear DNA, the SP6 promoter can be preferably used. The promoter may be a promoter of a virus. Alternatively, the promoter may be an inducible promoter. The inducible promoter is a promoter capable of inducing expression of a polynucleotide functionally linked to the promoter only in the presence of an inducer driving the promoter. An example of the inducible promoter includes a promoter inducing gene expression by heat such as a heat shock promoter. Another example of the inducible promoter includes a promoter using a drug as the inducer driving the promoter. Examples of such a drug inducible promoter include a Cumate operator sequence, a λ operator sequence (such as 12×λOp), and a tetracycline-based inducible promoter. An example of the tetracycline-based inducible promoter includes a promoter driving gene expression in the presence of tetracycline or a derivative thereof (such as doxycycline), or a reverse tetracycline controlled transactivator (rtTA). An example of the tetracycline-based inducible promoter includes a TRE3G promoter.

The protein expression vector is not especially limited, and can be a virus vector or a plasmid vector. The virus vector is not especially limited, and examples include a retrovirus vector, a lentivirus vector, an adenovirus vector, an adeno-associated virus vector, a herpes simplex virus vector, a vaccinia virus vector, a Sendai virus vector, and a vesicular stomatitis virus vector. From the viewpoint of changing infectiveness to a cell, these vectors may be of pseudo type. These vectors may be derived from attenuated strains. Such a vector can be appropriately prepared by known technique.

From the viewpoint of convenience in production of the protein expression vector, the protein expression vector may contain a nucleic acid encoding the regulatory sequence and the peptide tag of the present disclosure operably linked to the regulatory sequence, and have, on the downstream of the nucleic acid, a cloning site of a nucleic acid encoding the protein of interest. The cloning site has a restriction enzyme cleavage site uniquely present in the vector, and is suitable for introducing a fragment of a gene encoding the protein of interest. A gene encoding the fusion protein of the peptide tag and the protein of interest is obtained by linking a gene encoding the protein of interest in-frame to a gene encoding the peptide tag. Accordingly, the present disclosure provides a protein expression vector containing a nucleic acid encoding the regulatory sequence and the peptide tag of the present disclosure operably linked to the regulatory sequence. In one preferable embodiment, this vector has, on the downstream of the nucleic acid, a cloning site of a nucleic acid encoding the protein of interest. The present disclosure provides a protein expression vector containing: a nucleic acid encoding the regulatory sequence and the peptide tag of the present disclosure operably linked to the regulatory sequence; and a nucleic acid encoding the protein of interest linked in-frame to the former nucleic acid. In this manner, the fusion protein of the peptide tag and the protein of interest can be expressed in a cell.

The present disclosure provides a messenger RNA (mRNA) containing a nucleic acid encoding the peptide tag of the present disclosure. The mRNA further contains a nucleic acid encoding the protein of interest. The nucleic acid encoding the protein of interest is linked in-frame to the nucleic acid encoding the peptide tag. In one embodiment, at least one or more uridines may be changed to pseudouridines in the mRNA. The pseudouridine can be 1-methyl-pseudouridine. The mRNA can be one transcribed from a cDNA, namely, may not have an intron. The mRNA may have a cap structure at the 5′ end (Furuichi Y. & Miura K., Nature, 1975; 253 (5490): 374-5). As the cap structure, a Cap0 structure can be added to the mRNA by Anti-Reverse Cap Analogues (ARCA) method using a cap analogue (Stepinski J. et al., RNA, 2001 Oct; 7(10): 1486-95). When 2′-0 methyltransferase treatment is further performed, the Cap0 structure of the mRNA can be converted to a Cap1 structure. Such an operation can be performed by an ordinary method, and can be practiced using, for example, a commercially available kit, such as ScriptCap m7G Capping System, ScriptCap 2′-O-Methyltransferase Kit, or T7 mScript Standard mRNA Production System (AR Brown Co., Ltd.). The mRNA may have a poly A chain. The addition of a poly A chain can be performed by an ordinary method, and can be performed, for example, with A-Plus Poly(A) Polymerase Tailing Kit (AR Brown Co., Ltd.). Accordingly, in one embodiment, the mRNA can be an mRNA that has a cap structure at the 5′ end, has a poly A chain at the 3′ end, and preferably has pseudouridine (preferably 1-methyl-pseudouridine) as at least a part of uridines. The mRNA can be an isolated mRNA or a synthesized mRNA.

The mRNA can be encapsulated in a nanoparticle, such as a lipid nanoparticle (LNP). Thus, degradation of the mRNA in a living body is prevented, and efficiency of delivering the mRNA into a cell is improved. Accordingly, in one embodiment, the mRNA can be an mRNA that has a cap structure at the 5′ end, has a poly A chain at the 3′ end, and preferably has pseudouridine (preferably 1-methyl-pseudouridine) as at least a part of uridines. Such a lipid nanoparticle encapsulating the mRNA is also provided. The lipid nanoparticle is not especially limited, and lipid nanoparticles described in, for example, U.S. Pat. Nos. 9,364,435B, 8,822,668B, 8,802,644B, and 8,058,069B2 can be used. Alternatively, the mRNA may be encapsulated in a polyion complex micelle, or a polyion complex polymersome (Miyata et al., Chem. Soc. Rev., 2012, 41, 2562-2574). A nanoparticle refers to a particle having a diameter (for example, a hydrodynamic diameter) less than 1 μm.

Accordingly, the present disclosure provides a nanovesicle (such as a lipid nanovesicle, or a polyion complex polymersome) containing an mRNA at least containing a nucleic acid encoding the peptide tag of the present disclosure. The mRNA can contain a nucleic acid encoding the fusion protein of the peptide tag and the protein of interest.

The present disclosure provides a method for reducing, inhibiting, or improving aggregation tendency of a protein, including fusing, to the protein, the peptide tag of the present disclosure that reduces, inhibits, or improves the aggregation tendency. In this embodiment, the protein can be an aggregating protein. The aggregation tendency can be aggregation tendency under an intracellular environment. The fusion is conducted usually on the N terminal and/or the C terminal of the protein. The method can be an in vitro method.

The present disclosure provides a method for increasing, promoting, or improving a non-aggregation property a protein, including fusing, to the protein, the peptide tag of the present disclosure that reduces, inhibits, or improves the aggregation tendency. In this embodiment, the protein can be an aggregating protein. The non-aggregation property can be a non-aggregation property under an intracellular environment. The method can be an in vitro method.

The present disclosure provides a method for increasing, promoting, or improving stability of a protein, including fusing, to the protein, the peptide tag of the present disclosure that reduces, inhibits, or improves aggregation tendency. In this embodiment, the protein can be an aggregating protein. In this embodiment, the protein can be a non-aggregation property. The stability can be stability under an intracellular environment. The method can be an in vitro method.

The present disclosure provides use of the peptide tag of the present disclosure for reducing, inhibiting, or improving aggregation tendency of a protein. The present disclosure also provides use of the peptide tag of the present disclosure for increasing, promoting, or improving a non-aggregation property of a protein. The present disclosure also provides use of the peptide tag of the present disclosure for increasing, promoting, or improving stability of a protein. The use can be use in vitro.

It can be tested by in vitro assay how strongly the peptide tag of the present disclosure can reduce, inhibit, or improve the aggregation tendency of a protein; increase, promote, or improve the non-aggregation property; or increase, promote, or improve the stability. For example, a gene encoding the peptide tag of the present disclosure is fused, for introduction into a cell, to the N terminal or the C terminal of a gene encoding an aggregating protein such as a scFv having an amino acid sequence of SEQ ID NO: 1, and thus, the aggregating protein fused with the peptide tag of the present disclosure can be expressed in the cell. An aggregation formed by the aggregating protein is observed with an antibody against the aggregating protein, and thus, a rate (%) of cells having aggregations to cells expressing the aggregating protein can be calculated. This rate can be used for evaluating influence of the peptide tag of the present disclosure on the aggregation tendency, the non-aggregation tendency, and the stability of the protein.

The present disclosure provides a composition containing the peptide tag of the present disclosure. The peptide tag of the present disclosure can be linked to the protein of interest in a reaction solution by, for example, a click reaction. The click reaction can be a Huisgen reaction. One of the peptide tag and the protein of interest is modified with an alkyne and the other is modified with an azide compound, and thus, a 1,2,3-triazole ring is formed to obtain the link therebetween.

The present disclosure provides a composition containing the fusion protein of the peptide tag of the present invention and the protein of interest. The protein of interest can be an aggregating protein in one embodiment. The protein of interest can be an antigen-binding fragment of an antibody in one embodiment. The protein of interest can be a scFv in one embodiment.

The present disclosure provides an mRNA containing a nucleic acid encoding the fusion protein of the peptide tag of the present disclosure and the protein of interest, and a composition containing the mRNA. The present disclosure provides a vesicle containing an mRNA containing a nucleic acid encoding the fusion protein of the peptide tag of the present disclosure and the protein of interest, and a composition containing the vesicle.

The present disclosure provides a protein expression vector containing a nucleic acid encoding the fusion protein of the peptide tag of the present disclosure operably linked to a regulatory sequence and the protein of interest, and a composition containing the protein expression vector.

In one embodiment, the composition may further contain a pharmaceutically acceptable carrier, excipient, and/or additive. The composition can be a pharmaceutical composition in one embodiment.

In all embodiments of the present disclosure, the fusion protein having the peptide tag of the present disclosure linked thereto can be a non-natural protein.

In all embodiments of the present disclosure, the peptide and the protein can be respectively recombinant peptide and protein.

In all embodiments of the present disclosure, regarding a scFv having at least an amino acid sequence of SEQ ID NO: 1, the peptide tag of the present disclosure can reduce, inhibit, or improve aggregation tendency of the protein; increase, promote, or improve a non-aggregation property; or increase, promote, or improve stability.

A peptide, a protein, and a nucleic acid can be isolated, concentrated, or purified. Isolation means that one or more components of a system are separated from a given component. Purification means that a relative concentration of a given component is increased as compared with a concentration of one or more other components of a system. Concentration means that a concentration of a given component is increased.

One aspect of the present disclosure provides:
a method for acquiring (or selecting or identifying) an amino acid sequence (or a nucleic acid encoding the amino acid sequence), including acquiring an amino acid sequence in which:
(a) 5% or more and less than 45% of amino acids contained in the amino acid sequence are acidic amino acids; and
(b) 20% or more of the amino acids contained in the amino acid sequence are amino acids selected from the group consisting of F, P, Y, G, S, Q, N, and A.

One aspect of the present disclosure provides:
a method for acquiring (or selecting or identifying) an amino acid sequence (or a nucleic acid encoding the amino acid sequence) including:
acquiring an amino acid sequence satisfying any one of conditions (A) to (AE) and (AF) to (AU) described above, or any combination of these conditions.

In one aspect of the present disclosure, the method for acquiring an amino acid sequence may further include:
selecting or identifying an amino acid sequence of a peptide tag that, when a fusion protein of a peptide having the selected or identified amino acid sequence and a reference protein is expressed in a mammal cell (preferably in a human cell), provides reduction in a proportion of cells in which the fusion protein forms an aggregation (aggregation rate) (or the proportion which is not more than a predetermined value).

In one aspect of the present disclosure, the method for acquiring may further include obtaining a peptide having the amino acid sequence, or a nucleic acid encoding the peptide.

In the method for acquiring, the amino acid to be obtained may have a length of 10 to 200 amino acids (for example, a length of 10 to 90 amino acids).

One aspect of the present disclosure provides a method for acquiring (or selecting or identifying) an amino acid sequence (or a nucleic acid encoding the amino acid sequence) having a length of 10 to 200 amino acids (10 to 90 amino acids), including:
acquiring an amino acid sequence in which:
(a) 5% or more and less than 45% of amino acids contained in the amino acid sequence are acidic amino acids; and
(b) 20% or more of the amino acids contained in the amino acid sequence are amino acids selected from the group consisting of F, P, Y, G, S, Q, N, and A;

Selecting or identifying an amino acid sequence of a peptide tag that, when the fusion protein of a peptide having the selected or identified amino acid sequence and a reference protein (reference protein) is expressed in a mammal cell (preferably in a human cell), provides reduction of a proportion of cells in which a fusion protein forms an aggregation (aggregation rate) (or the proportion which is not more than a predetermined value); and
obtaining a peptide having the amino acid, or a nucleic acid encoding the peptide.

In this method, a peptide having a particularly excellent stabilizing action can be selected from peptides having a stabilizing action by increasing the extent of reduction or by reducing the predetermined value. In one aspect, the present method may further include expressing, in a mammal cell (preferably in a human cell), the fusion protein of the peptide having the selected or identified amino acid sequence and the reference protein. The present method may further include obtaining a nucleic acid encoding the selected or identified amino acid sequence. The predetermined value can be a numerical value based on a rate (%) of cells having an aggregation to cells expressing the aggregating protein. For example, the predetermined value can be a value of 30% or less, a value of 20% or less, a value of 15% or less, a value of 10% or less, a value of 5% or less, a value of 3% or less, a value of 2% or less, or a value of 1% or less, or 0%. The predetermined value can be a value in a range of 0% to 10%, a value in a range of 10% to 20%, or a value in a range of 20 to 30%. When a peptide having a higher effect is desired to be acquired, the predetermined value is preferably smaller. In this embodiment, a peptide tag exhibiting a stronger effect can be selected from, for example, the above-described peptide tags of the present disclosure (for example, any one of the peptide tags of (A) to (Z), (AA) to (AE), and (AF) to (AU) described above).

The reference protein can be, for example, a protein (aggregating protein, such as a scFv) in which the proportion of cells having an aggregation formed therein by the fusion protein is more than 30%, 40% to 50% or less, 50% to 60%, 60% to 70%, 70% to 80%, 80% to 90%, 90% to 95%, 95% to 99%, 99% to 99.9%, or 99.9% or more. The reference protein needs not have special functionality or binding property to an antigen but is used simply for evaluating the reduction of the aggregation rate, and therefore, the CDR sequence thereof may be any sequence. The aggregation rate of the scFv may be varied depending on the amino acid sequence of the CDR. It is possible to search for an amino acid sequence of a peptide tag that provides the aggregation rate not more than the predetermined value in the scFv having the varied aggregation rate. In one preferable embodiment, the reference protein can be a protein having an amino acid sequence of SEQ ID NO: 1.

In one embodiment, the amino acid sequence to be obtained further satisfies the condition defined in (B) described above. In one embodiment, the amino acid sequence to be acquired further satisfies the condition defined in (D) described above. In one embodiment, the amino acid sequence to be acquired further satisfies the condition defined in (E) described above. In one embodiment, the amino acid sequence to be acquired further satisfies the condition defined in (F) described above. In one embodiment, the amino acid sequence to be acquired further satisfies the condition defined in (G) described above. In one embodiment, the amino acid sequence to be acquired further satisfies the condition defined in (H) described above. In one embodiment, the amino acid sequence to be acquired further satisfies the condition defined in (I) described above. In one embodiment, the amino acid sequence to be acquired further satisfies the condition defined in (I) described above. In one embodiment, the amino acid sequence to be acquired further satisfies the condition defined in (J) described above. In one embodiment, the amino acid sequence to be acquired further satisfies the condition defined in (K) described above. In one embodiment, the amino acid sequence to be acquired further satisfies the condition defined in (L) described above. In one embodiment, the amino acid sequence to be acquired further satisfies the condition defined in (M) described above. In one embodiment, the amino acid sequence to be acquired further satisfies the condition defined in (N) described above. In one embodiment, the amino acid sequence to be acquired further satisfies the condition defined in (O) described above. In one embodiment, the amino acid sequence to be acquired further satisfies the condition defined in (P) described above. In one embodiment, the amino acid sequence to be acquired further satisfies the condition defined in (Q) described above. In one embodiment, the amino acid sequence to be acquired further satisfies the condition defined in (R) described above. In one embodiment, the amino acid sequence to be acquired further satisfies the condition defined in (S) described above. In one embodiment, the amino acid sequence to be acquired further satisfies the condition defined in (T) described above. In one embodiment, the amino acid sequence to be acquired further satisfies the condition defined in (U) described above. In one embodiment, the amino acid sequence to be acquired further satisfies the condition defined in (V) described above. In one embodiment, the amino acid sequence to be acquired further satisfies the condition defined in (W) described above. In one embodiment, the amino acid sequence to be acquired further satisfies the condition defined in (X) described above. In one embodiment, the amino acid sequence to be acquired further satisfies the condition defined in (Y) described above. In one embodiment, the amino acid sequence to be acquired further satisfies the condition defined in (Z) described above. In one embodiment, the amino acid sequence to be acquired further satisfies the condition defined in (AA) described above. In one embodiment, the amino acid sequence to be acquired further satisfies the condition defined in (AB) described above. In one embodiment, the amino acid sequence to be acquired further satisfies the condition defined in (AC) described above. In one embodiment, the amino acid sequence to be acquired further satisfies the condition defined in (AD) described above. In one embodiment, the amino acid sequence to be acquired further satisfies the condition defined in (AE) described above. In one embodiment, the amino acid sequence to be acquired further satisfies the condition defined in (AF) described above. In one embodiment, the amino acid sequence to be acquired further satisfies the condition defined in (AG) described above. In one embodiment, the amino acid sequence to be acquired further satisfies the condition defined in (AH) described above. In one embodiment, the amino acid sequence to be acquired further satisfies the condition defined in (AI) described above. In one embodiment, the amino acid sequence to be acquired further satisfies the condition defined in (AJ) described above. In one embodiment, the amino acid sequence to be acquired further satisfies the condition defined in (AK) described above. In one embodiment, the amino acid sequence to be acquired further satisfies the condition defined in (AL) described above. In one embodiment, the amino acid sequence to be acquired further satisfies the condition defined in (AM) described above. In one embodiment, the amino acid sequence to be acquired further satisfies the condition defined in (AN) described above. In one embodiment, the amino acid sequence to be acquired further satisfies the condition defined in (AO) described above. In one embodiment, the amino acid sequence to be acquired further satisfies the condition defined in (AP) described above. In one embodiment, the amino acid sequence to be acquired further satisfies the condition defined in (AQ) described above. In one embodiment, the amino acid sequence to be acquired further satisfies the condition defined in (AR) described above. In one embodiment, the amino acid sequence to be acquired further satisfies the condition defined in (AS) described above. In one embodiment, the amino acid sequence to be acquired further satisfies the condition defined in (AT) described above. In one embodiment, the amino acid sequence to be acquired further satisfies the condition defined in (AU) described above. In one embodiment, the amino acid sequence to be obtained satisfies the condition described in any one or more of (A) to (Z), (AA) to (AE), and (AF) to (AU) described above.

In one embodiment, the amino acid sequence to be acquired can be selected from an amino acid sequence group.

In one embodiment, the amino acid sequence group can be a group of the amino acid sequences defined in (A) above, or can include this group.

In one embodiment, the amino acid sequence group can be a group of the amino acid sequences defined in (B) above, or can include this group.

In one embodiment, the amino acid sequence group can be a group of the amino acid sequences defined in (C) above, or can include this group.

In one embodiment, the amino acid sequence group can be a group of the amino acid sequences defined in (D) above, or can include this group.

In one embodiment, the amino acid sequence group can be a group of the amino acid sequences defined in (E) above, or can include this group.

In one embodiment, the amino acid sequence group can be a group of the amino acid sequences defined in (F) above, or can include this group.

In one embodiment, the amino acid sequence group can be a group of the amino acid sequences defined in (G) above, or can include this group.

In one embodiment, the amino acid sequence group can be a group of the amino acid sequences defined in (H) above, or can include this group.

In one embodiment, the amino acid sequence group can be a group of the amino acid sequences defined in (I) above, or can include this group.

In one embodiment, the amino acid sequence group can be a group of the amino acid sequences defined in (J) above, or can include this group.

In one embodiment, the amino acid sequence group can be a group of the amino acid sequences defined in (K) above, or can include this group.

In one embodiment, the amino acid sequence group can be a group of the amino acid sequences defined in (L) above, or can include this group.

In one embodiment, the amino acid sequence group can be a group of the amino acid sequences defined in (M) above, or can include this group.

In one embodiment, the amino acid sequence group can be a group of the amino acid sequences defined in (N) above, or can include this group.

In one embodiment, the amino acid sequence group can be a group of the amino acid sequences defined in (O) above, or can include this group.

In one embodiment, the amino acid sequence group can be a group of the amino acid sequences defined in (P) above, or can include this group.

In one embodiment, the amino acid sequence group can be a group of the amino acid sequences defined in (Q) above, or can include this group.

In one embodiment, the amino acid sequence group can be a group of the amino acid sequences defined in (R) above, or can include this group.

In one embodiment, the amino acid sequence group can be a group of the amino acid sequences defined in (S) above, or can include this group.

In one embodiment, the amino acid sequence group can be a group of the amino acid sequences defined in (T) above, or can include this group.

In one embodiment, the amino acid sequence group can be a group of the amino acid sequences defined in (U) above, or can include this group.

In one embodiment, the amino acid sequence group can be a group of the amino acid sequences defined in (V) above, or can include this group.

In one embodiment, the amino acid sequence group can be a group of the amino acid sequences defined in (W) above, or can include this group.

In one embodiment, the amino acid sequence group can be a group of the amino acid sequences defined in (X) above, or can include this group.

In one embodiment, the amino acid sequence group can be a group of the amino acid sequences defined in (Y) above, or can include this group.

In one embodiment, the amino acid sequence group can be a group of the amino acid sequences defined in (Z) above, or can include this group.

In one embodiment, the amino acid sequence group can be a group of the amino acid sequences defined in (Ak) above, or can include this group.

In one embodiment, the amino acid sequence group can be a group of the amino acid sequences defined in (AB) above, or can include this group.

In one embodiment, the amino acid sequence group can be a group of the amino acid sequences defined in (AC) above, or can include this group.

In one embodiment, the amino acid sequence group can be a group of the amino acid sequences defined in (AD) above, or can include this group.

In one embodiment, the amino acid sequence group can be a group of the amino acid sequences defined in (AE) above, or can include this group.

In one embodiment, the amino acid sequence group can be a group of the amino acid sequences defined in (AF) above, or can include this group.

In one embodiment, the amino acid sequence group can be a group of the amino acid sequences defined in (AG) above, or can include this group.

In one embodiment, the amino acid sequence group can be a group of the amino acid sequences defined in (AH) above, or can include this group.

In one embodiment, the amino acid sequence group can be a group of the amino acid sequences defined in (AI) above, or can include this group.

In one embodiment, the amino acid sequence group can be a group of the amino acid sequences defined in (AJ) above, or can include this group.

In one embodiment, the amino acid sequence group can be a group of the amino acid sequences defined in (AK) above, or can include this group.

In one embodiment, the amino acid sequence group can be a group of the amino acid sequences defined in (AL) above, or can include this group.

In one embodiment, the amino acid sequence group can be a group of the amino acid sequences defined in (AM) above, or can include this group.

In one embodiment, the amino acid sequence group can be a group of the amino acid sequences defined in (AN) above, or can include this group.

In one embodiment, the amino acid sequence group can be a group of the amino acid sequences defined in (AO) above, or can include this group.

In one embodiment, the amino acid sequence group can be a group of the amino acid sequences defined in (AP) above, or can include this group.

In one embodiment, the amino acid sequence group can be a group of the amino acid sequences defined in (AQ) above, or can include this group.

In one embodiment, the amino acid sequence group can be a group of the amino acid sequences defined in (AR) above, or can include this group.

In one embodiment, the amino acid sequence group can be a group of the amino acid sequences defined in (AS) above, or can include this group.

In one embodiment, the amino acid sequence group can be a group of the amino acid sequences defined in (AT) above, or can include this group.

In one embodiment, the amino acid sequence group can be a group of the amino acid sequences defined in (AU) above, or can include this group.

In one preferable embodiment, the aggregation rate of the reference protein is 5 to 10%, and the aggregation rate of the fusion protein is lower than this aggregation rate.

In one preferable embodiment, the aggregation rate of the reference protein is 5 to 10%, and the aggregation rate of the fusion protein is not more than a predetermined value, which is lower than this aggregation rate by 1% or more.

In one preferable embodiment, the aggregation rate of the reference protein is 5 to 10%, and the aggregation rate of the fusion protein is not more than a predetermined value, which is lower than this aggregation rate by 2% or more.

In one preferable embodiment, the aggregation rate of the reference protein is 5 to 10%, and the aggregation rate of the fusion protein is not more than a rate by 3% or more.

In one preferable embodiment, the aggregation rate of the reference protein is 5 to 10%, and the aggregation rate of the fusion protein is not more than a predetermined value, which is lower than this aggregation rate by 4% or more.

In one preferable embodiment, the aggregation rate of the reference protein is 6 to 10%, and the aggregation rate of the fusion protein is not more than a predetermined value, which is lower than this aggregation rate by 5% or more.

In one preferable embodiment, the aggregation rate of the reference protein is 7 to 10%, and the aggregation rate of the fusion protein is not more than a predetermined value, which is lower than this aggregation rate by 6% or more.

In one preferable embodiment, the aggregation rate of the reference protein is 8 to 10%, and the aggregation rate of the fusion protein is not more than a predetermined value, which is lower than this aggregation rate by 7% or more.

In one preferable embodiment, the aggregation rate of the reference protein is 9 to 10%, and the aggregation rate of the fusion protein is not more than a predetermined value, which is lower than this aggregation rate by 8% or more.

In a preferable embodiment, a ratio of the aggregation rate of the fusion protein to the aggregation rate of the reference protein is not more than a predetermined value, which is 0.9 or less, 0.8 or less, 0.7 or less, 0.6 or less, 0, or less, 0.4 or less, 0.3 or less, 0.2 or less, or 0.1 or less.

In one preferable embodiment, the aggregation rate of the reference protein is 5 to 10%, and the ratio of the aggregation rate of the fusion protein to this aggregation rate is not more than a predetermined value, which is 0.9 or less.

In one preferable embodiment, the aggregation rate of the reference protein is 5 to 10%, and the ratio of the aggregation rate of the fusion protein to this aggregation rate is not more than a predetermined value, which is 0.8 or less.

In one preferable embodiment, the aggregation rate of the reference protein is 5 to 10%, and the ratio of the aggregation rate of the fusion protein to this aggregation rate is not more than a predetermined value, which is 0.7 or less.

In one preferable embodiment, the aggregation rate of the reference protein is 5 to 10%, and the ratio of the aggregation rate of the fusion protein to this aggregation rate is not more than a predetermined value, which is 0.6 or less.

In one preferable embodiment, the aggregation rate of the reference protein is 5 to 10%, and the ratio of the aggregation rate of the fusion protein to this aggregation rate is not more than a predetermined value, which is 0.5 or less.

In one preferable embodiment, the aggregation rate of the reference protein is 5 to 10%, and the ratio of the aggregation rate of the fusion protein to this aggregation rate is not more than a predetermined value, which is 0.4 or less.

In one preferable embodiment, the aggregation rate of the reference protein is 5 to 10%, and the ratio of the aggregation rate of the fusion protein to this aggregation rate is not more than a predetermined value, which is 0.3 or less.

In one preferable embodiment, the aggregation rate of the reference protein is 5 to 10%, and the ratio of the aggregation rate of the fusion protein to this aggregation rate is not more than a predetermined value, which is 0.2 or less.

In one preferable embodiment, the aggregation rate of the reference protein is 5 to 10%, and the ratio of the aggregation rate of the fusion protein to this aggregation rate is not more than a predetermined value, which is 0.1 or less.

In one embodiment, the aggregation rate of the reference protein is less than 5%, and the aggregation rate of the fusion protein is lower than this aggregation rate.

In one preferable embodiment, the aggregation rate of the reference protein is less than 5%, and the ratio of the aggregation rate of the fusion protein to this aggregation rate is not more than a predetermined value, which is 0.9 or less.

In one preferable embodiment, the aggregation rate of the reference protein is less than 5%, and the ratio of the aggregation rate of the fusion protein to this aggregation rate is not more than a predetermined value, which is 0.8 or less.

In one preferable embodiment, the aggregation rate of the reference protein is less than 5%, and the ratio of the aggregation rate of the fusion protein to this aggregation rate is not more than a predetermined value, which is 0.7 or less.

In one preferable embodiment, the aggregation rate of the reference protein is less than 5%, and the ratio of the aggregation rate of the fusion protein to this aggregation rate is not more than a predetermined value, which is 0.6 or less.

In one preferable embodiment, the aggregation rate of the reference protein is less than 5%, and the ratio of the aggregation rate of the fusion protein to this aggregation rate is not more than a predetermined value, which is 0.5 or less.

In one preferable embodiment, the aggregation rate of the reference protein is less than 5%, and the ratio of the aggregation rate of the fusion protein to this aggregation rate is not more than a predetermined value, which is 0.4 or less.

In one preferable embodiment, the aggregation rate of the reference protein is less than 5%, and the ratio of the aggregation rate of the fusion protein to this aggregation rate is not more than a predetermined value, which is 0.3 or less.

In one preferable embodiment, the aggregation rate of the reference protein is less than 5%, and the ratio of the aggregation rate of the fusion protein to this aggregation rate is not more than a predetermined value, which is 0.2 or less.

In one preferable embodiment, the aggregation rate of the reference protein is less than 5%, and the ratio of the aggregation rate of the fusion protein to this aggregation rate is not more than a predetermined value, which is 0.1 or less.

In one preferable embodiment, the aggregation rate of the reference protein is more than 10%, and the aggregation rate of the fusion protein is not more than a rate by 10% or more.

In one preferable embodiment, the aggregation rate of the reference protein is more than 20%, and the aggregation rate of the fusion protein is not more than a predetermined value, which is lower than this aggregation rate by 10% or more.

In one preferable embodiment, the aggregation rate of the reference protein is more than 20%, and the aggregation rate of the fusion protein is not more than a predetermined value, which is lower than this aggregation rate by 20% or more.

In one preferable embodiment, the aggregation rate of the reference protein is more than 30%, and the aggregation rate of the fusion protein is not more than a predetermined value, which is lower than this aggregation rate by 10% or more.

In one preferable embodiment, the aggregation rate of the reference protein is more than 30%, and the aggregation rate of the fusion protein is not more than a predetermined value, which is lower than this aggregation rate by 20% or more.

In one preferable embodiment, the aggregation rate of the reference protein is more than 30%, and the aggregation rate of the fusion protein is not more than a rate by 30% or more.

In one preferable embodiment, the aggregation rate of the reference protein is more than 40%, and the aggregation rate of the fusion protein is not more than a predetermined value, which is lower than this aggregation rate by 10% or more.

In one preferable embodiment, the aggregation rate of the reference protein is more than 40%, and the aggregation rate of the fusion protein is not more than a predetermined value, which is lower than this aggregation rate by 20% or more.

In one preferable embodiment, the aggregation rate of the reference protein is more than 40%, and the aggregation rate of the fusion protein is not more than a predetermined value, which is lower than this aggregation rate by 30% or more.

In one preferable embodiment, the aggregation rate of the reference protein is more than 40%, and the aggregation rate of the fusion protein is not more than a predetermined value, which is lower than this aggregation rate by 40% or more.

In one preferable embodiment, the aggregation rate of the reference protein is more than 50%, and the aggregation rate of the fusion protein is not more than a rate by 10% or more.

In one preferable embodiment, the aggregation rate of the reference protein is more than 50%, and the aggregation rate of the fusion protein is not more than a predetermined value, which is lower than this aggregation rate by 20% or more.

In one preferable embodiment, the aggregation rate of the reference protein is more than 50%, and the aggregation rate of the fusion protein is not more than a predetermined value, which is lower than this aggregation rate by 30% or more.

In one preferable embodiment, the aggregation rate of the reference protein is more than 50%, and the aggregation rate of the fusion protein is not more than a predetermined value, which is lower than this aggregation rate by 40% or more.

In one preferable embodiment, the aggregation rate of the reference protein is more than 50%, and the aggregation rate of the fusion protein is not more than a predetermined value, which is lower than this aggregation rate by 50% or more.

In one preferable embodiment, the aggregation rate of the reference protein is more than 60%, and the aggregation rate of the fusion protein is not more than a rate by 10% or more.

In one preferable embodiment, the aggregation rate of the reference protein is more than 60%, and the aggregation rate of the fusion protein is not more than a predetermined value, which is lower than this aggregation rate by 20% or more.

In one preferable embodiment, the aggregation rate of the reference protein is more than 60%, and the aggregation rate of the fusion protein is not more than a predetermined value, which is lower than this aggregation rate by 30% or more.

In one preferable embodiment, the aggregation rate of the reference protein is more than 60%, and the aggregation rate of the fusion protein is not more than a predetermined value, which is lower than this aggregation rate by 40% or more.

In one preferable embodiment, the aggregation rate of the reference protein is more than 60%, and the aggregation rate of the fusion protein is not more than a predetermined value, which is lower than this aggregation rate by 50% or more.

In one preferable embodiment, the aggregation rate of the reference protein is more than 60%, and the aggregation rate of the fusion protein is not more than a rate by 60% or more.

In one preferable embodiment, the aggregation rate of the reference protein is more than 70%, and the aggregation rate of the fusion protein is not more than a predetermined value, which is lower than this aggregation rate by 10% or more.

In one preferable embodiment, the aggregation rate of the reference protein is more than 70%, and the aggregation rate of the fusion protein is not more than a predetermined value, which is lower than this aggregation rate by 20% or more.

In one preferable embodiment, the aggregation rate of the reference protein is more than 70%, and the aggregation rate of the fusion protein is not more than a predetermined value, which is lower than this aggregation rate by 30% or more.

In one preferable embodiment, the aggregation rate of the reference protein is more than 70%, and the aggregation rate of the fusion protein is not more than a predetermined value, which is lower than this aggregation rate by 40% or more.

In one preferable embodiment, the aggregation rate of the reference protein is more than 70%, and the aggregation rate of the fusion protein is not more than a rate by 50% or more.

In one preferable embodiment, the aggregation rate of the reference protein is more than 70%, and the aggregation rate of the fusion protein is not more than a predetermined value, which is lower than this aggregation rate by 60% or more.

In one preferable embodiment, the aggregation rate of the reference protein is more than 70%, and the aggregation rate of the fusion protein is not more than a predetermined value, which is lower than this aggregation rate by 70% or more.

In one preferable embodiment, the aggregation rate of the reference protein is more than 80%, and the aggregation rate of the fusion protein is not more than a predetermined value, which is lower than this aggregation rate by 10% or more.

In one preferable embodiment, the aggregation rate of the reference protein is more than 80%, and the aggregation rate of the fusion protein is not more than a predetermined value, which is lower than this aggregation rate by 20% or more.

In one preferable embodiment, the aggregation rate of the reference protein is more than 80%, and the aggregation rate of the fusion protein is not more than a rate by 30% or more.

In one preferable embodiment, the aggregation rate of the reference protein is more than 80%, and the aggregation rate of the fusion protein is not more than a predetermined value, which is lower than this aggregation rate by 40% or more.

In one preferable embodiment, the aggregation rate of the reference protein is more than 80%, and the aggregation rate of the fusion protein is not more than a predetermined value, which is lower than this aggregation rate by 50% or more.

In one preferable embodiment, the aggregation rate of the reference protein is more than 80%, and the aggregation rate of the fusion protein is not more than a predetermined value, which is lower than this aggregation rate by 60% or more.

In one preferable embodiment, the aggregation rate of the reference protein is more than 80%, and the aggregation rate of the fusion protein is not more than a predetermined value, which is lower than this aggregation rate by 70% or more.

In one preferable embodiment, the aggregation rate of the reference protein is more than 80%, and the aggregation rate of the fusion protein is not more than a rate by 80% or more.

In one preferable embodiment, the aggregation rate of the reference protein is more than 90%, and the aggregation rate of the fusion protein is not more than a predetermined value, which is lower than this aggregation rate by 10% or more.

In one preferable embodiment, the aggregation rate of the reference protein is more than 90%, and the aggregation rate of the fusion protein is not more than a predetermined value, which is lower than this aggregation rate by 20% or more.

In one preferable embodiment, the aggregation rate of the reference protein is more than 90%, and the aggregation rate of the fusion protein is not more than a predetermined value, which is lower than this aggregation rate by 30% or more.

In one preferable embodiment, the aggregation rate of the reference protein is more than 90%, and the aggregation rate of the fusion protein is not more than a predetermined value, which is lower than this aggregation rate by 40% or more.

In one preferable embodiment, the aggregation rate of the reference protein is more than 90%, and the aggregation rate of the fusion protein is not more than a rate by 50% or more.

In one preferable embodiment, the aggregation rate of the reference protein is more than 90%, and the aggregation rate of the fusion protein is not more than a predetermined value, which is lower than this aggregation rate by 60% or more.

In one preferable embodiment, the aggregation rate of the reference protein is more than 90%, and the aggregation rate of the fusion protein is not more than a predetermined value, which is lower than this aggregation rate by 70% or more.

In one preferable embodiment, the aggregation rate of the reference protein is more than 90%, and the aggregation rate of the fusion protein is not more than a predetermined value, which is lower than this aggregation rate by 80% or more.

In one preferable embodiment, the aggregation rate of the reference protein is more than 90%, and the aggregation rate of the fusion protein is not more than a predetermined value, which is lower than this aggregation rate by 90% or more.

A nucleic acid encoding the obtained peptide tag is linked in-frame to a nucleic acid encoding the protein of interest, and thus, a nucleic acid encoding the fusion protein of the peptide tag and the protein of interest can be obtained. From the nucleic acid encoding the fusion protein, the fusion protein can be expressed. A protein expression vector containing a nucleic acid encoding a fusion protein operably linked to a regulatory sequence may be prepared.

In one aspect of the present disclosure, the method can be employed for determining whether or not a peptide tag having an amino acid sequence with a length of 10 to 200 amino acids (for example, 10 to 90 amino acids) has an effect of improving an aggregation property not less than a predetermined intensity against a tagged protein. In other words, in one aspect of the present disclosure, a method for determining whether or not a peptide tag having an amino acid sequence with a length of 10 to 90 amino acids has an effect of improving an aggregation property not less than a predetermined intensity against a tagged protein, and the method including:
acquiring an amino acid sequence in which:
(α) 5% or more and less than 45% of amino acids contained in the amino acid sequence are acidic amino acids; and
(β) 20% or more of the amino acids contained in the amino acid sequence are amino acids selected from the group consisting of F, P, Y, G, S, Q, N, and A;
determining that an amino acid sequence of a peptide tag that, when the fusion protein of a peptide having the selected or identified amino acid sequence and a reference protein is expressed in a mammal cell (preferably in a human cell), provides reduction of a proportion of cells in which a fusion protein forms an aggregation (aggregation rate) (or the proportion which is not more than a predetermined value), has the effect of improving an aggregation property (for example, an aggregation property not less than a predetermined intensity).

In one embodiment, the method may further include expressing, in a mammal cell (preferably in a human cell), the fusion protein of the peptide having the selected or identified amino acid sequence and the reference protein. In one embodiment, the amino acid sequence to be acquired can be selected from an amino acid sequence group. The details of the amino acid group are the same as those described above. In one aspect, the amino acid sequence to be acquired satisfies another one or more conditions. The conditions are the same as those described above. In one embodiment, the aggregation rate of the reference protein and the predetermined value are the same as those described above.

In one embodiment, in the method for selecting or identifying an amino acid sequence having a length of 10 to 200 amino acids (for example, 10 to 90 amino acids), the amino acid sequence group having a length of 10 to 200 amino acids (for example, 10 to 90 amino acids) can be a group of amino acid sequences encoded by the coding region of the human genome. In one embodiment, the amino acid sequence group having a length of 10 to 90 amino acids can be a group of amino acid sequences encoded by the coding region of the genome of a non-human living thing.

In one embodiment, the amino acid sequence having a length of 10 to 200 amino acids (for example, 10 to 90 amino acids) can be a neo-antigen. A neo-antigen was discovered as a mutant antigen newly caused by gene mutation peculiar to a cancer cell. The neo-antigen is not expressed in a non-cancer cell. It is expected that immunity can be induced specifically to cancer by inducing immunity to a neo-antigen by administering a peptide containing the neo-antigen. The neo-antigen can be different among cancer cells. The neo-antigen can be used, for example, for tagging a protein of interest to be expressed in a cell having the neo-antigen, and can be thus usable because it is a peptide originally expressed, and hence the cell is not or little adversely affected. The neo-antigen can be a naturally occurring mutant. The neo-antigen has one or more mutations selected from the group consisting of addition, insertion, deletion, and substitution in, for example, a wild type sequence thereof. For example, a neo-antigen of a human typically has one or more (for example, 1 to 10, for example, 1 to several, 1 to 5, 1 to 4, 1 to 3, 2 or 1) mutations selected from the group consisting of addition, insertion, deletion, and substitution in a wild type sequence of a human. The neo-antigen of a human can have, for example, 80% or more identity, 85% or more identity, 90% or more identity, or 95% or more identity to the wild type sequence of a human.

In one preferable embodiment, the reference protein is a scFv. In one embodiment, the scFv has the amino acid sequence of SEQ ID NO: 1.

Still another aspect of the present invention provides a method for modifying an amino acid sequence of a peptide tag, including:
preparing a peptide tag (that may be any one of the peptide tags disclosed herein) for producing a fusion protein; and
obtaining a modified amino acid sequence by substituting, with either of P and N, one or more (preferably, a plurality of) amino acids of the Element 2, 3, or 4 (preferably, any one of the Element 2, the Element 4, and A, G, Y, and F).
   The method of this aspect may further include:
determining that an amino acid sequence of a peptide tag that, when the fusion protein of a peptide having a selected or identified amino acid sequence and a reference protein is expressed in a mammal cell (preferably in a human cell), provides reduction of a proportion of cells in which a fusion protein forms an aggregation (aggregation rate) (or the proportion which is not more than a predetermined value), has the effect of improving an aggregation property (for example, an aggregation property not less than a predetermined intensity).
   In this manner, modification having particularly strong aggregation reducing action can be performed.

### EXAMPLES

### Example 1

### [Method]

### Construction of Gene Expression Vector

A gene fragment encoding a fusion protein containing a peptide tag and an aggregating protein was produced by Eurofins Genomics K.K. or VectorBuilder Japan, Inc. The thus synthesized gene fragment was cloned into a pEF-BOS vector (Mizushima and Nagata, Nucleic Acids Res. 1990 Sep. 11; 18 (17): 5322). As the aggregating protein, a protein (specifically, a scFv) having an amino acid sequence set forth in SEQ ID NO: 1 was used. This aggregating protein aggregates in the cytoplasm when expressed in a cell. The sequences of tags used here and SEQ ID NOs thereof were as shown in Table 1 below.

TABLE 1
List of Tags used in Experiment

| | | SEQ ID |
|---|---|---|
| Tag Name | Amino Acid Sequence | NO: |
| Tag-1-1 | AHSSSAESESTSDSDSSSDS | 2 |
| | ESESSSSDSEGS | |
| Tag-1-2 | AHSLSAELESTIDSDCSSDW | 3 |
| | ESELSSSDSEGS | |
| Tag-1-3 | AQSSSAESESGSDSDSSSDS | 4 |
| | ESESSSSDSEGS | |
| Tag-4-1 | NEGYREAFDEDYEQQDEDFA | 5 |
| | EQDPDGNEAFEGEYDGPNQD | |
| | EYPDEAQNFE | |
| Tag-2-1 | DEAGSSGAPADEAGSSGAPA | 6 |
| | DEAGSSGAPADEAGSSGAPA | |
| | DEAGSSGAPAGS | |
| Tag-2-2 | DEVGISLAPTDEVGISLAPT | 7 |
| | DEVGISLAPTDEVGISLAPT | |
| | DEVGISLAPTGS | |
| Tag-2-3 | DEVMISLWPTDEVMISLWPT | 8 |
| | DEVMISLWPTDEVMISLWPT | |
| | DEVMISLWPTGS | |
| Tag-3-1 | DEAGSSGAPADFAGSSGAPA | 9 |
| | DFAGSSGAPADFAGSSGAPA | |
| | DFAGSSGAPAGS | |
| Tag-3-2 | DTAVSSIAPLDTAVSSIAPL | 10 |
| | DTAVSSIAPLDTAVSSIAPL | |
| | DTAVSSIAPLGS | |
| Tag-3-3 | DTWVSDIAILDTWVSLIAIL | 11 |
| | DTWVSLIAILDTWVSLIAIL | |
| | DTWVSLIAILGS | |
| Myc | EQKLISEEDL | 12 |

Specific content ratios of amino acids were as follows.

TABLE 2
Amino Acid Content Ratios in Each Peptide and Influence of Tag
Addition on Intracellular Aggregation Formation of Protein

| | Element | Element | Element | Element | S | G | A | Aggregation |
|---|---|---|---|---|---|---|---|---|
| Tag Name | 1 (%) | 2 (%) | 3 (%) | 4 (%) | (%) | (%) | (%) | Rate (%) |
| Myc | 40.0 | 10.0 | 20.0 | 30.0 | 10.0 | 0 | 0 | 40.1 |
| (control) | | | | | | | | |
| Tag-1-1 | 28.1 | 3.1 | 65.6 | 3.1 | 56.3 | 3.1 | 6.3 | 11.4 |
| Tag-1-2 | 28.1 | 3.1 | 46.9 | 21.9 | 37.5 | 3.1 | 6.3 | 20.2 |
| Tag-1-3 | 28.1 | 0.0 | 71.9 | 0.0 | 56.3 | 6.3 | 6.3 | 15.7 |
| Tag-4-1 | 42.0 | 2.0 | 56.0 | 0.0 | 0.0 | 8.0 | 8.0 | 4.0 |
| Tag-2-1 | 19.2 | 0.0 | 80.8 | 0.0 | 21.2 | 21.2 | 28.8 | 29.7 |
| Tag-2-2 | 19.2 | 0.0 | 42.3 | 38.5 | 11.5 | 11.5 | 9.6 | 22.5 |
| Tag-2-3 | 19.2 | 0.0 | 23.1 | 57.7 | 11.5 | 1.9 | 0.0 | 41.2 |
| Tag-3-1 | 9.6 | 0.0 | 90.4 | 0.0 | 21.2 | 21.2 | 28.8 | 43.4 |
| Tag-3-2 | 9.6 | 0.0 | 51.9 | 38.5 | 21.2 | 1.9 | 19.2 | 26.1 |
| Tag-3-3 | 9.6 | 0.0 | 23.1 | 67.3 | 11.5 | 1.9 | 9.6 | 65.0 |

In Table 2, Element 1 refers to D and E, Element 2 refers to H, K, and R, Element 3 refers to F, P, Y, G, S, Q, N, and A, and Element 4 refers to the other amino acids.

The peptide tag was fused to the N terminal of an antibody fragment. The antibody fragment fused with the peptide tag was linked, for detection, to an HA tag of SEQ ID NO: 13 at the C terminal of the antibody fragment.

### Measurement of Intracellular Aggregation Rate of Intracellular Antibody

### (Cell Culture)

A HeLa cell derived from human cervical epithelial cancer was prepared. The HeLa cell was purchased from JCRB Cell Bank, National Institutes of Biomedical Innovation, Health and Nutrition (JCRB9004), and cultured in DMEM (D-MEM, FUJIFILM Wako Pure Chemical Corporation, 4548995066251) containing 10% FBS.

### (Transfection)

In a 35 mm glass bottom dish (IWAKI 3911-035, glass hole, inner diameter: 12 mm) coated with poly-L-lysine (Sigma-Aldrich P1399 Poly-L-lysine hydrobromide mol. Wt. 150000-300000), 4×10⁵ HeLa cells were plated, and after 24 hours, the antibody fragment gene described above was introduced into the HeLa cells with Lipofectamine 3000 (Invitrogen, L3000-008) based on a use method provided by the manufacturer, and thus, a tagged antibody fragment was expressed in the cells. A culture fluid was removed 24 hours after the transfection, and the resultant cells were fixed with 4% PFA. 20 minutes after the fixation, the resultant cells were washed with PBS(−), and thereafter, the antibody expressed in the cells (hereinafter, simply referred to as the “intracellular antibody”) was observed by immunostaining to measure the aggregation rate thereof.

### (Immunostaining)

The immunostaining was performed by a standard method. The cells were treated with 0.3% Triton X−100/PBS(−) for 2 minutes, and kept for 1 hour at room temperature in a blocking solution (1% BSA, 0.1% Triton X−100/PBS(−)). The cells were kept for 2 hours at room temperature in an anti-HA antibody (rabbit anti-Ha antibody: Sigma-Aldrich, H6908) diluted with the blocking solution, the resultant cells were washed with the blocking solution, and then the resultant cells were kept at room temperature in Alexa Fluor 488 Goat anti-rabbit IgG (H+L) (Invitrogen, A11034) for 2 hours. The resultant cells were washed with the blocking solution, kept for 15 minutes at room temperature in a nuclear staining probe (NucBlue Fixed cell stain ReadyProbes, Invitrogen, R37606), washed with PBS(−), and stored at 4° C. until fluorescence imaging.

### (Fluorescence Imaging)

The fluorescence imaging was performed with Keyence BZ-X700 or BZ-X800 using a 40× objective lens. An image including 200 or more cells per dish was acquired to count the number of cells having aggregation of the intracellular antibody therein (intracellular antibody aggregating cells). The number of the intracellular antibody aggregating cells was normalized with the total number of cells expressing the intracellular antibody to quantify an intracellular aggregation rate of the intracellular antibody.

### [Results]

The peptides having the amino acid sequences shown in Table 1 were fused with antigen-binding fragments (specifically, scFvs) of antibodies, and the thus obtained fusion proteins were expressed in the cytoplasm of the HeLa cell. The amino acid ratio in each peptide tag are shown in Table 2. The intracellular aggregation rates obtained based on fluorescence images were as shown in Table 2. In Table 2, a molecule provided with a Myc tag was used as a control, and the effect of each peptide tag was evaluated based on a difference in the aggregation rate from that of the Myc tag.

As shown in Table 2,
the aggregation rate tended to be reduced when the rate of the Element 3 was in a range of 40 to 75%;
the aggregation rate tended to be reduced as the rate of the Element 4 was lower;
the aggregation rate tended to be increased when the rate of alanine (A) was more than 10%;
the aggregation rate tended to be increased when the rate of glycine (G) was more than 10%; and
the intracellular aggregation rate tended to be reduced as the rate of the Element 1 was higher.

Serine (S) may not be present (see Tag-4-1), but did not adversely affect the reduction of the aggregation rate even present in a large amount (see Tag-1-1 to Tag-1-3).

In either case, a peptide tag having an acidic amino acid content less than 45% did not exhibit non-specific adsorption (particularly, non-specific adsorption due to negative charge of the peptide tag) to an intracellular protein or the like. It was suggested that the peptide tag of the present disclosure is useful from the viewpoint that free intracellular localization of a protein of interest is not restricted peculiarly to the tag.

It is understood, through comparison between the Myc tag (control) and Tag-4-1, that reduction of the Element 4 with increase of the Element 3 makes a strong contribution to the aggregation rate reducing action of the peptide tag. Similarly, it is understood, also through comparison between Tag-1-2 and Tag-1-1, that the reduction of the Element 4 with increase of the Element 3 makes a contribution to the aggregation rate reducing action of the peptide tag.

In particular, when the rate of the Element 1 was 20% or more and less than 45%, that of the Element 2 was 10% or less, that of the Element 3 was 40 to 75%, that of the Element 4 was 10% or less, that of alanine was 10% or less, and that of glycine was 10% or less, the aggregation rate was favorably reduced.

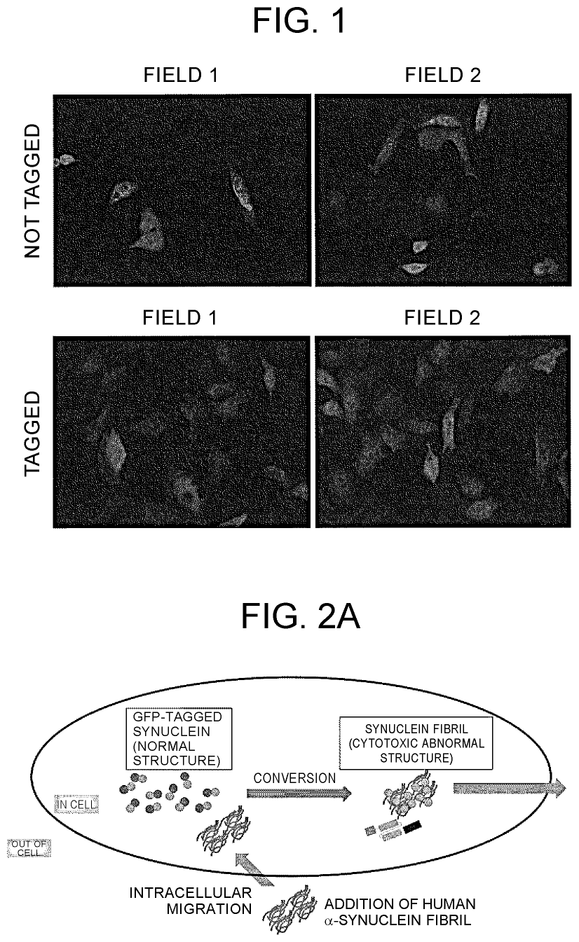

The HeLa cell expressing the fusion protein of the aggregating protein and the tag (Tag4-1) was observed under a fluorescence microscope. As a negative control, a HeLa cell expressing an aggregating protein without Tag4-1 was observed under a fluorescence microscope. Results were as illustrated in FIG. 1. As illustrated in FIG. 1, when tagged with Tag4-1 (fused with Tag-4-1), the aggregating protein homogeneously distributed in the cytoplasm, but the aggregating protein without Tag4-1 formed an aggregation in the cytoplasms of most of cells.

### Example 2: Experiment of Adding Amino Acid to Tag

Amino acid contents in a tag and the aggregation rate of a tagged protein were further analyzed. Serine (S) was randomly inserted into or added to Tag4-1 or Tag11-1, and the effect of the thus obtained modified tags on the protein aggregation rate was examined. The experiment was conducted in the same manner as described in Example above except that the tags were different. Results were as shown in Table 3. In Table 3, added or inserted amino acids are underlined.

TABLE 3
Sequences, S Contents, D/E Contents, and
Aggregation Rates of Modified Tags

| | | Aggre- | | D, |
|---|---|---|---|---|
| | | gation | S | E |
| Tag | sequence | Rate | (%) | (%) |
| Tag4-1 | NEGYREAFDEDYEQQDEDFAEQDPDGNEA | 4.57 | 0 | 42 |
| (SEQ ID NO: 59) | FEGEYDGPNQDEYPDEAQNFE | | | |
| Tag4-1-S10 | NEGSYREAFDSEDYEQSQDSEDFSAEQDPD | 5.06 | 16.7 | 35 |
| (SEQ ID NO: 60) | IGNSEAFEGSEYSDGPNSQDEYPDEAQNFS | | | |
| | E | | | |
| Tag4-1-S20 | NEGSYRESAFDSEDYEQSQDSSEDFSAEQD | 6.76 | 28.6 | 30 |
| (SEQ ID NO: 61) | SSPDGNSSEAFEGSSEYSDGPNSQDEYPDE | | | |
| | ASSQNFSSSE | | | |
| Tag11-1 | YDNPYFEPQYGFPPEEDEDE | 14.48 | 0 | 15 |
| (SEQ ID NO: 62) | | | | |
| Tag11-1-S10 | SYDSNPSYFSEPSQYGSFPPSESEDSEDSE | 11.29 | 33.3 | 10 |
| (SEQ ID NO: 63) | | | | |
| Tag11-1-S20 | SSYSDSNPSYFSEPSSQSSYGSSFPPSESE | 18.77 | 50 | 7.5 |
| (SEQ ID NO: 64) | DSSESDSSSE | | | |

As shown in Table 3 as results, increase of S in a tag did not largely affect the aggregation rate of the tagged peptide. Although it is known that increase of acidic amino acids in a tag largely affects reduction of the aggregation rate of the tagged peptide (US2020/0157210A), on the contrary, the reduction of the rate of acidic amino acids did not largely reduce the aggregation rate in this example.

Similarly, amino acid contents except for serine were changed to examine the influence of tagged peptides on the aggregation rate. In Tables 4 to 8, added or inserted amino acids are underlined.

TABLE 4
Sequences, Q Contents, D/E Contents,
and Aggregation Rates of Modified Tags

| | | Aggregation | Q | D, |
|---|---|---|---|---|
| Tag | sequence | Rate | (%) | E(%) |
| Tag 4-1 | NEGYREAFDEDYEQQDEDFAEQDPDGNEAF | 4.88 | 10 | 42 |
| | EGEYDGPNQDEYPDEAQNFE | | | |
| Tag4-1-Q10 | NEGQYREAFDQEDYEQQQDQEDFQAEQDP | 8.31 | 25 | 35 |
| (SEQ ID NO: 65) | DGNQEAFEGQEYDGPNQQDEYQPDEAQNF | | | |
| | QE | | | |
| Tag4-1-Q20 | NEGQYREQAFDQEDYEQQQDQQEDFQAEQ | 8.40 | 35.7 | 30 |
| (SEQ ID NO: 66) | DQQPDGNQQEAFEGQQEYQDGPNQQDEY | | | |
| | PDEAQQQNFQQQE | | | |
| Tag 11-1 | YDNPYFEPQYGFPPEEDEDE | 16.59 | 5 | 15 |
| Tag11-1-Q10 | QYDQNPQYFQEPQQYGQFPPQEQEDQED | 26.70 | 36.7 | 10 |
| (SEQ ID NO: 67) | QE | | | |
| Tag11-1-Q20 | QQYQDQNPQYFQEPQQQQYGQQFPQPQE | 17.27 | 52.5 | 7.5 |
| (SEQ ID NO: 68) | QEDQQEQDQQQE | | | |

TABLE 5
Sequences, N Contents, D/E Contents, and
Aggregation Rates of Modified Tags

| | | Aggre- | | |
|---|---|---|---|---|
| | | gation | N | D, E |
| Tag | sequence | Rate | (%) | (%) |
| Tag 4-1 | NEGYREAFDEDYEQQDEDFAEQDPDGNEA | 4.88 | 8.00 | 42 |
| | FEGEYDGPNQDEYPDEAQNFE | | | |
| Tag4-1-N10 | NEGNYREAFDNEDYEQNQDNEDFNAEQDP | 6.12 | 23.33 | 35 |
| (SEQ ID NO: 69) | DGNNEAFEGNEYNDGPNNQDEYPDEAQNF | | | |
| | NE | | | |
| Tag4-1-N20 | NEGNYRENAFDNEDYEQNQDNNEDFNAEQ | 6.86 | 34.29 | 30 |
| (SEQ ID NO: 70) | DNNPDGNNNEAFEGNNEYNDGPNNQDEYP | | | |
| | DEANNQNFNNNE | | | |
| Tag 11-1 | YDNPYFEPQYGFPPEEDEDE | 16.59 | 5.00 | 15 |
| Tag11-1-N10 | NYDNNPNYFNEPNQYGNFPPNENEDNE | 18.56 | 36.67 | 10 |
| (SEQ ID NO: 71) | DNE | | | |
| Tag11-1-N20 | NNYNDNNPNYFNEPNNQNNYGNNFPPNE | 17.68 | 52.50 | 7.5 |
| (SEQ ID NO: 72) | NEDNNENQNNNE | | | |

TABLE 6
Sequences, P Contents, D/E Contents,
and Aggregation Rates of Modified Tags

| | | Aggre- | | D, |
|---|---|---|---|---|
| | | gation | P | E |
| Tag | sequence | Rate | (%) | (%) |
| Tag 4-1 | NEGYREAFDEDYEQQDEDFAEQDPDGNEAF | 4.49 | 6.00 | 42 |
| | EGEYDGPNQDEYPDEAQNFE | | | |
| Tag4-1-P10 | NEGPYREAFDPEDYEQPQDPEDFPAEQDPD | 8.84 | 21.67 | 35 |
| (SEQ ID NO: 73) | GNPEAFEGPEYPDGPNPQDEYPDEAQNFPE | | | |
| Tag4-1-P20 | NEGPYREPAFDPEDYEQPQDPPEDFPAEQD | 6.44 | 32.86 | 30 |
| (SEQ ID NO: 74) | PPPDGNPPEAFEGPPEYPDGPNPQDEYPDE | | | |
| | APPQNFPPPE | | | |
| Tag 11-1 | YDNPYFEPQYGFPPEEDEDE | 17.75 | 20.00 | 15 |
| Tag11-1-P10 | PYDPNPPYFPEPPQYGLFPPEEPEDPEDPE | 15.57 | 46.67 | 10 |
| (SEQ ID NO: 75) | | | | |
| Tag11-1-P20 | PPYPDPNPPYFPEPPPQPPYGPPFPPPEPE | 18.12 | 60.00 | 7.5 |
| (SEQ ID NO: 76) | DPPEPDPPPE | | | |

TABLE 7
Sequences, F Contents, D/E Contents,
and Aggregation Rates of Modified Tags

| | | Aggre- | | D, |
|---|---|---|---|---|
| | | gation | F | E |
| Tag | sequence | Rate | (%) | (%) |
| Tag 4-1 | NEGYREAFDEDYEQQDEDFAEQDPDGNEAF | 4.90 | 8.00 | 42 |
| | EGEYDGPNQDEYPDEAQNFE | | | |
| Tag4-1-F10 | NEGFYREAFDFEDYEQFQDFEDFFAEQDPD | 35.13 | 34.29 | 35 |
| (SEQ ID NO: 77) | GNFEAFEGFEYFDGPNFQDEYPDEAQNFFE | | | |
| Tag4-1-F20 | NEGEYREFAFDEEDYEQEQDEEEDFEAEQD | 38.76 | 23.33 | 30 |
| (SEQ ID NO: 78) | EFPDGNFFEAFEGFFEYFDGPNFQDEYPDE | | | |
| | AFFQNFFFFE | | | |
| Tag 11-1 | YDNPYFEPQYGFPPEEDEDE | 16.59 | 10.00 | 15 |
| Tag11-1-F10 | FYDFNPFYFFEPFQYGFFPPFEFEDFEDFE | 43.58 | 55.00 | 10 |
| (SEQ ID NO: 79) | | | | |
| Tag11-1-F20 | FFYFDFNPFYFFEPFFQFFYGFFFPPFEFE | 27.43 | 40.00 | 7.5 |
| (SEQ ID NO: 80) | DFFEFDFFFE | | | |

TABLE 8
Sequences, Y Contents, D/E Contents, and
Aggregation Rates of Modified Tags

| | | Aggregation | Y | D, E |
|---|---|---|---|---|
| Tag | sequence | Rate | (%) | (%) |
| Tag 4-1 | NEGYREAFDEDYEQQDEDFAEQDPDGNEAF | 4.94 | 8.00 | 42 |
| | EGEYDGPNQDEYPDEAQNFE | | | |
| Tag4-1-Y10 | NEGYYREAFDYEDYEQYQDYEDFYAEQDPD | 29.59 | 23.33 | 35 |
| (SEQ ID NO: 81) | GNYEAFEGYEYYDGPNYQDEYPDEAQNFYE | | | |
| Tag4-1-Y20 | NEGYYREYAFDYEDYEQYQDYYEDFYAEQD | 37.93 | 34.29 | 30 |
| (SEQ ID NO: 82) | YYPDGNYYEAFEGYYEYYDGPNYQDEYPDE | | | |
| | AYYQNFYYYE | | | |
| Tag 11-1 | YDNPYFEPQYGFPPEEDEDE | 17.75 | 15.00 | 15 |
| Tag11-1-Y10 | YYDYNPYYFYEPYQYGYFPPYEYEDYED | 17.28 | 43.33 | 10 |
| (SEQ ID NO: 83) | YE | | | |
| Tag11-1-Y20 | YYYYDYNPYYFYEPYYQYYYGYYFPPYEY | 30.53 | 57.50 | 7.5 |
| (SEQ ID NO: 84) | EDYYEYDYYYE | | | |

As described above, when the influence on the protein aggregation rate of tags obtained by randomly adding or inserting 10 to 20 specific amino acids was examined, the addition or insertion of F and Y tended to worsen the aggregation rate of the tagged proteins. The adverse effect of the addition or insertion of the other amino acids on the aggregation rates of tagged proteins was restrictive even if D and E contents were reduced.

### Example 3: Effect on Aggregation Rate of Protein by Amino Acid Content Change by Substitution of Amino Acids in Tag

N, P, S, Q, F, and Y were respectively substituted with other amino acids, and thus, attempts were made to specify, among these amino acids, an amino acid exhibiting the effect on the protein aggregation rate. In Tables 9 and 10, substituted amino acids are underlined.

TABLE 9
Sequences, N Contents, D/E Contents, and Aggregation Rates of Modified Tags

| | | | | | Aggre- | | |
|---|---|---|---|---|---|---|---|
| N- | | C- | | | gation | N | D, E |
| terminus | scFv | terminus | Cell | sequence | Rate | (%) | (%) |
| Tag 9-1 | Y13-259 | HA | HeLa | GNNQDSSDSDNEADEASDDEONDGN | 8.14 | 20.00 | 44.00 |
| | | | | (SEQ ID NO: 85) | | | |
| Tag 4-8 | Y13-259 | HA | HeLa | NEGNREASDEDSEQQDEDNAEQDPDGNEANE | 3.67 | 16.00 | 42.00 |
| | | | | GESDGPNQDENPDEAQNSE | | | |
| | | | | (SEQ ID NO: 86) | | | |
| Tag 4-9 | Y13-259 | HA | HeLa | NEGNREANDEDNEQQDEDNAEQDPDGNEANE | 4.10 | 24.00 | 42.00 |
| | | | | GENDGPNQDENPDEAQNNE | | | |
| | | | | (SEQ ID NO: 87) | | | |
| Tag 4-10 | Y13-259 | HA | HeLa | NEGNREANDEDNEQQDEDNAEGDPDGNEANE | 5.51 | 32.00 | 42.00 |
| | | | | GENDGPNQDENPDEAQNNE | | | |
| | | | | (SEQ ID NO: 88) | | | |
| Tag 4-11 | Y13-259 | HA | HeLa | NEGNREANDEDNEQQDEDNAEQDQDGNEANE | 4.44 | 32.00 | 42.00 |
| | | | | GENDGQNQDENPDEAQNNE | | | |
| | | | | (SEQ ID NO: 89) | | | |
| Tag 18-1 | Y13-259 | HA | HeLa | DNNESADDNNENPEDNNKNTDDNEENPNNNEN | 4.82 | 43.75 | 37.50 |
| | | | | (SEQ ID NO: 90) | | | |
| Tag 4-8 | 6E | Tag 4-8, | SHSY5Y | NEGNREASDEDSEQQDEDNAEQDPDGNEANE | 0.96 | 16.00 | 42.00 |
| | | CMA, HA | | GESDGPNQDENPDEAQNSE | | | |
| Tag 18-1 | 6E | Tag 18-1, | SHSY5Y | DNNESADDNNENPEDNNKNTDDNEENPNNNEN | 1.14 | 43.75 | 37.50 |
| | | CMA, HA | | | | | |

TABLE 10
Sequences, P Contents, N Contents, and Aggregation Rates of Modified Tags

| | | Aggre- | | |
|---|---|---|---|---|
| | | gation | | |
| | | Rate | N (%) | P (%) |
| Tag 18-1 | DNNESADDNNENPEDNNKNTDDNEENPNNNEN | 4.76 | 43.75 | 6.25 |
| Tag 18-1-NS7 (SEQ ID NO: 91) | DNSESADDNSENPEDNSKNTDDSEENPSNSES | 6.37 | 21.88 | 6.25 |
| Tag 18-1-NS14 (SEQ ID NO: 92) | DSSESADDSSESPEDSSKSTDDSEESPSSSES | 5.72 | 0.00 | 6.25 |
| Tag 18-NQ7 (SEQ ID NO: 93) | DNQESADDNQENPEDNQKNTDDGEENPONGEQ | 6.60 | 21.88 | 6.25 |
| Tag 18-NQ14 (SEQ ID NO: 94) | DQQESADDQQEQPEDQQKQTDDQEEQPQQQEQ | 6.03 | 0.00 | 6.25 |
| Tag 18-1-NF7 (SEQ ID NO: 95) | DNFESADDNFENPEDNFKNTDDFEENPFNFEF | 13.50 | 21.88 | 6.25 |
| Tag 18-1-NF14 (SEQ ID NO: 96) | DFFESADDFFEFPEDFFKFTDDFEEFPFFFEF | 13.60 | 0.00 | 6.25 |
| Tax 18-1-NP7 (SEQ ID NO: 97) | DNPESADDNPENPEDNPKNTDDPEENPPNPEP | 4.48 | 21.88 | 28.13 |
| Tag 18-1-NP14 (SEQ ID NO: 98) | DPPESADDPPEPPEDPPKPTDDPEEPPPPPEP | 3.18 | 0.00 | 50.00 |
| Tag 18-1-NY7 (SEQ ID NO: 99) | DNYESADDNYENPEDNYKNTDDYEENPYNYEY | 11.60 | 21.88 | 6.25 |
| Tag 18-1-NY14 (SEQ ID NO: 100) | DYYESADDYYEYPEDYYKYTDDYEEYPYYYEY | 15.70 | 0.00 | 6.25 |
| Tag 11-1 | YDNPYFEPQYGFPPEEDEDE | 17.75 | 5.00 | 20.00 |
| Tag 11-1-FYPN (SEQ ID NO: 101) | NDNNNNENQNGNNNEEDEDE | 12.46 | 50.00 | 0.00 |
| Tag 11-1-FYN (SEQ ID NO: 102) | NDNPNNEPQNGNPPEEDEDE | 5.79 | 30.00 | 20.00 |
| 45Tag1 (SEQ ID NO: 1041) | NDEYSDFEDSDFDGDYKDSDEDYKDDSENFDDGFE | 13.91 | 3.71 | 0 |
| 45Tag1-m1 (SEQ ID NO: 1042) | PDEPPDPEDPDPDPDPKDPDEDPKDDPEPPDDPPE | 5.70 | 0 | 42.86 |

It was found, based on Tables 9 and 10, that the aggregation rate reducing action of a tagged protein is the greatest when the content of P or N in the tag was higher, and subsequently, the aggregation rate reducing action was exhibited in the order that the content of S and Q was higher, and the content of F and Y was higher. 45Tag1-ml peptide tag, which was produced by substituting, with P, all of the Element 3 in 45Tag1 having an acidic amino acid content of about 51%, largely improved the aggregation inhibiting action thereof through the amino acid substitution with P. In this manner, it was revealed that the increase of the aggregation rate reducing action by adding P and N does not depend on the acidic amino acid content. This result shows that most of constituent amino acids can be acidic amino acids, and N or P.

### Example 4: Sequence Shuffle

Two tags, Tag4-8 (Scr1) and Tag4-8 (Scr2), were synthesized by randomly shuffling the amino acid sequence of Tag4-8, and were tested for the aggregation rate reducing action against the scFv in the same manner as described above. Results were as shown in Table 11.

TABLE 11
Sequence Shuffling

| | | Aggre- |
|---|---|---|
| | | gation |
| | Sequence | Rate |
| Tag4-8 | NEGNREASDEDSEQQDEDNAEQDPD | 3.93 |
| | GNEANEGESDGPNQDENPDEAQNSE | |
| Tag4-8 (Scr1) | ENESEDNDEEEPNQNADDGDPNANP | 3.25 |
| (SEQ ID NO: | AAEQGGDSSDDEEGQENDQSRENEQ | |
| 103) | | |
| Tag4-8 (Scr2) | QNENNGDDQDQEEGSEEQQGESDRS | 4.52 |
| (SEQ ID NO: | NEESDNEPAADDNAPEGAEEPDDNN | |
| 104) | | |

As shown in Table 11, Tag4-8, Tag4-8 (Scr1) and Tag4-8 (Scr2) exhibited equivalent aggregation rate reducing actions. Even when only the order of amino acids was changed without changing the content ratios and the lengths of the amino acids, the aggregation rate reducing action was not affected.

### Example 5: Use as Tag of Human-Derived Peptide

From human proteome database (Proteome ID: UP000005640), peptides having specific amino acid content ratios were all extracted, these peptides were randomly selected to be used as tags, and thus, the aggregation rate reducing action against a tagged protein was examined.

### Extraction Condition 1:

length: 20 to 70 amino acids
group [D, E]: content of [30] or more
group [D, E]: content of less than [45]
group [H, K, R]: content of [5] or less
group [C, T, V, L, I, W, M]: content of [5] or less
group [G]: content of less than [10]
group [A]: content of less than [10]
group [F, Y]: content of [5] or less
group [N]: content of [15] or more
In the above-described extraction condition, the unit of each content is %. The amino acids are described by one letter codes.

TABLE 12
Aggregation Rates of scFvs having Tags Extracted under
Extraction Condition 1 Added

| | | Aggre- | | | | | | | |
|---|---|---|---|---|---|---|---|---|---|
| | | gation | | | | | | | |
| | | Rate | DE | N | HKR | CTVLIWM | G | A | FY |
| Tag Name | Sequence | % | Rate | Rate | Rate | Rate | Rate | Rate | Rate |
| Tag 18-1(465-1) | DNNESADDNNENPEDNNKNTDDNEENPNNNEN | 4.68 | 37.50 | 43.75 | 3.13 | 3.13 | 0 | 3.13 | 0 |
| Tag 465-2 | GNNQDSSDSDNEADEASDDEDNDGN | 5.04 | 44.00 | 20 | 0 | 0 | 8 | 8 | 0 |
| Tag 465-3 | SHGNNQDSSDSDNEADEASDDEDNDGNEGDNE | 3.98 | 43.75 | 18.75 | 3.13 | 0 | 9.38 | 6.25 | 0 |
| Tag 465-4 | ESADDNNENPEDNNKNTDDNE | 9.99 | 42.86 | 33.33 | 4.76 | 4.76 | 0 | 4.76 | 0 |
| Tag 465 5 | NNENPEDNNKNTDDNEENPNN | 13.94 | 33.33 | 47.62 | 4.76 | 4.76 | 0 | 0 | 0 |
| Tag 465-6 | TDNNESADDNNENPEDNNKN | 8.95 | 35.00 | 40 | 5 | 5 | 0 | 5 | 0 |
| Tag 465-7 | DNNESADDNNENPEDNNKNT | 7.77 | 35.00 | 40 | 5 | 5 | 0 | 5 | 0 |
| Tag 465-8 | NNESADDNNENPEDNNKNTD | 7.39 | 35.00 | 40 | 5 | 5 | 0 | 5 | 0 |
| Tag 465-9 | FWGSHGNNQDSSDSDNEADEASDDEDNDGNE | 7.36 | 38.71 | 16.13 | 3.23 | 3.23 | 9.68 | 6.45 | 3.23 |
| Tag 1121-1 | KPNNSNAPNEDQEEEIQQSE | 14.73 | 30 | 20 | 5 | 5 | 0 | 5 | 0 |
| Tag 1121-2 | NNSNAPNEDQEEEIQQSEQH | 13.76 | 30 | 20 | 5 | 5 | 0 | 5 | 0 |
| Tag 1121-3 | SEGEQQLKPNNSNAPNEDQEEE | 14.15 | 31.82 | 18.18 | 4.55 | 4.55 | 4.55 | 4.55 | 0 |
| Tag 2408-1 | EQLNFSDDDEQGSNSPKENNSEDQ | 7.56 | 33.33 | 16.67 | 4.17 | 4.17 | 4.17 | 0 | 4.17 |
| Tag 2408-2 | EQLNFSDDDEQGSNSPKENNSEDQG | 7.53 | 32.00 | 16 | 4 | 4 | 8 | 0 | 4 |
| Tag 2408-3 | EQLNFSDDDEQGSNSPKENNSEDQGS | 9.42 | 30.77 | 15.38 | 3.85 | 3.85 | 7.69 | 0 | 3.85 |
| Tag 6301-1 | EEKNENDENSLSSSSDSSED | 12.66 | 40.00 | 15 | 5 | 5 | 0 | 0 | 0 |
| Tag 6301-2 | NENDENSLSSSSDSSEDKDE | 8.85 | 40.00 | 15 | 5 | 5 | 0 | 0 | 0 |
| Tag 6626-1 | KETNNSNAQNPSEEEGEGQDE | 12.50 | 33.33 | 19.05 | 4.76 | 4.76 | 9.52 | 4.76 | 0 |
| Tag 6626-2 | ETNNSNAQNPSEEEGEGQDED | 8.04 | 38.10 | 19.05 | 0 | 4.76 | 9.52 | 4.76 | 0 |
| Tag 6626-3 | KETNNSNAQNPSEEEGEGGDED | 13.56 | 36.36 | 18.18 | 4.55 | 4.55 | 9.09 | 4.55 | 0 |
| Tag 6915-1 | ENANDSSDDSGEETDESFNP | 9.52 | 40.00 | 15 | 0 | 5 | 5 | 5 | 5 |
| Tag 7128-1 | DDNESNSESAENGWDSGSNFSEE | 12.10 | 34.78 | 17.39 | 0 | 4.35 | 8.7 | 4.35 | 4.35 |
| Tag 7128-2 | SDDNESNSESAENGWDSGSNFSEE | 8.21 | 33.33 | 16.67 | 0 | 4.17 | 8.33 | 4.17 | 4.17 |
| Tag 7128-3 | SSDDNESNSESAENGWDSGSNFSEE | 8.06 | 32.00 | 16 | 0 | 4 | 8 | 4 | 4 |
| Tag 7315-1 | EENASSGDSEENTNSDHESE | 10.74 | 40.00 | 15 | 5 | 5 | 5 | 5 | 0 |
| Tag 7315-2 | SEENASSGDSEENTNSDHES | 11.72 | 35.00 | 15 | 5 | 5 | 5 | 5 | 0 |
| Tag 7315-3 | ENASSGDSEENTNSDHESEQ | 10.07 | 35.00 | 15 | 5 | 5 | 5 | 5 | 0 |
| Tag 8482-1 | DDENSENNWRNEYPEEESSDG | 8.17 | 42.86 | 19.05 | 4.76 | 4.76 | 4.76 | 0 | 4.76 |
| Tag 8482-2 | ENSENNWRNEYPEEESSDGDE | 7.11 | 42.86 | 19.05 | 4.76 | 4.76 | 4.76 | 0 | 4.76 |
| Tag 8482-3 | NSENNWRNEYPEEESSDGDEDS | 5.08 | 40.91 | 18.18 | 4.55 | 4.55 | 4.55 | 0 | 4.55 |
| Tag 8974-1 | EQQNEASEENNDQQSQEVPE | 7.84 | 35.00 | 15 | 0 | 5 | 0 | 5 | 0 |
| Tag 8974-2 | QQNEASEENNDQQSQEVPEK | 6.39 | 30.00 | 15 | 5 | 5 | 0 | 5 | 0 |
| Tag 9333-1 | MQEDEFDQGNQEQEDNSNAE | 15.37 | 40.00 | 15 | 0 | 5 | 5 | 5 | 5 |
| Tag 9333-2 | SKMQEDEFDQGNQEQEDNSN | 9.62 | 35.00 | 15 | 5 | 5 | 5 | 0 | 5 |
| Tag 9333-3 | QEDEFDQGNQEQEDNSNAEMEEENASN | 7.13 | 40.74 | 18.52 | 0 | 3.7 | 3.7 | 7.41 | 3.7 |
| Tag 10381-1 | PSENENSQSEDSVGGDNDSEN | 5.59 | 33.33 | 19.05 | 0 | 4.76 | 9.52 | 0 | 0 |
| Tag 11717-1 | EVEESNPSAKEDSNPNSSGE | 10.48 | 30.00 | 15 | 5 | 5 | 5 | 5 | 0 |
| Tag 11717-2 | VEESNPSAKEDSNPNSSGED | 12.10 | 30.00 | 15 | 5 | 5 | 5 | 5 | 0 |
| Tag 12237-1 | KEENSESPLNENSDESYSEE | 9.98 | 40.00 | 15 | 5 | 5 | 0 | 0 | 5 |
| Tag 12809-1 | QPGPNHEEDADSYENMDNPD | 28.15 | 35.00 | 15 | 5 | 5 | 5 | 5 | 5 |
| Tag 12809-2 | PNHEEDADSYENMDNPDGPD | 20.94 | 40.00 | 15 | 5 | 5 | 5 | 5 | 5 |
| Tag 12809-3 | NHEEDADSYENMDNPDGPDP | 23.41 | 40.00 | 15 | 5 | 5 | 5 | 5 | 5 |
| Tag 12885-1 | DDPNSSDESNGNDDANSESD | 14.62 | 40.00 | 20 | 0 | 0 | 5 | 5 | 0 |
| Tag 12885-2 | NSSDESNGNDDANSESDNNS | 17.00 | 30.00 | 30 | 0 | 0 | 5 | 5 | 0 |
| Tag 12885-3 | DESNGNDDANSESDNNSSSRGD | 13.93 | 31.82 | 22.73 | 4.55 | 0 | 9.09 | 4.55 | 0 |
| Tag 12968-1 | DNNENAGEDGDNDFSPSDEEL | 13.42 | 42.86 | 19.05 | 0 | 4.76 | 9.52 | 4.76 | 4.76 |
| Tag 12968-2 | AELEEDDNNENAGEDGDNDFSPS | 10.18 | 43.48 | 17.39 | 0 | 4.35 | 8.7 | 8.7 | 4.35 |
| Tag 12968-3 | DNNENAGEDGDNDFSPSDEELAN | 14.63 | 39.13 | 21.74 | 0 | 4.35 | 8.7 | 8.7 | 4.35 |
| Tag 13648-1 | NPADDPNNQGEDEFEEAEGVREEN | 15.03 | 41.67 | 16.67 | 4.17 | 4.17 | 4.17 | 8.33 | 4.17 |
| Tag 14056-1 | NEENTEPGAESSENADDPNKD | 13.22 | 38.10 | 19.05 | 4.76 | 4.76 | 4.76 | 9.52 | 0 |
| Tag 14056-2 | SSENADDPNKDTSENADGQSDEN | 11.45 | 34.78 | 17.39 | 4.35 | 4.35 | 4.35 | 8.7 | 0 |
| Tag 14056-3 | ESSENADDPNKDTSENADGGSDEN | 10.38 | 37.50 | 16.67 | 4.17 | 4.17 | 4.17 | 8.33 | 0 |
| Tag 14681-1 | DRDPEMENEEQPSSENDSQN | 5.63 | 40.00 | 15 | 5 | 5 | 0 | 0 | 0 |
| Tag 14681-2 | PEMENEEQPSSENDSQNQSG | 11.50 | 30.00 | 15 | 0 | 5 | 5 | 0 | 0 |
| Tag 14681-3 | ENEEQPSSENDSQNQSGEQI | 17.15 | 30.00 | 15 | 0 | 5 | 5 | 0 | 0 |
| Tag 14844-1 | DSESANVSDKEAGSNENDDQN | 12.13 | 33.33 | 19.05 | 4.76 | 4.76 | 4.76 | 9.52 | 0 |
| Tag 15481-1 | NYNDGSQEDRDWQDDQSDNQ | 9.92 | 35.00 | 15 | 5 | 5 | 5 | 0 | 5 |
| Tag 16043-1 | RENTNEASSEGNSSDDSEDE | 13.45 | 40.00 | 15 | 5 | 5 | 5 | 5 | 0 |
| Tag 16043-2 | ENTNEASSEGNSSDDSEDER | 11.56 | 40.00 | 15 | 5 | 5 | 5 | 5 | 0 |
| Tag 16400-1 | QENDNGNETESEQPKESNENQ | 15.9 | 33.33 | 23.81 | 4.76 | 4.76 | 4.76 | 0 | 0 |
| Tag 16400-2 | ENDNGNETESEQPKESNENGE | 18.1 | 38.1 | 23.81 | 4.76 | 4.76 | 4.76 | 0 | 0 |
| Tag 16400-3 | QENDNGNETESEQPKESNENQE | 8.84 | 36.36 | 22.73 | 4.55 | 4.55 | 4.55 | 0 | 0 |
| Tag 16417-1 | QNEENPGDEEAKNQVNSESDSDSEE | 12.18 | 40.00 | 16 | 4 | 4 | 4 | 4 | 0 |
| Tag 16417-2 | NEENPGDEEAKNQVNSESDSDSEES | 11.00 | 40.00 | 16 | 4 | 4 | 4 | 4 | 0 |
| Tag 16417-3 | QNEENPGDEEAKNQVNSESDSDSEES | 12.37 | 38.46 | 15.38 | 3.85 | 3.85 | 3.85 | 3.85 | 0 |
| Tag 18137-1 | YNGGNANPRPANNEEEEDEEDE | 7.47 | 40.91 | 22.73 | 4.55 | 0 | 9.09 | 9.09 | 4.55 |
| Tag 18137-2 | NGGNANPRPANNEEEEDEEDEY | 8.17 | 40.91 | 22.73 | 4.55 | 0 | 9.09 | 9.09 | 4.55 |
| Tag 18137-3 | NGGNANPRPANNEEEEDEEDEYD | 9.86 | 43.48 | 21.74 | 4.35 | 0 | 8.7 | 8.7 | 4.35 |
| Tag 18347 1 | GASENEEEDDDYNKPLDPNS | 16.06 | 40.00 | 15 | 5 | 5 | 5 | 5 | 5 |
| Tag 18478-1 | ILQNQKEAEEPGPDSENSQEN | 16.82 | 30.00 | 15 | 5 | | 5 | 5 | 0 |
| Tag 18478-2 | QNGKEAEEPGPDSENSQENP | 14.77 | 30.00 | 15 | 5 | 0 | 5 | 5 | 0 |
| Tag 18478-3 | NGKEAEEPGPDSENSQENPP | 12.74 | 30.00 | 15 | 5 | 0 | 5 | 5 | 0 |
| Tag 20166-1 | KESVSPENNEEGGNDNQDNEN | 16.95 | 33.33 | 28.57 | 4.76 | 4.76 | 9.52 | 0 | 0 |
| Tag 20166-2 | ESVSPENNEEGGNDNQDNENP | 13.67 | 33.33 | 28.57 | 0 | 4.76 | 9.52 | 0 | 0 |
| Tag 20166-3 | KESVSPENNEEGGNDNQDNENP | 26.56 | 31.82 | 27.27 | 4.55 | 4.55 | 9.09 | 0 | 0 |
| Tag 41693-1 | ASPQPREPSDDENSDNSNEC | 7.83 | 30.00 | 15 | 5 | 5 | 0 | 5 | 0 |
| Tag 55443-1 | NNSQDEDGFQELNENGNAKDE | 19.76 | 33.33 | 23.81 | 4.76 | 4.76 | 9.52 | 4.76 | 4.76 |
| Tag 55443-2 | NSQDEDGFQELNENGNAKDEN | 22.25 | 33.33 | 23.81 | 4.76 | 4.76 | 9.52 | 4.76 | 4.76 |
| Tag 55443-3 | NNSQDEDGFGELNENGNAKDEN | 18.69 | 31.82 | 27.27 | 4.55 | 4.55 | 9.09 | 4.55 | 4.55 |

In Table 12, the N content ratio in the amino acid sequences of the extracted tags is 15% or more, and the content ratios of the other amino acids are as described above in Extraction Condition 1. In order to confirm that the aggregation inhibiting action does not depend on a specific amino acid sequence, tags were randomly selected from the tags extracted under Extraction Condition 1. As for some tags, an amino acid sequence satisfying the extraction condition was additionally selected from another portion of the same protein. The aggregation rates of scFvs tagged with the selected amino acid sequences were tested, and results as shown in Table 12 were obtained. As shown in Table 12, the aggregation rates of the tagged scFvs were low as a whole. Accordingly, it is obvious that the aggregation inhibiting action of a peptide tag largely depends on the amino acid content ratios thereof, and depends merely weakly on the specific amino acid sequence itself.

It is noted that human-derived amino acid sequences that can be extracted under Extraction Condition 1 were as follows.

TABLE 12-2
Examples of human-derived amino acid
sequences that can be extracted under
Extraction Condition 1

| | |
|---|---|
| 105 | TDNNESADDNNENPEDNNKN (Tag465-6) |
| 106 | DNNESADDNNENPEDNNKNT (Tag465-7) |
| 107 | NNESADDNNENPEDNNKNTD (Tag465-8) |
| 108 | NESADDNNENPEDNNKNTDD |
| 109 | ESADDNNENPEDNNKNTDDN |
| 110 | SADDNNENPEDNNKNTDDNE |
| 111 | DNNENPEDNNKNTDDNEENP |
| 112 | NNENPEDNNKNTDDNEENPN |
| 113 | NENPEDNNKNTDDNEENPNN |
| 114 | ENPEDNNKNTDDNEENPNNN |
| 115 | NPEDNNKNTDDNEENPNNNE |
| 116 | PEDNNKNTDDNEENPNNNEN |
| 117 | DNNESADDNNENPEDNNKNTD |
| 118 | NNESADDNNENPEDNNKNTDD |
| 119 | NESADDNNENPEDNNKNTDDN |
| 120 | ESADDNNENPEDNNKNTDDNE |
| | (Tag 465-4) |
| 121 | SADDNNENPEDNNKNTDDNEE |
| 122 | ADDNNENPEDNNKNTDDNEEN |
| 123 | DDNNENPEDNNKNTDDNEENP |
| 124 | DNNENPEDNNKNTDDNEENPN |
| 125 | NNENPEDNNKNTDDNEENPNN |
| | (Tag 465-5) |
| 126 | NENPEDNNKNTDDNEENPNNN |
| 127 | ENPEDNNKNTDDNEENPNNNE |
| 128 | NPEDNNKNTDDNEENPNNNEN |
| 129 | DNNESADDNNENPEDNNKNTDD |
| 130 | NNESADDNNENPEDNNKNTDDN |
| 131 | NESADDNNENPEDNNKNTDDNE |
| 132 | SADDNNENPEDNNKNTDDNEEN |
| 133 | ADDNNENPEDNNKNTDDNEENP |
| 134 | DDNNENPEDNNKNTDDNEENPN |
| 135 | DNNENPEDNNKNTDDNEENPNN |
| 136 | NNENPEDNNKNTDDNEENPNNN |
| 137 | NENPEDNNKNTDDNEENPNNNE |
| 138 | ENPEDNNKNTDDNEENPNNNEN |
| 139 | DNNESADDNNENPEDNNKNTDDN |
| 140 | NNESADDNNENPEDNNKNTDDNE |
| 141 | NESADDNNENPEDNNKNTDDNEE |
| 142 | ESADDNNENPEDNNKNTDDNEEN |
| 143 | SADDNNENPEDNNKNTDDNEENP |
| 144 | ADDNNENPEDNNKNTDDNEENPN |
| 145 | DDNNENPEDNNKNTDDNEENPNN |
| 146 | DNNENPEDNNKNTDDNEENPNNN |
| 147 | NNENPEDNNKNTDDNEENPNNNE |
| 148 | NENPEDNNKNTDDNEENPNNNEN |
| 149 | HGNNQDSSDSDNEADEASDDEDN |
| 150 | DNNESADDNNENPEDNNKNTDDNE |
| 151 | NNESADDNNENPEDNNKNTDDNEE |
| 152 | NESADDNNENPEDNNKNTDDNEEN |
| 153 | ESADDNNENPEDNNKNTDDNEENP |
| 154 | SADDNNENPEDNNKNTDDNEENPN |
| 155 | ADDNNENPEDNNKNTDDNEENPNN |
| 156 | DDNNENPEDNNKNTDDNEENPNNN |
| 157 | DNNENPEDNNKNTDDNEENPNNNE |
| 158 | NNENPEDNNKNTDDNEENPNNNEN |
| 159 | SHGNNQDSSDSDNEADEASDDEDN |
| 160 | DNNESADDNNENPEDNNKNTDDNEE |
| 161 | NNESADDNNENPEDNNKNTDDNEEN |
| 162 | NESADDNNENPEDNNKNTDDNEENP |
| 163 | ESADDNNENPEDNNKNTDDNEENPN |
| 164 | SADDNNENPEDNNKNTDDNEENPNN |
| 165 | ADDNNENPEDNNKNTDDNEENPNNN |
| 166 | DDNNENPEDNNKNTDDNEENPNNNE |
| 167 | DNNENPEDNNKNTDDNEENPNNNEN |
| 168 | GSHGNNQDSSDSDNEADEASDDEDN |
| 169 | SHGNNQDSSDSDNEADEASDDEDND |
| 170 | HGNNQDSSDSDNEADEASDDEDNDG |
| 171 | GNNQDSSDSDNEADEASDDEDNDGN |
| | (Tag 465-2) |
| 172 | DNNESADDNNENPEDNNKNTDDNEEN |
| 173 | NNESADDNNENPEDNNKNTDDNEENP |
| 174 | NESADDNNENPEDNNKNTDDNEENPN |
| 175 | ESADDNNENPEDNNKNTDDNEENPNN |
| 176 | SADDNNENPEDNNKNTDDNEENPNNN |
| 177 | ADDNNENPEDNNKNTDDNEENPNNNE |
| 178 | DDNNENPEDNNKNTDDNEENPNNNEN |
| 179 | WGSHGNNQDSSDSDNEADEASDDEDN |
| 180 | GSHGNNQDSSDSDNEADEASDDEDND |
| 181 | SHGNNQDSSDSDNEADEASDDEDNDG |
| 182 | HGNNQDSSDSDNEADEASDDEDNDGN |
| 183 | DNNESADDNNENPEDNNKNTDDNEENP |
| 184 | NNESADDNNENPEDNNKNTDDNEENPN |
| 185 | NESADDNNENPEDNNKNTDDNEENPNN |
| 186 | ESADDNNENPEDNNKNTDDNEENPNNN |
| 187 | SADDNNENPEDNNKNTDDNEENPNNNE |
| 188 | ADDNNENPEDNNKNTDDNEENPNNNEN |
| 189 | SHGNNQDSSDSDNEADEASDDEDNDGN |
| 190 | HGNNQDSSDSDNEADEASDDEDNDGNE |
| 191 | DNNESADDNNENPEDNNKNTDDNEENPN |
| 192 | NNESADDNNENPEDNNKNTDDNEENPNN |
| 193 | NESADDNNENPEDNNKNTDDNEENPNNN |
| 194 | ESADDNNENPEDNNKNTDDNEENPNNNE |
| 195 | SADDNNENPEDNNKNTDDNEENPNNNEN |
| 196 | SHGNNQDSSDSDNEADEASDDEDNDGNE |
| 197 | DNNESADDNNENPEDNNKNTDDNEENPNN |
| 198 | NNESADDNNENPEDNNKNTDDNEENPNNN |
| 199 | NESADDNNENPEDNNKNTDDNEENPNNN |
| | E |
| 200 | ESADDNNENPEDNNKNTDDNEENPNNNE |
| | N |
| 201 | DNNESADDNNENPEDNNKNTDDNEENPN |
| | NN |
| 202 | NNESADDNNENPEDNNKNTDDNEENPNN |
| | NE |
| 203 | NESADDNNENPEDNNKNTDDNEENPNNN |
| | EN |
| 204 | DNNESADDNNENPEDNNKNTDDNEENPN |
| | NNE |
| 205 | NNESADDNNENPEDNNKNTDDNEENPNN |
| | NEN |
| 206 | FWGSHGNNQDSSDSDNEADEASDDEDN |
| | DGNE (Tag465-9) |
| 207 | SHGNNQDSSDSDNEADEASDDEDNDGNE |
| | GDN |
| 208 | DNNESADDNNENPEDNNKNTDDNEENPN |
| | NNEN (18-1) |
| 209 | SHGNNQDSSDSDNEADEASDDEDNDGNE |
| | GDNE (Tag 465-3) |
| 210 | LEDNNEEPRDPQSPPDPPNE (Tag656-1) |
| 211 | EDNNEEPRDPQSPPDPPNEF (Tag656-2) |
| 212 | DNNEEPRDPQSPPDPPNEFG (Tag656-3) |
| 213 | EGEQQLKPNNSNAPNEDQEE |
| 214 | GEQQLKPNNSNAPNEDQEEE |
| 215 | KPNNSNAPNEDQEEEIQQSE (Tag1121-1) |
| 216 | PNNSNAPNEDQEEEIQQSEQ |
| 217 | NNSNAPNEDQEEEIQQSEQH (Tag1121-2) |
| 218 | EGEQQLKPNNSNAPNEDQEEE |
| 219 | SEGEQQLKPNNSNAPNEDQEEE |
| | (Tag1121-3) |
| 220 | EQLNFSDDDEQGSNSPKENN |
| 221 | LNFSDDDEQGSNSPKENNSE |
| 222 | NFSDDDEQGSNSPKENNSED |
| 223 | FSDDDEQGSNSPKENNSEDQ |
| 224 | LNFSDDDEQGSNSPKENNSED |
| 225 | NFSDDDEQGSNSPKENNSEDQ |
| 226 | EQLNFSDDDEQGSNSPKENNSE |
| 227 | QLNFSDDDEQGSNSPKENNSED |
| 228 | LNFSDDDEQGSNSPKENNSEDQ |
| 229 | NFSDDDEQGSNSPKENNSEDQG |
| 230 | EQLNFSDDDEQGSNSPKENNSED |
| 231 | QLNFSDDDEQGSNSPKENNSEDQ |
| 232 | LNFSDDDEQGSNSPKENNSEDQG |
| 233 | NFSDDDEQGSNSPKENNSEDQGS |
| 234 | EQLNFSDDDEQGSNSPKENNSEDQ |
| | (Tag2408-1) |
| 235 | EQLNFSDDDEQGSNSPKENNSEDQG |
| | (Tag2408-2) |
| 236 | EQLNFSDDDEQGSNSPKENNSEDQGS |
| | (Tag2408-3) |
| 237 | EEKNENDENSLSSSSDSSED (Tag6301-1) |
| 238 | NENDENSLSSSSDSSEDKDE (Tag6301-1) |
| 239 | KETNNSNAQNPSEEEGEGQDE (Tag6626- |
| | 1) |
| 240 | ETNNSNAQNPSEEEGEGQDED (Tag6626- |
| | 2) |
| 241 | KETNNSNAQNPSEEEGEGQDED |
| | (Tag6626-3) |
| 242 | ENANDSSDDSGEETDESFNP (Tag6915-1) |
| 243 | DDNESNSESAENGWDSGSNFSE |
| 244 | DNESNSESAENGWDSGSNFSEE |
| 245 | SDDNESNSESAENGWDSGSNFSE |
| 246 | DDNESNSESAENGWDSGSNFSEE |
| | (Tag7128-1) |
| 247 | SDDNESNSESAENGWDSGSNFSEE |
| | (Tag7128-2) |
| 248 | SSDDNESNSESAENGWDSGSNFSEE |
| | (Tag7128-3) |
| 249 | SEENASSGDSEENTNSDHES (Tag7135-2) |
| 250 | EENASSGDSEENTNSDHESE (Tag 7315-1) |
| 251 | ENASSGDSEENTNSDHESEQ (Tag7135-3) |
| 252 | DENSENNWRNEYPEEESSDG |
| 253 | ENSENNWRNEYPEEESSDGD |
| 254 | NSENNWRNEYPEEESSDGDE |
| 255 | DDENSENNWRNEYPEEESSDG (Tag8482- |
| | 1) |
| 256 | DENSENNWRNEYPEEESSDGD |
| 257 | ENSENNWRNEYPEEESSDGDE (Tag8482- |
| | 2) |
| 258 | NSENNWRNEYPEEESSDGDED |
| 259 | NSENNWRNEYPEEESSDGDEDS |
| | (Tag8482-3) |
| 260 | ENSENNWRNEYPEEESSDGDEDS |
| 261 | EQQNEASEENNDQQSQEVPE (Tag8974-1) |
| 262 | QQNEASEENNDQQSQEVPEK (Tag8974-2) |
| 263 | SKMQEDEFDQGNQEQEDNSN (Tag9333- |
| | 2) |
| 264 | KMQEDEFDQGNQEQEDNSNA |
| 265 | MQEDEFDQGNQEQEDNSNAE (Tag 9333- |
| | 1) |
| 266 | QEDEFDQGNQEQEDNSNAEM |
| 267 | FDQGNQEQEDNSNAEMEEEN |
| 268 | EFDQGNQEQEDNSNAEMEEEN |
| 269 | FDQGNQEQEDNSNAEMEEENA |
| 270 | DQGNQEQEDNSNAEMEEENAS |
| 271 | QGNQEQEDNSNAEMEEENASN |
| 272 | EFDQGNQEQEDNSNAEMEEENA |
| 273 | FDQGNQEQEDNSNAEMEEENAS |
| 274 | DQGNQEQEDNSNAEMEEENASN |
| 275 | DEFDQGNQEQEDNSNAEMEEENA |
| 276 | EFDQGNQEQEDNSNAEMEEENAS |
| 277 | FDQGNQEQEDNSNAEMEEENASN |
| 278 | DEFDQGNQEQEDNSNAEMEEENAS |
| 279 | EFDQGNQEQEDNSNAEMEEENASN |
| 280 | QEDEFDQGNQEQEDNSNAEMEEENA |
| 281 | EDEFDQGNQEQEDNSNAEMEEENAS |
| 282 | DEFDQGNQEQEDNSNAEMEEENASN |
| 283 | QEDEFDQGNQEQEDNSNAEMEEENAS |
| 284 | EDEFDQGNQEQEDNSNAEMEEENASN |
| 285 | QEDEFDQGNQEQEDNSNAEMEEENASN |
| | (Tag9333-3) |
| 286 | PSENENSQSEDSVGGDNDSEN |
| | (Tag10381-1) |
| 287 | EVEESNPSAKEDSNPNSSGE (Tag11717-1) |
| 288 | VEESNPSAKEDSNPNSSGED (Tag11717-2) |
| 289 | KEENSESPLNENSDESYSEE (Tag12237-1) |
| 290 | QPGPNHEEDADSYENMDNPD (Tag12809- |
| | 1) |
| 291 | PNHEEDADSYENMDNPDGPD (Tag12809- |
| | 2) |
| 292 | NHEEDADSYENMDNPDGPDP (Tag12809- |
| | 3) |
| 293 | DDPNSSDESNGNDDANSESD (Tag 12885- |
| | 1) |
| 294 | DPNSSDESNGNDDANSESDN |
| 295 | PNSSDESNGNDDANSESDNN |
| 296 | NSSDESNGNDDANSESDNNS (Tag 12885- |
| | 2) |
| 297 | SSDESNGNDDANSESDNNSS |
| 298 | SDESNGNDDANSESDNNSSS |
| 299 | DESNGNDDANSESDNNSSSR |
| 300 | MQGDDPNSSDESNGNDDANSE |
| 301 | QGDDPNSSDESNGNDDANSES |
| 302 | GDDPNSSDESNGNDDANSESD |
| 303 | DDPNSSDESNGNDDANSESDN |
| 304 | DPNSSDESNGNDDANSESDNN |
| 305 | DKSMQGDDPNSSDESNGNDDAN |
| 306 | SMQGDDPNSSDESNGNDDANSE |
| 307 | MQGDDPNSSDESNGNDDANSES |
| 308 | QGDDPNSSDESNGNDDANSESD |
| 309 | GDDPNSSDESNGNDDANSESDN |
| 310 | DDPNSSDESNGNDDANSESDNN |
| 311 | DPNSSDESNGNDDANSESDNNS |
| 312 | DESNGNDDANSESDNNSSSRGD |
| | (Tag12885-3) |
| 313 | DDKSMQGDDPNSSDESNGNDDAN |
| 314 | DKSMQGDDPNSSDESNGNDDANS |
| 315 | KSMQGDDPNSSDESNGNDDANSE |
| 316 | SMQGDDPNSSDESNGNDDANSES |
| 317 | MQGDDPNSSDESNGNDDANSESD |
| 318 | QGDDPNSSDESNGNDDANSESDN |
| 319 | GDDPNSSDESNGNDDANSESDNN |
| 320 | DDPNSSDESNGNDDANSESDNNS |
| 321 | DPNSSDESNGNDDANSESDNNSS |
| 322 | SDESNGNDDANSESDNNSSSRGD |
| 323 | DESNGNDDANSESDNNSSSRGDA |
| 324 | DDANSESDNNSSSRGDASYNSDE |
| 325 | FDDKSMQGDDPNSSDESNGNDDAN |
| 326 | DDKSMQGDDPNSSDESNGNDDANS |
| 327 | DKSMQGDDPNSSDESNGNDDANSE |
| 328 | SMQGDDPNSSDESNGNDDANSESD |
| 329 | MQGDDPNSSDESNGNDDANSESDN |
| 330 | QGDDPNSSDESNGNDDANSESDNN |
| 331 | GDDPNSSDESNGNDDANSESDNNS |
| 332 | DDPNSSDESNGNDDANSESDNNSS |
| 333 | DFDDKSMQGDDPNSSDESNGNDDAN |
| 334 | FDDKSMQGDDPNSSDESNGNDDANS |
| 335 | DDKSMQGDDPNSSDESNGNDDANSE |
| 336 | DKSMQGDDPNSSDESNGNDDANSES |
| 337 | KSMQGDDPNSSDESNGNDDANSESD |
| 338 | SMQGDDPNSSDESNGNDDANSESDN |
| 339 | MQGDDPNSSDESNGNDDANSESDNN |
| 340 | QGDDPNSSDESNGNDDANSESDNNS |
| 341 | GDDPNSSDESNGNDDANSESDNNSS |
| 342 | DDPNSSDESNGNDDANSESDNNSSS |
| 343 | DFDDKSMQGDDPNSSDESNGNDDANS |
| 344 | FDDKSMQGDDPNSSDESNGNDDANSE |
| 345 | DDKSMQGDDPNSSDESNGNDDANSES |
| 346 | DKSMQGDDPNSSDESNGNDDANSESD |
| 347 | KSMQGDDPNSSDESNGNDDANSESDN |
| 348 | SMQGDDPNSSDESNGNDDANSESDNN |
| 349 | MQGDDPNSSDESNGNDDANSESDNNS |
| 350 | QGDDPNSSDESNGNDDANSESDNNSS |
| 351 | GDDPNSSDESNGNDDANSESDNNSSS |
| 352 | DDPNSSDESNGNDDANSESDNNSSSR |
| 353 | DKSMQGDDPNSSDESNGNDDANSESDN |
| 354 | DDKSMQGDDPNSSDESNGNDDANSESD |
| | N |
| 355 | DKSMQGDDPNSSDESNGNDDANSESDN |
| | N |
| 356 | DDPNSSDESNGNDDANSESDNNSSSRGD |
| 357 | FDDKSMQGDDPNSSDESNGNDDANSESD |
| | N |
| 358 | DDKSMQGDDPNSSDESNGNDDANSESD |
| | NN |
| 359 | DKSMQGDDPNSSDESNGNDDANSESDN |
| | NS |
| 360 | DDPNSSDESNGNDDANSESDNNSSSRGD |
| | A |
| 361 | DESNGNDDANSESDNNSSSRGDASYNSD |
| | E |
| 362 | DFDDKSMQGDDPNSSDESNGNDDANSES |
| | DN |
| 363 | FDDKSMQGDDPNSSDESNGNDDANSESD |
| | NN |
| 364 | DDKSMQGDDPNSSDESNGNDDANSESD |
| | NNS |
| 365 | DKSMQGDDPNSSDESNGNDDANSESDN |
| | NSS |
| 366 | DDPNSSDESNGNDDANSESDNNSSSRGD |
| | AS |
| 367 | SDESNGNDDANSESDNNSSSRGDASYNS |
| | DE |
| 368 | DESNGNDDANSESDNNSSSRGDASYNSD |
| | ES |
| 369 | DFDDKSMQGDDPNSSDESNGNDDANSES |
| | DNN |
| 370 | FDDKSMQGDDPNSSDESNGNDDANSESD |
| | NNS |
| 371 | DDKSMQGDDPNSSDESNGNDDANSESD |
| | NNSS |
| 372 | DFDDKSMQGDDPNSSDESNGNDDANSES |
| | DNNS |
| 373 | FDDKSMQGDDPNSSDESNGNDDANSESD |
| | NNSS |
| 374 | DDKSMQGDDPNSSDESNGNDDANSESD |
| | NNSSS |
| 375 | DFDDKSMQGDDPNSSDESNGNDDANSES |
| | DNNSS |
| 376 | FDDKSMQGDDPNSSDESNGNDDANSESD |
| | NNSSS |
| 377 | DFDDKSMQGDDPNSSDESNGNDDANSES |
| | DNNSSS |
| 378 | DDPNSSDESNGNDDANSESDNNSSSRGD |
| | ASYNSDE |
| 379 | GDDPNSSDESNGNDDANSESDNNSSSRG |
| | DASYNSDE |
| 380 | DDPNSSDESNGNDDANSESDNNSSSRGD |
| | ASYNSDES |
| 381 | DSYDFDDKSMQGDDPNSSDESNGNDDA |
| | NSESDNNSSSRGD |
| 382 | SYDFDDKSMQGDDPNSSDESNGNDDANS |
| | ESDNNSSSRGDA |
| 383 | YDFDDKSMQGDDPNSSDESNGNDDANSE |
| | SDNNSSSRGDAS |
| 384 | DFDDKSMQGDDPNSSDESNGNDDANSES |
| | DNNSSSRGDASY |
| 385 | QGDDPNSSDESNGNDDANSESDNNSSSR |
| | GDASYNSDESKD |
| 386 | GDDPNSSDESNGNDDANSESDNNSSSRG |
| | DASYNSDESKDN |
| 387 | DDPNSSDESNGNDDANSESDNNSSSRGD |
| | ASYNSDESKDNG |
| 388 | DDKSMQGDDPNSSDESNGNDDANSESD |
| | NNSSSRGDASYNSDE |
| 389 | DFDDKSMQGDDPNSSDESNGNDDANSES |
| | DNNSSSRGDASYNSD |
| 390 | FDDKSMQGDDPNSSDESNGNDDANSESD |
| | NNSSSRGDASYNSDE |
| 391 | DDKSMQGDDPNSSDESNGNDDANSESD |
| | NNSSSRGDASYNSDES |
| 392 | DFDDKSMQGDDPNSSDESNGNDDANSES |
| | DNNSSSRGDASYNSDE |
| 393 | DFDDKSMQGDDPNSSDESNGNDDANSES |
| | DNNSSSRGDASYNSDES |
| 394 | DDPNSSDESNGNDDANSESDNNSSSRGD |
| | ASYNSDESKDNGNGSDSKGAEDDDSDST |
| | SDTN |
| 395 | DPNSSDESNGNDDANSESDNNSSSRGDA |
| | SYNSDESKDNGNGSDSKGAEDDDSDSTS |
| | DTNN |
| 396 | NSSDESNGNDDANSESDNNSSSRGDASY |
| | NSDESKDNGNGSDSKGAEDDDSDSTSDT |
| | NNSD |
| 397 | SSDESNGNDDANSESDNNSSSRGDASYN |
| | SDESKDNGNGSDSKGAEDDDSDSTSDTN |
| | NSDS |
| 398 | SDESNGNDDANSESDNNSSSRGDASYNS |
| | DESKDNGNGSDSKGAEDDDSDSTSDTNN |
| | SDSN |
| 399 | AEDDDSDSTSDTNNSDSNGNGNNGNDDN |
| | DKSDSGKGKSDSSDSDSSDSSNSSDSSD |
| | SSDS |
| 400 | EDDDSDSTSDTNNSDSNGNGNNGNDDN |
| | DKSDSGKGKSDSSDSDSSDSSNSSDSSD |
| | SSDSD |
| 401 | DDDSDSTSDTNNSDSNGNGNNGNDDND |
| | KSDSGKGKSDSSDSDSSDSSNSSDSSDS |
| | SDSDS |
| 402 | GDDPNSSDESNGNDDANSESDNNSSSRG |
| | DASYNSDESKDNGNGSDSKGAEDDDSDS |
| | TSDTN |
| 403 | DDPNSSDESNGNDDANSESDNNSSSRGD |
| | ASYNSDESKDNGNGSDSKGAEDDDSDST |
| | SDTNN |
| 404 | QGDDPNSSDESNGNDDANSESDNNSSSR |
| | GDASYNSDESKDNGNGSDSKGAEDDDSD |
| | STSDTN |
| 405 | GDDPNSSDESNGNDDANSESDNNSSSRG |
| | DASYNSDESKDNGNGSDSKGAEDDDSDS |
| | TSDTNN |
| 406 | DDPNSSDESNGNDDANSESDNNSSSRGD |
| | ASYNSDESKDNGNGSDSKGAEDDDSDST |
| | SDTNNS |
| 407 | DPNSSDESNGNDDANSESDNNSSSRGDA |
| | SYNSDESKDNGNGSDSKGAEDDDSDSTS |
| | DTNNSD |
| 408 | MQGDDPNSSDESNGNDDANSESDNNSS |
| | SRGDASYNSDESKDNGNGSDSKGAEDDD |
| | SDSTSDTN |
| 409 | QGDDPNSSDESNGNDDANSESDNNSSSR |
| | GDASYNSDESKDNGNGSDSKGAEDDDSD |
| | STSDTNN |
| 410 | GDDPNSSDESNGNDDANSESDNNSSSRG |
| | DASYNSDESKDNGNGSDSKGAEDDDSDS |
| | TSDTNNS |
| 411 | DDPNSSDESNGNDDANSESDNNSSSRGD |
| | ASYNSDESKDNGNGSDSKGAEDDDSDST |
| | SDTNNSD |
| 412 | DPNSSDESNGNDDANSESDNNSSSRGDA |
| | SYNSDESKDNGNGSDSKGAEDDDSDSTS |
| | DTNNSDS |
| 413 | GDDPNSSDESNGNDDANSESDNNSSSRG |
| | DASYNSDESKDNGNGSDSKGAEDDDSDS |
| | TSDTNNSD |
| 414 | DDPNSSDESNGNDDANSESDNNSSSRGD |
| | ASYNSDESKDNGNGSDSKGAEDDDSDST |
| | SDTNNSDS |
| 415 | QGDDPNSSDESNGNDDANSESDNNSSSR |
| | GDASYNSDESKDNGNGSDSKGAEDDDSD |
| | STSDTNNSD |
| 416 | GDDPNSSDESNGNDDANSESDNNSSSRG |
| | DASYNSDESKDNGNGSDSKGAEDDDSDS |
| | TSDTNNSDS |
| 417 | DDPNSSDESNGNDDANSESDNNSSSRGD |
| | ASYNSDESKDNGNGSDSKGAEDDDSDST |
| | SDTNNSDSN |
| 418 | EDDDSDSTSDTNNSDSNGNGNNGNDDN |
| | DKSDSGKGKSDSSDSDSSDSSNSSDSSD |
| | SSDSDSSDSN |
| 419 | MQGDDPNSSDESNGNDDANSESDNNSS |
| | SRGDASYNSDESKDNGNGSDSKGAEDDD |
| | SDSTSDTNNSD |
| 420 | QGDDPNSSDESNGNDDANSESDNNSSSR |
| | GDASYNSDESKDNGNGSDSKGAEDDDSD |
| | STSDTNNSDS |
| 421 | GDDPNSSDESNGNDDANSESDNNSSSRG |
| | DASYNSDESKDNGNGSDSKGAEDDDSDS |
| | TSDTNNSDSN |
| 422 | DDPNSSDESNGNDDANSESDNNSSSRGD |
| | ASYNSDESKDNGNGSDSKGAEDDDSDST |
| | SDTNNSDSNG |
| 423 | AEDDDSDSTSDTNNSDSNGNGNNGNDDN |
| | DKSDSGKGKSDSSDSDSSDSSNSSDSSD |
| | SSDSDSSDSN |
| 424 | EDDDSDSTSDTNNSDSNGNGNNGNDDN |
| | DKSDSGKGKSDSSDSDSSDSSNSSDSSD |
| | SSDSDSSDSNS |
| 425 | LEEDDNNENAGEDGDNDFSPS |
| 426 | DNNENAGEDGDNDFSPSDEEL (Tag |
| | 12968-1) |
| 427 | NNENAGEDGDNDFSPSDEELA |
| 428 | NENAGEDGDNDFSPSDEELAN |
| 429 | DNNENAGEDGDNDFSPSDEELA |
| 430 | NNENAGEDGDNDFSPSDEELAN |
| 431 | AELEEDDNNENAGEDGDNDFSPS (Tag |
| | 12968-2) |
| 432 | DDNNENAGEDGDNDFSPSDEELA |
| 433 | DNNENAGEDGDNDFSPSDEELAN (Tag |
| | 12968-3) |
| 434 | DDNNENAGEDGDNDFSPSDEELAN |
| 435 | EDDNNENAGEDGDNDFSPSDEELAN |
| 436 | NPADDPNNQGEDEFEEAEQVREEN (Tag |
| | 13648-1) |
| 437 | NEENTEPGAESSENADDPNKD (Tag |
| | 14056-1) |
| 438 | ENADDPNKDTSENADGQSDEN |
| 439 | SENADDPNKDTSENADGQSDEN |
| 440 | SSENADDPNKDTSENADGQSDEN (Tag |
| | 14056-2) |
| 441 | ESSENADDPNKDTSENADGQSDEN (Tag |
| | 14056-3) |
| 442 | DRDPEMENEEQPSSENDSQN (Tag 14681- |
| | 1) |
| 443 | RDPEMENEEQPSSENDSQNQ |
| 444 | DPEMENEEQPSSENDSQNQS |
| 445 | PEMENEEQPSSENDSQNQSG (Tag 14681- |
| | 2) |
| 446 | EMENEEQPSSENDSQNQSGE |
| 447 | MENEEQPSSENDSQNQSGEQ |
| 448 | ENEEQPSSENDSQNQSGEQI (Tag 14681- |
| | 3) |
| 449 | DSESANVSDKEAGSNENDDQN (Tag |
| | 14844-1) |
| 450 | NYNDGSQEDRDWQDDQSDNQ (Tag |
| | 15481-1) |
| 451 | RENTNEASSEGNSSDDSEDE (Tag 16043- |
| | 1) |
| 452 | ENTNEASSEGNSSDDSEDER (Tag 16043- |
| | 2) |
| 453 | QENDNGNETESEQPKESNEN |
| 454 | ENDNGNETESEQPKESNENQ |
| 455 | NDNGNETESEQPKESNENQE |
| 456 | QENDNGNETESEQPKESNENQ (Tag |
| | 16400-1) |
| 457 | ENDNGNETESEQPKESNENQE (Tag |
| | 16400-2) |
| 458 | QENDNGNETESEQPKESNENQE (Tag |
| | 16400-3) |
| 459 | QNEENPGDEEAKNQVNSESD |
| 460 | NEENPGDEEAKNQVNSESDS |
| 461 | EENPGDEEAKNQVNSESDSD |
| 462 | ENPGDEEAKNQVNSESDSDS |
| 463 | NPGDEEAKNQVNSESDSDSE |
| 464 | QNEENPGDEEAKNQVNSESDS |
| 465 | NEENPGDEEAKNQVNSESDSD |
| 466 | QNEENPGDEEAKNQVNSESDSD |
| 467 | NEENPGDEEAKNQVNSESDSDS |
| 468 | QNEENPGDEEAKNQVNSESDSDS |
| 469 | NEENPGDEEAKNQVNSESDSDSE |
| 470 | QNEENPGDEEAKNQVNSESDSDSE |
| 471 | NEENPGDEEAKNQVNSESDSDSEE |
| 472 | QNEENPGDEEAKNQVNSESDSDSEE (Tag |
| | 16417-1) |
| 473 | NEENPGDEEAKNQVNSESDSDSEES (Tag |
| | 16417-2) |
| 474 | QNEENPGDEEAKNQVNSESDSDSEES |
| | (Tag 16417-3) |
| 475 | YNGGNANPRPANNEEEEDEED |
| 476 | NGGNANPRPANNEEEEDEEDE |
| 477 | GGNANPRPANNEEEEDEEDEY |
| 478 | YNGGNANPRPANNEEEEDEEDE (Tag |
| | 18137-1) |
| 479 | NGGNANPRPANNEEEEDEEDEY (Tag |
| | 18137-2) |
| 480 | NGGNANPRPANNEEEEDEEDEYD (Tag |
| | 18137-3) |
| 481 | GASENEEEDDDYNKPLDPNS (Tag 18347- |
| | 1) |
| 482 | LQNQKEAEEPGPDSENSQEN (Tag 18478- |
| | 1) |
| 483 | QNQKEAEEPGPDSENSQENP (Tag 18478- |
| | 2) |
| 484 | NQKEAEEPGPDSENSQENPP (Tag 18478- |
| | 3) |
| 485 | KESVSPENNEEGGNDNQDNEN (Tag 20166-1) |
| 486 | ESVSPENNEEGGNDNQDNENP (Tag 20166-2) |
| 487 | KESVSPENNEEGGNDNQDNENP (Tag 20166-3) |
| 488 | ASPQPREPSDDENSDNSNEC (Tag 41693- |
| 489 | NNSQDEDGFQELNENGNAKDE (Tag 55443-1) |
| 490 | NSQDEDGFQELNENGNAKDEN (Tag |
| | 55443-2) |
| 491 | NNSQDEDGFQELNENGNAKDEN (Tag |
| | 55443-3) |

### Extraction Condition 2:

length: 20 to 70 amino acids
group [D, E]: content of [30] or more
group [D, E]: content of less than [45]
group [P]: content of [10] or more
group [H, K, R]: content of [5] or less
group [G]: content of less than [10]
group [A]: content of less than [10]
group [C, T, V, L, I, M, W]: content of [0]
group [F, Y]: content of [0]
In the above-described extraction condition, the unit of each content is. The amino acids are described by one letter codes.

TABLE 13
Aggregation Rates of scFvs having Tags Extracted
under Extraction Condition 2 Added

| | | Aggre- | | | | | |
|---|---|---|---|---|---|---|---|
| | | gation | | | | | |
| | | Rate | DE | P | HKR | G | A |
| Tag Name | Sequence | % | Rate | Rate | Rate | Rate | Rate |
| tag47-1 | SSDSPKDQSPPEDSGESEAD | 9.48 | 35 | 15 | 5 | 5 | 5 |
| tag1784-1 | IGPEPEPEPEPEPEPAPEPEPE | 15.91 | 42.86 | 47.62 | 0 | 4.76 | 4.76 |
| tag1784-2 | PEPEPEPEPEPEPAPEPEPEP | 14.03 | 42.86 | 52.38 | 0 | 0 | 4.76 |
| tag1784-3 | EPEPEPEPEPEPAPEPEPEPK | 23.67 | 42.86 | 47.62 | 4.76 | 0 | 4.76 |
| tag2257-1 | PEEDGSPDPEPSPEPEPKPS | 22.33 | 35 | 40 | 5 | 5 | 0 |
| tag2257-2 | EDPEEDGSPDPEPSPEPEPKP | 14.76 | 42.86 | 38.1 | 4.76 | 4.76 | 0 |
| tag2257-3 | IDPEEDGSPDPEPSPEPEPKPS | 17.21 | 38.1 | 38.1 | 4.76 | 4.76 | 0 |
| tag4398-1 | PDDDGSDDSSPPSASPAESEP | 35.71 | 33.33 | 23.81 | 0 | 4.76 | 9.52 |
| tag4398-2 | DDDGSDDSSPPSASPACSCPQ | 18.64 | 33.33 | 19.05 | 0 | 4.76 | 9.52 |
| tag4398-3 | PDDDGSDDSSPPSASPAESEPQ | 18.58 | 31.82 | 22.73 | 0 | 4.55 | 9.09 |
| tag4898-1 | PPPEEQGQGDAPPQHEDEEPA | 9.9 | 33.33 | 28.57 | 4.76 | 9.52 | 9.52 |
| tag5533-1 | IGAPSPAPSPDSDSDSDSDGEE | 19.43 | 33.33 | 19.05 | 0 | 9.52 | 9.52 |
| tag5533-2 | APSPAPSPDSDSDSDSDGEEE | 16.43 | 38.1 | 19.05 | 0 | 4.76 | 9.52 |
| tag5533-3 | IPSPAPSPDSDSDSDSDGEEEE | 8.76 | 42.86 | 19.05 | 0 | 4.76 | 4.76 |
| tag5601-1 | PAPPPPPPPEEDPEQDSGPE | 33.03 | 30 | 50 | 0 | 5 | 5 |
| tag5601-2 | APPPPPPPEEDPEQDSGPED | 20.10 | 35 | 45 | 0 | 5 | 5 |
| tag5601-3 | PAPPPPPPPEEDPEQDSGPED | 24.34 | 33.33 | 47.62 | 0 | 4.76 | 4.76 |
| tag6354-1 | PPPPPQAPPEEENESEPEEPS | 7.87 | 33.33 | 42.86 | 0 | 0 | 4.76 |
| tag6354-2 | PPPPQAPPEEENESEPEEPSG | 10.68 | 33.33 | 38.1 | 0 | 4.76 | 4.76 |
| tag6354-3 | PPPPPQAPPEEENESEPEEPSG | 7.98 | 31.82 | 40.91 | 0 | 4.55 | 4.55 |
| tag6681-1 | PQDSSSKSPEPSADESPDND | 8.07 | 30 | 20 | 5 | 0 | 5 |
| tag7124-1 | IQSSDNSEDEEEPPDNADSKS | 5.56 | 40 | 10 | 5 | 0 | 5 |
| tag7702-1 | EEEEQPGKAPDPQDPQDAESD | 13.15 | 42.86 | 19.05 | 4.76 | 4.76 | 9.52 |
| tag7702-2 | EEEQPGKAPDPQDPQDAESDS | 17.29 | 38.1 | 19.05 | 4.76 | 4.76 | 9.52 |
| tag7702-3 | EEEEQPGKAPDPQDPQDAESDS | 17.57 | 40.91 | 18.18 | 4.55 | 4.55 | 9.09 |
| tag8341-1 | PPPSEEEGPEEPPKASPESE | 23.65 | 35 | 35 | 5 | 5 | 5 |
| tag8341-2 | PPPPSEEEGPEEPPKASPESE | 23.58 | 33.33 | 38.1 | 1.76 | 1.76 | 4.76 |
| tag8341-3 | PPPSEEEGPEEPPKASPESEA | 24.91 | 33.33 | 33.33 | 4.76 | 4.76 | 9.52 |
| tag10102-1 | DDAEEPESPPPPPRSPSPEP | 19.00 | 30 | 45 | 5 | 0 | 5 |
| tag11508-1 | SGEASSSEEEPPSPDDKENQA | 14.91 | 33.33 | 14.29 | 4.76 | 4.76 | 9.52 |
| tag11508-2 | GEASSSEEEPPSPDDKENQAP | 14.49 | 33.33 | 19.05 | 4.76 | 4.76 | 9.52 |
| tag11508-3 | SGEASSSEEEPPSPDDKENQAP | 13.03 | 31.82 | 18.18 | 4.55 | 4.55 | 9.09 |
| tag13088-1 | PPPPPPPEESSDSEPEAEPG | 14.98 | 30 | 45 | 0 | 5 | 5 |
| tag13088-2 | PPPPPLESSDSEPLAEPGSP | 16.42 | 30 | 40 | 0 | 5 | 5 |
| tag13088-3 | PPPEESSDSEPEAEPGSPQK | 20.58 | 30 | 30 | 5 | 5 | 5 |
| tag13619-1 | SDPEPPDAGEDSKSENGENAP | 11.52 | 33.33 | 19.05 | 4.76 | 9.52 | 9.52 |
| tag14205-1 | SDSESEDPPRNOASDSENEE | 10.73 | 40 | 10 | 5 | 0 | 5 |
| tag14666-1 | GPGEDAEPDEDPQSEDSEAPS | 10.21 | 42.86 | 19.05 | 0 | 9.52 | 9.52 |
| tag14666-2 | PGEDAEPDEDPQSEDSEAPSS | 10.04 | 42.86 | 19.05 | 0 | 4.76 | 9.52 |
| tag14666-3 | GPGEDAEPDEDPOSEDSEAPSS | 12.08 | 40.91 | 18.18 | 0 | 9.09 | 9.09 |
| tag15430-1 | ESESSSSDSEANEPSQSASPEPE | 14.06 | 30.43 | 13.04 | 0 | 0 | 8.7 |
| tag15430-2 | SDSESESSSSDSEANEPSQSAS | 10.87 | 30.77 | 11.54 | 0 | 0 | 7.69 |
| | PEPE | | | | | | |
| tag15430-3 | DSESESSSSDSEANEPSQSASPE | 7.54 | 30.77 | 15.38 | 0 | 0 | 7.69 |
| | PEP | | | | | | |
| tag16604-1 | HQEDSEEESQEEEAEGASEPPPP | 12.72 | 43.48 | 17.39 | 4.35 | 4.35 | 8.7 |
| tag16604-2 | GDHQEDSEEESQEEEAEGASEP | 12.95 | 44 | 16 | 1 | 8 | 8 |
| | PPP | | | | | | |
| tag17053-1 | PSQPPEEPEPDEAESSPDPQ | 15.35 | 35 | 35 | 0 | 0 | 5 |
| tag17053-2 | PAPSQPPEEPEPDEAESSPDP | 16.53 | 33.33 | 38.1 | 0 | 0 | 9.52 |
| tag17053-3 | DPAPSQPPEEPEPDEAESSPDP | 14.03 | 36.36 | 36.36 | 0 | 0 | 9.09 |
| tag17170 | PGSQPQASSGPEAEEEEEDDE | 11.11 | 42.86 | 14.29 | 0 | 9.52 | 9.52 |
| tag17514-1 | SPDSQEEQKGESSASSPEEP | 13.45 | 30 | 15 | 5 | 5 | 5 |
| tag17514-2 | PADSPDSQEEQKGESSASSPEEP | 15.02 | 30.43 | 17.39 | 4.35 | 4.35 | 8.7 |
| tag17514-3 | ADSPDSQLEQKGESSASSPLEPE | 16.14 | 34.78 | 13.04 | 4.35 | 4.35 | 8.7 |
| tag17603-1 | PSPCDCSSSSSSSSSSCDCD | 6.01 | 35 | 10 | 0 | 0 | 0 |
| tag17603-2 | PRSPSPEDESSSSSSSSSSEDEE | 5.96 | 30.43 | 13.04 | 4.35 | | 0 |
| tag17603-3 | PRSPSPEDESSSSSSSSSSEDE | 7.22 | 33.33 | 12.5 | 4.17 | 0 | 0 |
| | EE | | | | | | |
| tag18253-1 | SSSDSSDSDSSEDDCAPSKP | 3.59 | 35 | 10 | 5 | 0 | 5 |
| tag18453-1 | PSPGSPRGQPQDODDDEDDEE | 6.12 | 42.86 | 19.05 | 4.76 | 9.52 | 0 |
| tag18467-1 | PAGDGEAGPQQAEDHPQNPPED | 9.87 | 32.26 | 25.81 | 3.23 | 9.68 | 9.68 |
| | PNQDPPEDD | | | | | | |
| tag18467-2 | AGDGEAGPQQAEDHPQNPPEDP | 5.73 | 32.26 | 22.58 | 3.23 | 9.68 | 9.68 |
| | NQDPPEDDS | | | | | | |
| tag18467-3 | PAGDGEAGPQQAEDHPQNPPED | 8.73 | 31.25 | 25 | 3.13 | 9.38 | 9.38 |
| | PNQDPPEDDS | | | | | | |
| tag18478-1 | QNQKEAEEPGPDSENSQENP | 14.94 | 30 | 15 | 5 | 5 | 5 |
| tag18478-2 | NQKEAEEPGPDSENSQENPP | 11.47 | 30 | 20 | 5 | 5 | 5 |
| tag19033-1 | DQNESQSPQEPEEGPSEDDKA | 12.69 | 38.1 | 14.29 | 4.76 | 4.76 | 4.76 |
| tag19033-2 | QNESQSPQEPEEGPSEDDKAE | 12.4 | 38.1 | 14.29 | 4.76 | 4.76 | 4.76 |
| tag19033-3 | NESQSPQEPEEGPSEDDKAEG | 12.28 | 38.1 | 14.29 | 4.76 | 9.52 | 4.76 |
| tag29487-1 | PASSSSNPEEGPEEDREAESE | 12.77 | 38.1 | 14.29 | 4.76 | 4.76 | 9.52 |
| tag34831-1 | DKPEEEDDEAQQPQPQSGPEEAE | 7.58 | 43.48 | 17.39 | 4.35 | 4.35 | 8.7 |
| tag34831-2 | KPEEEDDEAQQPQPQSGPEEAEE | 8.02 | 43.48 | 17.39 | 4.35 | 4.35 | 8.7 |
| tag34831-3 | PEEEDDEAQQPQPQSGPEEAEEG | 9.14 | 43.48 | 17.39 | 0 | 8.7 | 8.7 |
| tag34858-1 | PEEEHAPGEDESSPQPSQPS | 17.52 | 30 | 25 | 5 | 5 | 5 |

In Table 13, the P content ratio in the amino acid sequences of the extracted tags is 15% or more, and the content ratios of the other amino acids are as described above in Extraction Condition 2. In order to confirm that the aggregation inhibiting action does not depend on a specific amino acid sequence, tags were randomly selected from the tags extracted under Extraction Condition 2. As for some tags, an amino acid sequence satisfying the extraction condition was additionally selected from another portion of the same protein. The aggregation rates of scFvs tagged with the selected amino acid sequences were tested, and results as shown in Table 13 were obtained. As shown in Table 13, the aggregation rates of the tagged scFvs were low as a whole.

It is noted that human-derived amino acid sequences that can be extracted under Extraction Condition 2 were as follows.

TABLE 13-2
Examples of human-derived amino acid
sequences that can be extracted under
Extraction Condition 2

| SEQ | |
|---|---|
| ID | |
| NO: | Sequence |
| 492 | SSDSPKDQSPPEDSGESEAD (tag47-1) |
| 493 | PGPEPEPEPEPEPEPAPEPE |
| 494 | GPEPEPEPEPEPEPAPEPEP |
| 495 | PEPEPEPEPEPAPEPEPEPK |
| 496 | EPEPEPEPEPAPEPEPEPKP |
| 497 | PEPEPEPEPAPEPEPEPKPG |
| 498 | AGPGPEPEPEPEPEPEPAPEP |
| 499 | GPGPEPEPEPEPEPEPAPEPE |
| 500 | PGPEPEPEPEPEPEPAPEPEP |
| 501 | GPEPEPEPEPEPEPAPEPEPE (tag1784-1) |
| 502 | PEPEPEPEPEPEPAPEPEPEP (tag1784-2) |
| 503 | EPEPEPEPEPEPAPEPEPEPK (tag1784-3) |
| 504 | PEPEPEPEPEPAPEPEPEPKP |
| 505 | EPEPEPEPEPAPEPEPEPKPG |
| 506 | PEPEPEPEPAPEPEPEPKPGA |
| 507 | EPEPEPEPAPEPEPEPKPGAG |
| 508 | AGPGPEPEPEPEPEPEPAPEPE |
| 509 | GPGPEPEPEPEPEPEPAPEPEP |
| 510 | PGPEPEPEPEPEPEPAPEPEPE |
| 511 | GPEPEPEPEPEPEPAPEPEPEP |
| 512 | PEPEPEPEPEPEPAPEPEPEPK |
| 513 | EPEPEPEPEPEPAPEPEPEPKP |
| 514 | PEPEPEPEPEPAPEPEPEPKPG |
| 515 | EPEPEPEPEPAPEPEPEPKPGA |
| 516 | PEPEPEPEPAPEPEPEPKPGAG |
| 517 | AGPGPEPEPEPEPEPEPAPEPEP |
| 518 | GPGPEPEPEPEPEPEPAPEPEPE |
| 519 | PGPEPEPEPEPEPEPAPEPEPEP |
| 520 | GPEPEPEPEPEPEPAPEPEPEPK |
| 521 | PEPEPEPEPEPEPAPEPEPEPKP |
| 522 | EPEPEPEPEPEPAPEPEPEPKPG |
| 523 | PEPEPEPEPEPAPEPEPEPKPGA |
| 524 | EPEPEPEPEPAPEPEPEPKPGAG |
| 525 | AGPGPEPEPEPEPEPEPAPEPEPE |
| 526 | GPGPEPEPEPEPEPEPAPEPEPEP |
| 527 | PGPEPEPEPEPEPEPAPEPEPEPK |
| 528 | GPEPEPEPEPEPEPAPEPEPEPKP |
| 529 | PEPEPEPEPEPEPAPEPEPEPKPG |
| 530 | EPEPEPEPEPEPAPEPEPEPKPGA |
| 531 | PEPEPEPEPEPAPEPEPEPKPGAG |
| 532 | AGPGPEPEPEPEPEPEPAPEPEPEP |
| 533 | GPGPEPEPEPEPEPEPAPEPEPEPK |
| 534 | PGPEPEPEPEPEPEPAPEPEPEPKP |
| 535 | GPEPEPEPEPEPEPAPEPEPEPKPG |
| 536 | PEPEPEPEPEPEPAPEPEPEPKPGA |
| 537 | EPEPEPEPEPEPAPEPEPEPKPGAG |
| 538 | AGPGPEPEPEPEPEPEPAPEPEPEPK |
| 539 | GPGPEPEPEPEPEPEPAPEPEPEPKP |
| 540 | PGPEPEPEPEPEPEPAPEPEPEPKPG |
| 541 | GPEPEPEPEPEPEPAPEPEPEPKPGA |
| 542 | PEPEPEPEPEPEPAPEPEPEPKPGAG |
| 543 | AGPGPEPEPEPEPEPEPAPEPEPEPKP |
| 544 | PGPEPEPEPEPEPEPAPEPEPEPKPGA |
| 545 | DPEEDGSPDPEPSPEPEPKP |
| 546 | PEEDGSPDPEPSPEPEPKPS (tag2257-1) |
| 547 | EDPEEDGSPDPEPSPEPEPKP (tag2257-2) |
| 548 | DPEEDGSPDPEPSPEPEPKPS (tag2257-3) |
| 549 | EDPEEDGSPDPEPSPEPEPKPS |
| 550 | DEDPEEDGSPDPEPSPEPEPKPS |
| 551 | PDDDGSDDSSPPSASPAESEP (tag4398-1) |
| 552 | DDDGSDDSSPPSASPAESEPQ (tag4398-2) |
| 553 | PDDDGSDDSSPPSASPAESEPQ (tag4398- |
| | 3) |
| 554 | PPPEEQGQGDAPPQHEDEEPA (tag4898- |
| | 1) |
| 555 | PSPAPSPDSDSDSDSDGEEE |
| 556 | GAPSPAPSPDSDSDSDSDGEE (tag5533-1) |
| 557 | APSPAPSPDSDSDSDSDGEEE (tag5533-2) |
| 558 | PSPAPSPDSDSDSDSDGEEEE (tag5533-3) |
| 559 | NGAPSPAPSPDSDSDSDSDGEE |
| 560 | GAPSPAPSPDSDSDSDSDGEEE |
| 561 | APSPAPSPDSDSDSDSDGEEEE |
| 562 | QNGAPSPAPSPDSDSDSDSDGEE |
| 563 | NGAPSPAPSPDSDSDSDSDGEEE |
| 564 | GAPSPAPSPDSDSDSDSDGEEEE |
| 565 | APSPAPSPDSDSDSDSDGEEEEE |
| 566 | QNGAPSPAPSPDSDSDSDSDGEEE |
| 567 | NGAPSPAPSPDSDSDSDSDGEEEE |
| 568 | GAPSPAPSPDSDSDSDSDGEEEEE |
| 569 | QNGAPSPAPSPDSDSDSDSDGEEEE |
| 570 | NGAPSPAPSPDSDSDSDSDGEEEEE |
| 571 | GAPSPAPSPDSDSDSDSDGEEEEEE |
| 572 | QNGAPSPAPSPDSDSDSDSDGEEEEE |
| 573 | NGAPSPAPSPDSDSDSDSDGEEEEEE |
| 574 | QNGAPSPAPSPDSDSDSDSDGEEEEEE |
| 575 | NGAPSPAPSPDSDSDSDSDGEEEEEEE |
| 576 | QNGAPSPAPSPDSDSDSDSDGEEEEEEE |
| 577 | QNGAPSPAPSPDSDSDSDSDGEEEEEEE |
| | GER |
| 578 | QNGAPSPAPSPDSDSDSDSDGEEEEEEE |
| | GERD |
| 579 | EPPAPPPPPPPEEDPEQDSG |
| 580 | PAPPPPPPPEEDPEQDSGPE (tag5601-1) |
| 581 | APPPPPPPEEDPEQDSGPED (tag5601-2) |
| 582 | PAPPPPPPPEEDPEQDSGPED (tag5601-3) |
| 583 | EPPAPPPPPPPEEDPEQDSGPE |
| 584 | PPAPPPPPPPEEDPEQDSGPED |
| 585 | AEPPAPPPPPPPEEDPEQDSGPE |
| 586 | EPPAPPPPPPPEEDPEQDSGPED |
| 587 | AEPPAPPPPPPPEEDPEQDSGPED |
| 588 | PPPPPQAPPEEENESEPEEP |
| 589 | PPPPQAPPEEENESEPEEPS |
| 590 | PPPQAPPEEENESEPEEPSG |
| 591 | PPPPPQAPPEEENESEPEEPS (tag6354-1) |
| 592 | PPPPQAPPEEENESEPEEPSG (tag6354-2) |
| 593 | PPPPPQAPPEEENESEPEEPSG (tag6354- |
| | 3) |
| 594 | PQDSSSKSPEPSADESPDND (tag6681-1) |
| 595 | QSSDNSEDEEEPPDNADSKS (tag7124-1) |
| 596 | EEEEEQPGKAPDPQDPQDAES |
| 597 | EEEEQPGKAPDPQDPQDAESD (tag7702- |
| | 1) |
| 598 | EEEQPGKAPDPQDPQDAESDS (tag7702- |
| | 2) |
| 599 | EEEEQPGKAPDPQDPQDAESDS |
| | (tag7702-3) |
| 600 | EEEEEQPGKAPDPQDPQDAESDS |
| 601 | PPPPPSEEEGPEEPPKASPE |
| 602 | PPPPSEEEGPEEPPKASPES |
| 603 | PPPSEEEGPEEPPKASPESE (tag8341-1) |
| 604 | PPPPSEEEGPEEPPKASPESE (tag8341-2) |
| 605 | PPPSEEEGPEEPPKASPESEA (tag8341-3) |
| 606 | PPPPPSEEEGPEEPPKASPESE |
| 607 | PPPPSEEEGPEEPPKASPESEA |
| 608 | PPPPPSEEEGPEEPPKASPESEA |
| 609 | DDAEEPESPPPPPRSPSPEP (tag10102-1) |
| 610 | SGEASSSEEEPPSPDDKENQ |
| 611 | SGEASSSEEEPPSPDDKENQA (tag11508- |
| | 1) |
| 612 | GEASSSEEEPPSPDDKENQAP (tag11508- |
| | 2 |
| 613 | SGEASSSEEEPPSPDDKENQAP |
| | (tag11508-3) |
| 614 | PQPPPPPPPEESSDSEPEAE |
| 615 | QPPPPPPPEESSDSEPEAEP |
| 616 | PPPPPPPEESSDSEPEAEPG (tag13088-1) |
| 617 | PPPPPPEESSDSEPEAEPGS |
| 618 | PPPPPEESSDSEPEAEPGSP (tag13088-2) |
| 619 | PPPPEESSDSEPEAEPGSPQ |
| 620 | PPPEESSDSEPEAEPGSPQK (tag13088-3) |
| 621 | SDPEPPDAGEDSKSENGENAP (tag13619- |
| | 1) |
| 622 | SDSESEDPPRNQASDSENEE (tag14205-1) |
| 623 | GPGEDAEPDEDPQSEDSEAPS (tag14666- |
| | 1) |
| 624 | PGEDAEPDEDPQSEDSEAPSS (tag14666- |
| | 2) |
| 625 | GPGEDAEPDEDPQSEDSEAPSS |
| | (tag14666-3) |
| 626 | ESESSSSDSEANEPSQSASPEPE |
| | (tag15430-1) |
| 627 | DSESESSSSDSEANEPSQSASPEPE |
| 628 | SDSESESSSSDSEANEPSQSASPEPE |
| | (tag15430-2) |
| 629 | DSESESSSSDSEANEPSQSASPEPEP |
| | (tag15430-3) |
| 630 | HQEDSEEESQEEEAEGASEPPPP |
| | (tag16604-1) |
| 631 | GDHQEDSEEESQEEEAEGASEPPPP |
| | (tag16604-2) |
| 632 | PSQPPEEPEPDEAESSPDPQ (tag17053-1) |
| 633 | DPAPSQPPEEPEPDEAESSPD |
| 634 | PAPSQPPEEPEPDEAESSPDP (tag17053- |
| | 2) |
| 635 | APSQPPEEPEPDEAESSPDPQ |
| 636 | PSQPPEEPEPDEAESSPDPQA |
| 637 | DPAPSQPPEEPEPDEAESSPDP |
| | (tag17053-3) |
| 638 | PAPSQPPEEPEPDEAESSPDPQ |
| 639 | DPAPSQPPEEPEPDEAESSPDPQ |
| 640 | PGSQPQASSGPEAEEEEEDDE (tag17170) |
| 641 | DSPDSQEEQKGESSASSPEE |
| 642 | SPDSQEEQKGESSASSPEEP (tag17514-1) |
| 643 | PDSQEEQKGESSASSPEEPE |
| 644 | DSQEEQKGESSASSPEEPEE |
| 645 | DSPDSQEEQKGESSASSPEEP |
| 646 | SPDSQEEQKGESSASSPEEPE |
| 647 | PDSQEEQKGESSASSPEEPEE |
| 648 | PADSPDSQEEQKGESSASSPEE |
| 649 | ADSPDSQEEQKGESSASSPEEP |
| 650 | DSPDSQEEQKGESSASSPEEPE |
| 651 | SPDSQEEQKGESSASSPEEPEE |
| 652 | PADSPDSQEEQKGESSASSPEEP |
| | (tag17514-2) |
| 653 | ADSPDSQEEQKGESSASSPEEPE |
| | (tag17514-3) |
| 654 | DSPDSQEEQKGESSASSPEEPEE |
| 655 | PADSPDSQEEQKGESSASSPEEPE |
| 656 | ADSPDSQEEQKGESSASSPEEPEE |
| 657 | PADSPDSQEEQKGESSASSPEEPEE |
| 658 | SPSPEDESSSSSSSSSSEDE |
| 659 | PSPEDESSSSSSSSSSEDEE (tag17603-1) |
| 660 | PRSPSPEDESSSSSSSSSSEDEE |
| | (tag17603-2) |
| 661 | PRSPSPEDESSSSSSSSSSEDEEE |
| | (tag17603-3) |
| 662 | SSSDSSDSDSSEDDEAPSKP (tag18253-1) |
| 663 | PSPGSPRGQPQDQDDDEDDEE |
| | (tag18453-1) |
| 664 | QQAEDHPQNPPEDPNQDPPE |
| 665 | QAEDHPQNPPEDPNQDPPED |
| 666 | AEDHPQNPPEDPNQDPPEDD |
| 667 | EDHPQNPPEDPNQDPPEDDS |
| 668 | QQAEDHPQNPPEDPNQDPPED |
| 669 | QAEDHPQNPPEDPNQDPPEDD |
| 670 | AEDHPQNPPEDPNQDPPEDDS |
| 671 | PQQAEDHPQNPPEDPNQDPPED |
| 672 | QQAEDHPQNPPEDPNQDPPEDD |
| 673 | QAEDHPQNPPEDPNQDPPEDDS |
| 674 | DGEAGPQQAEDHPQNPPEDPNQD |
| 675 | GPQQAEDHPQNPPEDPNQDPPED |
| 676 | PQQAEDHPQNPPEDPNQDPPEDD |
| 677 | QQAEDHPQNPPEDPNQDPPEDDS |
| 678 | GPQQAEDHPQNPPEDPNQDPPEDD |
| 679 | PQQAEDHPQNPPEDPNQDPPEDDS |
| 680 | EAGPQQAEDHPQNPPEDPNQDPPED |
| 681 | AGPQQAEDHPQNPPEDPNQDPPEDD |
| 682 | GPQQAEDHPQNPPEDPNQDPPEDDS |
| 683 | DGEAGPQQAEDHPQNPPEDPNQDPPE |
| 684 | GEAGPQQAEDHPQNPPEDPNQDPPED |
| 685 | EAGPQQAEDHPQNPPEDPNQDPPEDD |
| 686 | AGPQQAEDHPQNPPEDPNQDPPEDDS |
| 687 | DGEAGPQQAEDHPQNPPEDPNQDPPED |
| 688 | GEAGPQQAEDHPQNPPEDPNQDPPEDD |
| 689 | EAGPQQAEDHPQNPPEDPNQDPPEDDS |
| 690 | DGEAGPQQAEDHPQNPPEDPNQDPPED |
| | D |
| 691 | GEAGPQQAEDHPQNPPEDPNQDPPEDD |
| | S |
| 692 | DGEAGPQQAEDHPQNPPEDPNQDPPED |
| | DS |
| 693 | PAGDGEAGPQQAEDHPQNPPEDPNQDP |
| | PEDD (tag18467-1) |
| 694 | AGDGEAGPQQAEDHPQNPPEDPNQDPP |
| | EDDS (tag18467-2) |
| 695 | PAGDGEAGPQQAEDHPQNPPEDPNQDP |
| | PEDDS (tag18467-3) |
| 696 | QNQKEAEEPGPDSENSQENP |
| | (tag18478-1) |
| 697 | NQKEAEEPGPDSENSQENPP (tag18478-2) |
| 698 | DQNESQSPQEPEEGPSEDDK |
| 699 | QNESQSPQEPEEGPSEDDKA |
| 700 | NESQSPQEPEEGPSEDDKAE |
| 701 | DQNESQSPQEPEEGPSEDDKA (tag19033- |
| | 1) |
| 702 | QNESQSPQEPEEGPSEDDKAE (tag19033- |
| | 2) |
| 703 | NESQSPQEPEEGPSEDDKAEG (tag19033- |
| | 3) |
| 704 | ESQSPQEPEEGPSEDDKAEGE |
| 705 | SQSPQEPEEGPSEDDKAEGEE |
| 706 | DQNESQSPQEPEEGPSEDDKAE |
| 707 | QNESQSPQEPEEGPSEDDKAEG |
| 708 | NESQSPQEPEEGPSEDDKAEGE |
| 709 | DQNESQSPQEPEEGPSEDDKAEG |
| 710 | QNESQSPQEPEEGPSEDDKAEGE |
| 711 | NESQSPQEPEEGPSEDDKAEGEE |
| 712 | DQNESQSPQEPEEGPSEDDKAEGE |
| 713 | QNESQSPQEPEEGPSEDDKAEGEE |
| 714 | DQNESQSPQEPEEGPSEDDKAEGEE |
| 715 | QNESQSPQEPEEGPSEDDKAEGEEE |
| 716 | XEASSSEEEPPSPDDKENQAP (tag25919- |
| | 1) |
| 717 | PASSSSNPEEGPEEDREAESE (tag29487- |
| | 1) |
| 718 | DKPEEEDDEAQQPQPQSGPE |
| 719 | KPEEEDDEAQQPQPQSGPEE |
| 720 | AGEGDKPEEEDDEAQQPQPQS |
| 721 | EGDKPEEEDDEAQQPQPQSGP |
| 722 | GDKPEEEDDEAQQPQPQSGPE |
| 723 | DKPEEEDDEAQQPQPQSGPEE |
| 724 | KPEEEDDEAQQPQPQSGPEEA |
| 725 | PEEEDDEAQQPQPQSGPEEAE |
| 726 | EEDDEAQQPQPQSGPEEAEEG |
| 727 | EDDEAQQPQPQSGPEEAEEGE |
| 728 | DDEAQQPQPQSGPEEAEEGEE |
| 729 | DEAQQPQPQSGPEEAEEGEEE |
| 730 | EAQQPQPQSGPEEAEEGEEEE |
| 731 | QQPQPQSGPEEAEEGEEEEAE |
| 732 | QPQPQSGPEEAEEGEEEEAER |
| 733 | EGDKPEEEDDEAQQPQPQSGPE |
| 734 | GDKPEEEDDEAQQPQPQSGPEE |
| 735 | DKPEEEDDEAQQPQPQSGPEEA |
| 736 | KPEEEDDEAQQPQPQSGPEEAE |
| 737 | QQPQPQSGPEEAEEGEEEEAER |
| 738 | EGDKPEEEDDEAQQPQPQSGPEE |
| 739 | GDKPEEEDDEAQQPQPQSGPEEA |
| 740 | DKPEEEDDEAQQPQPQSGPEEAE |
| | (tag34831-1) |
| 741 | KPEEEDDEAQQPQPQSGPEEAEE |
| | (tag34831-2) |
| 742 | PEEEDDEAQQPQPQSGPEEAEEG |
| | (tag34831-3) |
| 743 | EGDKPEEEDDEAQQPQPQSGPEEA |
| 744 | GDKPEEEDDEAQQPQPQSGPEEAE |
| 745 | KPEEEDDEAQQPQPQSGPEEAEEG |
| 746 | EGDKPEEEDDEAQQPQPQSGPEEAE |
| 747 | GDKPEEEDDEAQQPQPQSGPEEAEE |
| 748 | DKPEEEDDEAQQPQPQSGPEEAEEG |
| 749 | KPEEEDDEAQQPQPQSGPEEAEEGE |
| 750 | PEEEHAPGEDESSPQPSQPS (tag34858-1) |
| 751 | XPPPEESSDSEPEAEPGSPQ (tag) |
| 752 | PPPEESSDSEPEAEPGSPQK (tag) |

### Extraction Condition 3:

length: 20 to 70 amino acids
group [D, E]: content of [45] or more
group [G]: content of less than [10]
group [A]: content of less than [10]
group [F, Y]: content of [0]
group [C, M, L, I, W, T, V]: content of [0]
group [P]: content of [15] or more

TABLE 14
Aggregation Rates of scFvs having Tags Extracted under Extraction Condition 3 Added

| | | Aggre- | | | | |
|---|---|---|---|---|---|---|
| Tag | | gation | DE | P | G | A |
| Name | Sequence | Rate % | Rate | Rate | Rate | Rate |
| Tag 167-1 | KEPKEEKKDDDEEAPKPSSD | 8.02 | 45 | 15 | 0 | 5 |
| Tag 1034-1 | SEEEKPPEEDKEEEEEKKAP | 8.91 | 55 | 15 | 0 | 5 |
| Tag 1409-1 | PAEEDEDDPEQEKEAGEPGRP | 5.17 | 47.62 | 19.05 | 9.52 | 9.52 |
| Tag 1784-1 | EPEPEPEPEPEPAPEPEPEP | 12.97 | 45 | 50 | 0 | 5 |
| Tag 2257-1 | EDPEEDGSPDPEPSPEPEPK | 12.61 | 45 | 35 | 5 | 0 |
| Tag 2257-2 | DEDPEEDGSPDPEPSPEPEPK | 11.50 | 47.62 | 33.33 | 4.76 | 0 |
| Tag 2257-3 | DEDPEEDGSPDPEPSPEPEPKP | 8.62 | 45.45 | 36.36 | 4.55 | 0 |
| Tag 2740-1 | KPEDKDPRDPEESKEPKEEK | 13.22 | 45 | 20 | 0 | 0 |
| Tag 2740-2 | PEDKDPRDPEESKEPKEEKQ | 11.68 | 45 | 20 | 0 | 0 |
| Tag 2740-3 | EDKDPRDPEESKEPKEEKQR | 8.79 | 45 | 15 | 0 | 0 |
| Tag 3227-1 | KRNDSEEEERERDEEQEPPP | 20.63 | 50 | 15 | 0 | 0 |
| Tag 4898-1 | PEEEPDDQDAPDEHEPSPSED | 10.71 | 52.38 | 23.81 | 0 | 4.76 |
| Tag 4898-2 | EEEPDDQDAPDEHEPSPSEDA | 7.01 | 52.38 | 19.05 | 0 | 9.52 |
| Tag 4898-3 | EEPDDQDAPDEHEPSPSEDAP | 19.90 | 47.62 | 23.81 | 0 | 9.52 |
| Tag 5533-1 | PSPAPSPDSDSDSDSDGEEEEEEE | 1.89 | 50 | 16.67 | 4.17 | 4.17 |
| Tag 5533-2 | APSPAPSPDSDSDSDSDGEEEEEEE | 1.00 | 48 | 16 | 4 | 8 |
| Tag 5533-3 | PSPAPSPDSDSDSDSDGEEEEEEEG | 0.90 | 48 | 16 | 8 | 4 |
| Tag 6236-1 | EKNDEDEPQKPEDKGDPEGPE | 10.19 | 47.62 | 19.05 | 9.52 | 0 |
| Tag 6236-2 | EKNDEDEPQKPEDKGDPEGPEA | 8.40 | 45.45 | 18.18 | 9.09 | 4.55 |
| Tag 6755-1 | EDEEEEEEEEEEEDEGPAPP | 4.10 | 75 | 15 | 5 | 5 |
| Tag 6755-2 | DEEEEEEEEEEEDEGPAPPS | 2.38 | 70 | 15 | 5 | 5 |
| Tag 7167-1 | GEREPDPPDDRDASDGEDEKP | 12.37 | 47.62 | 19.05 | 9.52 | 4.76 |
| Tag 7167-2 | EREPDPPDDRDASDGEDEKPP | 8.77 | 47.62 | 23.81 | 4.76 | 4.76 |
| Tag 7167-3 | EGEREPDPPDDRDASDGEDEKP | 9.97 | 50 | 18.18 | 9.09 | 4.55 |
| Tag 7702-1 | EEEEEQPGKAPDPQDPQDAESD | 13.30 | 45.45 | 18.18 | 4.55 | 9.09 |
| Tag 8243-1 | EPEEKQEPEEKQEPEEKQKPE | 11.90 | 47.62 | 19.05 | 0 | 0 |
| Tag 8243-2 | QEPEEKQEPEEKQEPEEKQKPE | 12.44 | 45.45 | 18.18 | 0 | 0 |
| Tag 8243-3 | EPEEKQEPEEKQEPEEKQKPEA | 12.60 | 45.45 | 18.18 | 0 | 4.55 |
| Tag 8818-1 | DDDDDDDSPDPESPDDSESD | 2.58 | 65 | 15 | 0 | 0 |
| Tag 8818-2 | DDDDDDSPDPESPDDSESDS | 2.19 | 60 | 15 | 0 | 0 |
| Tag 8818-3 | DDSPDPESPDDSESDSESEK | 8.33 | 50 | 15 | 0 | 0 |
| Tag 9050-1 | DPDQPREDPAEEEKEEKDAPE | 15.60 | 52.38 | 19.05 | 0 | 9.52 |
| Tag 9166-1 | PENESEPKHEEEPKPEEKPEEE | 17.13 | 50 | 22.73 | 0 | 0 |
| Tag 9166-2 | NESEPKHEEEPKPEEKPEEEEK | 7.62 | 50 | 18.18 | 0 | 0 |
| Tag 9166-3 | PENESEPKHEEEPKPEEKPEEEE | 13.00 | 52.17 | 21.74 | 0 | 0 |
| Tag 9590-1 | PPEEDPEEQAEENPEGEQPE | 13.99 | 50 | 25 | 5 | 5 |
| Tag 9590-2 | KPPEEDPEEQAEENPEGEQPE | 10.18 | 47.62 | 23.81 | 4.76 | 4.76 |
| Tag 9590-3 | KPPEEDPEEQAEENPEGEQPEE | 9.67 | 50 | 22.73 | 4.55 | 4.55 |
| Tag 9704-1 | PDDDDESEDHDDPDNAHESP | 7.65 | 55 | 15 | 0 | 5 |
| Tag 9749-1 | PEPEPEPEPEPESEPEPEPE | 15.74 | 50 | 45 | 0 | 0 |
| Tag 9749-2 | GGEPEPEPEPEPEPEPESEPEPE | 12.24 | 47.83 | 39.13 | 8.7 | 0 |
| Tag 9749-3 | GGEPEPEPEPEPEPEPESEPEPEPE | 14.29 | 48 | 40 | 8 | 0 |
| Tag 10346-1 | PEEEPDDQDAPDEHESPPPE | 8.16 | 50 | 30 | 0 | 5 |
| Tag 10346-2 | EFEPDDQDAPDEHESPPPEDA | 6.88 | 52.38 | 23.81 | 0 | 9.52 |
| Tag 10346-3 | PEEEPDDQDAPDEHESPPPEDAP | 8.97 | 47.83 | 30.43 | 0 | 8.7 |
| Tag 11099-1 | GPSSDDENEEESKPEKEDEP | 7.21 | 50 | 15 | 5 | 0 |
| Tag 11099-2 | PSSDDENEEESKPEKEDEPQ | 5.09 | 50 | 15 | 0 | 0 |
| Tag 12127-1 | SDDSDSEKRRPEEQEEEPQP | 17.51 | 45 | 15 | 0 | 0 |
| Tag 12127-2 | DDSDSEKRRPEEQEEEPQPR | 17.44 | 45 | 15 | 0 | 0 |
| Tag 13036-1 | PEEEDEEPGDPREGEEEEEEDEPDP | 3.17 | 68 | 20 | 8 | 0 |
| Tag 13036-2 | PEEEDEEPGDPREGEEEEEEDEPDPEAP | 5.60 | 64.29 | 21.43 | 7.14 | 3.57 |
| Tag 13036-3 | AAPEEEDEEPGDPREGEEEEEEDEPDPEAPENGS | 6.19 | 55.88 | 17.65 | 8.82 | 8.82 |
| Tag 14128-1 | PPPSEGSDEEEEEEDEEDEE | 2.43 | 70 | 15 | 5 | 0 |
| Tag 14128-2 | KPPPSEGSDEEEEEEDEEDEEERKP | 10.86 | 60 | 16 | 4 | 0 |
| Tag 14128-3 | KPPPSEGSDEEEEEEDEEDEEERKPQ | 9.76 | 57.69 | 15.38 | 3.85 | 0 |
| Tag 16549-1 | EEEEEEEEEEEEEEEAPPPP | 4.95 | 75 | 20 | 0 | 5 |
| Tag 16549-2 | EEEEEEEEEEEEEEEEEAPPPPR | 3.01 | 73.9 | 17.39 | 0 | 4.35 |
| Tag 16549-3 | PDDDEEDEEEEEEEEEEEEEEEEEEEEEAPPPP | 0.89 | 81.82 | 15.15 | 0 | 3.03 |
| Tag 16604-1 | EDSEEESQEEEAEGASEPPPP | 6.00 | 47.62 | 19.05 | 4.76 | 9.52 |
| Tag 16604-2 | QEDSEEESQEEEAEGASEPPPP | 7.39 | 45.45 | 18.18 | 4.55 | 9.09 |
| Tag 16604-3 | DHQEDSEEESQEEEAEGASEPPPP | 10.59 | 45.83 | 16.67 | 4.17 | 8.33 |
| Tag 16741-1 | DQSEEEEEEEKHPPKPAKPE | 10.62 | 45 | 20 | 0 | 5 |
| Tag 16991-1 | PAPAHRPPEDEGEENEGEEDE | 14.97 | 47.62 | 19.05 | 9.52 | 9.52 |
| Tag 16991-2 | PAPAHRPPEDEGEENEGEEDEE | 7.12 | 50 | 18.18 | 9.09 | 9.09 |
| Tag 17199-1 | QENGQREEEEEEKEPEAEPP | 9.09 | 50 | 15 | 5 | 5 |
| Tag 17199-2 | PAEGQENGQREEEEEEKEPEAEPP | 10.29 | 45.83 | 16.67 | 8.33 | 8.33 |
| Tag 17936-1 | EQEPEPEPEPEPEPEPEPEP | 6.37 | 50 | 45 | 0 | 0 |
| Tag 17936-2 | EPEPEPEPEPEPEPEPEPEQ | 10.94 | 50 | 45 | 0 | 0 |
| Tag 17936-3 | EQEPEPEPEPEPEPEPEPEPEQ | 16.61 | 50 | 40.91 | 0 | 0 |
| Tag 18132-1 | PEEEEEEEEEEEPASPPERK | 3.36 | 60 | 20 | 0 | 5 |
| Tag 18453-1 | PSPGSPRGQPQDQDDDEDDEED | 4.31 | 45.45 | 18.18 | 9.09 | 0 |
| Tag 18453-2 | RPSPGSPRGQPQDQDDDEDDEEDE | 13.65 | 45.83 | 16.67 | 8.33 | 0 |
| Tag 18453-3 | PSPGSPRGQPQDQDDDEDDEEDEA | 6.54 | 45.83 | 16.67 | 8.33 | 4.17 |
| Tag 18866-1 | AEDDDEEDEEEEEEEPDPDP | 2.24 | 80 | 15 | 0 | 5 |
| Tag 18866-2 | EDDDEEDEEEEEEEPDPDPE | 2.29 | 85 | 15 | 0 | 0 |
| Tag 19350-1 | KQEPPDPEEDKEENKDDSAS | 14.73 | 45 | 15 | 0 | 5 |
| Tag 19350-2 | QEPPDPEEDKEENKDDSASK | 14.12 | 45 | 15 | 0 | 5 |
| Tag 19511-1 | EDEDEDESSEEDSEDEEPPP | 1.70 | 70 | 15 | 0 | 0 |
| Tag 19511-2 | PDDSRDEDEDEDESSEEDSEDEEPPP | 4.37 | 65.38 | 15.38 | 0 | 0 |
| Tag 19511-3 | PKKEPDDSRDEDEDEDESSEEDSEDEEPPPKRR | 3.87 | 54.55 | 15.15 | 0 | 0 |
| Tag 22900-1 | PEEEAAEEEEEEEERPKPSRP | 10.93 | 52.38 | 19.05 | 0 | 9.52 |
| Tag 22900-2 | EQPEEEAAEEEEEEEERPKPSRP | 6.90 | 52.17 | 17.39 | 0 | 8.7 |
| Tag 22900-3 | EEEQPEEEAAEEEEEEEERPKPSRP | 9.67 | 56 | 16 | 0 | 8 |
| Tag 34831-1 | PEEEDDEAQQPQPQSGPEEAEE | 10.16 | 45.45 | 18.18 | 4.55 | 9.09 |
| Tag 34831-2 | EGDKPEEEDDEAQQPQPQSGPEEAEE | 10.78 | 46.15 | 15.38 | 7.69 | 7.69 |
| Tag 34831-3 | KPEEEDDEAQQPQPQSGPEEAEEGEEEEAERGP | 8.33 | 45.45 | 15.15 | 9.09 | 9.09 |
| Tag 39056-1 | NNSEEEEDDDDEEEEPDKPP | 0.44 | 85 | 15 | 0 | 0 |
| Tag 39056-2 | NSEEEEDDDDEEEEPDKPPA | 0.99 | 65 | 15 | 0 | 5 |
| Tag 39056-3 | SEEEEDDDDEEEEPDKPPAN | 1.23 | 65 | 15 | 0 | 5 |

In Table 14, the acidic amino acid ratio in the amino acid sequences of the extracted tags is 45% or more, the P content ratio is 15% or more, and the content ratios of the other amino acids are as described above in Extraction Condition 3. In order to confirm that the aggregation inhibiting action does not depend on a specific amino acid sequence, tags were randomly selected from the tags extracted under Extraction Condition 3. As for some tags, an amino acid sequence satisfying the extraction condition was additionally selected from another portion of the same protein. The aggregation rates of scFvs tagged with the selected amino acid sequences were tested, and results as shown in Table 14 were obtained. As shown in Table 14, the aggregation rates of the tagged scFvs were low as a whole. It is understood, from Table 14-1, that a strategy of reducing the G, A, F, Y, C, M, L, I, W, T, and V contents and increasing the P content works on a peptide tag having a high acidic amino acid content.

It is noted that human-derived amino acid sequences that can be extracted under Extraction Condition 3 were as follows.

TABLE 14-2
Examples of human-derived amino acid
sequences that can be extracted under
Extraction Condition 3

| SEQ | |
|---|---|
| ID | |
| NO: | Sequence |
| 753 | KEPKEEKKDDDEEAPKPSSD (Tag 167-1) |
| 754 | SEEEKPPEEDKEEEEEKKAP (Tag 1034-1) |
| 755 | PAEEDEDDPEQEKEAGEPGRP (Tag |
| | 1409-1) |
| 756 | PEPEPEPEPEPEPAPEPEPE |
| 757 | EPEPEPEPEPEPAPEPEPEP (Tag 1784-1) |
| 758 | DEDPEEDGSPDPEPSPEPEP |
| 759 | EDPEEDGSPDPEPSPEPEPK (Tag 2257- |
| | 1) |
| 760 | DEDPEEDGSPDPEPSPEPEPK (Tag 2257- |
| | 2) |
| 761 | DEDPEEDGSPDPEPSPEPEPKP (Tag |
| | 2257-3) |
| 762 | KPEDKDPRDPEESKEPKEEK (Tag 2740- |
| | 1) |
| 763 | PEDKDPRDPEESKEPKEEKQ (Tag 2740- |
| | 2) |
| 764 | EDKDPRDPEESKEPKEEKQR (Tag 2740- |
| | 3) |
| 765 | KRNDSEEEERERDEEQEPPP (Tag 3227- |
| | 1) |
| 766 | PEEEPDDQDAPDEHEPSPSE |
| 767 | EEEPDDQDAPDEHEPSPSED |
| 768 | PEEEPDDQDAPDEHEPSPSED (Tag |
| | 4898-1) |
| 769 | EEEPDDQDAPDEHEPSPSEDA (Tag |
| | 4898-2) |
| 770 | EEPDDQDAPDEHEPSPSEDAP (Tag |
| | 4898-3) |
| 771 | PEEEPDDQDAPDEHEPSPSEDA |
| 772 | EEEPDDQDAPDEHEPSPSEDAP |
| 773 | PEEEPDDQDAPDEHEPSPSEDAP |
| 774 | SPAPSPDSDSDSDSDGEEEE |
| 775 | PAPSPDSDSDSDSDGEEEEE |
| 776 | PSPAPSPDSDSDSDSDGEEEEE |
| 777 | PSPAPSPDSDSDSDSDGEEEEEE |
| 778 | APSPAPSPDSDSDSDSDGEEEEEE |
| 779 | PSPAPSPDSDSDSDSDGEEEEEEE (Tag |
| | 5533-1) |
| 780 | APSPAPSPDSDSDSDSDGEEEEEEE (Tag |
| | 5533-2) |
| 781 | PSPAPSPDSDSDSDSDGEEEEEEEG (Tag |
| | 5533-3) |
| 782 | GAPSPAPSPDSDSDSDSDGEEEEEEE |
| 783 | APSPAPSPDSDSDSDSDGEEEEEEEG |
| 784 | PSPAPSPDSDSDSDSDGEEEEEEEGE |
| 785 | EKNDEDEPQKPEDKGDPEGPE |
| | (Tag 6236-1) |
| 786 | EKNDEDEPQKPEDKGDPEGPEA (Tag |
| | 6236-2) |
| 787 | EDEEEEEEEEEEEDEGPAPP (Tag 6755-1) |
| 788 | DEEEEEEEEEEEDEGPAPPS (Tag 6755-2) |
| 789 | EREPDPPDDRDASDGEDEKP |
| 790 | REPDPPDDRDASDGEDEKPP |
| 791 | GEREPDPPDDRDASDGEDEKP (Tag |
| | 7167-1) |
| 792 | EREPDPPDDRDASDGEDEKPP (Tag |
| | 7167-2) |
| 793 | EGEREPDPPDDRDASDGEDEKP (Tag |
| | 7167-3) |
| 794 | GEREPDPPDDRDASDGEDEKPP |
| 795 | EGEREPDPPDDRDASDGEDEKPP |
| 796 | EEEEEQPGKAPDPQDPQDAESD (Tag |
| | 7702-1) |
| 797 | QEPEEKQEPEEKQEPEEKQK |
| 798 | EPEEKQEPEEKQEPEEKQKP |
| 799 | PEEKQEPEEKQEPEEKQKPE |
| 800 | EEKQEPEEKQEPEEKQKPEA |
| 801 | EPEEKQEPEEKQEPEEKQKPE (Tag 8243- |
| | 1) |
| 802 | QEPEEKQEPEEKQEPEEKQKPE (Tag |
| | 8243-2) |
| 803 | EPEEKQEPEEKQEPEEKQKPEA (Tag |
| | 8243-3) |
| 804 | AGDDDDDDDDSPDPESPDDS |
| 805 | GDDDDDDDDSPDPESPDDSE |
| 806 | DDDDDDDDSPDPESPDDSES |
| 807 | DDDDDDDSPDPESPDDSESD (Tag 8818- |
| | 1) |
| 808 | DDDDDDSPDPESPDDSESDS (Tag 8818- |
| | 2) |
| 809 | DDDDDSPDPESPDDSESDSE |
| 810 | DDDDSPDPESPDDSESDSES |
| 811 | DDDSPDPESPDDSESDSESE |
| 812 | DDSPDPESPDDSESDSESEK (Tag 8818- |
| | 3) |
| 813 | DSPDPESPDDSESDSESEKE |
| 814 | SPDPESPDDSESDSESEKEE |
| 815 | PDPESPDDSESDSESEKEES |
| 816 | DPDQPREDPAEEEKEEKDAPE |
| | (Tag 9050-1) |
| 817 | EGKPENESEPKHEEEPKPEE |
| 818 | PENESEPKHEEEPKPEEKPE |
| 819 | ENESEPKHEEEPKPEEKPEE |
| 820 | NESEPKHEEEPKPEEKPEEE |
| 821 | ESEPKHEEEPKPEEKPEEEE |
| 822 | SEPKHEEEPKPEEKPEEEEK |
| 823 | PENESEPKHEEEPKPEEKPEE |
| 824 | ENESEPKHEEEPKPEEKPEEE |
| 825 | NESEPKHEEEPKPEEKPEEEE |
| 826 | ESEPKHEEEPKPEEKPEEEEK |
| 827 | KPENESEPKHEEEPKPEEKPEE |
| 828 | PENESEPKHEEEPKPEEKPEEE (Tag |
| | 9166-1) |
| 829 | ENESEPKHEEEPKPEEKPEEEE |
| 830 | NESEPKHEEEPKPEEKPEEEEK (Tag |
| | 9166-2) |
| 831 | KPENESEPKHEEEPKPEEKPEEE |
| 832 | PENESEPKHEEEPKPEEKPEEEE (Tag |
| | 9166-3) |
| 833 | ENESEPKHEEEPKPEEKPEEEEK |
| 834 | EGKPENESEPKHEEEPKPEEKPEE |
| 835 | GKPENESEPKHEEEPKPEEKPEEE |
| 836 | KPENESEPKHEEEPKPEEKPEEEE |
| 837 | PENESEPKHEEEPKPEEKPEEEEK |
| 838 | EGKPENESEPKHEEEPKPEEKPEEE |
| 839 | GKPENESEPKHEEEPKPEEKPEEEE |
| 840 | KPENESEPKHEEEPKPEEKPEEEEK |
| 841 | EGKPENESEPKHEEEPKPEEKPEEEE |
| 842 | GKPENESEPKHEEEPKPEEKPEEEEK |
| 843 | EGKPENESEPKHEEEPKPEEKPEEEEK |
| 844 | KPPEEDPEEQAEENPEGEQP |
| 845 | PPEEDPEEQAEENPEGEQPE (Tag 9590- |
| | 1) |
| 846 | PEEDPEEQAEENPEGEQPEE |
| 847 | KPPEEDPEEQAEENPEGEQPE (Tag |
| | 9590-2) |
| 848 | PPEEDPEEQAEENPEGEQPEE |
| 849 | KPPEEDPEEQAEENPEGEQPEE (Tag |
| | 9590-3) |
| 850 | PDDDDESEDHDDPDNAHESP (Tag 9704- |
| | 1) |
| 851 | GEPEPEPEPEPEPEPESEPE |
| 852 | EPEPEPEPEPEPEPESEPEP |
| 853 | PEPEPEPEPEPEPESEPEPE |
| 854 | EPEPEPEPEPEPESEPEPEP |
| 855 | PEPEPEPEPEPESEPEPEPE (Tag 9749-1) |
| 856 | GGEPEPEPEPEPEPEPESEPE |
| 857 | GEPEPEPEPEPEPEPESEPEP |
| 858 | EPEPEPEPEPEPEPESEPEPE |
| 859 | PEPEPEPEPEPEPESEPEPEP |
| 860 | EPEPEPEPEPEPESEPEPEPE |
| 861 | GGEPEPEPEPEPEPEPESEPEP |
| 862 | GEPEPEPEPEPEPEPESEPEPE |
| 863 | EPEPEPEPEPEPEPESEPEPEP |
| 864 | PEPEPEPEPEPEPESEPEPEPE |
| 865 | GGEPEPEPEPEPEPEPESEPEPE (Tag |
| | 9749-2) |
| 866 | GEPEPEPEPEPEPEPESEPEPEP |
| 867 | EPEPEPEPEPEPEPESEPEPEPE |
| 868 | GGEPEPEPEPEPEPEPESEPEPEP |
| 869 | GEPEPEPEPEPEPEPESEPEPEPE |
| 870 | GGEPEPEPEPEPEPEPESEPEPEPE (Tag |
| | 9749-3) |
| 871 | PEEEPDDQDAPDEHESPPPE (Tag 10346- |
| | 1) |
| 872 | EEEPDDQDAPDEHESPPPED |
| 873 | PEEEPDDQDAPDEHESPPPED |
| 874 | EEEPDDQDAPDEHESPPPEDA (Tag |
| | 10346-2) |
| 875 | EEPDDQDAPDEHESPPPEDAP |
| 876 | PEEEPDDQDAPDEHESPPPEDA |
| 877 | EEEPDDQDAPDEHESPPPEDAP |
| 878 | PEEEPDDQDAPDEHESPPPEDAP (Tag |
| | 10346-3) |
| 879 | GPSSDDENEEESKPEKEDEP (Tag 11099- |
| | 1) |
| 880 | PSSDDENEEESKPEKEDEPQ (Tag 11099- |
| | 2) |
| 881 | SDDSDSEKRRPEEQEEEPQP (Tag |
| | 12127-1) |
| 882 | DDSDSEKRRPEEQEEEPQPR (Tag |
| | 12127-2) |
| 883 | DPREGEEEEEEDEPDPEAPE |
| 884 | PREGEEEEEEDEPDPEAPEN |
| 885 | DEEPGDPREGEEEEEEDEPDP |
| 886 | EEPGDPREGEEEEEEDEPDPE |
| 887 | EPGDPREGEEEEEEDEPDPEA |
| 888 | PGDPREGEEEEEEDEPDPEAP |
| 889 | GDPREGEEEEEEDEPDPEAPE |
| 890 | DPREGEEEEEEDEPDPEAPEN |
| 891 | PREGEEEEEEDEPDPEAPENG |
| 892 | EDEEPGDPREGEEEEEEDEPDP |
| 893 | DEEPGDPREGEEEEEEDEPDPE |
| 894 | EEPGDPREGEEEEEEDEPDPEA |
| 895 | EPGDPREGEEEEEEDEPDPEAP |
| 896 | PGDPREGEEEEEEDEPDPEAPE |
| 897 | GDPREGEEEEEEDEPDPEAPEN |
| 898 | DPREGEEEEEEDEPDPEAPENG |
| 899 | PREGEEEEEEDEPDPEAPENGS |
| 900 | PEEEDEEPGDPREGEEEEEEDEP |
| 901 | EEDEEPGDPREGEEEEEEDEPDP |
| 902 | EDEEPGDPREGEEEEEEDEPDPE |
| 903 | DEEPGDPREGEEEEEEDEPDPEA |
| 904 | EEPGDPREGEEEEEEDEPDPEAP |
| 905 | EPGDPREGEEEEEEDEPDPEAPE |
| 906 | PGDPREGEEEEEEDEPDPEAPEN |
| 907 | DPREGEEEEEEDEPDPEAPENGS |
| 908 | APEEEDEEPGDPREGEEEEEEDEP |
| 909 | PEEEDEEPGDPREGEEEEEEDEPD |
| 910 | EEEDEEPGDPREGEEEEEEDEPDP |
| 911 | EEDEEPGDPREGEEEEEEDEPDPE |
| 912 | EDEEPGDPREGEEEEEEDEPDPEA |
| 913 | DEEPGDPREGEEEEEEDEPDPEAP |
| 914 | EEPGDPREGEEEEEEDEPDPEAPE |
| 915 | EPGDPREGEEEEEEDEPDPEAPEN |
| 916 | AAPEEEDEEPGDPREGEEEEEEDEP |
| 917 | APEEEDEEPGDPREGEEEEEEDEPD |
| 918 | PEEEDEEPGDPREGEEEEEEDEPDP |
| | (Tag 13036-1) |
| 919 | EEEDEEPGDPREGEEEEEEDEPDPE |
| 920 | EEDEEPGDPREGEEEEEEDEPDPEA |
| 921 | EDEEPGDPREGEEEEEEDEPDPEAP |
| 922 | DEEPGDPREGEEEEEEDEPDPEAPE |
| 923 | EEPGDPREGEEEEEEDEPDPEAPEN |
| 924 | AAPEEEDEEPGDPREGEEEEEEDEPD |
| 925 | APEEEDEEPGDPREGEEEEEEDEPDP |
| 926 | PEEEDEEPGDPREGEEEEEEDEPDPE |
| 927 | EEEDEEPGDPREGEEEEEEDEPDPEA |
| 928 | EEDEEPGDPREGEEEEEEDEPDPEAP |
| 929 | EDEEPGDPREGEEEEEEDEPDPEAPE |
| 930 | DEEPGDPREGEEEEEEDEPDPEAPEN |
| 931 | AAPEEEDEEPGDPREGEEEEEEDEPDP |
| 932 | APEEEDEEPGDPREGEEEEEEDEPDPE |
| 933 | PEEEDEEPGDPREGEEEEEEDEPDPEA |
| 934 | EEEDEEPGDPREGEEEEEEDEPDPEAP |
| 935 | EEDEEPGDPREGEEEEEEDEPDPEAPE |
| 936 | EDEEPGDPREGEEEEEEDEPDPEAPEN |
| 937 | AAPEEEDEEPGDPREGEEEEEEDEPDPE |
| 938 | APEEEDEEPGDPREGEEEEEEDEPDPEA |
| 939 | PEEEDEEPGDPREGEEEEEEDEPDPEAP |
| | (Tag 13036-2) |
| 940 | EEEDEEPGDPREGEEEEEEDEPDPEAPE |
| 941 | EEDEEPGDPREGEEEEEEDEPDPEAPEN |
| 942 | APEEEDEEPGDPREGEEEEEEDEPDPEA |
| | P |
| 943 | PEEEDEEPGDPREGEEEEEEDEPDPEAP |
| | E |
| 944 | EEEDEEPGDPREGEEEEEEDEPDPEAPE |
| | N |
| 945 | APEEEDEEPGDPREGEEEEEEDEPDPEA |
| | PE |
| 946 | PEEEDEEPGDPREGEEEEEEDEPDPEAP |
| | EN |
| 947 | PRGAAAPEEEDEEPGDPREGEEEEEED |
| | EPDP |
| 948 | RGAAAPEEEDEEPGDPREGEEEEEEDE |
| | PDPE |
| 949 | AAPEEEDEEPGDPREGEEEEEEDEPDPE |
| | APE |
| 950 | APEEEDEEPGDPREGEEEEEEDEPDPEA |
| | PEN |
| 951 | PEEEDEEPGDPREGEEEEEEDEPDPEAP |
| | ENG |
| 952 | EEEDEEPGDPREGEEEEEEDEPDPEAPE |
| | NGS |
| 953 | PRGAAAPEEEDEEPGDPREGEEEEEED |
| | EPDPE |
| 954 | AAPEEEDEEPGDPREGEEEEEEDEPDPE |
| | APEN |
| 955 | APEEEDEEPGDPREGEEEEEEDEPDPEA |
| | PENG |
| 956 | PEEEDEEPGDPREGEEEEEEDEPDPEAP |
| | ENGS |
| 957 | AAPEEEDEEPGDPREGEEEEEEDEPDPE |
| | APENG |
| 958 | APEEEDEEPGDPREGEEEEEEDEPDPEA |
| | PENGS |
| 959 | AAPEEEDEEPGDPREGEEEEEEDEPDPE |
| | APENGS (Tag 13036-3) |
| 960 | KPPPSEGSDEEEEEEDEEDE |
| 961 | PPPSEGSDEEEEEEDEEDEE (Tag 14128- |
| | 1) |
| 962 | PPPSEGSDEEEEEEDEEDEEERKP |
| 963 | KPPPSEGSDEEEEEEDEEDEEERKP (Tag |
| | 14128-2) |
| 964 | PPPSEGSDEEEEEEDEEDEEERKPQ |
| 965 | KPPPSEGSDEEEEEEDEEDEEERKPQ |
| | (Tag 14128-3) |
| 966 | EEEEEEEEEEEEEEEEAPPP |
| 967 | EEEEEEEEEEEEEEEAPPPP (Tag 16549- |
| | 1) |
| 968 | EEEEEEEEEEEEEEAPPPPR |
| 969 | EEEEEEEEEEEEEEEEAPPPP |
| 970 | EEEEEEEEEEEEEEEAPPPPR |
| 971 | EEEEEEEEEEEEEEEEAPPPP |
| 972 | EEEEEEEEEEEEEEEAPPPPR |
| 973 | EEEEEEEEEEEEEEEEEAPPPP |
| 974 | EEEEEEEEEEEEEEEEAPPPPR (Tag |
| | 16549-2) |
| 975 | EEEEEEEEEEEEEEEEEEAPPPP |
| 976 | EEEEEEEEEEEEEEEEEAPPPPR |
| 977 | EEEEEEEEEEEEEEEEEEEAPPPP |
| 978 | EEEEEEEEEEEEEEEEEEAPPPPR |
| 979 | EEEEEEEEEEEEEEEEEEEEAPPPP |
| 980 | EEEEEEEEEEEEEEEEEEEAPPPPR |
| 981 | PDDDEEDEEEEEEEEEEEEEEEEEEEEE |
| | APPPP (Tag 16549-3) |
| 982 | EDSEEESQEEEAEGASEPPPP (Tag |
| | 16604-1) |
| 983 | QEDSEEESQEEEAEGASEPPPP (Tag |
| | 16604-2) |
| 984 | DHQEDSEEESQEEEAEGASEPPPP (Tag |
| | 16604-3) |
| 985 | DQSEEEEEEEKHPPKPAKPE (Tag 16741- |
| | 1) |
| 986 | PAPAHRPPEDEGEENEGEEDE |
| | (Tag16991-1) |
| 987 | PAPAHRPPEDEGEENEGEEDEE |
| | (Tag16991-2) |
| 988 | QENGQREEEEEEKEPEAEPP (Tag 17199- |
| | 1) |
| 989 | PAEGQENGQREEEEEEKEPEAEPP (Tag |
| | 17199-2) |
| 990 | EQEPEPEPEPEPEPEPEPEP (Tag 17936- |
| | 1) |
| 991 | QEPEPEPEPEPEPEPEPEPE |
| 992 | EPEPEPEPEPEPEPEPEPEQ (Tag 17936- |
| | 2) |
| 993 | EQEPEPEPEPEPEPEPEPEPE |
| 994 | QEPEPEPEPEPEPEPEPEPEQ |
| 995 | EQEPEPEPEPEPEPEPEPEPEQ (Tag |
| | 17936-3) |
| 996 | PEEEEEEEEEEEPASPPERK (Tag 18132- |
| | 1) |
| 997 | PSPGSPRGQPQDQDDDEDDEED (Tag |
| | 18453-1) |
| 998 | PSPGSPRGQPQDQDDDEDDEEDE |
| 999 | RPSPGSPRGQPQDQDDDEDDEEDE (Tag |
| | 18453-2) |
| 1000 | PSPGSPRGQPQDQDDDEDDEEDEA (Tag |
| | 18453-3) |
| 1001 | AEDDDEEDEEEEEEEPDPDP (Tag 18866- |
| | 1) |
| 1002 | EDDDEEDEEEEEEEPDPDPE (Tag 18866- |
| | 2) |
| 1003 | KQEPPDPEEDKEENKDDSAS (Tag 19350- |
| | 1) |
| 1004 | QEPPDPEEDKEENKDDSASK (Tag 19350- |
| | 2) |
| 1005 | EDEDEDESSEEDSEDEEPPP (Tag 19511- |
| | 1) |
| 1006 | DEDEDESSEEDSEDEEPPPK |
| 1007 | EDEDESSEEDSEDEEPPPKR |
| 1008 | DEDESSEEDSEDEEPPPKRR |
| 1009 | PDDSRDEDEDEDESSEEDSEDEEPPP |
| | (Tag19511-2) |
| 1010 | PKKEPDDSRDEDEDEDESSEEDSEDEEP |
| | PP |
| 1011 | PKKEPDDSRDEDEDEDESSEEDSEDEEP |
| | PPK |
| 1012 | PKKEPDDSRDEDEDEDESSEEDSEDEEP |
| | PPKR |
| 1013 | PKKEPDDSRDEDEDEDESSEEDSEDEEP |
| | PPKRR (Tag 19511-3) |
| 1014 | PEEEAAEEEEEEEERPKPSRP (Tag |
| | 22900-1) |
| 1015 | QPEEEAAEEEEEEEERPKPSRP |
| 1016 | EQPEEEAAEEEEEEEERPKPSRP (Tag |
| | 22900-2) |
| 1017 | EEQPEEEAAEEEEEEEERPKPSRP |
| 1018 | EEEQPEEEAAEEEEEEEERPKPSRP (Tag |
| | 22900-3) |
| 1019 | PEEEDDEAQQPQPQSGPEEAEE (Tag |
| | 34831-1) |
| 1020 | DKPEEEDDEAQQPQPQSGPEEAEE |
| 1021 | PEEEDDEAQQPQPQSGPEEAEEGE |
| 1022 | PEEEDDEAQQPQPQSGPEEAEEGEE |
| 1023 | EGDKPEEEDDEAQQPQPQSGPEEAEE |
| | (Ta 34831-2) |
| 1024 | DKPEEEDDEAQQPQPQSGPEEAEEGE |
| 1025 | KPEEEDDEAQQPQPQSGPEEAEEGEE |
| 1026 | PEEEDDEAQQPQPQSGPEEAEEGEEE |
| 1027 | PEEEDDEAQQPQPQSGPEEAEEGEEEE |
| | AERGP |
| 1028 | KPEEEDDEAQQPQPQSGPEEAEEGEEE |
| | EAERGP (Tag 34831-3) |
| 1029 | NNSEEEEDDDDEEEEPDKPP (Tag 39056- |
| | 1) |
| 1030 | NSEEEEDDDDEEEEPDKPPA (Tag 39056- |
| | 2) |
| 1031 | SEEEEDDDDEEEEPDKPPAN (Tag 39056- |
| | 3) |

As shown by these examples, the protein aggregation rate reducing action of a peptide tag had low dependency on a specific amino acid sequence, also had low dependency on a protein from which it is derived, but had high dependency on amino acid contents.

### Example 6: Addition to Various Proteins and Aggregation Inhibiting Action

In the above Example, Y13-259 was used as the scFv. In this example, a VHH antibody (a heavy chain variable domain of a heavy chain antibody) was used. As the VHH antibody, iDab #6 binding to Ras was used. As the tag, Tag4-8 was used. The other conditions were the same as those employed in Example 1. As a result, the aggregation rate of the VHH antibody not having the tag was 57.89%, the aggregation rate of the VHH antibody having Tag4-8 at the C terminal was 8.77%, and thus, a strong aggregation inhibiting action was exhibited by the tag addition.

When SHSY5Y (human dopamine-like cell) that is a neuroblastoma cell line was used as the cell, scFv-6E (6E) was used as the scFv, and Tag4-8 or Tag18-1 was added as the tag to the C terminal and the N terminal, the aggregation rate of the Tag4-8 added scFv was 0.96%, the aggregation rate of the Tag18-1 added scFv was 1.14%, and thus, it was revealed that the tag addition makes a contribution to the low aggregation rate.

When D4 binding to botulinum toxin type A (SEQ ID NO: 1032: QVQLQQSGGGLVQPGGSLRLSCAASGFTLDYYAIGWFRQAPGKEREGVLCISSSGGS TNYADSVKGRFTISRDNAKNTVYLQMNSLKPEDTAVYYCAADDLRCGSNWSSYFRGS WGQGTQVTVSS) was used as the VHH antibody, and the Tag4-8 was added as the tag to the C terminal of the VHH antibody, the aggregation rate of the Tag4-8 added D4 was 7.8%, and the aggregation rate of D4 not having the tag was 81.5%. This result reveals that the tag addition makes a contribution to the reduction of the aggregation rate of the VHH antibody.

### Example 7: Action Enhancement by Stabilization of Intracellular Antibody (Effect of Enhancement of Antibody Action on Amyloid Accumulation)

It is known that a central nervous system disease is caused by accumulation of amyloid in a nerve cell. When human α-synuclein fibril is extracellularly introduced into a nerve cell, synuclein fibril is formed with synuclein having a normal structure in the cell involved. When GFP-tagged synuclein has been expressed in the cell, the synuclein forms synuclein fibril together with the introduced human α-synuclein fibril, and the thus formed synuclein fibril can be observed with fluorescence of GFP. In this example, GFP-tagged synuclein was expressed in SHSY-5Y cell, and α-synuclein fibril (Cosmo Bio Co., Ltd., SYNO3) was extracellularly introduced into the SHSY-5Y cell. In this example, the ability of an antibody to reduce the synuclein fibril was tested. Specifically, as the antibody, scFv-6E binding to fibrilized synuclein (SEQ ID NO: 1033: AEVQLLESGGGLVQPGGSLRLSCAASGFTFSSYAMSWVRQAPGKGLEWVSYIASGGD TTNYADSVKGRFTISRDNSKNTLYLQMNSLRAEDTAVYYCAKGASAFDYWGQGTLVT VSSGGGGSGGGGSGGGGSTDIQMTQSPSSLSASVGDRVTITCRASQSISSYLNWYQQ KPGKAPKLLIYAASYLQSGVPSRFSGSGSGTDFTLTISSLQPEDFATYYCQQSSNDP YTFGQGTKVEIKR) was used. The scFv-6E is an antibody not having a significant binding property to a monomer or oligomer of synuclein, and such an antibody selective or specific to synuclein fibril is suitable for selectively removing synuclein fibril. To the scFv-6E, a tag having the aggregation rate reducing action (Tag4-8 or Tag18-1) and a degradation-inducing sequence (CMA (SEQ ID NO: 1034): MARVKKDQAEPLHRKFERQPPG) were added. An expression plasmid vector for the protein and the synuclein fibril were introduced into the cell respectively with X-trem GENE9 and Multifectam (Merck). The antibody was provided with an HA tag or a myc tag, and detected with an anti-HA tag antibody. The synuclein fibril was detected with an anti-phosphorylated α-synuclein antibody. These antibodies were specifically detected with Alexa 555 labeled antibody and Alexa 633 labeled antibody. Fluorescent stained cells were observed with Keyence BZ-X800.

The tag was added to the N terminal and the C terminal. In consideration that the aggregation rate of one having Tag4-8 added to the N terminal and the C terminal was 0.96% in the SHSY-5Y cell, that the aggregation rate of one having TAG18-1 added to the N terminal and the C terminal was 1.14% in the SHSY-5 cell, and that the aggregation rate of the scFv-E6 having no tag added was 41.6% in the HeLa cell, it seems that favorable aggregation inhibition was exhibited.

When a scFv binds to synuclein fibril, the lysosome is caused to target the synuclein fibril for a degradation-inducing sequence to degrade the synuclein fibril, and as a result, the amount of synuclein fibril in the cell is expected to be reduced. It was evaluated whether or not the reduction amount of the synuclein fibril is increased when the aggregation rate of the scFv was reduced by using the tag to increase the amount of functional scFv in the cell.

The antibody was expressed in the cell where the synuclein fibril was formed, and the number of synuclein fibril-positive cells was counted. A positive rate of the synuclein fibril was compared between a cell in which the antibody was expressed and a cell in which it was not expressed. The result was obtained as a rate of synuclein fibril-positive cells in antibody-positive cells/a rate of synuclein fibril-positive cells in antibody-negative cells (P/N).

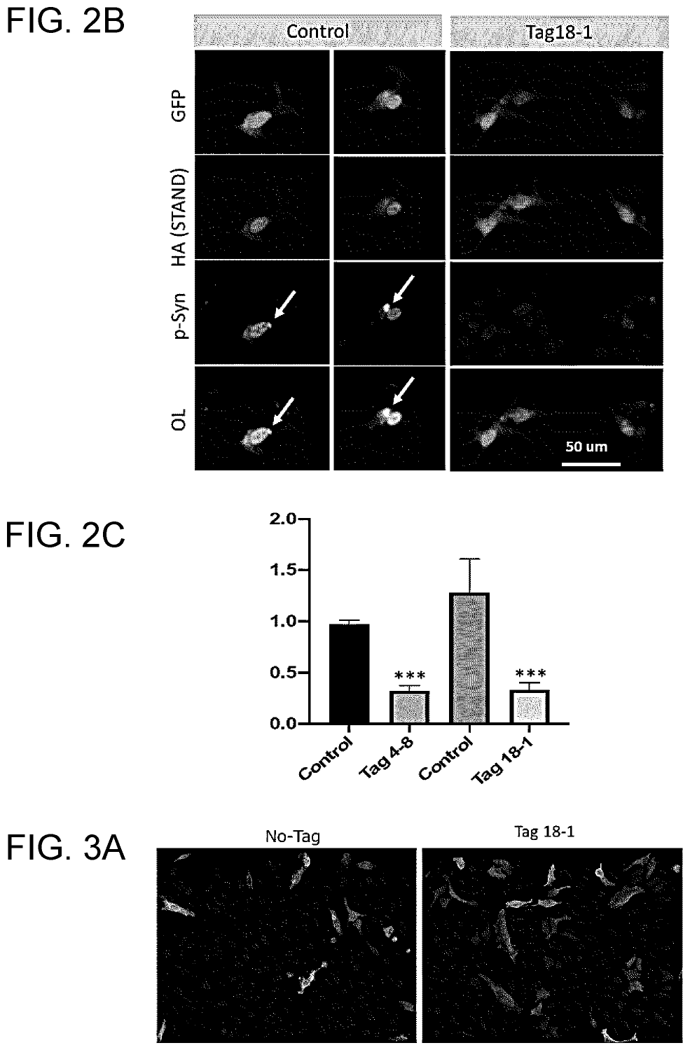

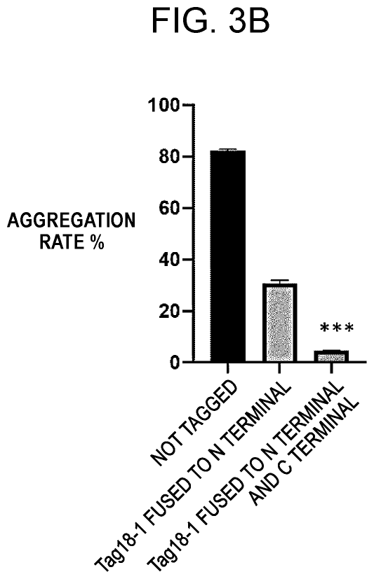

As illustrated in FIG. 2, phosphorylated synuclein was lost in the cell into which the tagged scFv-E6 was introduced. The P/N was as illustrated in FIG. 3. As illustrated in FIG. 3, both the scFv-E6 tagged with Tag4-8 and the scFv-E6 tagged with Tag18-1 largely reduced the rate of synuclein-positive cells.

### Example 8: Enhancement of Action by Stabilization of Intracellular Antibody (Effect of Enhancement of Antibody Action for Recovering Function of Cftr)

The cystic fibrosis transmembrane conductance regulator (CFTR; UniprotKB/Swiss-Prot: P13569.3) is a negative ion channel expressed in epithelial membrane cells of the whole body, and abnormality thereof causes cystic fibrosis. F508 deletion mutation of CFTR (CFTRAF508) is known as the most common mutation of CFTR, in which the 508th phenylalanine is deleted due to deletion of three nucleotides. As a result, CFTRAF508 cannot be normally folded, strongly tends to form an aggregation, and is deemed to move onto the membrane in a smaller amount than the wild type. The scFv-C2 (SEQ ID NO: 1035: EVQLLESGGGLVQPGGSLRLSCAASGFTFSSYAMSWVRQAPGKGLEWVSAISGSGGS TYYADSVKGRFTISRDNSKNTLYLQMNSLRAEDTAVYYCAKMRLGLFDYWGQGTLVT VSSGGGGSGGGGSGGGGEIVLTQSPGTLSLSPGERATLSCRASQSVSSSYLAWYQQK PGQAPRLLIYGASSRATGIPDRFSGSGSGTDFTLTISRLEPEDFAVYYCQQRGDVPP TFGQGTKVEIKAAA) binds to the NBD1 domain of CFTR, and thus can inhibit formation of an aggregation by the NBD1 (Lovato et al., Protein Engineering, Design and Selection, 20(12): 607-614, 2007). The scFv-C2 exhibits an effect of increasing the amount of CFTRAF508 moving onto the membrane. In this example, it was evaluated whether or not aggregation of the mutant AF508 in the NBD1 domain in the HeLa cell can be inhibited by adding a tag (Tag18-1) to the scFv-C2. Plasmid vectors for expressing these proteins were introduced into HeLa cell with Lipo3000 (TM). An antibody was tagged with a myc tag to be detected with a rabbit anti-myc tag antibody, and the NBD1 was tagged with a His tag to be detected with a mouse anti-His tag antibody. The mouse anti-His tag antibody was detected with Alexa 633-labeled anti-mouse IgG antibody, and the rabbit anti-myc tag antibody was detected with Alexa 488-labeled anti-rabbit IgG antibody. Fluorescent stained cells were observed with Keyence BZ-X800.

First, the aggregation rate of the scFv-C2 in HeLa cell was 82%. On the contrary, the aggregation rate of the scFv-C2 having Tag18-1 at the N terminal was 31%, and the aggregation rate of the scFv-C2 having Tag18-1 at the N terminal and the C terminal was 4.6% (see FIG. 3A and FIG. 3B). The aggregation rate of wild type NBD1 domain was 74% in a cell expressing scFv-C2, and the aggregation rate of the AF508 mutant of the NBD1 was 85% in a cell expressing scFv-C2. On the contrary, in a cell expressing scFv-C2 having Tag18-1 at the N terminal and the C terminal, the aggregation rate of the wild type NBD1 domain was 32%, and the aggregation rate of the AF508 mutant of NBD1 was 43%. In this manner, aggregation formation of the scFv-C2 itself could be inhibited by tagging the scFv-C2, and thus, the formation of an aggregation by the NBD1 could be inhibited. The inhibition of the aggregation of the NBD1 is expected to make a contribution to improvement of expression level of the NBD1 in cell membrane.

### Example 9: Effect of Amino Acid Substitution in Existing Tag

Through Examples described above, it was revealed that amino acid substitution for satisfying the condition needed for the peptide tag of the present disclosure, particularly amino acid substitution with P or N increases the aggregation rate reducing action of the tag. In this example, with some amino acids of PEST sequence substituted with N, the aggregation rates of the scFvs (Y14-259) having tags before and after the substitution were examined.

TABLE 15
Comparison of sequence between before
and after substitution

| | |
|---|---|
| PEST(before | YPYDVPDYAGSPQPVEDGEDEF |
| substitution) | CTPMACEANIQSGDSAAPMSAV |
| SEQ ID NO: 1036 | HRHRL |
| PEST(after | NNYDVPDNAGSPQPQEDGEDEF |
| substitution) | NNPQANEANQQSGDSNNPNSAV |
| SEQ ID NO: 1037 | NRHNN |

After these scFvs were expressed in a cell, the aggregation rates of the scFvs were evaluated in the same manner as in Example 1, and the aggregation rate of the scFv having the tag before the substitution was 50.3%, but the aggregation rate of the scFv having the tag after the substitution was 18.0%. In this manner, it was revealed that the amino acid substitution for satisfying the condition needed for the peptide tag of the present disclosure, particularly the amino acid substitution with P or N increases the effect of inhibiting protein aggregation of the tag.

As described so far, various proteins including antibodies form aggregation in a cell, and thus, the functions can be partially or entirely impaired. A protein tag for inhibiting the formation of an aggregation can inhibit the aggregation formation of these proteins, and thus, can cause the proteins to exhibit their actions to be originally exhibited. A tag in which an acidic amino acid ratio is relatively low can be helpful in a scene where a tag having a high acidic amino acid ratio is difficult to use.

1. A peptide, wherein
(a) 5% or more and less than 45% of amino acids contained in an amino acid sequence thereof are acidic amino acids, and
(b) 20% or more of the amino acids contained in the amino acid sequence are amino acids selected from the group consisting of F, P, Y, G, S, Q, N, and A, and
the peptide is capable of reducing an aggregation property in a cell of a protein linked to the peptide.

2. The peptide according to claim 1, wherein
(c) 30% or less of the amino acids contained in the amino acid sequence are amino acids selected from the group consisting of M, T, W, C, I, V, and L.

3. The peptide according to claim 1, wherein
(d) each of A and G constitutes less than 10% of the amino acids contained in the amino acid sequence.

4. The peptide according to claim 1, wherein
(a) 20% or more and less than 45% of the amino acids contained in the amino acid sequence are acidic amino acids,
(b) 30% or more and less than 70% of the amino acids contained in the amino acid sequence are amino acids selected from the group consisting of F, P, Y, G, S, Q, N, and A,
(c) 20% or less of the amino acids contained in the amino acid sequence are amino acids selected from the group consisting of M, T, W, C, I, V, and L, and
(d) each A and G constitutes less than 10% of the amino acids contained in the amino acid sequence.

5. A peptide having an amino acid sequence set forth in any one of SEQ ID NOs: 2 to 11.

6. A nucleic acid encoding the peptide according claim 1.

7. A protein expression vector comprising: the nucleic acid according to claim 6 operably linked to a regulatory sequence; and a nucleic acid encoding a protein of interest in-frame to the nucleic acid according to claim 6.

8. The protein expression vector according to claim 7, wherein the protein of interest is an antibody, or an antigen-binding fragment of an antibody.

9. The protein expression vector according to claim 8, wherein the antigen-binding fragment of the antibody is a single chain Fv (scFv).

10. A fusion protein of the peptide according to claim 1 and a protein of interest.

11. The fusion protein according to claim 10, wherein the protein of interest is an antibody, or an antigen-binding fragment of an antibody.

12. The fusion protein according to claim 11, wherein the antigen-binding fragment of the antibody is a single chain Fv (scFv).

13. A protein-producing cell comprising: the nucleic acid according to claim 6 operably linked to a regulatory sequence; and a nucleic acid encoding a protein of interest in-frame to the nucleic acid according to claim 6.

14. A method for selecting or identifying an amino acid sequence, comprising:
acquiring an amino acid sequence in which:
(a) 5% or more and less than 45% of amino acids contained in the amino acid sequence are acidic amino acids; and
(b) 20% or more of the amino acids contained in the amino acid sequence are amino acids selected from the group consisting of F, P, Y, G, S, Q, N, and A;
selecting or identifying an amino acid sequence of a peptide tag that, when a fusion protein of the peptide tag having the selected or identified amino acid sequence and a reference protein is expressed in a mammal cell, provides reduction of a proportion of cells in which the fusion protein forms an aggregation, or the proportion which is not more than a predetermined value; and
obtaining the peptide tag having the amino acid sequence or a nucleic acid encoding the peptide tag.

15. The method according to claim 14, wherein the amino acid sequence to be acquired a peptide wherein 30% or more of the amino acids contained in the amino acid sequence are amino acids selected from the group consisting of F, P, Y, G, S, Q, N, and A.

16. The method according to claim 14, wherein the amino acid sequence to be acquired is a group of amino acid sequences encoded by coding regions of human genome.

17. The method according to claim 14, wherein the amino acid sequence to be acquired contains a neo-antigen.

18. The peptide of claim 1, wherein 30% or more of the amino acids contained in the amino acid sequence are amino acids selected from the group consisting of F, P, Y, G, S, Q, N, and A.

19. The peptide according to claim 1, wherein 20% or less of the amino acids contained in the amino acid sequence are amino acids selected from the group consisting of M, T, W, C, I, V, and L.

20. The peptide according to claim 1, wherein 15% or less of the amino acids contained in the amino acid sequence are amino acids selected from the group consisting of M, T, W, C, I, V, and L.

21. The peptide according to claim 1, wherein 10% or less of the amino acids contained in the amino acid sequence are amino acids selected from the group consisting of M, T, W, C, I, V, and L.

22. The method of claim 14, wherein the fusion protein of the peptide tag having the selected or identified amino acid sequence and a reference protein is expressed in a human cell.
